(19) United States
(12) Patent Application Publication
(10) Pub. No.: US 2014/0094465 A1
(43) Pub. Date: Apr. 3, 2014

(54) Compounds as S-Nitrosoglutathione Reductase Inhibitors

(71) Applicant: N30 PHARMACEUTICALS, INC., Boulder, CO (US)

(72) Inventors: Xicheng Sun, Broomfield, CO (US); Jian Qiu, Longmont, CO (US); Adam Stout, Denver, CO (US)

(21) Appl. No.: 14/123,220

(22) PCT Filed: Jun. 5, 2012
(86) PCT No.: PCT/US12/40821

§ 371 Date: Dec. 1, 2013

Related U.S. Application Data
(60) Provisional application No. 61/510,722, filed on Jul. 22, 2011.
(60) Provisional application No. 61/495,709, filed on Jun. 10, 2011.

(51) Int. Cl. C07D 417/10; C07D 401/10; C07D 413/10; C07D 409/04; C07D 215/46; C07D 215/60; C07D 239/74; C07D 401/04; C07D 241/42; C07D 403/10; C07D 403/04; C07D 253/10; C07D 217/02; C07C 59/115; C07D 213/55; C07D 241/12; C07D 239/26; C07D 295/155; C07D 237/24; C07D 311/22; C07D 215/20

(52) U.S. Cl. CPC C07D 417/10; C07D 215/20; C07D 401/10; C07D 413/10; C07D 409/04; C07D 215/46; C07D 215/60; C07D 239/74; C07D 401/04; C07D 241/42; C07D 403/10; C07D 403/04; C07D 253/10; C07D 217/02; C07C 59/115; C07D 213/55; C07D 241/12; C07D 239/26; C07D 295/155; C07D 237/24; C07D 311/22
USPC 514/239.5; 514/311; 514/314; 514/313; 514/312; 514/266.1; 514/266.22; 514/249; 514/243; 514/307; 514/569; 514/354; 514/255.06; 514/256; 514/247; 514/456

(57) ABSTRACT

The present invention is directed to compounds useful as S-nitrosoglutathione reductase (GSNOR) inhibitors, pharmaceutical compositions comprising such compounds, and methods of making and using the same.

### FIELD OF THE INVENTION

The present invention is directed to compounds useful as inhibitors of S-nitrosoglutathione reductase (GSNOR), pharmaceutical compositions comprising such compounds, and methods of making and using the same.

### BACKGROUND

The chemical compound nitric oxide is a gas with chemical formula NO. NO is one of the few gaseous signaling molecules known in biological systems, and plays an important role in controlling various biological events. For example, the endothelium uses NO to signal surrounding smooth muscle in the walls of arterioles to relax, resulting in vasodilation and increased blood flow to hypoxic tissues. NO is also involved in regulating smooth muscle proliferation, platelet function, and neurotransmission, and plays a role in host defense. Although NO is highly reactive and has a lifetime of a few seconds, it can both diffuse freely across membranes and bind to many molecular targets. These attributes make NO an ideal signaling molecule capable of controlling biological events between adjacent cells and within cells.

NO is a free radical gas, which makes it reactive and unstable, thus NO is short lived in vivo, having a half life of 3-5 seconds under physiologic conditions. In the presence of oxygen, NO can combine with thiols to generate a biologically important class of stable NO adducts called S-nitrosothiols (SNO's). This stable pool of NO has been postulated to act as a source of bioactive NO and as such appears to be critically important in health and disease, given the centrality of NO in cellular homeostasis (Stamler et al., Proc. Natl. Acad. Sci. USA, 89:7674-7677 (1992)). Protein SNO's play broad roles in the function of cardiovascular, respiratory, metabolic, gastrointestinal, immune, and central nervous system (Foster et al., Trends in Molecular Medicine, 9 (4):160-168, (2003)). One of the most studied SNO's in biological systems is S-nitrosoglutathione (GSNO) (Gaston et al., Proc. Natl. Acad. Sci. USA 90:10957-10961 (1993)), an emerging key regulator in NO signaling since it is an efficient trans-nitrosating agent and appears to maintain an equilibrium with other S-nitrosated proteins (Liu et al., Nature, 410:490-494 (2001)) within cells. Given this pivotal position in the NO—SNO continuum, GSNO provides a therapeutically promising target to consider when NO modulation is pharmacologically warranted.

In light of this understanding of GSNO as a key regulator of NO homeostasis and cellular SNO levels, studies have focused on examining endogenous production of GSNO and SNO proteins, which occurs downstream from the production of the NO radical by the nitric oxide synthetase (NOS) enzymes. More recently there has been an increasing understanding of enzymatic catabolism of GSNO which has an important role in governing available concentrations of GSNO and consequently available NO and SNO's.

Central to this understanding of GSNO catabolism, researchers have recently identified a highly conserved S-nitrosoglutathione reductase (GSNOR) (Jensen et al., Biochem J., 331:659-668 (1998); Liu et al., (2001)). GSNOR is also known as glutathione-dependent formaldehyde dehydrogenase (GSH-FDH), alcohol dehydrogenase 3 (ADH-3) (Uotila and Koivusalo, Coenzymes and Cofactors., D. Dolphin, ed. pp. 517-551 (New York, John Wiley & Sons, (1989)), and alcohol dehydrogenase 5 (ADH-5). Importantly GSNOR shows greater activity toward GSNO than other substrates (Jensen et al., (1998); Liu et al., (2001)) and appears to mediate important protein and peptide denitrosating activity in bacteria, plants, and animals. GSNOR appears to be the major GSNO-metabolizing enzyme in eukaryotes (Liu et al., (2001)). Thus, GSNO can accumulate in biological compartments where GSNOR activity is low or absent (e.g., airway lining fluid) (Gaston et al., (1993)).

Yeast deficient in GSNOR accumulate S-nitrosylated proteins which are not substrates of the enzyme, which is strongly suggestive that GSNO exists in equilibrium with SNO-proteins (Liu et al., (2001)). Precise enzymatic control over ambient levels of GSNO and thus SNO-proteins raises the possibility that GSNO/GSNOR may play roles across a host of physiological and pathological functions including protection against nitrosative stress wherein NO is produced in excess of physiologic needs. Indeed, GSNO specifically has been implicated in physiologic processes ranging from the drive to breathe (Lipton et al., Nature, 413:171-174 (2001)) to regulation of the cystic fibrosis transmembrane regulator (Zaman et al., Biochem Biophys Res Commun, 284:65-70 (2001)), to regulation of vascular tone, thrombosis, and platelet function (de Belder et al., Cardiovasc Res.; 28(5):691-4 (1994)), Z. Kaposzta, et al., Circulation; 106(24): 3057-3062, (2002)) as well as host defense (de Jesus-Berrios et al., Curr. Biol., 13:1963-1968 (2003)). Other studies have found that GSNOR protects yeast cells against nitrosative stress both in vitro (Liu et al., (2001)) and in vivo (de Jesus-Berrios et al., (2003)).

Collectively, data suggest GSNO as a primary physiological ligand for the enzyme S-nitrosoglutathione reductase (GSNOR), which catabolizes GSNO and consequently reduces available SNO's and NO in biological systems (Liu et al., (2001)), (Liu et al., Cell, 116(4), 617-628 (2004)), and (Que et al., Science, 308, (5728):1618-1621 (2005)). As such, this enzyme plays a central role in regulating local and systemic bioactive NO. Since perturbations in NO bioavailability has been linked to the pathogenesis of numerous disease states, including hypertension, atherosclerosis, thrombosis, asthma, gastrointestinal disorders, inflammation, and cancer, agents that regulate GSNOR activity are candidate therapeutic agents for treating diseases associated with NO imbalance.

Nitric oxide (NO), S-nitrosoglutathione (GSNO), and S-nitrosoglutathione reductase (GSNOR) regulate normal lung physiology and contribute to lung pathophysiology. Under normal conditions, NO and GSNO maintain normal lung physiology and function via their anti-inflammatory and bronchodilatory actions. Lowered levels of these mediators in pulmonary diseases such as asthma, chronic obstructive pulmonary disease (COPD) may occur via up-regulation of GSNOR enzyme activity. These lowered levels of NO and GSNO, and thus lowered anti-inflammatory capabilities, are key events that contribute to pulmonary diseases and which can potentially be reversed via GSNOR inhibition.

S-nitrosoglutathione (GSNO) has been shown to promote repair and/or regeneration of mammalian organs, such as the heart (Lima et al., 2010), blood vessels (Lima et al., 2010) skin (Georgii et al., 2010), eye or ocular structures (Haq et al., 2007) and liver (Prince et al., 2010;). S-nitrosoglutathione reductase (GSNOR) is the major catabolic enzyme of GSNO. Inhibition of GSNOR is thought to increase endogenous GSNO.

Inflammatory bowel diseases (IBD's), including Crohn's and ulcerative colitis, are chronic inflammatory disorders of the gastrointestinal (GI) tract, in which NO, GSNO, and GSNOR can exert influences. Under normal conditions, NO and GSNO function to maintain normal intestinal physiology via anti-inflammatory actions and maintenance of the intestinal epithelial cell barrier. In IBD, reduced levels of GSNO and NO are evident and likely occur via up-regulation of GSNOR activity. The lowered levels of these mediators contribute to the pathophysiology of IBD via disruption of the epithelial barrier via dysregulation of proteins involved in maintaining epithelial tight junctions. This epithelial barrier dysfunction, with the ensuing entry of micro-organisms from the lumen, and the overall lowered anti-inflammatory capabilities in the presence of lowered NO and GSNO, are key events in IBD progression that can be potentially influenced by targeting GSNOR.

Cystic fibrosis (CF) is one of the most common lethal genetic diseases in Caucasians. Approximately one in 3,500 children in the US is born with CF each year. It is a disease that affects all racial and ethnic groups, but is more common among Caucasians. An estimated 30,000 American adults and children have CF, and the median predicted age of survival is 37.4 years (CFF Registry Report 2007, Cystic Fibrosis Foundation, Bethesda, Md.). CF is an autosomal recessive hereditary disease caused by a mutation in the gene for the cystic fibrosis transmembrane regulator (CFTR) protein. More than 1,000 disease-associated mutations have been discovered in the CFTR gene with the most common mutation being a deletion of the amino acid phenylalanine at position 508 (F508del). The CFTR protein is located on the apical membrane and is responsible for chloride transport across epithelial cells on mucosal surfaces. GSNO has been identified as a positive modulator of CFTR. As GSNOR is the primary catabolizing enzyme of GSNO, it is hypothesized that inhibition of GSNOR may improve F508del-CFTR function via nitrosation of chaperone proteins, prevention of CFTR proteosomal degradation, promotion of CFTR maturation, and maintenance of epithelial tight junctions. Currently there is no curative treatment for CF; therefore, new therapies are needed for the disease.

Cell death is the crucial event leading to clinical manifestation of hepatotoxicity from drugs, viruses and alcohol. Glutathione (GSH) is the most abundant redox molecule in cells and thus the most important determinant of cellular redox status. Thiols in proteins undergo a wide range of reversible redox modifications during times of exposure to reactive oxygen and reactive nitrogen species, which can affect protein activity. The maintenance of hepatic GSH is a dynamic process achieved by a balance between rates of GSH synthesis, GSH and GSSG efflux, GSH reactions with reactive oxygen species and reactive nitrogen species and utilization by GSH peroxidase. Both GSNO and GSNOR play roles in the regulation of protein redox status by GSH.

Acetaminophen overdoses are the leading cause of acute liver failure (ALF) in the United States, Great Britain and most of Europe. More than 100,000 calls to the U.S. Poison Control Centers, 56,000 emergency room visits, 2600 hospitalizations, nearly 500 deaths are attributed to acetaminophen in this country annually. Approximately, 60% recover without the needing a liver transplant, 9% are transplanted and 30% of patients succumb to the illness. The acetaminophen-related death rate exceeds by at least three-fold the number of deaths due to all other idiosyncratic drug reactions combined (Lee, Hepatol Res 2008; 38 (Suppl. 1):S3-S8).

Nonalcoholic steatohepatitis (NASH) effecting 7-9% of Americans is caused by fat in the liver, along with inflammation and damage. Most people with NASH feel well and are not aware that they have a liver problem. NASH can be severe and as the disease progresses it can lead to cirrhosis, in which the liver is permanently damaged and scarred and no longer able to work properly. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

Liver transplantation has become the primary treatment for patients with fulminant hepatic failure and end-stage chronic liver disease, as well as certain metabolic liver diseases. Thus, the demand for transplantation now greatly exceeds the availability of donor organs. It has been estimated that more than 18,000 patients are currently registered with the United Network for Organ Sharing (UNOS) and that an additional 9,000 patients are added to the liver transplant waiting list each year, yet less than 5,000 cadaveric donors are available for transplantation.

Currently, there is a great need in the art for diagnostics, prophylaxis, ameliorations, and treatments for medical conditions relating to increased NO synthesis and/or increased NO bioactivity. In addition, there is a significant need for novel compounds, compositions, and methods for preventing, ameliorating, or reversing other NO-associated disorders. The present invention satisfies these needs.

### SUMMARY

The present invention provides compounds that are useful as S-nitrosoglutathione reductase (“GSNOR”) inhibitors. The invention encompasses pharmaceutically acceptable salts, stereoisomers, prodrugs, metabolites, and N-oxides of the described compounds. Also encompassed by the invention are pharmaceutical compositions comprising at least one compound of the invention and at least one pharmaceutically acceptable carrier.

The present invention also includes novel compounds described herein.

The compositions of the present invention can be prepared in any suitable pharmaceutically acceptable dosage form.

The present invention provides a method for inhibiting GSNOR in a subject in need thereof. Such a method comprises administering a therapeutically effective amount of a pharmaceutical composition comprising at least one GSNOR inhibitor or a pharmaceutically acceptable salt, stereoisomer, prodrug, metabolite or N-oxide thereof, in combination with at least one pharmaceutically acceptable carrier. The GSNOR inhibitor can be a novel compound according to the invention, or it can be a known compound which previously was not known to be an inhibitor of GSNOR.

The present invention also provides a method of treating a disorder ameliorated by NO donor therapy in a subject in need thereof. Such a method comprises administering a therapeutically effective amount of a pharmaceutical composition comprising at least one GSNOR inhibitor or a pharmaceutically acceptable salt, stereoisomer, prodrug, metabolite, or N-oxide thereof, in combination with at least one pharmaceutically acceptable carrier. The GSNOR inhibitor can be a novel compound according to the invention, or it can be a known compound which previously was not known to be an inhibitor of GSNOR.

The present invention also provides a method of treating a cell proliferative disorder in a subject in need thereof. Such a method comprises administering a therapeutically effective amount of a pharmaceutical composition comprising at least one GSNOR inhibitor or a pharmaceutically acceptable salt, stereoisomer, prodrug, metabolite, or N-oxide thereof, in combination with at least one pharmaceutically acceptable carrier. The GSNOR inhibitor can be a novel compound according to the invention, or it can be a known compound which previously was not known to be an inhibitor of GSNOR.

The methods of the invention encompass administration with one or more secondary active agents. Such administration can be sequential or in a combination composition.

Although methods and materials similar or equivalent to those described herein can be used in the practice or testing of the present invention, suitable methods and materials are described below. All publicly available publications, patent applications, patents, and other references mentioned herein are incorporated by reference in their entirety. In the case of conflict, the present specification, including definitions, will control.

Both the foregoing summary and the following detailed description are exemplary and explanatory and are intended to provide further details of the compositions and methods as claimed. Other objects, advantages, and novel features will be readily apparent to those skilled in the art from the following detailed description.

### DETAILED DESCRIPTION

### A. Overview of the Invention

Until recently, S-nitrosoglutathione reductase (GSNOR) was known to oxidize the formaldehyde glutathione adduct, S-hydroxymethylglutathione. GSNOR has since been identified in a variety of bacteria, yeasts, plants, and animals and is well conserved. The proteins from E. coli, S. cerevisiae and mouse macrophages share over 60% amino acid sequence identity. GSNOR activity (i.e., decomposition of GSNO when NADH is present as a required cofactor) has been detected in E. coli, in mouse macrophages, in mouse endothelial cells, in mouse smooth muscle cells, in yeasts, and in human HeLa, epithelial, and monocyte cells. Human GSNOR nucleotide and amino acid sequence information can be obtained from the National Center for Biotechnology Information (NCBI) databases under Accession Nos. M29872, NM_{—}000671. Mouse GSNOR nucleotide and amino acid sequence information can be obtained from NCBI databases under Accession Nos. NM_{—}007410. In the nucleotide sequence, the start site and stop site are underlined. CDS designates coding sequence. SNP designates single nucleotide polymorphism. Other related GSNOR nucleotide and amino acid sequences, including those of other species, can be found in U.S. Patent Application 2005/0014697.

In accord with the present invention, GSNOR has been shown to function in vivo and in vitro to metabolize S-nitrosoglutathione (GSNO) and protein S-nitrosothiols (SNOs) to modulate NO bioactivity, by controlling the intracellular levels of low mass NO donor compounds and preventing protein nitrosylation from reaching toxic levels.

Based on this, it follows that inhibition of this enzyme potentiates bioactivity in diseases in which NO donor therapy is indicated, inhibits the proliferation of pathologically proliferating cells, and increases NO bioactivity in diseases where this is beneficial.

The present invention provides pharmaceutical agents that are potent inhibitors of GSNOR. In particular, provided are substituted and unsubstituted multi-cyclic analogs possessing two key pharmacophores, a hydroxyl group on one of the cycles and an acidic moiety on another cycle. These two cyclic groups are joined by a linker such that the distance between the two pharmacophores is appropriate for binding within the GSNOR active site. In particular, provided are compounds having the structure depicted below (Formula I), or a pharmaceutically acceptable salt, stereoisomer, prodrug, metabolite, or N-oxide thereof.

HO-Cy₁-linker-Cy₂-acidic moiety Formula 1

wherein
Cy₁ is selected from the group consisting of substituted and unsubstituted monocyclic aryl, substituted and unsubstituted bicyclic aryl, substituted and unsubstituted monocyclic heterocycle, substituted and unsubstituted bicyclic heterocycle, substituted and unsubstituted monocyclic heteroaryl, substituted and unsubstituted bicyclic heteroaryl, substituted and unsubstituted monocyclic saturated cycloalkyl, and substituted and unsubstituted bicyclic cycloalkyl;
linker is selected from the group consisting of a direct bond, O, S, SO, SO₂, C═O, CR₅R₆, NR₇, substituted and unsubstituted (C₂-C₃) alkyl, substituted and unsubstituted (C₂-C₃) heteroalkyl, substituted and unsubstituted (C₂-C₃) alkene, substituted and unsubstituted 5 or 6 membered aryl, substituted and unsubstituted 5 or 6 membered heteroaryl, substituted and unsubstituted 3-6 membered cycloalkyl, and substituted and unsubstituted 3-6 membered saturated heterocyclyl; wherein R₅ and R₆ are independently selected from the group consisting of hydrogen, (C₁-C₆) alkyl, (C₁-C₆) heteroalkyl, halogen, (C₁-C₆) haloalkyl, cyano, and hydroxyl; R₇ is selected from the group consisting of hydrogen, (C₁-C₆)alkyl, (C₁-C₆) haloalkyl, and (C₁-C₆) heteroalkyl; substitutions for the (C₂-C₃) alkyl, (C₂-C₃) heteroalkyl, and (C₂-C₃) alkene are selected from the group consisting of ═O, halogen, (C₁-C₃) alkyl, (C₁-C₃) haloalkyl, (C₁-C₃) heteroalkyl, cyano, and hydroxyl, and wherein when the heteroalkyl group contains nitrogen or sulfur, the N and S atoms may be optionally oxidized; substitutions for aryl, heteroaryl, cycloalkyl and saturated heterocyclyl are selected from the group consisting of hydrogen, (C₁-C₈)alkyl, (C₁-C₈) haloalkyl, and (C₁-C₈)heteroalkyl; and
Cy₂ is selected from the group consisting of substituted and unsubstituted monocyclic aryl, substituted and unsubstituted monocyclic saturated heterocycle, substituted and unsubstituted monocyclic heteroaryl, and substituted and unsubstituted monocyclic cycloalkyl.

As used in this context, the term “analog” refers to a compound having similar chemical structure and function as compounds of Formula I.

Some analogs of the invention can also exist in various isomeric forms, including configurational, geometric, and conformational isomers, as well as existing in various tautomeric forms, particularly those that differ in the point of attachment of a hydrogen atom. As used herein, the term “isomer” is intended to encompass all isomeric forms of a compound including tautomeric forms of the compound.

Illustrative compounds having asymmetric centers can exist in different enantiomeric and diastereomeric forms. A compound can exist in the form of an optical isomer or a diastereomer. Accordingly, the invention encompasses compounds in the forms of their optical isomers, diastereomers and mixtures thereof, including racemic mixtures.

It should be noted that if there is a discrepancy between a depicted structure and a name given to that structure, the depicted structure controls. In addition, if the stereochemistry of a structure or a portion of a structure is not indicated with, for example, bold, wedged, or dashed lines, the structure or portion of the structure is to be interpreted as encompassing all stereoisomers of the described compound.

### B. S-Nitrosoglutathione Reductase Inhibitors

1. Inventive Compounds

In one of its aspects the present invention provides compounds having the structure shown in Formula I, or a pharmaceutically acceptable salt, stereoisomer, prodrug, metabolite, or N-oxide thereof:

HO-Cy₁-linker-Cy₂-acidic moiety Formula 1

wherein
Cy₁ is selected from the group consisting of substituted and unsubstituted monocyclic aryl, substituted and unsubstituted bicyclic aryl, substituted and unsubstituted monocyclic heterocycle, substituted and unsubstituted bicyclic heterocycle, substituted and unsubstituted monocyclic heteroaryl, substituted and unsubstituted bicyclic heteroaryl, substituted and unsubstituted monocyclic cycloalkyl, and substituted and unsubstituted bicyclic cycloalkyl;
linker is selected from the group consisting of a direct bond, O, S, SO, SO₂, C═O, CR₅R₆, NR₇, substituted and unsubstituted (C₂-C₃) alkyl, substituted and unsubstituted (C₂-C₃) heteroalkyl, substituted and unsubstituted (C₂-C₃) alkene, substituted and unsubstituted 5 or 6 membered aryl, substituted and unsubstituted 5 or 6 membered heteroaryl, substituted and unsubstituted 3-6 membered cycloalkyl, and substituted and unsubstituted 3-6 membered saturated heterocyclyl; wherein R₅ and R₆ are independently selected from the group consisting of hydrogen, (C₁-C₆) alkyl, (C₁-C₆) heteroalkyl, halogen, (C₁-C₆) haloalkyl, cyano, and hydroxyl; R₇ is selected from the group consisting of hydrogen, (C₁-C₆)alkyl, (C₁-C₆) haloalkyl, and (C₁-C₆) heteroalkyl; substitutions for the (C₂-C₃) alkyl, (C₂-C₃) heteroalkyl, and (C₂-C₃) alkene are selected from the group consisting of ═O, halogen, (C₁-C₃) alkyl, (C₁-C₃) haloalkyl, (C₁-C₃) heteroalkyl, cyano, and hydroxyl, and wherein when the heteroalkyl group contains nitrogen or sulfur, the N and S atoms may be optionally oxidized; substitutions for aryl, heteroaryl, cycloalkyl and saturated heterocyclyl are selected from the group consisting of hydrogen, (C₁-C₈)alkyl, (C₁-C₈) haloalkyl, and (C₁-C₈)heteroalkyl; and
Cy₂ is selected from the group consisting of substituted and unsubstituted monocyclic aryl, substituted and unsubstituted monocyclic saturated heterocycle, substituted and unsubstituted monocyclic heteroaryl, and substituted and unsubstituted monocyclic cycloalkyl.

In a further aspect of the invention, linker is selected from the group consisting of a direct bond, O, S, SO, SO₂, C═O, CH₂, NH, NMe, substituted and unsubstituted (C₂-C₃) alkyl, substituted and unsubstituted (C₂-C₃) heteroalkyl, a 5 or 6 membered aryl, and a 5 or 6 membered heteroaryl group; wherein substitutions for the (C₂-C₃) alkyl and (C₂-C₃) heteroalkyl are selected from the group consisting of ═O, halogen, (C₁-C₃) alkyl, (C₁-C₃) haloalkyl, and hydroxyl, and wherein if the heteroalkyl group contains nitrogen or sulfur, they may be optionally oxidized.

In another aspect of the invention, Cy₂-Acidic moiety is selected from the group consisting of

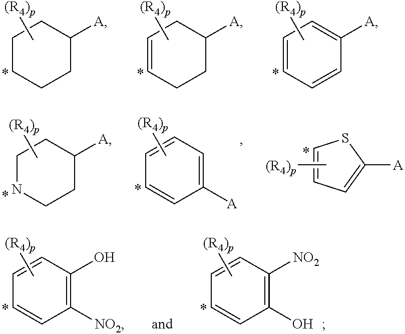

wherein
* represents the position on Cy₂ that is connected to the linker;
A is an acidic moiety, and is selected from the group consisting of

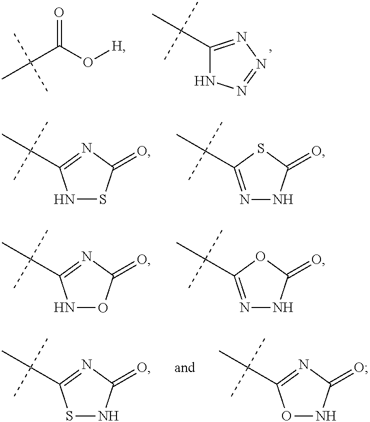

R₄ is selected from the group consisting of halogen, (C₁-C₆) alkyl, (C₁-C₆) haloalkyl, (C₁-C₆) alkoxy, cyano, and NR₈R_{8′} where R₈ and R_{8′} are independently selected from the group consisting of (C₁-C₃) alkyl, or R₈ when taken together with R_{8′} form a ring with 3 to 6 members; and p is selected from the group consisting of 0, 1, 2, 3, and 4.

In another aspect of the invention, Cy₂-Acidic moiety is selected from the group consisting of

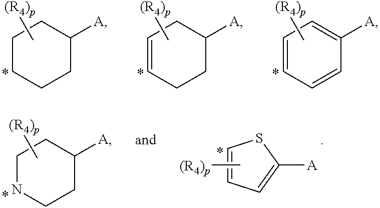

In another aspect of the invention, linker is selected from the group consisting of a direct bond, O, S, SO, SO₂, C═O, CR₅R₆, NR₇, wherein R₅ and R₆ are independently selected from the group consisting of hydrogen, (C₁-C₆) alkyl, (C₁-C₆) heteroalkyl, halogen, (C₁-C₆) haloalkyl, cyano, and hydroxyl; R₇ is selected from the group consisting of hydrogen, (C₁-C₆)alkyl, (C₁-C₆) haloalkyl, and (C₁-C₆) heteroalkyl; and Cy₁ is selected from the group consisting of substituted and unsubstituted bicyclic aryl, substituted and unsubstituted bicyclic heterocycle, substituted and unsubstituted bicyclic heteroaryl, and substituted and unsubstituted bicyclic cycloalkyl.

In a further aspect of the invention, HO-Cy₁ is selected from the group consisting of

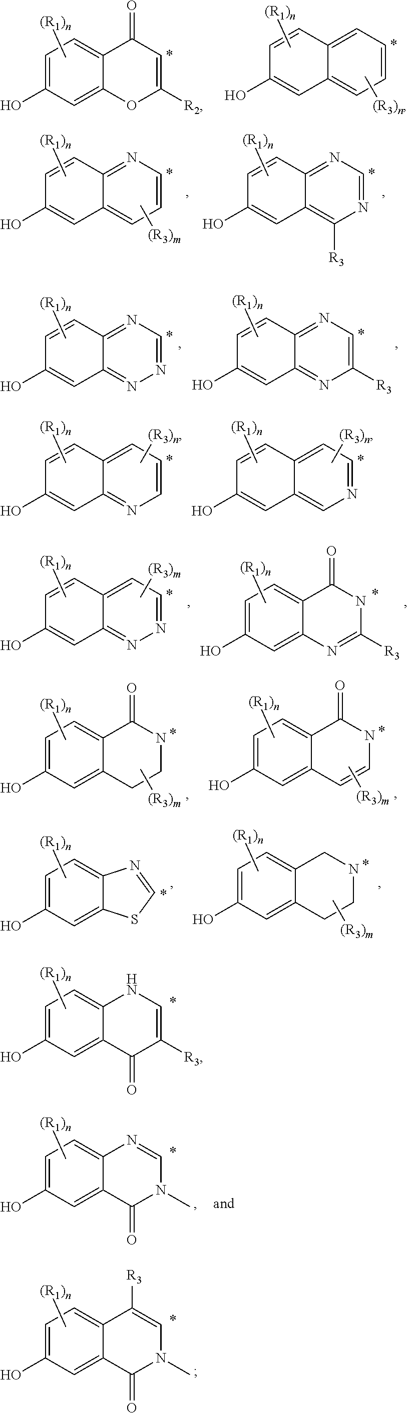

### Wherein

* represents the position on Cy₁ that is connected to the linker;
R₁ is selected from the group consisting of halogen, methoxy, and cyano;
R₂ is selected from the group consisting of hydrogen, (C₁-C₆)alkyl, (C₃-C₇)cycloalkyl, (C₁-C₆)haloalkyl, unsubstituted aryl(C₁-C₄)alkyl, substituted aryl(C₁-C₆)alkyl, (C₁-C₆)heteroalkyl, substituted or unsubstituted aryl, and substituted or unsubstituted heteroaryl;
R₃ is selected from the group consisting of hydrogen, halogen, (C₁-C₃) alkyl, fluorinated (C₁-C₃) alkyl, cyano, C₁-C₃ alkoxy, SMe, and N(CH₃)₂;
n is selected from the group consisting of 0, 1, 2, and 3; and
m is selected from the group consisting of 0, 1, and 2.

In a further aspect of the invention, HO-Cy₁ is selected from the group consisting of

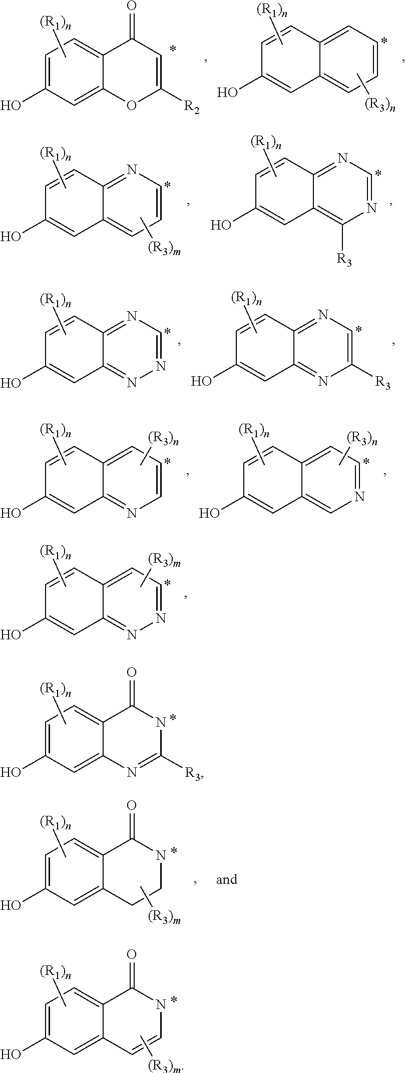

Wherein *, R₁, R₂, R₃, m, and n are as previously defined.

In a further aspect of the invention, linker is selected from the group consisting of substituted and unsubstituted (C₂-C₃) alkyl, substituted and unsubstituted (C₂-C₃) heteroalkyl, and substituted and unsubstituted (C₂-C₃) alkene, wherein substitutions for (C₂-C₃) alkyl, (C₂-C₃) heteroalkyl, and (C₂-C₃) alkene are selected from the group consisting of ═O, halogen, (C₁-C₃) alkyl, (C₁-C₃) haloalkyl, (C₁-C₃) heteroalkyl, cyano, and hydroxyl, and wherein when the heteroalkyl group contains nitrogen or sulfur, the N and S atoms may be optionally oxidized; and Cy₁ is selected from the group consisting of substituted and unsubstituted monocyclic aryl, substituted and unsubstituted monocyclic saturated heterocycle, substituted and unsubstituted monocyclic heteroaryl, and substituted and unsubstituted monocyclic cycloalkyl.

In a further aspect of the invention, linker is selected from the group consisting of substituted and unsubstituted C₂ alkyl, and substituted and unsubstituted C₂ heteroalkyl; wherein heteroalkyl consists of one heteroatom selected from the group consisting of O, S, SO, SO₂, NH, NMe, and wherein suitable alkyl substitutions are selected from the group consisting of ═O, R₅ and R₆ (previously defined).

In yet another aspect of the invention, the GSNOR inhibitor of Formula 1 has the structure shown in Formula 2

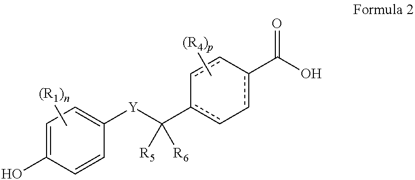

wherein Y is selected from the group consisting of CH₂, O, S, SO, SO₂, NH, NR₇; wherein R₁, R₄, R₅, R₆, R₇, are as defined previously; and --- indicates the bond can be saturated or unsaturated.

In yet another aspect of the invention, the GSNOR inhibitor of Formula 1 has the structure shown in Formula 3

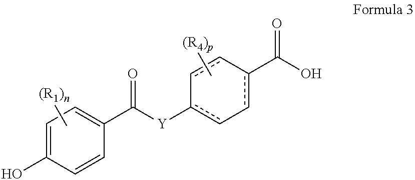

wherein Y is selected from the group consisting of CH₂ and NH; wherein R₁, R₄, R₅, R₆, R₇, are as defined previously; and --- indicates the bond can be saturated or unsaturated.

In a yet another aspect of the invention, linker is selected from the group consisting of substituted and unsubstituted 5 or 6 membered aryl, substituted and unsubstituted 5 or 6 membered heteroaryl; substitutions for aryl and heteroaryl are selected from the group consisting of hydrogen, (C₁-C₈)alkyl, (C₁-C₈) haloalkyl, and (C₁-C₈)heteroalkyl; and Cy₁ is selected from the group consisting of a substituted or unsubstituted monocyclic aryl group, and a substituted or unsubstituted monocyclic heteroaryl group.

In yet another aspect of the invention, the GSNOR inhibitor of Formula 1 has the structure shown in Formula 4

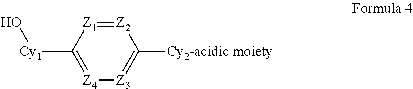

wherein Z₁, Z₂, Z₃, and Z₄ are independently selected from the group consisting of CH and N, and Cy₁ and Cy₂ are as previously defined.

In yet another aspect of the invention, the GSNOR inhibitor of Formula 1 has the structure shown in Formula 5

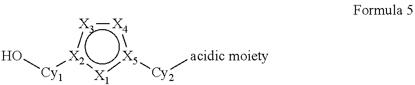

wherein
X₁, X₃, and X₄ are independently selected from the group consisting of N, NR₉, CR₁₀, S, and O;
X₂ and X₅ are independently selected from the group consisting of C, CH, and N;
R₉ and R₁₀ are independently selected from the group consisting of hydrogen, (C₁-C₈)alkyl, (C₁-C₈) haloalkyl, and (C₁-C₈)heteroalkyl;
and Cy₁, Cy₂ and acidic moiety are as previously defined.

In yet another aspect of the invention, the GSNOR inhibitor of Formula 1 has the structure shown in Formula 6

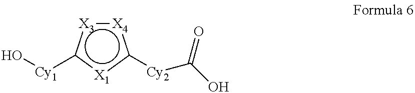

wherein X₁, X₃, and X₄ are independently selected from the group consisting of N, NR₉, CR₁₀, S, and O;
and Cy₁, Cy₂, R₉, and R₁₀ and are as previously defined.

In yet another aspect of the invention, the GSNOR inhibitor of Formula 1 has the structure shown in Formula 7

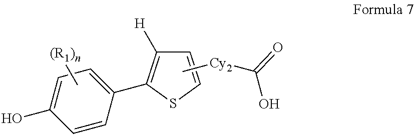

wherein R₁ is selected from the group consisting of F, Cl, Br, OMe, and CN; n is selected from the group consisting of 0 and 1; Cy₂-COOH is selected from the group consisting of

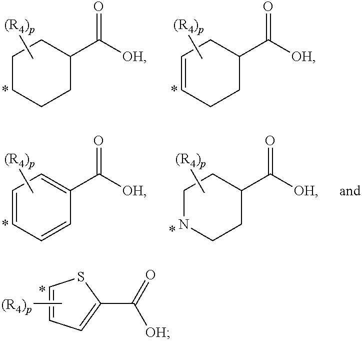

R₄ is selected from the group consisting of F, Cl, Br, CN, Me, OMe, N(Me)₂; p is selected from the group consisting of 0, 1, and 2; and * represents the position on Cy₂ that is connected to the compound of Formula 7.

In yet another aspect of the invention, Cy₂-COOH of the compound of Formula 7 is selected from the group consisting of

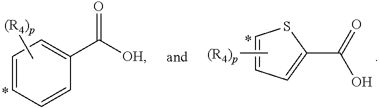

In yet another aspect of the invention, the compound of Formula 7 is selected from the group consisting of
4-(5-(4-hydroxyphenyl)thiophen-2-yl)benzoic acid;
4-(5-(3-fluoro-4-hydroxyphenyl)thiophen-2-yl)benzoic acid;
4-(5-(2-fluoro-4-hydroxyphenyl)thiophen-2-yl)benzoic acid;
5′-(4-hydroxyphenyl)-[2,2′-bithiophene]-5-carboxylic acid;
5′-(3-fluoro-4-hydroxyphenyl)-[2,2′-bithiophene]-5-carboxylic acid; and
5′-(4-hydroxyphenyl)-[2,3′-bithiophene]-5-carboxylic acid.

In yet another aspect of the invention, the GSNOR inhibitor of Formula 1 has the structure shown in Formula 8

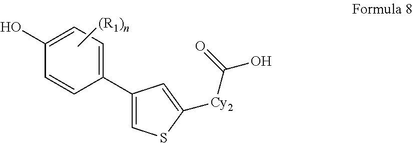

Wherein R₁ is selected from the group consisting of F, Cl, Br, OMe, and CN; n is selected from the group consisting of 0 and 1; Cy₂-COOH is selected from the group consisting of

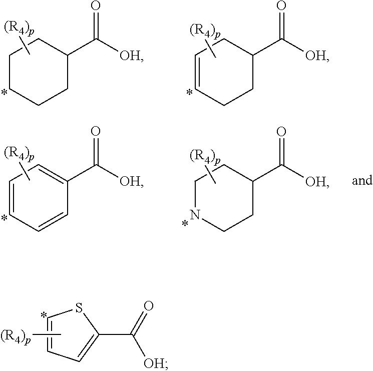

R₄ is selected from the group consisting of F, Cl, Br, CN, Me, OMe, N(Me)₂; p is selected from the group consisting of 0, 1, and 2, and * represents the position on Cy₂ that is connected to the compound of Formula 8.

In yet another aspect of the invention, Cy₂-COOH of the compound of Formula 8 is selected from the group consisting of

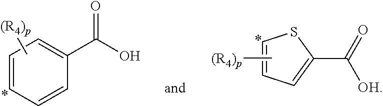

In yet another aspect of the invention, the compound of Formula 8 is 4′-(4-hydroxyphenyl)-[2,2′-bithiophene]-5-carboxylic acid.

In yet another aspect of the invention, the GSNOR inhibitor of Formula 1 has the structure shown in Formula 9

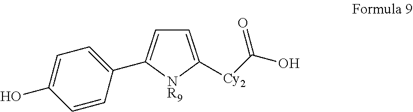

Wherein R₁ is selected from the group consisting of F, Cl, Br, OMe, and CN; n is selected from the group consisting of 0 and 1; Cy₂-COOH is selected from the group consisting of

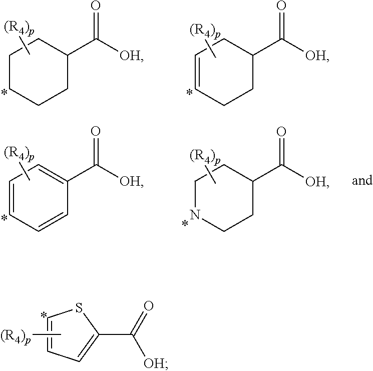

R₄ is selected from the group consisting of F, Cl, Br, CN, Me, OMe, N(Me)₂; p is selected from the group consisting of 0, 1, and 2; * represents the position on Cy₂ that is connected to the compound of formula 9; and R₉ is selected from the group consisting of hydrogen, (C₁-C₈)alkyl, (C₁-C₈) haloalkyl, and (C₁-C₈) heteroalkyl.

In yet another aspect of the invention, Cy₂-COOH of the compound of Formula 9 is selected from the group consisting of

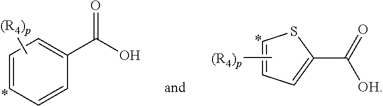

In yet another aspect of the invention, the compound of Formula 9 is selected from the group consisting of
4-(5-(4-hydroxyphenyl)-1H-pyrrol-2-yl)benzoic acid;
5-(5-(4-hydroxyphenyl)-1-methyl-1H-pyrrol-2-yl)thiophene-2-carboxylic acid;
5-(5-(4-hydroxyphenyl)-1-(2-methoxyethyl)-1H-pyrrol-2-yl)thiophene-2-carboxylic acid;
5-(1-butyl-5-(4-hydroxyphenyl)-1H-pyrrol-2-yl)thiophene-2-carboxylic acid;
5-(5-(4-hydroxyphenyl)-1-isopentyl-1H-pyrrol-2-yl)thiophene-2-carboxylic acid;
4-(5-(4-hydroxyphenyl)-1-isopentyl-1H-pyrrol-2-yl)benzoic acid;
4-(5-(4-hydroxyphenyl)-1-isobutyl-1H-pyrrol-2-yl)benzoic acid;
4-(5-(4-hydroxyphenyl)-1-(4-methylpentyl)-1H-pyrrol-2-yl)benzoic acid;
4-(5-(4-hydroxyphenyl)-1-(3-isopropoxypropyl)-1H-pyrrol-2-yl)benzoic acid;
4-(5-(4-hydroxyphenyl)-1-isopropyl-1H-pyrrol-2-yl)benzoic acid;
4-(5-(4-hydroxyphenyl)-1-(2-methoxyethyl)-1H-pyrrol-2-yl)benzoic acid;
4-(1-hexyl-5-(4-hydroxyphenyl)-1H-pyrrol-2-yl)benzoic acid;
4-(1-(2-ethoxyethyl)-5-(4-hydroxyphenyl)-1H-pyrrol-2-yl)benzoic acid;
4-(1-(3-ethoxypropyl)-5-(4-hydroxyphenyl)-1H-pyrrol-2-yl)benzoic acid; and
4-(5-(4-hydroxyphenyl)-1-propyl-1H-pyrrol-2-yl)benzoic acid.

In yet another aspect of the invention, the GSNOR inhibitor of Formula 1 has the structure shown in Formula 10

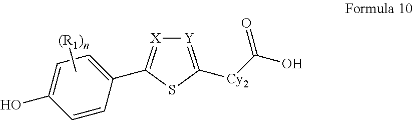

Wherein X is selected from CH and N, Y is selected from CH and N, and wherein when X is CH, then Y is N, and when X is N, then Y is CH; R₁ is selected from the group consisting of F, Cl, Br, OMe, and CN; n is selected from the group consisting of 0 and 1; Cy₂-COOH is selected from the group consisting of

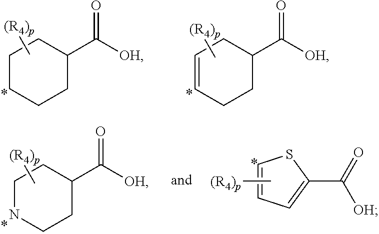

R₄ is selected from the group consisting of F, Cl, Br, CN, Me, OMe, N(Me)₂; p is selected from the group consisting of 0, 1, and 2; * represents the position on Cy₂ that is connected to the compound of formula 10.

In yet another aspect of the invention, Cy₂-COOH of the compound of Formula 10 is

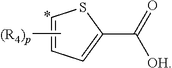

In yet another aspect of the invention, the compound of Formula 10 is selected from the group consisting of
5-(2-(3-fluoro-4-hydroxyphenyl)thiazol-5-yl)thiophene-2-carboxylic acid;
5-(5-(4-hydroxyphenyl)thiazol-2-yl)thiophene-2-carboxylic acid; and
5-(2-(4-hydroxyphenyl)thiazol-5-yl)thiophene-2-carboxylic acid.
In yet another aspect of the invention, the GSNOR inhibitor of Formula 1 has the structure shown in Formula 11

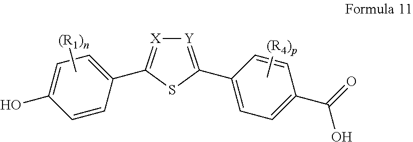

wherein X is selected from CH and N, Y is selected from CH and N, and wherein when X is CH, Y is N, and when X is N, then Y is CH; R₁ is selected from the group consisting of F, Cl, Br, OMe, and CN; n is selected from the group consisting of 0 and 1; R₄ is selected from the group consisting of F, Cl, Br, CN, Me, OMe, N(Me)₂; p is selected from the group consisting of 0, 1, and 2; and n+p is greater than 0.

In yet another aspect of the invention, the GSNOR inhibitor of Formula 1 has the structure shown in Formula 12

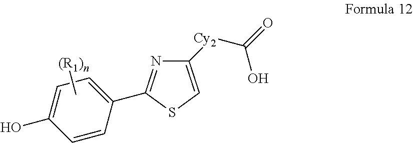

wherein R₁ is selected from the group consisting of F, Cl, Br, OMe, and CN; n is selected from the group consisting of 0 and 1; Cy₂-COOH is selected from the group consisting of

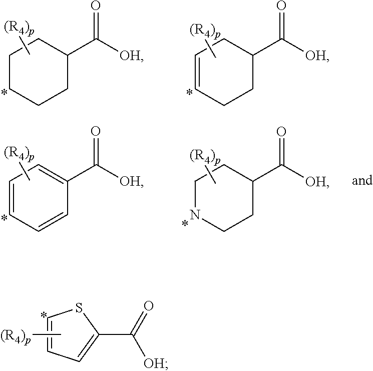

R₄ is selected from the group consisting of F, Cl, Br, H, CN, Me, OMe, N(Me)₂; p is selected from the group consisting of 0, 1, and 2; * represents the position on Cy₂ that is connected to the compound of formula 12.

In yet another aspect of the invention, Cy₂-COOH of the compound of Formula 12 is selected from the group consisting of

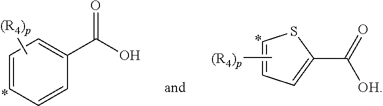

In yet another aspect of the invention, the compound of Formula 12 is selected from the group consisting of
4-(2-(4-hydroxyphenyl)thiazol-4-yl)benzoic acid;
5-(2-(3-fluoro-4-hydroxyphenyl)thiazol-4-yl)thiophene-2-carboxylic acid;
4-(2-(3-fluoro-4-hydroxyphenyl)thiazol-4-yl)benzoic acid; and
5-(2-(4-hydroxyphenyl)thiazol-4-yl)thiophene-2-carboxylic acid.

In yet another aspect of the invention, the GSNOR inhibitor of Formula 1 has the structure shown in Formula 13

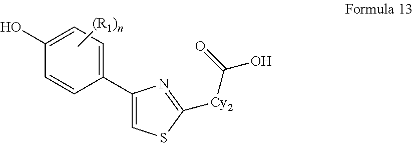

wherein R₁ is selected from the group consisting of F, Cl, Br, OMe, and CN; n is selected from the group consisting of 0 and 1; Cy₂-COOH is selected from the group consisting of

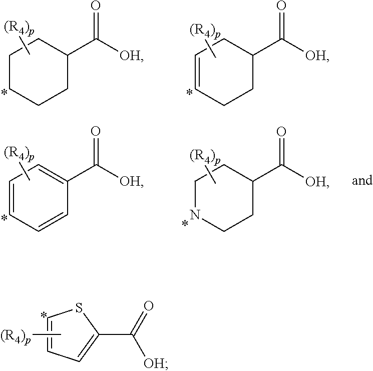

R₄ is selected from the group consisting of F, Cl, Br, CN, Me, OMe, N(Me)₂; p is selected from the group consisting of 0, 1, and 2; * represents the position on Cy₂ that is connected to the compound of formula 13.

In yet another aspect of the invention, Cy₂-COOH of the compound of Formula 13 is selected from the group consisting of

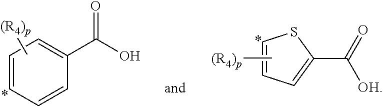

In yet another aspect of the invention, the compound of Formula 13 is 4-(4-(4-hydroxyphenyl)thiazol-2-yl)benzoic acid.

In yet another aspect of the invention, the GSNOR inhibitor of Formula 1 has the structure shown in Formula 14

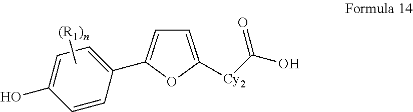

wherein R₁ is selected from the group consisting of F, Cl, Br, OMe, and CN; n is selected from the group consisting of 0 and 1; Cy₂-COOH is selected from the group consisting of

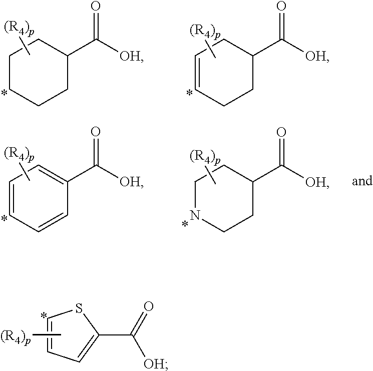

R₄ is selected from the group consisting of F, Cl, Br, CN, Me, OMe, N(Me)₂; p is selected from the group consisting of 0, 1, and 2; * represents the position on Cy₂ that is connected to the compound of formula 14.

In yet another aspect of the invention, Cy₂-COOH of the compound of Formula 14 is selected from the group consisting of

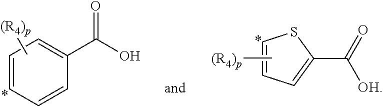

In yet another aspect of the invention, the compound of Formula 14 is 4-(5-(4-hydroxyphenyl)furan-2-yl)benzoic acid.

In a further aspect of the invention, HO-Cy₁ of the GSNOR inhibitor of Formula 1 is

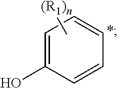

wherein * represents the position on Cy₁ that is connected to the linker; R₁ is selected from the group consisting of halogen, methoxy, and cyano; and n is selected from the group consisting of 0, 1, 2, and 3.

In yet another aspect of the invention, R₁ is F.

In yet another aspect of the invention, the acidic moiety of a GSNOR inhibitor of Formula 1 is a carboxylic acid.

In yet another aspect of the invention, Cy₂-COOH of Formula 1 is selected from the group consisting of

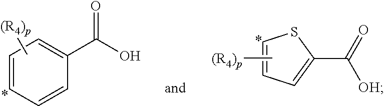

wherein R₄ is selected from the group consisting of F, Cl, Br, CN, Me, OMe, N(Me)₂; and p is selected from the group consisting of 0, 1, and 2.

In yet another aspect of the invention, R₄ is selected from the group consisting of F, Cl, and Me.

In yet another aspect of the invention, the compound of Formula 1 is selected from the group consisting of
3-fluoro-4-(5-fluoro-6-hydroxyquinolin-2-yl)benzoic acid;
3-chloro-4-(5-fluoro-6-hydroxyquinolin-2-yl)benzoic acid;
4-(5-fluoro-6-hydroxyquinolin-2-yl)-3-methylbenzoic acid;
4-(6-hydroxyquinolin-2-yl)-2-methoxybenzoic acid;
2-hydroxy-4-(6-hydroxyquinolin-2-yl)benzoic acid;
2-(4-hydroxy-3-nitrophenyl)quinolin-6-ol;
4-(1-cyano-5-fluoro-6-hydroxynaphthalen-2-yl)benzoic acid;
4-(1-cyano-6-hydroxynaphthalen-2-yl)-3-fluorobenzoic acid;
3-chloro-4-(5-fluoro-6-hydroxynaphthalen-2-yl)benzoic acid;
4-(5-fluoro-6-hydroxynaphthalen-2-yl)benzoic acid;
3-fluoro-4-(5-fluoro-6-hydroxynaphthalen-2-yl)benzoic acid;
4-(5-fluoro-6-hydroxynaphthalen-2-yl)-3-methylbenzoic acid;
4-(6-hydroxynaphthalen-2-yl)-2-methoxybenzoic acid;
2-hydroxy-4-(6-hydroxynaphthalen-2-yl)benzoic acid;
6-(4-hydroxy-3-nitrophenyl)naphthalen-2-ol;
4-(6-hydroxynaphthalen-2-yl)-3-methylbenzoic acid;
3-fluoro-4-(4-fluoro-6-hydroxynaphthalen-2-yl)benzoic acid;
4-(4-fluoro-6-hydroxynaphthalen-2-yl)benzoic acid;
4-(4,5-difluoro-6-hydroxynaphthalen-2-yl)-3-fluorobenzoic acid;
4-(4,5-difluoro-6-hydroxynaphthalen-2-yl)-3-methylbenzoic acid;
4-(4,5-difluoro-6-hydroxynaphthalen-2-yl)benzoic acid; and
4-(4,5-difluoro-6-hydroxynaphthalen-2-yl)-3-methoxybenzoic acid.

The GSNOR inhibitors described herein were found to have two key pharmacophores: the hydroxyl group and the acidic moiety. In particular, provided are substituted and unsubstituted multi-cyclic analogs, possessing a hydroxyl group on one of the cycles and an acidic moiety on another cycle. These two cyclic groups are joined by a linker such that the distance between the two pharmacophores is appropriate for binding within the GSNOR active site. The importance of the pharmacophores is confirmed by the potency of the compounds as inhibitors of GSNOR. It was also confirmed through information gained from x-ray crystallography. The structures of two inhibitors bound to GSNOR and NAD+ were determined by X-ray crystallography (see Example 4 for methodology). From these solved structures, the importance of the two pharmacophores (the hydroxyl group and the acidic moiety) is evident.

Single crystal x-ray diffraction is a method useful to understanding the 3-dimensional structure of the binding pocket. The solved crystal structures offer significant information about the GSNOR binding pocket and the interactions of the inhibitors within the binding site. The crystal structure of the ternary complex of GSNOR, NAD+, and Compound IV-10 (Table 4, Example 1) (4-(7-hydroxy-4-oxo-2-(trifluoromethyl)-4H-chromen-3-yl)benzoic acid) was solved. The enzyme-ligand complex crystallized as a homodimer with Compound IV-10 binding to each GSNOR monomer. The key pharmacophores identified from the ternary complex of GSNOR inhibitor with GSNOR and NAD+ are the hydroxyl group on the bicyclic ring of Compound IV-10, which hydrogen bonds to histidine 66, cysteine 44, cysteine 173 and threonine 46. This hydroxyl group is also part of the Zn complex to histidine 66, cysteine 44, and cysteine 173 and threonine 46. The carboxylic acid of Compound IV-10 hydrogen bonds to the glutamine 111 and forms a salt bridge with lysine 283. The phenyl ring connecting to the carboxylic acid forms pi-pi interaction with the arginine 114.

A second crystal structure, with the inhibitor 4-(7-hydroxy-4-oxo-2-(trifluoromethyl)-4H-chromen-3-yloxy)benzoic acid (Compound V-1, Example 1) bound to GSNOR and NAD+ was solved. The enzyme-ligand complex crystallized as a homodimer with Compound V-1 binding to each GSNOR monomer. A key pharmacophore identified from the ternary complex of GSNOR inhibitor with GSNOR and NAD+ is the hydroxyl group on the bicyclic ring of Compound V-1, which hydrogen bonds similarly to Compound IV-10 to histidine 66, cysteine 44, cysteine 173 and threonine 46. This hydroxyl group is also part of the Zn complex to histidine 66, cysteine 44, and cysteine 173 and threonine 46. Another key pharmacophore identified is the carboxylic acid of Compound V-1. The carboxylic acid of Compound V-1 interacts with the enzyme in a slightly different way than in the case of Compound IV-10. The carboxylic acid of Compound V-1 forms salt bridges with lysine 283 and arginine 114, whereas in the case of Compound IV-10, it forms pi-pi interaction with the phenyl ring connecting to the carboxylic acid. In the case of Compound V-1, glutamine 111 is not involved in direct interaction with the inhibitor.

Once the crystal structures were solved, a distance measurement was made. For Compound IV-10 bound to GSNOR and NAD+, the distance between the hydroxyl group and the carboxylic acid was found to be 12.07 Å (this value was determined by measuring the distance between the O atom of hydroxyl, and the C atom of the carbonyl). For the second crystal structure with Compound V-1 bound to GSNOR and NAD+, the distance between the hydroxyl group and the carboxylic acid found to be 12.34 Å (measured in the same manner as described above). These two solved structures provide empirical distance values that take into account the interactions within the binding site.

Also, it has been empirically shown that the compounds of Formula 1 in Tables 1-8 of Example 1 have GSNOR inhibiting properties, all with IC₅₀ values <10 μM, and with many having <100 nM activity (see Tables in Example 1, and methodology for IC₅₀ in Example 3).

Simple Molecular Mechanics (MM2) minimization is a computational tool often used to predict 3-dimensional structure of molecules. For a review of MM2 and applications of molecular mechanics methods in general, see Molecular Mechanics, by U. Burkert and N. L. Allinger, ACS Monograph 177, American Chemical Society, Washington, D.C., USA, 1982. The measurement of distance between the hydroxyl group and the acidic moiety is a predictor of whether the molecule is capable of making the proper interactions within the GSNOR binding pocket. Therefore, a meaningful distance value can be obtained for a molecule that undergoes MM2 minimization, followed by a distance measurement between the key pharmacophores.

ChemBio3D ultra 11.0 software (purchased from CambridgeS oft) was used to perform MM2 energy minimizations for the compounds of Tables 1-8 of Example 1. Molecules were drawn in 2D in ChemDraw in an orientation similar to that shown in the tables 1-8, copied and pasted into ChemBio3D ultra 11.0, then the MM2 minimization was performed. After the minimization, a distance measurement was made between the two pharmacophores. This value as reported herein is obtained by measuring the distance between the O of the hydroxyl group and the atom of the acidic function connected to the Cy₂. For example, when the acidic moiety is carboxylic acid, the value is measured from the O of hydroxyl to the C of the acid. Another example is when the acidic moiety is tetrazole, then the value is measured from the O of the hydroxyl to the C of tetrazole.

It will be appreciated by those skilled in the art that MM2 minimizations, while useful, are a limited representation of how the inhibitors may fit in the enzyme binding site. The distance measurement taken after a MM2 calculation does not take into account the ability of the molecule to stretch or bend (meaning changes in bond lengths, angles, etc.) within the binding site to achieve an appropriate distance for binding. Therefore, a second parameter that is useful in analyzing the reliability of the distance measurement is the determination of the number of rotatable bonds between the acidic moiety and the hydroxyl groups of the molecule, called “linear rotatable bonds” herein.

An example of a compound with 2 linear rotatable bonds is shown below as Compound IV-2 (see Table 4 of Example 1). While overall this molecule has 4 rotatable bonds, as defined herein, the “linear rotatable bonds” are those directly between the hydroxyl and the acidic moiety and therefore, Compound IV-2 has 2 linear rotatable bonds as shown below.

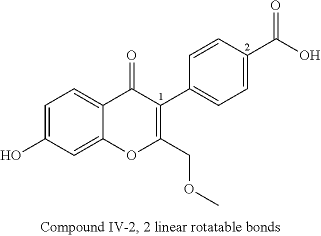 [SMILES: COCc1oc2cc(O)ccc2c(=O)c1-c1ccc(C(=O)O)cc1]

An example of a compound with 3 linear rotatable bonds is shown here: Compound VII-1 in Table 7 of Example 1.

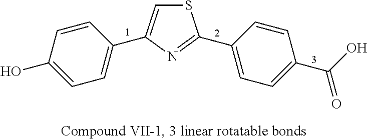 [SMILES: O=C(O)c1ccc(-c2nc(-c3ccc(O)cc3)cs2)cc1]

Compounds with “linear rotatable bonds” <4, meaning compounds with less than 4 rotatable bonds between the hydroxyl and acidic moiety, more closely fit a proper distance within the binding site without having to stretch or bend to make the proper interactions. Therefore without being bound by theory, the distance measurement obtained after MM2 minimization for compounds with “linear rotatable bonds” <4 is likely similar to what it would be within the binding site, around 12±2 Å, or in the range of 10 to 14 Å.

The molecules with more “linear rotatable bonds” between the key pharmacophores have greater ability to deviate from the MM2 minimized value to obtain a proper distance for binding. For example, Compound VI-2 after MM2 minimization has a measured distance value of 7 Å.

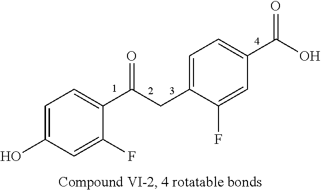 [SMILES: O=C(O)c1ccc(CC(=O)c2ccc(O)cc2F)c(F)c1]

This is a much lower measurement than the crystal structures obtained (approx. 12 Å) would indicate as necessary for binding. However, this molecule has an IC₅₀<100 nM, suggesting strong binding to the enzyme. Compound VI-2 has 4 rotatable bonds (shown as numbers 1-4) between the key pharmacophores (linear rotatable bonds), indicating that the molecule has greater ability to deviate from the calculated minimum by bending, stretching, etc. to obtain a proper binding distance when in the presence of the GSNOR enzyme.

Compounds of the invention can have distance measurements after MM2 minimization from 6-16 Å. This range takes into account that compounds with linear rotatable bonds ≧4 have a large degree of freedom to deviate from the MM2 minimized structure to properly bind within the GSNOR pocket. The active inhibitors of GSNOR that have a measured distance of 6-10 Å, likely have 4 or more linear rotatable bonds.

When a bond to a substituent is shown to cross a bond connecting two atoms in a ring, then such substituent may be bonded to any atom in the ring. When a substituent is listed without indicating the atom via which such substituent is bonded to the rest of the compound of a given Formula, then such substituent may be bonded via any atom in such substituent. Combinations of substituents and/or variables are permissible, but only if such combinations result in stable compounds.

The compounds described herein may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, and geometric isomeric forms of a structure are intended, unless the specific stereochemistry or isomeric form is specifically indicated. All tautomers of shown or described compounds are also considered to be part of the present invention.

It is to be understood that isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

2. Representative Compounds

Example 1 lists representative analogs of the invention. The synthetic methods that can be used to prepare each compound are detailed in Example 2 or in prior patent applications described before each table in Example 1. Supporting mass spectrometry data and/or proton NMR data is also found in Example 2 for compounds not previously described. GSNOR inhibitor activity was determined by the assay described in Example 3 and IC₅₀ values were obtained. Ranges of IC₅₀ values denoted as a: IC₅₀<100 nM, b: 100 nM-1 μM, and c: 1 μM-10 μM are found in Tables 1-8 in Example 1.

### C. Definitions

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “acyl” includes compounds and moieties that contain the acetyl radical (CH₃CO—) or a carbonyl group to which a straight or branched chain lower alkyl residue is attached.

The term “alkyl” as used herein refers to a straight or branched chain, saturated hydrocarbon having the indicated number of carbon atoms. For example, (C₁-C₆) alkyl is meant to include, but is not limited to methyl, ethyl, propyl, isopropyl, butyl, sec-butyl, tert-butyl, pentyl, isopentyl, neopentyl, hexyl, isohexyl, and neohexyl. An alkyl group can be unsubstituted or optionally substituted with one or more substituents as described herein.

The term “alkenyl” as used herein refers to a straight or branched chain unsaturated hydrocarbon having the indicated number of carbon atoms and at least one double bond. Examples of a (C₂-C₈) alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents as described herein.

The term “alkynyl” as used herein refers to a straight or branched chain unsaturated hydrocarbon having the indicated number of carbon atoms and at least one triple bond. Examples of a (C₂-C₈) alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne, and 4-octyne. An alkynyl group can be unsubstituted or optionally substituted with one or more substituents as described herein.

The term “alkoxy” as used herein refers to an —O-alkyl group having the indicated number of carbon atoms. For example, a (C₁-C₆) alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “aminoalkyl” as used herein, refers to an alkyl group (typically one to six carbon atoms) wherein one or more of the C₁-C₆ alkyl group's hydrogen atoms is replaced with an amine of formula —N(R^{c})₂, wherein each occurrence of R^{c} is independently —H or (C₁-C₆) alkyl. Examples of aminoalkyl groups include, but are not limited to, —CH₂NH₂, —CH₂CH₂NH₂, —CH₂CH₂CH₂NH₂, —CH₂CH₂CH₂CH₂NH₂, —CH₂CH₂CH₂CH₂CH₂NH₂, —CH₂CH₂CH₂CH₂CH₂CH₂NH₂, —CH₂CH₂CH₂N(CH₃)₂, t-butylaminomethyl, isopropylaminomethyl, and the like.

The term “aryl” as used herein refers to a 5- to 14-membered monocyclic, bicyclic, or tricyclic aromatic ring system. Examples of an aryl group include phenyl and naphthyl. An aryl group can be unsubstituted or optionally substituted with one or more substituents as described herein below. Examples of aryl groups include phenyl or aryl heterocycles such as, pyrrole, furan, thiophene, thiazole, isothiazole, imidazole, triazole, tetrazole, pyrazole, oxazole, isoxazole, pyridine, pyrazine, pyridazine, and pyrimidine, and the like.

As used herein, the term “bioactivity” indicates an effect on one or more cellular or extracellular process (e.g., via binding, signaling, etc.) which can impact physiological or pathophysiological processes.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxy” or “carboxyl” means a —COOH group or carboxylic acid.

“Acidic moiety” as used herein is defined as a carboxylic acid or a carboxylic acid bioisostere. Bioisosteres are substituents or groups with similar physical or chemical properties which produce broadly similar biological properties to a chemical compound. For a review of bioisosteres, see J. Med. Chem, 2011, 54, 2529-2591. Examples of “acidic moiety” include but are not limited to

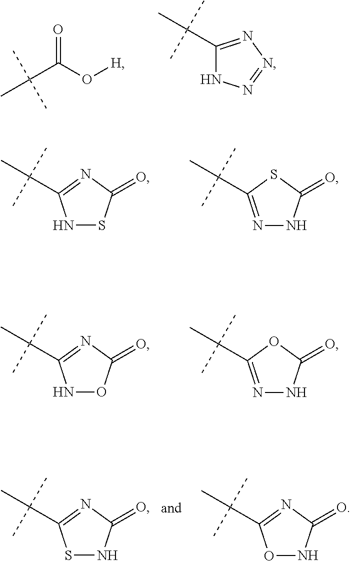

“Pharmacophore” is defined as “a set of structural features in a molecule that is recognized at a receptor site and is responsible for that molecule's biological activity” (Gund, Prog. Mol. Subcell. Biol., 5: pp 117-143 (1977)).

The term “Cₘ-Cₙ” means “m” number of carbon atoms to “n” number of carbon atoms. For example, the term “C₁-C₆” means one to six carbon atoms (C₁, C₂, C₃, C₄, C₅, or C₆). The term “C₂-C₆” includes two to six carbon atoms (C₂, C₃, C₄, C₅, or C₆). The term “C₃-C₆” includes three to six carbon atoms (C₃, C₄, C₅, or C₆).

The term “cycloalkyl” as used herein refers to a 3- to 14-membered saturated or unsaturated non-aromatic monocyclic, bicyclic, or tricyclic hydrocarbon ring system. Included in this class are cycloalkyl groups which are fused to a benzene ring. Representative cycloalkyl groups include, but are not limited to, cyclopropyl, cyclobutyl, cyclobutenyl, cyclopentyl, cyclopentenyl, cyclopentadienyl, cyclohexyl, cyclohexenyl, 1,3-cyclohexadienyl, cycloheptyl, cycloheptenyl, 1,3-cycloheptadienyl, 1,4-cycloheptadienyl, -1,3,5-cycloheptatrienyl, cyclooctyl, cyclooctenyl, 1,3-cyclooctadienyl, 1,4-cyclooctadienyl, -1,3,5-cyclooctatrienyl, decahydronaphthalene, octahydronaphthalene, hexahydronaphthalene, octahydroindene, hexahydroindene, tetrahydroinden, decahydrobenzocycloheptene, octahydrobenzocycloheptene, hexahydrobenzocycloheptene, tetrahydrobenzocyclopheptene, dodecahydroheptalene, decahydroheptalene, octahydroheptalene, hexahydroheptalene, tetrahydroheptalene, (1s,3s)-bicyclo[1.1.0]butane, bicyclo[1.1.1]pentane, bicyclo[2.1.1]hexane, Bicyclo[2.2.1]heptane, bicyclo[2.2.2]octane, bicyclo[3.1.1]heptane, bicyclo[3.2.1]octane, bicyclo[3.3.1]nonane, bicyclo[3.3.2]decane, bicyclo[3.3.]undecane, bicyclo[4.2.2]decane, and bicyclo[4.3.1]decane. A cycloalkyl group can be unsubstituted or optionally substituted with one or more substituents as described herein below.

The term “halogen” includes fluorine, bromine, chlorine, iodine, etc.

The term “haloalkyl,” as used herein, refers to a C₁-C₆ alkyl group wherein from one or more of the C₁-C₆ alkyl group's hydrogen atom is replaced with a halogen atom, which can be the same or different. Examples of haloalkyl groups include, but are not limited to, trifluoromethyl, 2,2,2-trifluoroethyl, 4-chlorobutyl, 3-bromopropyl, pentachloroethyl, and 1,1,1-trifluoro-2-bromo-2-chloroethyl.

The term “heteroalkyl,” by itself or in combination with another term, means, unless otherwise stated, a stable straight or branched chain alkyl, or combinations thereof, consisting of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N, and S can be placed at any position of the heteroalkyl group. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, and —CH₂—CH═N—OCH₃. Up to two heteroatoms can be consecutive, for example, —CH₂—NH—OCH₃. When a prefix such as (C₂-C₈) is used to refer to a heteroalkyl group, the number of carbons (2 to 8, in this example) is meant to include the heteroatoms as well. For example, a C₂-heteroalkyl group is meant to include, for example, —CH₂OH (one carbon atom and one heteroatom replacing a carbon atom) and —CH₂SH.

To further illustrate the definition of a heteroalkyl group, where the heteroatom is oxygen, a heteroalkyl group can be an oxyalkyl group. For instance, (C₂-C₅) oxyalkyl is meant to include, for example —CH₂—O—CH₃ (a C₃-oxyalkyl group with two carbon atoms and one oxygen replacing a carbon atom), —CH₂CH₂CH₂CH₂OH, —OCH₂CH₂OCH₂CH₂OH, —OCH₂CH(OH)CH₂OH, and the like.

The term “heteroaryl” as used herein refers to an aromatic heterocycle ring of 5 to 14 members and having at least one heteroatom selected from nitrogen, oxygen, and sulfur, and containing at least 1 carbon atom, including monocyclic, bicyclic, and tricyclic ring systems. Representative heteroaryls are triazolyl, tetrazolyl, oxadiazolyl, pyridyl, furyl, benzofuranyl, thienyl, benzothienyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, azepinyl, oxepinyl, quinoxalinyl and oxazolyl. A heteroaryl group can be unsubstituted or optionally substituted with one or more substituents as described herein below.

As used herein, the term “heteroatom” is meant to include oxygen (O), nitrogen (N), and sulfur (S).

As used herein, the term “heterocycle” refers to 3- to 14-membered ring systems which are either saturated, unsaturated, or aromatic, and which contains from 1 to 4 heteroatoms independently selected from nitrogen, oxygen, and sulfur, and wherein the nitrogen and sulfur heteroatoms can be optionally oxidized, and the nitrogen heteroatom can be optionally quaternized, including monocyclic, bicyclic, and tricyclic ring systems. The bicyclic and tricyclic ring systems may encompass a heterocycle or heteroaryl fused to a benzene ring. The heterocycle can be attached via any heteroatom or carbon atom, where chemically acceptable. Heterocycles include heteroaryls as defined above. Representative examples of heterocycles include, but are not limited to, aziridinyl, oxiranyl, thiiranyl, triazolyl, tetrazolyl, azirinyl, diaziridinyl, diazirinyl, oxaziridinyl, azetidinyl, azetidinonyl, oxetanyl, thietanyl, piperidinyl, piperazinyl, morpholinyl, pyrrolyl, oxazinyl, thiazinyl, diazinyl, dioxanyl, triazinyl, tetrazinyl, imidazolyl, tetrazolyl, pyrrolidinyl, isoxazolyl, furanyl, furazanyl, pyridinyl, oxazolyl, benzoxazolyl, benzisoxazolyl, thiazolyl, benzthiazolyl, thienyl, pyrazolyl, triazolyl, pyrimidinyl, benzimidazolyl, isoindolyl, indazolyl, benzodiazolyl, benzotriazolyl, benzoxazolyl, benzisoxazolyl, purinyl, indolyl, isoquinolinyl, quinolinyl, and quinazolinyl. A heterocycle group can be unsubstituted or optionally substituted with one or more substituents as described herein below.

The term “heterocycloalkyl,” by itself or in combination with other terms, represents, unless otherwise stated, cyclic versions of “heteroalkyl.” Additionally, a heteroatom can occupy the position at which the heterocycle is attached to the remainder of the molecule. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “hydroxyalkyl,” as used herein, refers to an alkyl group having the indicated number of carbon atoms wherein one or more of the hydrogen atoms in the alkyl group is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “hydroxy” or “hydroxyl” includes groups with an —OH or —O⁻.

As used herein, N-oxide, or amine oxide, refers to a compound derived from a tertiary amine by the attachment of one oxygen atom to the nitrogen atom, R₃N⁺—O⁻. By extension the term includes the analogous derivatives of primary and secondary amines.

As used herein and unless otherwise indicated, the term “stereoisomer” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. In some embodiments, a stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

As used herein, “protein” is used synonymously with “peptide,” “polypeptide,” or “peptide fragment”. A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

As used herein, “modulate” is meant to refer to an increase or decrease in the levels of a peptide or a polypeptide, or to increase or decrease the stability or activity of a peptide or a polypeptide. The term “inhibit” is meant to refer to a decrease in the levels of a peptide or a polypeptide or to a decrease in the stability or activity of a peptide or a polypeptide. In preferred embodiments, the peptide which is modulated or inhibited is S-nitrosoglutathione (GSNO) or protein S-nitrosothiols (SNOs).

As used here, the terms “nitric oxide” and “NO” encompass uncharged nitric oxide and charged nitric oxide species, particularly including nitrosonium ion (NO⁺) and nitroxyl ion (NO⁻). The reactive form of nitric oxide can be provided by gaseous nitric oxide. Compounds having the structure X—NO_{y} wherein X is a nitric oxide releasing, delivering, or transferring moiety, including any and all such compounds which provide nitric oxide to its intended site of action in a form active for their intended purpose, and Y is 1 or 2.

Repair” means recovering of structural integrity and normal physiologic function. By way of example, the oral and upper airway respiratory epithelium can repair damage done by thermal injury or viral infection.

“Regeneration” means the ability of an organ to enter non-malignant cellular, vascular and stromal growth to restore functional organ tissue. By way of example, wound healing involves regeneration of tissue and organs (e.g. skin, gastric and intestinal mucosa), as does bone following fracture, and the liver following partial surgical removal and exposure to infectious or toxic insult.

As utilized herein, the term “pharmaceutically acceptable” means approved by a regulatory agency of a federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils.

A “pharmaceutically acceptable salt” or “salt” of a compound of the invention is a product of the disclosed compound that contains an ionic bond, and is typically produced by reacting the disclosed compound with either an acid or a base, suitable for administering to a subject. A pharmaceutically acceptable salt can include, but is not limited to, acid addition salts including hydrochlorides, hydrobromides, phosphates, sulphates, hydrogen sulphates, alkylsulphonates, arylsulphonates, arylalkylsulfonates, acetates, benzoates, citrates, maleates, fumarates, succinates, lactates, and tartrates; alkali metal cations such as Li, Na, and K, alkali earth metal salts such as Mg or Ca, or organic amine salts.

A “pharmaceutical composition” is a formulation comprising the disclosed compounds in a form suitable for administration to a subject. A pharmaceutical composition of the invention is preferably formulated to be compatible with its intended route of administration. Examples of routes of administration include, but are not limited to, oral and parenteral, e.g., intravenous, intradermal, subcutaneous, inhalation, topical, transdermal, transmucosal, and rectal administration.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Ring double bonds, as used herein, are double bonds that are formed between two adjacent ring atoms (e.g., C═C, C═N, or N═N).

Substituents for the groups referred to as alkyl, heteroalkyl, alkylene, alkenyl, alkynyl, cycloalkyl, heterocycloalkyl, cycloalkenyl, and heterocycloalkenyl can be selected from a variety of groups including —OR^{d}, ═O, ═NR^{d}′, ═N—OR^{d′}, —NR^{d}′R^{d}″, —SR^{d′}, -halo, —SiR^{d}′R^{d}″R^{d}′″, —OC(O)R^{d}′, —C(O)R^{d}′, —CO₂R^{d}′, —CONR^{d}′R^{d}″, —OC(O)NR^{d}′R^{d}″, —NR^{d}″C(O)R^{d}′, —NR^{d}′″C(O)NR^{d}′R^{d}″, —NR^{d}′″SO₂NR^{d}′R^{d}′, —NR^{d}″CO₂R^{d}′, —NHC(NH₂)═NH, —NR^{a}′C(NH₂)═NH, —NHC(NH₂)═NR^{d}′, —S(O)R^{d}′, —SO₂R^{d}′, —SO₂NR^{d}′R^{d}″, —NR^{d}″SO₂R^{d}′, —CN, and —NO₂, in a number ranging from zero to three, with those groups having zero, one or two substituents being exemplary.

R^{d}′, R^{d}″, and R^{d}′″ each independently refer to hydrogen, unsubstituted (C₁-C₈)alkyl, unsubstituted hetero(C₁-C₈) alkyl, unsubstituted aryl, and aryl substituted with one to three substituents selected from -halo, unsubstituted alkyl, unsubstituted alkoxy, unsubstituted thioalkoxy, and unsubstituted aryl (C₁-C₄)alkyl. When R^{d}′ and R^{d}″ are attached to the same nitrogen atom, they can be combined with the nitrogen atom to form a 5-, 6-, or 7-membered ring. For example, —NR^{d}′R^{d}″ can represent 1-pyrrolidinyl or 4-morpholinyl.

Typically, an alkyl or heteroalkyl group will have from zero to three substituents, with those groups having two or fewer substituents being exemplary of the present invention. An alkyl or heteroalkyl radical can be unsubstituted or monosubstituted. In some embodiments, an alkyl or heteroalkyl radical will be unsubstituted.

Exemplary substituents for the alkyl and heteroalkyl radicals include, but are not limited to —OR^{d}″, ═O, ═NR^{d}′, ═N—OR^{d}′, —NR^{d}′R^{d}″, —SR^{d}′, -halo, —SiR^{d}′R^{d}″R^{d}′″, —OC(O)R^{d}′, —C(O)R^{d}′, —CO₂R^{d}′, —CONR^{d}′R^{d}″, —OC(O)NR^{d}′R^{d}″, —NR^{d}″C(O)R^{d}′, —NR^{d}′″C(O)NR^{d}′R^{d}″, —NR^{d}′″SO₂NR^{d}′R^{d}″, —NR^{d}″CO₂R^{d}′, —NHC(NH₂)═NH, —NR^{a}′C(NH₂)═NH, —NHC(NH₂)═NR^{d}′, —S(O)R^{d}′, —SO₂R^{d}′, —SO₂NR^{d}′R^{d}″, —NR^{d}″SO₂R^{d}′, —CN, and —NO₂, where R^{d}′, R^{d}″, and R^{d}′″ are as defined above. Typical substituents can be selected from: —OR^{d}″, ═O, —NR^{d}′R^{d}″, -halo, —OC(O)R^{d}′, —CO₂R^{d}′, —C(O)NR^{d}′R^{d}″, —OC(O)NR^{d}′R^{d}″, NR^{d}″C(O)R^{d}′, —NR^{d}″CO₂R^{d}′, —NR^{d}′″SO₂NR^{d}′R^{d}″, —SO₂R^{d}′, —SO₂NR^{d}′R^{d}″, —NR^{d}″SO₂R^{d}′, —CN, and —NO₂.

Similarly, substituents for the aryl and heteroaryl groups are varied and selected from: -halo, —OR^{e}′, —OC(O)R^{e}′, —NR^{e}′R^{e}″, —SR^{e}′, —R^{e}′, —CN, —NO₂, —CO₂R^{e}′, —C(O)NR^{e}′R^{e}″, —C(O)R^{e}′, —OC(O)NR^{e}′R^{e}″, —NR^{e}″C(O)R^{e}′, —NR^{e}″CO₂R^{e}′, —NR^{e}′″C(O)NR^{e}′R^{e}″, —NR^{e}′″SO₂NR^{e}′R^{e}″, —NHC(NH₂)═NH, —NR^{e}′C(NH₂)═NH, —NH—C(NH₂)═NR^{e}′, —S(O)R^{e}′, —SO₂R^{e}′, —SO₂NR^{e}′R^{e}″, —NR^{e}″SO₂R^{e}′, —N₃, —CH(Ph)₂, perfluoroalkoxy, and perfluoro(C₁-C₄)alkyl, in a number ranging from zero to the total number of open valences on the aromatic ring system.

R^{e}′, R^{e}″ and R^{e}′″ are independently selected from hydrogen, unsubstituted (C₁-C₈) alkyl, unsubstituted hetero(C₁-C₈) alkyl, unsubstituted aryl, unsubstituted heteroaryl, unsubstituted aryl(C₁-C₄) alkyl, and unsubstituted aryloxy(C₁-C₄) alkyl. Typically, an aryl or heteroaryl group will have from zero to three substituents, with those groups having two or fewer substituents being exemplary in the present invention. In one embodiment of the invention, an aryl or heteroaryl group will be unsubstituted or monosubstituted. In another embodiment, an aryl or heteroaryl group will be unsubstituted.

Two of the substituents on adjacent atoms of an aryl or heteroaryl ring in an aryl or heteroaryl group as described herein may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -J-(CH₂)ᵣ—K—, wherein J and K are independently —CH₂—, —O—, —NH—, —S—, —S(O)—, —S(O)₂—, —S(O)₂NR^{f}′—, or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR^{f}′—, —S—, —S(O)—, —S(O)₂—, or —S(O)₂NR^{a}′—. The substituent R^{f}′ in —NR^{f}′— and —S(O)₂NR^{f}′— is selected from hydrogen or unsubstituted (C₁-C₆) alkyl.

“Stable compound” and “stable structure” are meant to indicate a compound that is sufficiently robust to survive isolation to a useful degree of purity from a reaction mixture, and formulation into an efficacious therapeutic agent.

As used herein the term “therapeutically effective amount” generally means the amount necessary to ameliorate at least one symptom of a disorder to be prevented, reduced, or treated as described herein. The phrase “therapeutically effective amount” as it relates to the GSNOR inhibitors of the present invention shall mean the GSNOR inhibitor dosage that provides the specific pharmacological response for which the GSNOR inhibitor is administered in a significant number of subjects in need of such treatment. It is emphasized that a therapeutically effective amount of a GSNOR inhibitor that is administered to a particular subject in a particular instance will not always be effective in treating the conditions/diseases described herein, even though such dosage is deemed to be a therapeutically effective amount by those of skill in the art.

The term “biological sample” includes, but is not limited to, samples of blood (e.g., serum, plasma, or whole blood), urine, saliva, sweat, breast milk, vaginal secretions, semen, hair follicles, skin, teeth, bones, nails, or other secretions, body fluids, tissues, or cells. In accordance with the invention, the levels of the GSNOR in the biological sample can be determined by the methods described in U.S. Patent Application Publication No. 2005/0014697.

### D. Pharmaceutical Compositions

The invention encompasses pharmaceutical compositions comprising at least one compound of the invention described herein and at least one pharmaceutically acceptable carrier. Suitable carriers are described in “Remington: The Science and Practice, Twentieth Edition,” published by Lippincott Williams & Wilkins, which is incorporated herein by reference. Pharmaceutical compositions according to the invention may also comprise one or more non-inventive compound active agents.

The pharmaceutical compositions of the invention can comprise novel compounds described herein, the pharmaceutical compositions can comprise known compounds which previously were not known to have GSNOR inhibitor activity, or a combination thereof.

The compounds of the invention can be utilized in any pharmaceutically acceptable dosage form, including, but not limited to injectable dosage forms, liquid dispersions, gels, aerosols, ointments, creams, lyophilized formulations, dry powders, tablets, capsules, controlled release formulations, fast melt formulations, delayed release formulations, extended release formulations, pulsatile release formulations, mixed immediate release and controlled release formulations, etc. Specifically, the compounds of the invention described herein can be formulated: (a) for administration selected from the group consisting of oral, pulmonary, intravenous, intra-arterial, intrathecal, intra-articular, rectal, ophthalmic, colonic, parenteral, intracisternal, intravaginal, intraperitoneal, local, buccal, nasal, and topical administration; (b) into a dosage form selected from the group consisting of liquid dispersions, gels, aerosols, ointments, creams, tablets, sachets, and capsules; (c) into a dosage form selected from the group consisting of lyophilized formulations, dry powders, fast melt formulations, controlled release formulations, delayed release formulations, extended release formulations, pulsatile release formulations, and mixed immediate release and controlled release formulations; or (d) any combination thereof.

For respiratory infections, an inhalation formulation can be used to achieve high local concentrations. Formulations suitable for inhalation include dry power or aerosolized or vaporized solutions, dispersions, or suspensions capable of being dispensed by an inhaler or nebulizer into the endobronchial or nasal cavity of infected patients to treat upper and lower respiratory bacterial infections.

Solutions or suspensions used for parenteral, intradermal, or subcutaneous application can comprise one or more of the following components: (1) a sterile diluent such as water for injection, saline solution, fixed oils, polyethylene glycols, glycerine, propylene glycol, or other synthetic solvents; (2) antibacterial agents such as benzyl alcohol or methyl parabens; (3) antioxidants such as ascorbic acid or sodium bisulfite; (4) chelating agents such as ethylenediaminetetraacetic acid; (5) buffers such as acetates, citrates, or phosphates; and (5) agents for the adjustment of tonicity such as sodium chloride or dextrose. The pH can be adjusted with acids or bases, such as hydrochloric acid or sodium hydroxide. A parenteral preparation can be enclosed in ampoules, disposable syringes, or multiple dose vials made of glass or plastic.

Pharmaceutical compositions suitable for injectable use may comprise sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). In all cases, the composition must be sterile and should be fluid to the extent that easy syringability exists. The pharmaceutical composition should be stable under the conditions of manufacture and storage and should be preserved against the contaminating action of microorganisms such as bacteria and fungi.

The carrier can be a solvent or dispersion medium comprising, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol or sorbitol, and inorganic salts such as sodium chloride in the composition. Prolonged absorption of the injectable compositions can be brought about by including in the composition an agent which delays absorption, for example, aluminum monostearate and gelatin.

Sterile injectable solutions can be prepared by incorporating the active reagent in the required amount in an appropriate solvent with one or a combination of ingredients enumerated above, as required, followed by filtered sterilization. Generally, dispersions are prepared by incorporating at least one compound of the invention into a sterile vehicle that contains a basic dispersion medium and any other required ingredients. In the case of sterile powders for the preparation of sterile injectable solutions, exemplary methods of preparation include vacuum drying and freeze-drying, both of which yield a powder of a compound of the invention plus any additional desired ingredient from a previously sterile-filtered solution thereof.

Oral compositions generally include an inert diluent or an edible carrier. They can be enclosed, for example, in gelatin capsules or compressed into tablets. For the purpose of oral therapeutic administration, the compound of the invention can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition.

For administration by inhalation, the compounds are delivered in the form of an aerosol spray from pressured container or dispenser that contains a suitable propellant, e.g., a gas such as carbon dioxide, a nebulized liquid, or a dry powder from a suitable device. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active reagents are formulated into ointments, salves, gels, or creams as generally known in the art. The reagents can also be prepared in the form of suppositories (e.g., with conventional suppository bases such as cocoa butter and other glycerides) or retention enemas for rectal delivery.

In one embodiment, the compounds of the invention are prepared with carriers that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of the compounds of the invention may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of the compound of the invention calculated to produce the desired therapeutic effect in association with the required pharmaceutical carrier. The specification for the dosage unit forms of the invention are dictated by and directly dependent on the unique characteristics of the compound of the invention and the particular therapeutic effect to be achieved, and the limitations inherent in the art of compounding such an active agent for the treatment of individuals.

Pharmaceutical compositions according to the invention comprising at least one compound of the invention can comprise one or more pharmaceutical excipients. Examples of such excipients include, but are not limited to binding agents, filling agents, lubricating agents, suspending agents, sweeteners, flavoring agents, preservatives, buffers, wetting agents, disintegrants, effervescent agents, and other excipients. Such excipients are known in the art. Exemplary excipients include: (1) binding agents which include various celluloses and crosslinked polyvinylpyrrolidone, microcrystalline cellulose, such as Avicel® PH101 and Avicel® PH102, silicified microcrystalline cellulose (ProSolv SMCC™), gum tragacanth and gelatin; (2) filling agents such as various starches, lactose, lactose monohydrate, and lactose anhydrous; (3) disintegrating agents such as alginic acid, Primogel, corn starch, lightly crosslinked polyvinyl pyrrolidone, potato starch, maize starch, and modified starches, croscarmellose sodium, cross-povidone, sodium starch glycolate, and mixtures thereof; (4) lubricants, including agents that act on the flowability of a powder to be compressed, include magnesium stearate, colloidal silicon dioxide, such as Aerosil® 200, talc, stearic acid, calcium stearate, and silica gel; (5) glidants such as colloidal silicon dioxide; (6) preservatives, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride; (7) diluents such as pharmaceutically acceptable inert fillers, such as microcrystalline cellulose, lactose, dibasic calcium phosphate, saccharides, and/or mixtures of any of the foregoing; examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress; mannitol; starch; sorbitol; sucrose; and glucose; (8) sweetening agents, including any natural or artificial sweetener, such as sucrose, saccharin sucrose, xylitol, sodium saccharin, cyclamate, aspartame, and acesulfame; (9) flavoring agents, such as peppermint, methyl salicylate, orange flavoring, Magnasweet® (trademark of MAFCO), bubble gum flavor, fruit flavors, and the like; and (10) effervescent agents, including effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

### E. Kits Comprising the Compositions of the Invention

The present invention also encompasses kits comprising the compositions of the invention. Such kits can comprise, for example, (1) at least one compound of the invention; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial, or similar apparatus for holding and/or mixing the kit components; and (3) delivery apparatus, such as an inhaler, nebulizer, syringe, etc.

### F. Methods of Preparing Compounds of the Invention

The compounds of the invention can readily be synthesized using known synthetic methodologies or via a modification of known synthetic methodologies. As would be readily recognized by a skilled artisan, the methodologies described below allow the synthesis of compounds having a variety of substituents. Exemplary synthetic methods are described in the Examples below.

If needed, further purification and separation of enantiomers and diastereomers can be achieved by routine procedures known in the art. Thus, for example, the separation of enantiomers of a compound can be achieved by the use of chiral HPLC and related chromatographic techniques. Diastereomers can be similarly separated. In some instances, however, diastereomers can simply be separated physically, such as, for example, by controlled precipitation or crystallization.

The process of the invention, when carried out as prescribed herein, can be conveniently performed at temperatures that are routinely accessible in the art. In one embodiment, the process is performed at a temperature in the range of about 25° C. to about 110° C. In another embodiment, the temperature is in the range of about 40° C. to about 100° C. In yet another embodiment, the temperature is in the range of about 50° C. to about 95° C.

Synthetic steps that require a base are carried out using any convenient organic or inorganic base. Typically, the base is not nucleophilic. Thus, in one embodiment, the base is selected from carbonates, phosphates, hydroxides, alkoxides, salts of disilazanes, and tertiary amines.

The process of the invention, when performed as described herein, can be substantially complete after several minutes to after several hours depending upon the nature and quantity of reactants and reaction temperature. The determination of when the reaction is substantially complete can be conveniently evaluated by ordinary techniques known in the art such as, for example, HPLC, LCMS, TLC, and ¹H NMR.

### G. Methods of Treatment

The invention encompasses methods of preventing or treating (e.g., alleviating one or more symptoms of) medical conditions through use of one or more of the disclosed compounds. The methods comprise administering a therapeutically effective amount of a compound of the invention to a patient in need. The compositions of the invention can also be used for prophylactic therapy.

The compound of the invention used in the methods of treatment according to the invention can be: (1) a novel compound described herein, or a pharmaceutically acceptable salt thereof, a stereoisomer thereof, a prodrug thereof, a metabolite thereof, or an N-oxide thereof; (2) a compound which was known prior to the present invention, but wherein it was not known that the compound is a GSNOR inhibitor, or a pharmaceutically acceptable salt thereof, a stereoisomer thereof, a prodrug thereof, a metabolite thereof, or an N-oxide thereof; or (3) a compound which was known prior to the present invention, and wherein it was known that the compound is a GSNOR inhibitor, but wherein it was not known that the compound is useful for the methods of treatment described herein, or a pharmaceutically acceptable salt, a stereoisomer, a prodrug, a metabolite, or an N-oxide thereof.

The patient can be any animal, domestic, livestock, or wild, including, but not limited to cats, dogs, horses, pigs, and cattle, and preferably human patients. As used herein, the terms patient and subject may be used interchangeably.

As used herein, “treating” describes the management and care of a patient for the purpose of combating a disease, condition, or disorder and includes the administration of a compound of the present invention to prevent the onset of the symptoms or complications, alleviating the symptoms or complications, or eliminating the disease, condition, or disorder. More specifically, “treating” includes reversing, attenuating, alleviating, minimizing, suppressing, or halting at least one deleterious symptom or effect of a disease (disorder) state, disease progression, disease causative agent (e.g., bacteria or viruses), or other abnormal condition. Treatment is continued as long as symptoms and/or pathology ameliorate.

In general, the dosage, i.e., the therapeutically effective amount, ranges from 1 μg/kg to 10 g/kg and often ranges from 10 μg/kg to 1 g/kg or 10 μg/kg to 100 mg/kg body weight of the subject being treated, per day.

### H. GSNOR Uses

In subjects with deleteriously high levels of GSNOR or GSNOR activity, modulation may be achieved, for example, by administering one or more of the disclosed compounds that disrupts or down-regulates GSNOR function, or decreases GSNOR levels. These compounds may be administered with other GSNOR inhibitor agents, such as anti-GSNOR antibodies or antibody fragments, GSNOR antisense, iRNA, or small molecules, or other inhibitors, alone or in combination with other agents as described in detail herein.

The present invention provides a method of treating a subject afflicted with a disorder ameliorated by NO donor therapy. Such a method comprises administering to a subject a therapeutically effective amount of a GSNOR inhibitor.

The disorders can include pulmonary disorders associated with hypoxemia and/or smooth muscle constriction in the lungs and airways and/or lung infection and/or lung inflammation and/or lung injury (e.g., pulmonary hypertension, ARDS, asthma, pneumonia, pulmonary fibrosis/interstitial lung diseases, cystic fibrosis, COPD); cardiovascular disease and heart disease (e.g., hypertension, ischemic coronary syndromes, atherosclerosis, heart failure, glaucoma); diseases characterized by angiogenesis (e.g., coronary artery disease); disorders where there is risk of thrombosis occurring; disorders where there is risk of restenosis occurring; inflammatory diseases (e.g., AIDS related dementia, inflammatory bowel disease (IBD), Crohn's disease, colitis, and psoriasis); functional bowel disorders (e.g., irritable bowel syndrome (IBS)); diseases where there is risk of apoptosis occurring (e.g., heart failure, atherosclerosis, degenerative neurologic disorders, arthritis, and liver injury (e.g., drug induced, ischemic or alcoholic)); impotence; sleep apnea; diabetic wound healing; cutaneous infections; treatment of psoriasis; obesity caused by eating in response to craving for food; stroke; reperfusion injury (e.g., traumatic muscle injury in heart or lung or crush injury); and disorders where preconditioning of heart or brain for NO protection against subsequent ischemic events is beneficial, central nervous system (CNS) disorders (e.g., anxiety, depression, psychosis, and schizophrenia); and infections caused by bacteria (e.g., tuberculosis, C. difficile infections, among others).

In one embodiment, the disorder is cystic fibrosis. Compounds of the invention are capable of treating and/or slowing the progression of cystic fibrosis. For approximately 90% of patients with CF, death results from progressive respiratory failure associated with impaired mucus clearance and excessive overgrowth of bacteria and fungi in the airways (Gibson et al., 2003, Proesmans et al., 2008). Compounds of the invention are capable of preserving endogenous s-nitrosothiol (SNO) pools via inhibiting GSNO catabolism and therefore may positively modulate CFTR. Compounds of the present invention are distinguished by their ability to demonstrate preservation of GSNO, potent bronchodilatory and anti-inflammatory effects in animal models of COPD (porcine pancreatic elastase) (Blonder et al., ATS 2011 abstract reference) and asthma. Compounds of the invention are capable of treating and/or slowing the progression of CF. In this embodiment, appropriate amounts of compounds of the present invention are an amount sufficient to treat and/or slow the progression of CF and can be determined without undue experimentation by preclinical and/or clinical trials.

In one embodiment, the disorder is liver injury. Liver injury can include, for example, acute liver toxicity. Acute liver toxicity can result in acute liver failure. Acute liver failure (ALF) is an uncommon but potentially lethal drug-related adverse effect that often leads to liver transplantation (LT) or death. Acetoaminophen is the most common cause of acute liver toxicity and acute liver failure, although acute liver toxicity can be due to other agents, such as alcohol and other drugs. Regardless of whether it occurs as a result of a single overdose or after repeated supratherapeutic ingestion, the progression of acetaminophen poisoning can be categorized into four stages: preclinical toxic effects (a normal serum alanine aminotransferase concentration), hepatic injury (an elevated alanine aminotransferase concentration), hepatic failure (hepatic injury with hepatic encephalopathy), and recovery. As long as sufficient glutathione is present, the liver is protected from injury. Overdoses of acetaminophen (either a single large ingestion or repeated supratherapeutic ingestion) can deplete hepatic glutathione stores and allow liver injury to occur. Compounds of the invention are capable of treating and/or preventing liver injury and/or acute liver toxicity. In this embodiment, appropriate amounts of compounds of the present invention are an amount sufficient to treat and/or prevent liver injury and can be determined without undue experimentation by preclinical and/or clinical trials. In one embodiment, the amount to treat is at least 0.001 mg/kg, at least 0.002 mg/kg, at least 0.003 mg/kg, at least 0.004 mg/kg, at least 0.005 mg/kg, at least 0.006 mg/kg, at least 0.007 mg/kg, at least 0.008 mg/kg, at least 0.009 mg/kg, at least 0.01 mg/kg, at least 0.02 mg/kg, at least 0.03 mg/kg, at least 0.04 mg/kg, at least 0.05 mg/kg, at least at least 0.06 mg/kg, at least 0.07 mg/kg, at least 0.08 mg/kg, at least 0.09 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 1.5 mg/kg, at least 2 mg/kg, at least 2.5 mg/kg, at least 3 mg/kg, at least 3.5 mg/kg, at least 4 mg/kg, at least 4.5 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, at least 100 mg/kg. The dosing can be hourly, four times, twice, or once daily, or four times, twice, or once per week, or weekly, or every other week, every third week, or monthly.

In one embodiment, the disorder is nonalcoholic steatohepatitis (NASH). Progression of this disease can lead to cirrhosis and eventually the need for liver transplantation. Compounds of the present invention may reverse fibrotic activity in nonalcoholic steatohepatitis (NASH)-induced liver disease. We have shown that inhibition of GSNOR by the use of highly specific small molecules treats, repairs, and promotes regeneration of mammalian tissue. Compounds of the invention are capable of treating and/or slowing the progression of NASH. In this embodiment, appropriate amounts of compounds of the present invention are an amount sufficient to treat NASH and can be determined without undue experimentation by preclinical and/or clinical trials.

In one embodiment, the disorder is trauma (including surgery and thermal), infectious, toxic, aging, and ischemic damage to organs of known regenerative capacity, such as skin, gastric mucosa, airway epithelial and cartilaginous structures, liver, neuronal structures such as the spinal cord, bone marrow and bone. We have shown that inhibition of GSNOR by the use of highly specific small molecules treats, repairs, and promotes regeneration of mammalian tissue. By way of example, small molecule inhibitors are effective in treating, and promoting repair and regeneration of mammalian lung tissue damaged by instillation of a chemical agent known to cause severe lung injury (porcine pancreatic elastase) (Blonder et al., ATS 2011 abstract reference). In this embodiment, appropriate amounts of compounds of the present invention are an amount sufficient to regenerate tissue/organs and can be determined without undue experimentation by preclinical and/or clinical trials.

In one embodiment the disorder is trauma (including surgery and thermal), infectious, toxic, aging, and ischemic damage to organs of not commonly known to have regenerative capacity. Examples include regeneration of: the heart, the lung, the kidney, the central nervous system, the peripheral nervous system, peripheral vascular tissue, liver, pancreas, adrenal gland, thyroid, testes, ovary, retina, tongue, bone, bladder, esophagus, larynx, thymus, spleen, cartilaginous structures of the head, and cartilaginous structures of the joints. In this embodiment, appropriate amounts of compounds of the present invention are an amount sufficient to regenerate tissue/organs and can be determined without undue experimentation by preclinical and/or clinical trials.

In one embodiment ex and in vivo implantation and regeneration of organs and structures, including stem cells. In this embodiment, appropriate amounts of compounds of the present invention are an amount sufficient to regenerate tissue/organs and can be determined without undue experimentation by preclinical and/or clinical trials.

In one embodiment, the compounds of the present invention or a pharmaceutically acceptable salt thereof, or a prodrug, stereoisomer, metabolite, or N-oxide thereof, can be administered in combination with an NO donor. An NO donor donates nitric oxide or a related redox species and more generally provides nitric oxide bioactivity, that is activity which is identified with nitric oxide, e.g., vasorelaxation or stimulation or inhibition of a receptor protein, e.g., ras protein, adrenergic receptor, NFκB. NO donors including S-nitroso, O-nitroso, C-nitroso, and N-nitroso compounds and nitro derivatives thereof and metal NO complexes, but not excluding other NO bioactivity generating compounds, useful herein are described in “Methods in Nitric Oxide Research,” Feelisch et al. eds., pages 71-115 (J. S., John Wiley & Sons, New York, 1996), which is incorporated herein by reference. NO donors which are C-nitroso compounds where nitroso is attached to a tertiary carbon which are useful herein include those described in U.S. Pat. No. 6,359,182 and in WO 02/34705. Examples of S-nitroso compounds, including S-nitrosothiols useful herein, include, for example, S-nitrosoglutathione, S-nitroso-N-acetylpenicillamine, S-nitroso-cysteine and ethyl ester thereof, S-nitroso cysteinyl glycine, S-nitroso-gamma-methyl-L-homocysteine, S-nitroso-L-homocysteine, S-nitroso-gamma-thio-L-leucine, S-nitroso-delta-thio-L-leucine, and S-nitrosoalbumin. Examples of other NO donors useful herein are sodium nitroprusside (nipride), ethyl nitrite, isosorbide, nitroglycerin, SIN 1 which is molsidomine, furoxamines, N-hydroxy (N-nitrosamine), and perfluorocarbons that have been saturated with NO or a hydrophobic NO donor.

The combination of a GSNOR inhibitor with R(+) enantiomer of amlodipine, a known NO releaser (Zhang at al., J. Cardiovasc. Pharm. 39: 208-214 (2002)) is also an embodiment of the present invention.

The present invention also provides a method of treating a subject afflicted with pathologically proliferating cells where the method comprises administering to said subject a therapeutically effective amount of an inhibitor of GSNOR. The inhibitors of GSNOR are the compounds as defined above, or a pharmaceutically acceptable salt thereof, or a stereoisomer, prodrug, metabolite, or N-oxide thereof, in combination with a pharmaceutically acceptable carrier. Treatment is continued as long as symptoms and/or pathology ameliorate.

In another embodiment, the pathologically proliferating cells can be pathologically proliferating microbes. The microbes involved can be those where GSNOR is expressed to protect the microbe from nitrosative stress or where a host cell infected with the microbe expresses the enzyme, thereby protecting the microbe from nitrosative stress. The term “pathologically proliferating microbes” is used herein to mean pathologic microorganisms including, but not limited to, pathologic bacteria, pathologic viruses, pathologic Chlamydia, pathologic protozoa, pathologic Rickettsia, pathologic fungi, and pathologic mycoplasmata. More detail on the applicable microbes is set forth at columns 11 and 12 of U.S. Pat. No. 6,057,367. The term “host cells infected with pathologic microbes” includes not only mammalian cells infected with pathologic viruses but also mammalian cells containing intracellular bacteria or protozoa, e.g., macrophages containing Mycobacterium tuberculosis, Mycobacterium leper (leprosy), or Salmonella typhi (typhoid fever).

In another embodiment, the pathologically proliferating cells can be pathologic helminths. The term “pathologic helminths” is used herein to refer to pathologic nematodes, pathologic trematodes and pathologic cestodes. More detail on the applicable helminths is set forth at column 12 of U.S. Pat. No. 6,057,367.

In another embodiment, the pathologically proliferating cells can be pathologically proliferating mammalian cells. The term “pathologically proliferating mammalian cells” as used herein means cells of the mammal that grow in size or number in said mammal so as to cause a deleterious effect in the mammal or its organs. The term includes, for example, the pathologically proliferating or enlarging cells causing restenosis, the pathologically proliferating or enlarging cells causing benign pro static hypertrophy, the pathologically proliferating cells causing myocardial hypertrophy, and proliferating cells at inflammatory sites such as synovial cells in arthritis or cells associated with a cell proliferation disorder.

As used herein, the term “cell proliferative disorder” refers to conditions in which the unregulated and/or abnormal growth of cells can lead to the development of an unwanted condition or disease, which can be cancerous or non-cancerous, for example a psoriatic condition. As used herein, the term “psoriatic condition” refers to disorders involving keratinocyte hyperproliferation, inflammatory cell infiltration, and cytokine alteration. The cell proliferative disorder can be a precancerous condition or cancer. The cancer can be primary cancer or metastatic cancer, or both.

As used herein, the term “cancer” includes solid tumors, such as lung, breast, colon, ovarian, pancreas, prostate, adenocarcinoma, squamous carcinoma, sarcoma, malignant glioma, leiomyosarcoma, hepatoma, head and neck cancer, malignant melanoma, non-melanoma skin cancers, as well as hematologic tumors and/or malignancies, such as leukemia, childhood leukemia and lymphomas, multiple myeloma, Hodgkin's disease, lymphomas of lymphocytic and cutaneous origin, acute and chronic leukemia such as acute lymphoblastic, acute myelocytic, or chronic myelocytic leukemia, plasma cell neoplasm, lymphoid neoplasm, and cancers associated with AIDS.

In addition to psoriatic conditions, the types of proliferative diseases which may be treated using the compositions of the present invention are epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses, and the like. In one embodiment, proliferative diseases include dysplasias and disorders of the like.

In one embodiment, treating cancer comprises a reduction in tumor size, decrease in tumor number, a delay of tumor growth, decrease in metastatic lesions in other tissues or organs distant from the primary tumor site, an improvement in the survival of patients, or an improvement in the quality of patient life, or at least two of the above.

In another embodiment, treating a cell proliferative disorder comprises a reduction in the rate of cellular proliferation, reduction in the proportion of proliferating cells, a decrease in size of an area or zone of cellular proliferation, or a decrease in the number or proportion of cells having an abnormal appearance or morphology, or at least two of the above.

In yet another embodiment, the compounds of the present invention or a pharmaceutically acceptable salt thereof, a stereoisomer thereof, a prodrug thereof, a metabolite thereof, or an N-oxide thereof can be administered in combination with a second chemotherapeutic agent. In a further embodiment, the second chemotherapeutic agent is selected from the group consisting of tamoxifen, raloxifene, anastrozole, exemestane, letrozole, cisplatin, carboplatin, paclitaxel, cyclophosphamide, lovastatin, minosine, gemcitabine, araC, 5-fluorouracil, methotrexate, docetaxel, goserelin, vincristin, vinblastin, nocodazole, teniposide, etoposide, epothilone, navelbine, camptothecin, daunonibicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab.

In one embodiment, the compounds of the present invention or a pharmaceutically acceptable salt thereof, a stereoisomer thereof, a prodrug thereof, a metabolite thereof, or an N-oxide thereof, can be administered in combination with an agent that imposes nitrosative or oxidative stress. Agents for selectively imposing nitrosative stress to inhibit proliferation of pathologically proliferating cells in combination therapy with GSNOR inhibitors herein and dosages and routes of administration therefor include those disclosed in U.S. Pat. No. 6,057,367, which is incorporated herein. Supplemental agents for imposing oxidative stress (i.e., agents that increase GSSG (oxidized glutathione) over GSH (glutathione) ratio or NAD(P) over NAD(P)H ratio or increase thiobarbituric acid derivatives) in combination therapy with GSNOR inhibitors herein include, for example, L-buthionine-S-sulfoximine (BSO), glutathione reductase inhibitors (e.g., BCNU), inhibitors or uncouplers of mitochondrial respiration, and drugs that increase reactive oxygen species (ROS), e.g., adriamycin, in standard dosages with standard routes of administration.

GSNOR inhibitors may also be co-administered with a phosphodiesterase inhibitor (e.g., rolipram, cilomilast, roflumilast, Viagra® (sildenifil citrate), Cialis® (tadalafil), Levitra® (vardenifil), etc.), a β-agonist, a steroid, or a leukotriene antagonist (LTD-4). Those skilled in the art can readily determine the appropriate therapeutically effective amount depending on the disorder to be ameliorated.

GSNOR inhibitors may be used as a means to improve β-adrenergic signaling. In particular, inhibitors of GSNOR alone or in combination with β-agonists could be used to treat or protect against heart failure, or other vascular disorders such as hypertension and asthma. GSNOR inhibitors can also be used to modulate G protein coupled receptors (GPCRs) by potentiating Gs G-protein, leading to smooth muscle relaxation (e.g., airway and blood vessels), and by attenuating Gq G-protein, and thereby preventing smooth muscle contraction (e.g., in airway and blood vessels).

The therapeutically effective amount for the treatment of a subject afflicted with a disorder ameliorated by NO donor therapy is the GSNOR inhibiting amount in vivo that causes amelioration of the disorder being treated or protects against a risk associated with the disorder. For example, for asthma, a therapeutically effective amount is a bronchodilating effective amount; for cystic fibrosis, a therapeutically effective amount is an airway obstruction ameliorating effective amount; for ARDS, a therapeutically effective amount is a hypoxemia ameliorating effective amount; for heart disease, a therapeutically effective amount is an angina relieving or angiogenesis inducing effective amount; for hypertension, a therapeutically effective amount is a blood pressure reducing effective amount; for ischemic coronary disorders, a therapeutic amount is a blood flow increasing effective amount; for atherosclerosis, a therapeutically effective amount is an endothelial dysfunction reversing effective amount; for glaucoma, a therapeutic amount is an intraocular pressure reducing effective amount; for diseases characterized by angiogenesis, a therapeutically effective amount is an angiogenesis inhibiting effective amount; for disorders where there is risk of thrombosis occurring, a therapeutically effective amount is a thrombosis preventing effective amount; for disorders where there is risk of restenosis occurring, a therapeutically effective amount is a restenosis inhibiting effective amount; for chronic inflammatory diseases, a therapeutically effective amount is an inflammation reducing effective amount; for disorders where there is risk of apoptosis occurring, a therapeutically effective amount is an apoptosis preventing effective amount; for impotence, a therapeutically effective amount is an erection attaining or sustaining effective amount; for obesity, a therapeutically effective amount is a satiety causing effective amount; for stroke, a therapeutically effective amount is a blood flow increasing or a TIA protecting effective amount; for reperfusion injury, a therapeutically effective amount is a function increasing effective amount; and for preconditioning of heart and brain, a therapeutically effective amount is a cell protective effective amount, e.g., as measured by troponin or CPK.

The therapeutically effective amount for the treatment of a subject afflicted with pathologically proliferating cells means a GSNOR inhibiting amount in vivo which is an antiproliferative effective amount. Such antiproliferative effective amount as used herein means an amount causing reduction in rate of proliferation of at least about 20%, at least about 10%, at least about 5%, or at least about 1%.

### I. Uses in an Apparatus

The compounds of the present invention or a pharmaceutically acceptable salt thereof, or a stereoisomer, prodrug, metabolite, or N-oxide thereof, can be applied to various apparatus in circumstances when the presence of such compounds would be beneficial. Such apparatus can be any device or container, for example, implantable devices in which a compound of the invention can be used to coat a surgical mesh or cardiovascular stent prior to implantation in a patient. The compounds of the invention can also be applied to various apparatus for in vitro assay purposes or for culturing cells.

The compounds of the present invention or a pharmaceutically acceptable salt thereof, or a stereoisomer, a prodrug, a metabolite, or an N-oxide thereof, can also be used as an agent for the development, isolation or purification of binding partners to compounds of the invention, such as antibodies, natural ligands, and the like. Those skilled in the art can readily determine related uses for the compounds of the present invention.

### EXAMPLES

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1

### Compounds

Example 1 includes 8 tables that list representative analogs of Formula I useful as GSNOR inhibitors of the invention. Methods of making analogs of Formula I are described in prior patent applications as referenced before the tables or are described in Example 2. Supporting mass spectrometry data and/or proton NMR data for compounds not previously described is also included for such compounds. GSNOR inhibitor activity was determined by the assay described in Example 3 and IC₅₀ values were obtained. GSNOR activity is described as a range in the tables in the following manner: an IC₅₀ value <100 nM is designated the letter a, an IC₅₀ range of 100 nM-1 μM is designated the letter b, and an IC₅₀ range of 1 μM-10 μM is designated the letter c.

The tables also include the distance measurement taken after MM2 calculation, as discussed in the Inventive Compounds section of the present application.

In the tables below, the * is defined as the point of connection between portions of the molecule. The * on Cy₁ and Cy₂ show where on each ring the linker is connected to the ring. The *s on the linker signify connection to the Cy₁ and Cy₂, whereby the left side of the linker is connected to Cy₁ at the * on Cy₁, and the right side of the linker is connected to the Cy₂ at the * on Cy₂. For example, the structure of compound I-1 found in Table 1 below is

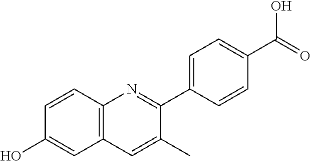 [SMILES: Cc1cc2cc(O)ccc2nc1-c1ccc(C(=O)O)cc1]

and is represented in Table 1 by the following description:

| HO—Cy₁ | linker | Cy₂-acidic moiety |
|---|---|---|
| 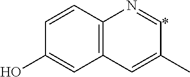 | *—* | 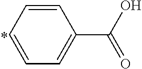 |

The * on the Cy₁ is joined with the * on the left side of the linker, which in this case is a bond. The * on the Cy₂ is connected to the * on the right side of the linker, the other side of the bond.

Another example is compound VII-1 found in Table 7 below is

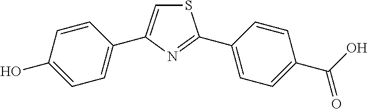 [SMILES: O=C(O)c1ccc(-c2nc(-c3ccc(O)cc3)cs2)cc1]

and is represented by the following description:

| HO—Cy₁ | linker | Cy₂-acidic moiety |
|---|---|---|
| 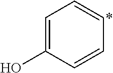 | 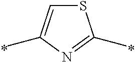 | 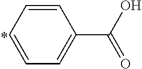 |

The * on the Cy₁ is joined with the * on the left side of the linker as shown, which in this case is the 4 position of the thiazole ring. The * on the Cy₂ is connected to the * on the right side of the linker, which in this case is the 2 position of the thiazole ring.

Table 1 below lists example compounds of Formula 1 with a quinoline core that have been synthesized and an IC₅₀ value was obtained for each (see Example 3 for method) and is represented below by a, b, or c as described in Example 1 before the tables. Many of the compounds in Table 1 were previously described in PCT US2011/055200 filed on Oct. 8, 2011 and PCT US2011/065490 filed on Dec. 16, 2011. The synthetic descriptions for compounds not previously described are found in Example 2.

TABLE 1

| | | | | | linear | Distance |
|---|---|---|---|---|---|---|
| | | | | IC₅₀ | rotatable | after MM2 |
| # | HO—Cy₁ | linker | Cy₂-acidic moiety | range | bonds | min. |
| I-1 | 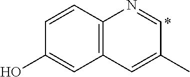 | *—* | 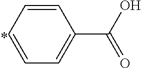 | a | 2 | 12.0 |
| I-2 | 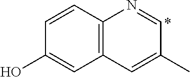 | *—* | 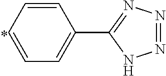 [SMILES: c1ccc(-c2nnn[nH]2)cc1] | a | 2 | 12 |
| I-3 | 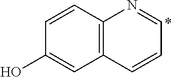 [SMILES: Oc1ccc2ncccc2c1] | *—* | 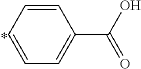 | a | 2 | 12.1 |
| I-4 | 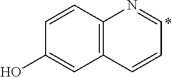 [SMILES: Oc1ccc2ncccc2c1] | *—* | 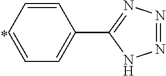 [SMILES: c1ccc(-c2nnn[nH]2)cc1] | a | 2 | 12.1 |
| I-5 | 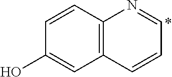 [SMILES: Oc1ccc2ncccc2c1] | *—* | 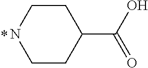 | c | 2 | 11.2 |
| I-6 | 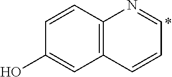 [SMILES: Oc1ccc2ncccc2c1] | 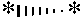 | 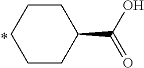 | b | 2 | 11.8 |
| I-7 | 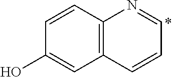 [SMILES: Oc1ccc2ncccc2c1] | 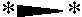 | 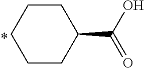 | c | 2 | 9.1 |
| I-8 | 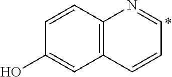 [SMILES: Oc1ccc2ncccc2c1] | *—* | 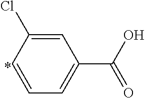 | a | 2 | 12.1 |
| I-9 | 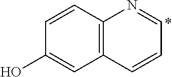 [SMILES: Oc1ccc2ncccc2c1] | *—* | 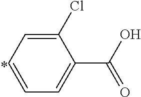 | c | 2 | 12.1 |
| I-10 | 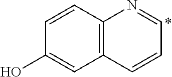 [SMILES: Oc1ccc2ncccc2c1] | *—* | 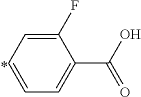 | a | 2 | 12.1 |
| I-11 | 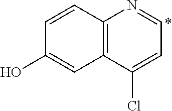 [SMILES: Oc1ccc2nccc(Cl)c2c1] | *—* | 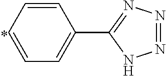 [SMILES: c1ccc(-c2nnn[nH]2)cc1] | a | 2 | 12.1 |
| I-12 | 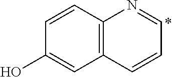 [SMILES: Oc1ccc2ncccc2c1] | *—* | 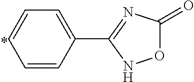 [SMILES: O=c1nc(-c2ccccc2)[nH]o1] | a | 2 | 12.1 |
| I-13 | 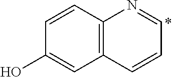 [SMILES: Oc1ccc2ncccc2c1] | *—* | 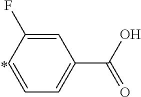 | a | 2 | 12.1 |
| I-14 | 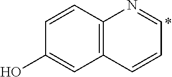 [SMILES: Oc1ccc2ncccc2c1] | *—* | 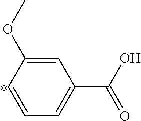 | a | 2 | 12.1 |
| I-15 | 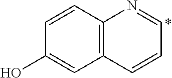 [SMILES: Oc1ccc2ncccc2c1] | *—* | 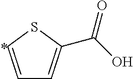 | c | 2 | 11.7 |
| I-16 | 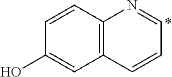 [SMILES: Oc1ccc2ncccc2c1] | *—* | 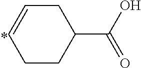 | b | 2 | 10.9 |
| I-17 | 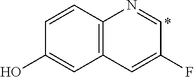 [SMILES: Oc1ccc2ncc(F)cc2c1] | *—* | 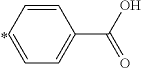 | a | 2 | 12.1 |
| I-18 | 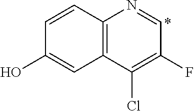 [SMILES: Oc1ccc2ncc(F)c(Cl)c2c1] | *—* | 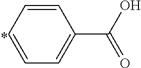 | a | 2 | 12 |
| I-19 | 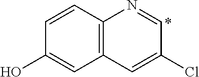 [SMILES: Oc1ccc2ncc(Cl)cc2c1] | *—* | 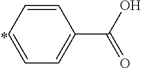 | a | 2 | 12.1 |
| I-20 | 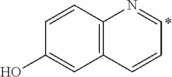 [SMILES: Oc1ccc2ncccc2c1] | *—* | 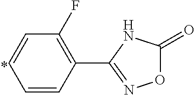 [SMILES: O=c1[nH]c(-c2ccccc2F)no1] | a | 2 | 12.1 |
| I-21 | 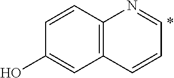 [SMILES: Oc1ccc2ncccc2c1] | *—* | 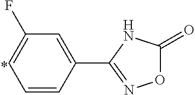 [SMILES: O=c1[nH]c(-c2cccc(F)c2)no1] | a | 2 | 12.1 |
| I-22 | 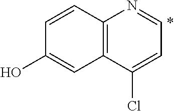 [SMILES: Oc1ccc2nccc(Cl)c2c1] | *—* | 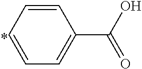 | a | 2 | 12.1 |
| I-23 | 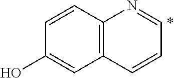 [SMILES: Oc1ccc2ncccc2c1] | *—* | 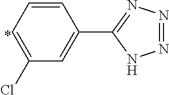 [SMILES: Clc1cccc(-c2nnn[nH]2)c1] | a | 2 | 12 |
| I-24 | 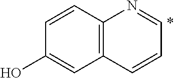 [SMILES: Oc1ccc2ncccc2c1] | *—* | 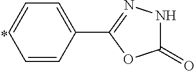 [SMILES: O=c1[nH]nc(-c2ccccc2)o1] | a | 2 | 12.1 |
| I-25 | 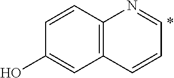 [SMILES: Oc1ccc2ncccc2c1] | *—* | 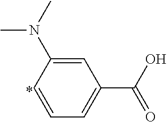 | a | 2 | 12 |
| I-26 | 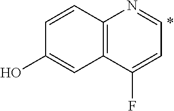 [SMILES: Oc1ccc2nccc(F)c2c1] | *—* | 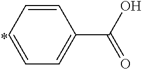 | a | 2 | 12.1 |
| I-27 | 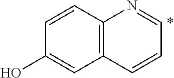 [SMILES: Oc1ccc2ncccc2c1] | *—* | 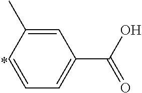 | a | 2 | 12.1 |
| I-28 | 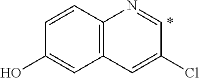 [SMILES: Oc1ccc2ncc(Cl)cc2c1] | *—* | 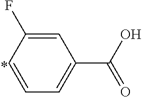 | a | 2 | 12.1 |
| I-29 | 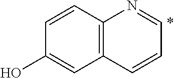 [SMILES: Oc1ccc2ncccc2c1] | *—* | 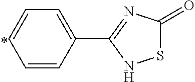 [SMILES: O=c1nc(-c2ccccc2)[nH]s1] | b | 2 | 12.1 |
| I-30 | 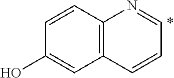 [SMILES: Oc1ccc2ncccc2c1] | *—* | 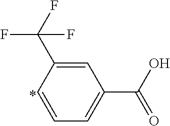 [SMILES: O=C(O)c1cccc(C(F)(F)F)c1] | a | 2 | 12.1 |
| I-31 | 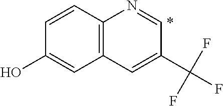 [SMILES: Oc1ccc2ncc(C(F)(F)F)cc2c1] | *—* | 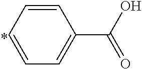 | a | 2 | 12.1 |
| I-32 | 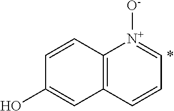 [SMILES: [O-][n+]1cccc2cc(O)ccc21] | *—* | 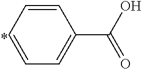 | b | 2 | 12.2 |
| I-33 | 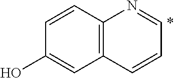 [SMILES: Oc1ccc2ncccc2c1] | *—* | 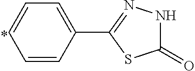 [SMILES: O=c1[nH]nc(-c2ccccc2)s1] | b | 2 | 12.1 |
| I-34 | 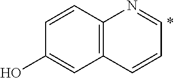 [SMILES: Oc1ccc2ncccc2c1] | *—* | 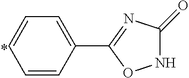 [SMILES: O=c1nc(-c2ccccc2)o[nH]1] | b | 2 | 12.1 |
| I-35 | 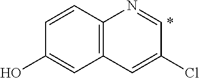 [SMILES: Oc1ccc2ncc(Cl)cc2c1] | 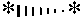 | 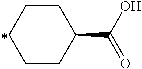 | b | 2 | 11.8 |
| I-36 | 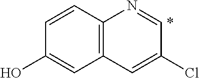 [SMILES: Oc1ccc2ncc(Cl)cc2c1] | 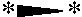 | 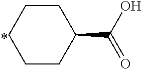 | c | 2 | 8.9 |
| I-37 | 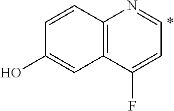 [SMILES: Oc1ccc2nccc(F)c2c1] | *—* | 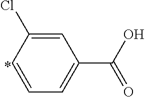 | a | 2 | 12.1 |
| I-38 | 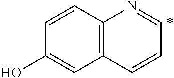 [SMILES: Oc1ccc2ncccc2c1] | *—* | 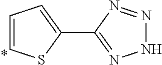 | b | 2 | 11.7 |
| I-39 | 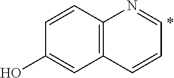 [SMILES: Oc1ccc2ncccc2c1] | *—* | 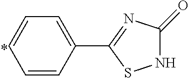 [SMILES: O=c1nc(-c2ccccc2)s[nH]1] | b | 2 | 12.1 |
| I-40 | 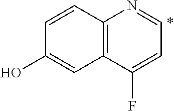 [SMILES: Oc1ccc2nccc(F)c2c1] | *—* | 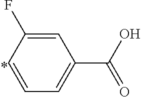 | a | 2 | 12.1 |
| I-41 | 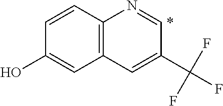 [SMILES: Oc1ccc2ncc(C(F)(F)F)cc2c1] | *—* | 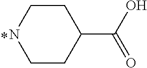 | a | 2 | 11.1 |
| I-42 | 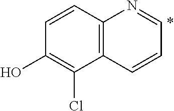 [SMILES: Oc1ccc2ncccc2c1Cl] | *—* | 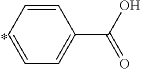 | b | 2 | 12.1 |
| I-43 | 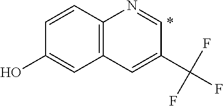 [SMILES: Oc1ccc2ncc(C(F)(F)F)cc2c1] | 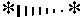 | 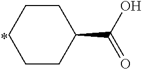 | a | 2 | 11.8 |
| I-44 | 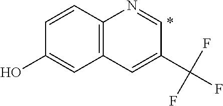 [SMILES: Oc1ccc2ncc(C(F)(F)F)cc2c1] | 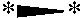 | 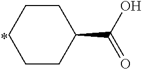 | c | 2 | 8.9 |
| I-45 | 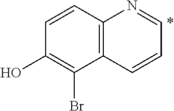 [SMILES: Oc1ccc2ncccc2c1Br] | *—* | 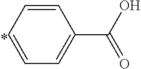 | b | 2 | 12.1 |
| I-46 | 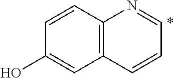 [SMILES: Oc1ccc2ncccc2c1] | *—* | 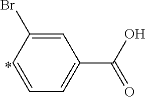 | a | 2 | 12.1 |
| I-47 | 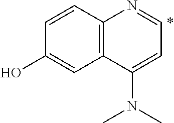 | *—* | 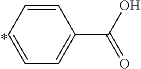 | b | 2 | 12 |
| I-48 | 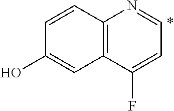 [SMILES: Oc1ccc2nccc(F)c2c1] | *—* | 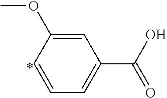 | a | 2 | 12.1 |
| I-49 | 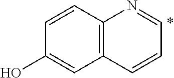 [SMILES: Oc1ccc2ncccc2c1] | *—* | 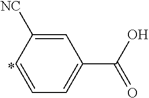 [SMILES: N#Cc1cccc(C(=O)O)c1] | a | 2 | 12.1 |
| I-50 | 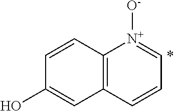 [SMILES: [O-][n+]1cccc2cc(O)ccc21] | *—* | 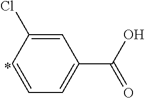 | b | 2 | 12.2 |
| I-51 | 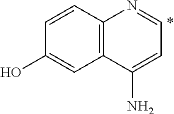 [SMILES: Nc1ccnc2ccc(O)cc12] | *—* | 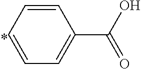 | c | 2 | 12.1 |
| I-52 | 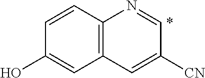 [SMILES: N#Cc1cnc2ccc(O)cc2c1] | *—* | 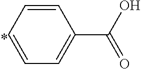 | a | 2 | 12 |
| I-53 | 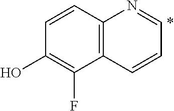 [SMILES: Oc1ccc2ncccc2c1F] | *—* | 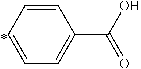 | a | 2 | 12.1 |
| I-54 | 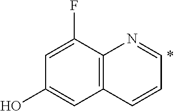 [SMILES: Oc1cc(F)c2ncccc2c1] | *—* | 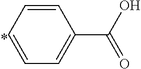 | a | 2 | 12.1 |
| I-55 | 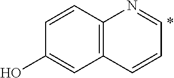 [SMILES: Oc1ccc2ncccc2c1] | *—* | 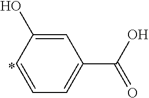 | a | 2 | 12.1 |
| I-56 | 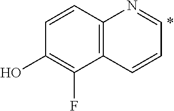 [SMILES: Oc1ccc2ncccc2c1F] | *—* | 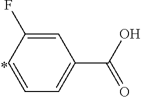 | a | 2 | 12.0 |
| I-57 | 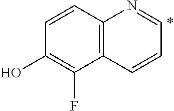 [SMILES: Oc1ccc2ncccc2c1F] | *—* | 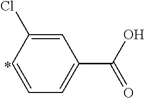 | a | 2 | 12.0 |
| I-58 | 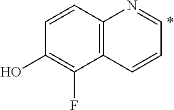 [SMILES: Oc1ccc2ncccc2c1F] | *—* | 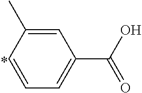 | a | 2 | 12.0 |
| I-59 | 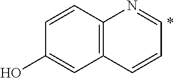 [SMILES: Oc1ccc2ncccc2c1] | *—* | 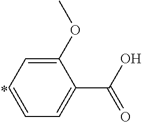 | b | 2 | 12.1 |
| I-60 | 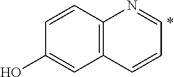 [SMILES: Oc1ccc2ncccc2c1] | *—* | 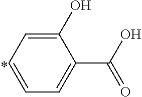 | a | 2 | 12.1 |
| I-61 | 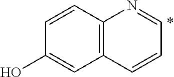 [SMILES: Oc1ccc2ncccc2c1] | *—* | 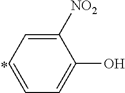 | c | 2 | 12.0^{†} |

^{†}signifies that for the cases where the acidic moiety is nitrophenol, the distance calculation is taken from the O of the hydroxyl on the bicyclic ring to the atom connected to the 4 position of phenyl.

Table 2 below lists compounds of Formula 1 that have been synthesized and an IC₅₀ value was obtained for each (see Example 3 for method) and is represented below by a, b, or c as described in Example 1 before the tables. Many of the compounds in Table 2 were previously described in PCT US2011/065490 filed on Dec. 16, 2011. The synthetic descriptions for compounds not previously described are found in Example 2.

TABLE 2

| | | | | | linear | Distance |
|---|---|---|---|---|---|---|
| | | | | | rotatable | after MM2 |
| # | HO—Cy₁ | linker | Cy₂-acidic moiety | IC₅₀ | bonds | min. |
| II-1 | 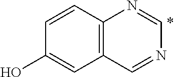 [SMILES: Oc1ccc2ncncc2c1] | *—* | 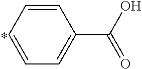 | a | 2 | 12.1 |
| II-2 | 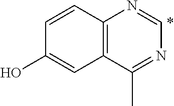 | *—* | 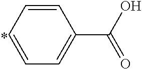 | c | 2 | 12.1 |
| II-3 | 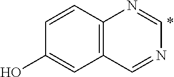 [SMILES: Oc1ccc2ncncc2c1] | *—* | 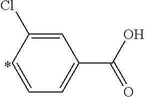 | a | 2 | 12.1 |
| II-4 | 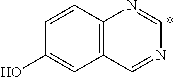 [SMILES: Oc1ccc2ncncc2c1] | *—* | 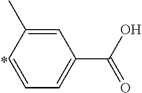 | a | 2 | 12.1 |
| II-5 | 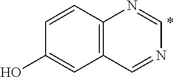 [SMILES: Oc1ccc2ncncc2c1] | *—* | 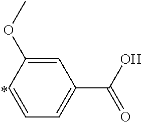 | a | 2 | 12.1 |
| II-6 | 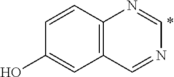 [SMILES: Oc1ccc2ncncc2c1] | *—* | 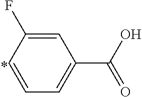 | a | 2 | 12.1 |
| II-7 | 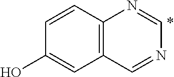 [SMILES: Oc1ccc2ncncc2c1] | *—* | 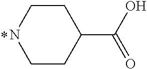 | c | 2 | 11.3 |
| II-8 | 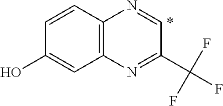 [SMILES: Oc1ccc2ncc(C(F)(F)F)nc2c1] | *—* | 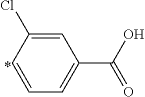 | a | 2 | 12 |
| II-9 | 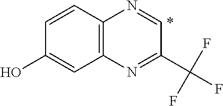 [SMILES: Oc1ccc2ncc(C(F)(F)F)nc2c1] | *—* | 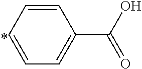 | a | 2 | 12 |
| II-10 | 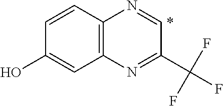 [SMILES: Oc1ccc2ncc(C(F)(F)F)nc2c1] | *—* | 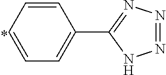 [SMILES: c1ccc(-c2nnn[nH]2)cc1] | a | 2 | 12 |
| II-11 | 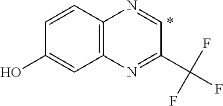 [SMILES: Oc1ccc2ncc(C(F)(F)F)nc2c1] | *—* | 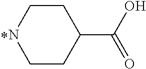 | a | 2 | 11.4 |
| II-12 | 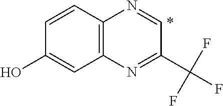 [SMILES: Oc1ccc2ncc(C(F)(F)F)nc2c1] | *—* | 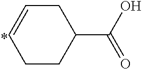 | a | 2 | 10.6 |
| II-13 | 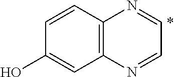 [SMILES: Oc1ccc2nccnc2c1] | *—* | 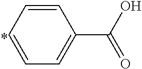 | a | 2 | 12.1 |
| II-14 | 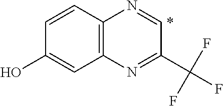 [SMILES: Oc1ccc2ncc(C(F)(F)F)nc2c1] | *—* | 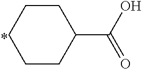 | ND | 2 | 11.6 |
| II-15 | 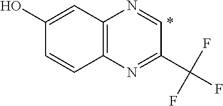 [SMILES: Oc1ccc2nc(C(F)(F)F)cnc2c1] | *—* | 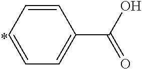 | b | 2 | 11.2 |
| II-16 | 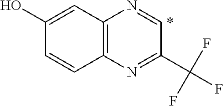 [SMILES: Oc1ccc2nc(C(F)(F)F)cnc2c1] | *—* | 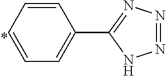 [SMILES: c1ccc(-c2nnn[nH]2)cc1] | b | 2 | 11 |
| II-17 | 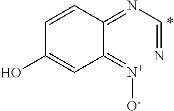 [SMILES: N=C/N=C1/C=CC(O)=C/C1=[NH+]\[O-]] | *—* | 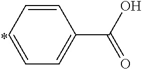 | a | 2 | 12.1 |
| II-18 | 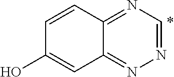 [SMILES: Oc1ccc2ncnnc2c1] | *—* | 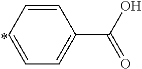 | b | 2 | 12 |
| II-19 | 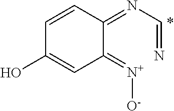 [SMILES: N=C/N=C1/C=CC(O)=C/C1=[NH+]\[O-]] | *—* | 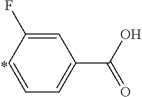 | a | 2 | 12.1 |
| II-20 | 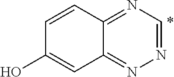 [SMILES: Oc1ccc2ncnnc2c1] | *—* | 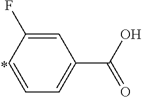 | a | 2 | 12 |
| II-21 | 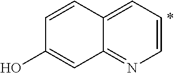 [SMILES: Oc1ccc2cccnc2c1] | *—* | 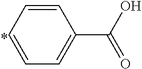 | c | 2 | 12.2 |
| II-22 | 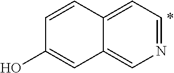 [SMILES: Oc1ccc2ccncc2c1] | *—* | 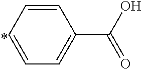 | b | 2 | 12.2 |
| II-23 | 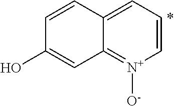 [SMILES: [O-][n+]1cccc2ccc(O)cc21] | *—* | 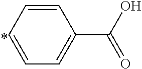 | c | 2 | 12.2 |
| II-24 | 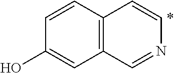 [SMILES: Oc1ccc2ccncc2c1] | *—* | 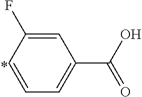 | a | 2 | 12.2 |
| II-25 | 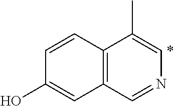 | *—* | 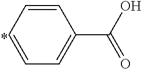 | b | 2 | 12.2 |
| II-26 | 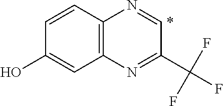 [SMILES: Oc1ccc2ncc(C(F)(F)F)nc2c1] | *—* | 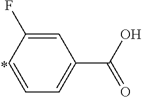 | a | 2 | 12 |
| II-27 | | 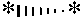 | | a | 2 | 12.2 |
| II-28 | 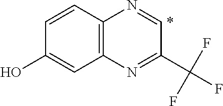 [SMILES: Oc1ccc2ncc(C(F)(F)F)nc2c1] | 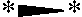 | 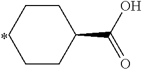 | c | 2 | 12.1 |

Table 3 below lists compounds of Formula 1 that have been synthesized and an IC₅₀ value was obtained for each (see Example 3 for method) and is represented below by a, b, or c as described in Example 1 before the tables. Many of the compounds in Table 3 were previously described in PCT US2011/065502 filed on Dec. 16, 2010. The synthetic descriptions for compounds not previously described are found in Example 2.

TABLE 3

| | | | | | linear | Distance |
|---|---|---|---|---|---|---|
| | | | | | rotatable | after MM2 |
| # | HO—Cy₁ | linker | Cy₂-acidic moiety | IC₅₀ | bonds | min. |
| III-1 | 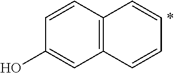 [SMILES: Oc1ccc2ccccc2c1] | *—* | 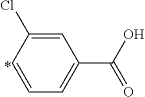 | a | 2 | 11.8 |
| III-2 | 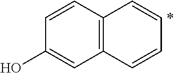 [SMILES: Oc1ccc2ccccc2c1] | *—* | 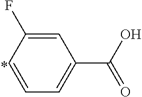 | a | 2 | 11.8 |
| III-3 | 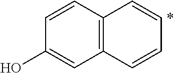 [SMILES: Oc1ccc2ccccc2c1] | *—* | 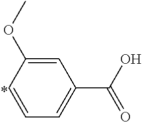 | a | 2 | 11.7 |
| III-4 | 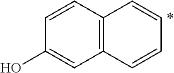 [SMILES: Oc1ccc2ccccc2c1] | *—* | 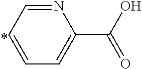 | c | 2 | 11.7 |
| III-5 | 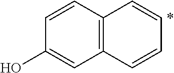 [SMILES: Oc1ccc2ccccc2c1] | *—* | 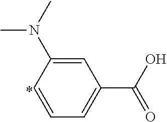 | a | 2 | 11.5 |
| III-6 | 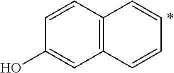 [SMILES: Oc1ccc2ccccc2c1] | *—* | 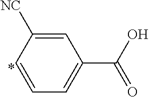 [SMILES: N#Cc1cccc(C(=O)O)c1] | a | 2 | 11.8 |
| III-7 | 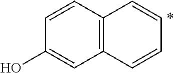 [SMILES: Oc1ccc2ccccc2c1] | *—* | 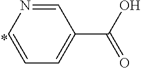 | b | 2 | 11.6 |
| III-8 | 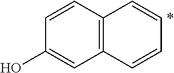 [SMILES: Oc1ccc2ccccc2c1] | *—* | 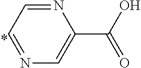 | b | 2 | 11.6 |
| III-9 | 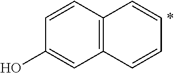 [SMILES: Oc1ccc2ccccc2c1] | *—* | 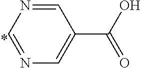 | b | 2 | 11.6 |
| III-10 | 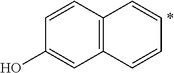 [SMILES: Oc1ccc2ccccc2c1] | *—* | 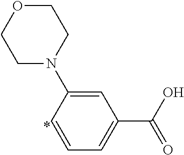 [SMILES: O=C(O)c1cccc(N2CCOCC2)c1] | b | 2 | 11.5 |
| III-11 | 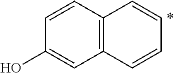 [SMILES: Oc1ccc2ccccc2c1] | *—* | 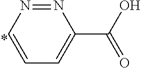 | c | 2 | 11.5 |
| III-12 | 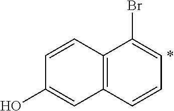 [SMILES: Oc1ccc2c(Br)cccc2c1] | *—* | 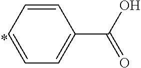 | a | 2 | 11.8 |
| III-13 | 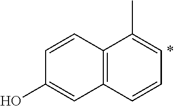 | *—* | 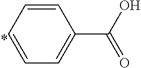 | b | 2 | 11.7 |
| III-14 | 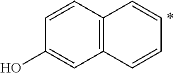 [SMILES: Oc1ccc2ccccc2c1] | *—* | 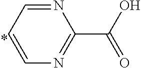 | c | 2 | 11.7 |
| III-15 | 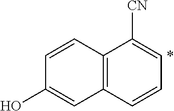 [SMILES: N#Cc1cccc2cc(O)ccc12] | *—* | 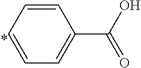 | a | 2 | 11.8 |
| III-16 | 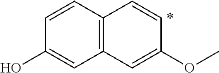 | *—* | 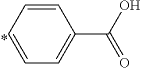 | c | 2 | 11.8 |
| III-17 | 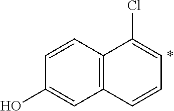 [SMILES: Oc1ccc2c(Cl)cccc2c1] | *—* | 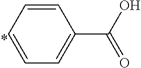 | a | 2 | 11.8 |
| III-18 | 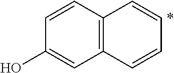 [SMILES: Oc1ccc2ccccc2c1] | *—* | 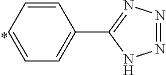 [SMILES: c1ccc(-c2nnn[nH]2)cc1] | a | 2 | 11.7 |
| III-19 | 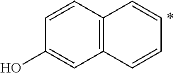 [SMILES: Oc1ccc2ccccc2c1] | *—* | 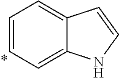 | c | 2 | 12 |
| III-20 | 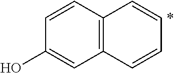 [SMILES: Oc1ccc2ccccc2c1] | *—* | 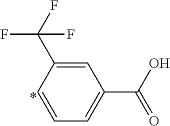 [SMILES: O=C(O)c1cccc(C(F)(F)F)c1] | a | 2 | 12.1 |
| III-21 | 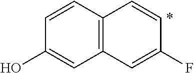 [SMILES: Oc1ccc2ccc(F)cc2c1] | *—* | 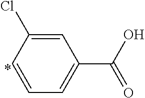 | a | 2 | 12.1 |
| III-22 | 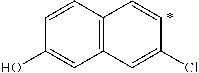 [SMILES: Oc1ccc2ccc(Cl)cc2c1] | *—* | 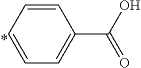 | a | 2 | 12.2 |
| III-23 | 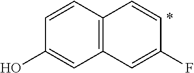 [SMILES: Oc1ccc2ccc(F)cc2c1] | *—* | 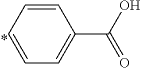 | a | 2 | 12.2 |
| III-24 | 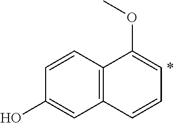 | *—* | 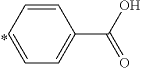 | a | 2 | 12.2 |
| III-25 | 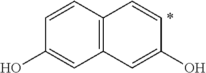 [SMILES: Oc1ccc2ccc(O)cc2c1] | *—* | 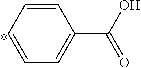 | c | 2 | 12.2 |
| III-26 | 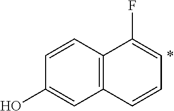 [SMILES: Oc1ccc2c(F)cccc2c1] | *—* | 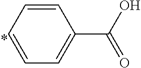 | a | 2 | 12.1 |
| III-27 | 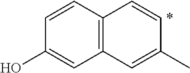 | *—* | 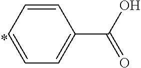 | a | 2 | 12.3 |
| III-28 | 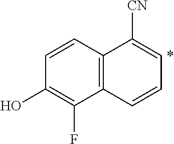 [SMILES: N#Cc1cccc2c(F)c(O)ccc12] | *—* | 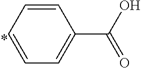 | a | 2 | 12.4 |
| III-29 | 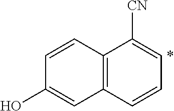 [SMILES: N#Cc1cccc2cc(O)ccc12] | *—* | 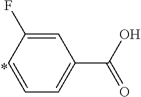 | a | 2 | 12.2 |
| III-30 | 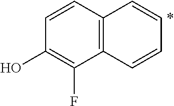 [SMILES: Oc1ccc2ccccc2c1F] | *—* | 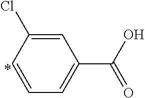 | a | 2 | 12.2 |
| III-31 | 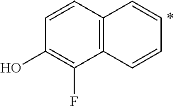 [SMILES: Oc1ccc2ccccc2c1F] | *—* | 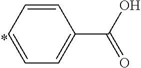 | a | 2 | 12.3 |
| III-32 | 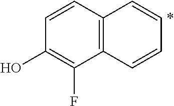 [SMILES: Oc1ccc2ccccc2c1F] | *—* | 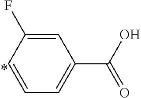 | a | 2 | 12.3 |
| III-33 | 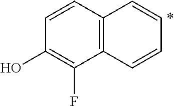 [SMILES: Oc1ccc2ccccc2c1F] | *—* | 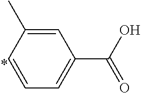 | a | 2 | 12.3 |
| III-34 | 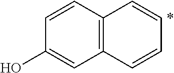 [SMILES: Oc1ccc2ccccc2c1] | *—* | 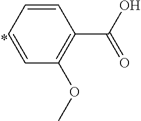 | b | 2 | 12.3 |
| III-35 | 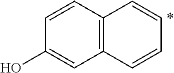 [SMILES: Oc1ccc2ccccc2c1] | *—* | 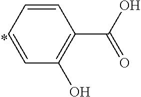 | a | 2 | 12.3 |
| III-36 | 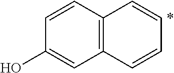 [SMILES: Oc1ccc2ccccc2c1] | *—* | 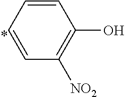 | b | 2 | 12.2 |
| III-37 | 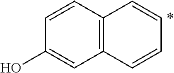 [SMILES: Oc1ccc2ccccc2c1] | *—* | 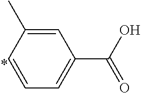 | a | 2 | 12.3 |
| III-38 | 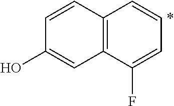 [SMILES: Oc1ccc2cccc(F)c2c1] | *—* | 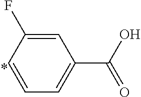 | a | 2 | 12.3 |
| III-39 | 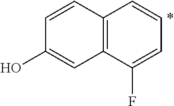 [SMILES: Oc1ccc2cccc(F)c2c1] | *—* | 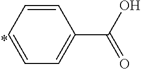 | a | 2 | 12.3 |
| III-40 | 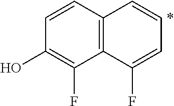 [SMILES: Oc1ccc2cccc(F)c2c1F] | *—* | 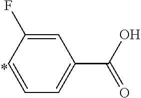 | a | 2 | 12.3 |
| III-41 | 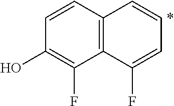 [SMILES: Oc1ccc2cccc(F)c2c1F] | *—* | 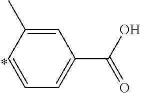 | a | 2 | 12.1 |
| III-42 | 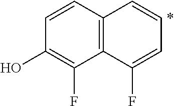 [SMILES: Oc1ccc2cccc(F)c2c1F] | *—* | 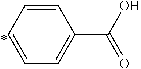 | a | 2 | 12.3 |
| III-43 | 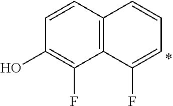 [SMILES: Oc1ccc2cccc(F)c2c1F] | *—* | 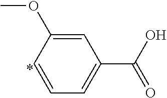 | a | 2 | 12.2 |

Table 4 below lists compounds of Formula 1 that have been synthesized and an IC₅₀ value was obtained for each (see Example 3 for method) and is represented below by a, b, or c as described in Example 1 before the tables. Many of the compounds in Table 4 were previously described in PCT US2010/024035 filed Feb. 12, 2010 and in PCT US2011/024353 filed Feb. 10, 2011. The synthetic descriptions for compounds not previously described are found in Example 2.

TABLE 4

| | | | | | linear | Distance |
|---|---|---|---|---|---|---|
| | | | Cy₂-acidic | | rotatable | after MM2 |
| # | HO—Cy₁ | linker | moiety | IC₅₀ | bonds | min. |
| IV-1 | 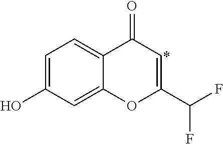 [SMILES: O=c1cc(C(F)F)oc2cc(O)ccc12] | *—* | 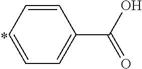 | a | 2 | 12.1 |
| IV-2 | 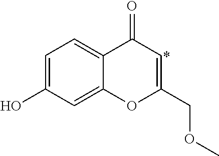 | *—* | 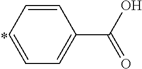 | b | 2 | 12.1 |
| IV-3 | 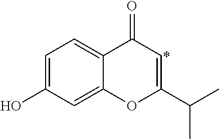 | *—* | 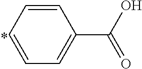 | b | 2 | 12.1 |
| IV-4 | 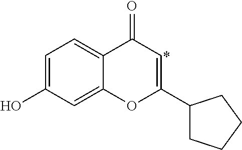 [SMILES: O=c1cc(C2CCCC2)oc2cc(O)ccc12] | *—* | 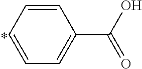 | c | 2 | 12.1 |
| IV-5 | 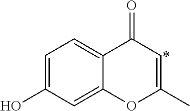 | *—* | 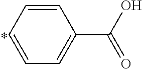 | b | 2 | 12.1 |
| IV-6 | 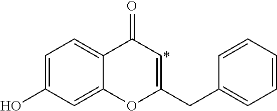 [SMILES: O=c1cc(Cc2ccccc2)oc2cc(O)ccc12] | *—* | 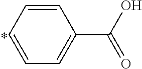 | b | 2 | 12.1 |
| IV-7 | 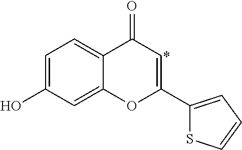 [SMILES: O=c1cc(-c2cccs2)oc2cc(O)ccc12] | *—* | 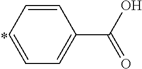 | c | 2 | 12.2 |
| IV-8 | 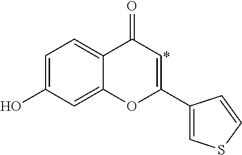 [SMILES: O=c1cc(-c2ccsc2)oc2cc(O)ccc12] | *—* | 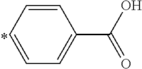 | c | 2 | 12.2 |
| IV-9 | 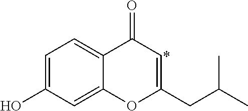 [SMILES: CC(C)Cc1cc(=O)c2ccc(O)cc2o1] | *—* | 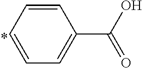 | b | 2 | 12.1 |
| IV-10 | 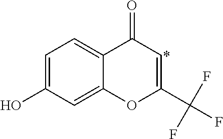 [SMILES: O=c1cc(C(F)(F)F)oc2cc(O)ccc12] | *—* | 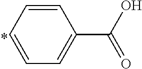 | a | 2 | 12.2 |
| IV-11 | 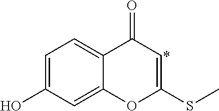 | *—* | 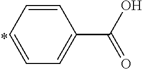 | b | 2 | 12.1 |
| IV-12 | 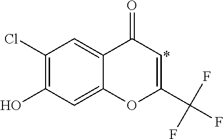 [SMILES: O=c1cc(C(F)(F)F)oc2cc(O)c(Cl)cc12] | *—* | 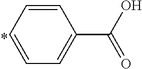 | c | 2 | 12.2 |
| IV-13 | 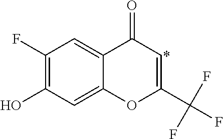 [SMILES: O=c1cc(C(F)(F)F)oc2cc(O)c(F)cc12] | *—* | 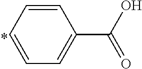 | b | 2 | 12.2 |
| IV-14 | 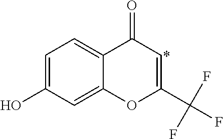 [SMILES: O=c1cc(C(F)(F)F)oc2cc(O)ccc12] | *—* | 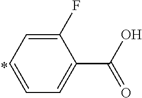 | a | 2 | 12.2 |
| IV-15 | 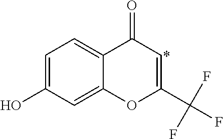 [SMILES: O=c1cc(C(F)(F)F)oc2cc(O)ccc12] | *—* | 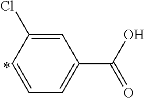 | a | 2 | 12.1 |
| IV-16 | 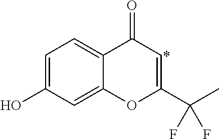 [SMILES: CC(F)(F)c1cc(=O)c2ccc(O)cc2o1] | *—* | 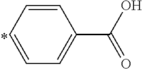 | a | 2 | 12.3 |
| IV-17 | 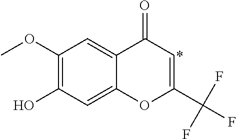 [SMILES: COc1cc2c(=O)cc(C(F)(F)F)oc2cc1O] | *—* | 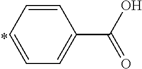 | b | 2 | 12.0 |
| IV-18 | 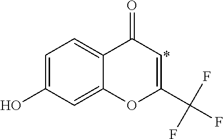 [SMILES: O=c1cc(C(F)(F)F)oc2cc(O)ccc12] | *—* | 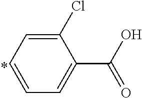 | a | 2 | 12.2 |
| IV-19 | 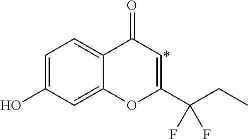 [SMILES: CCC(F)(F)c1cc(=O)c2ccc(O)cc2o1] | *—* | 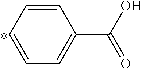 | b | 2 | 12.0 |
| IV-20 | 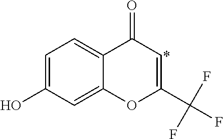 [SMILES: O=c1cc(C(F)(F)F)oc2cc(O)ccc12] | *—* | 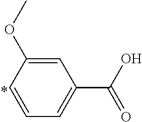 | a | 2 | 12.1 |
| IV-21 | 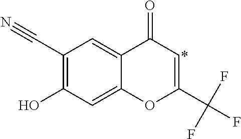 [SMILES: N#Cc1cc2c(=O)cc(C(F)(F)F)oc2cc1O] | *—* | 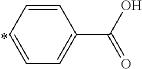 | c | 2 | 12.1 |
| IV-22 | 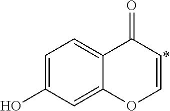 [SMILES: O=c1ccoc2cc(O)ccc12] | *—* | 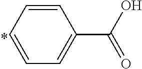 | b | 2 | 12.2 |
| IV-23 | 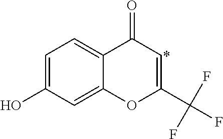 [SMILES: O=c1cc(C(F)(F)F)oc2cc(O)ccc12] | *—* | 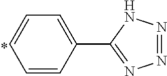 [SMILES: c1ccc(-c2nnn[nH]2)cc1] | a | 2 | 12.1 |
| IV-24 | 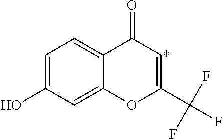 [SMILES: O=c1cc(C(F)(F)F)oc2cc(O)ccc12] | *—* | 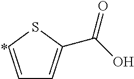 | a | 2 | 11.6 |
| IV-25 | 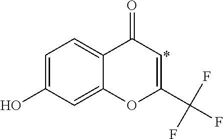 [SMILES: O=c1cc(C(F)(F)F)oc2cc(O)ccc12] | 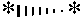 | 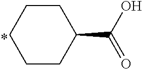 | a | 2 | 12.3 |
| IV-26 | 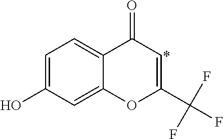 [SMILES: O=c1cc(C(F)(F)F)oc2cc(O)ccc12] | 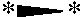 | 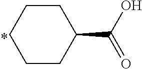 | b | 2 | 12.3 |
| IV-27 | 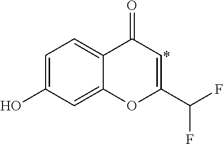 [SMILES: O=c1cc(C(F)F)oc2cc(O)ccc12] | *—* | 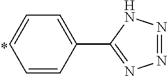 [SMILES: c1ccc(-c2nnn[nH]2)cc1] | a | 2 | 12.1 |
| IV-28 | 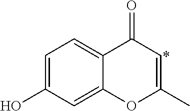 | *—* | 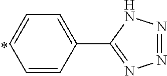 [SMILES: c1ccc(-c2nnn[nH]2)cc1] | a | 2 | 12.1 |
| IV-29 | 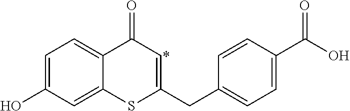 [SMILES: O=C(O)c1ccc(Cc2cc(=O)c3ccc(O)cc3s2)cc1] | *—* | 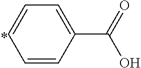 | b | 2 | 11.1 |
| IV-30 | 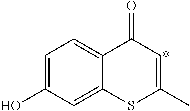 | *—* | 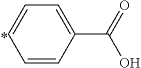 | a | 2 | 12.0 |
| IV-31 | 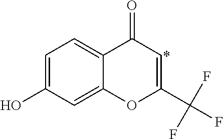 [SMILES: O=c1cc(C(F)(F)F)oc2cc(O)ccc12] | *—* | 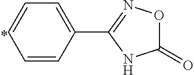 [SMILES: O=c1[nH]c(-c2ccccc2)no1] | a | 2 | 12.1 |
| IV-32 | 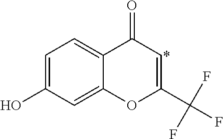 [SMILES: O=c1cc(C(F)(F)F)oc2cc(O)ccc12] | *—* | 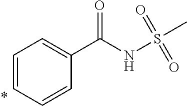 | b | 2 | 12.1 |
| IV-33 | 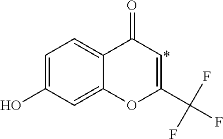 [SMILES: O=c1cc(C(F)(F)F)oc2cc(O)ccc12] | *—* | 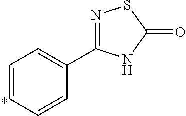 [SMILES: O=c1[nH]c(-c2ccccc2)ns1] | a | 2 | 12.2 |
| IV-34 | 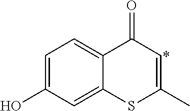 | *—* | 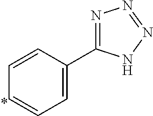 [SMILES: c1ccc(-c2nnn[nH]2)cc1] | a | 2 | 12.1 |
| IV-35 | 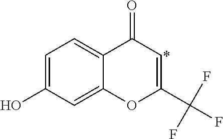 [SMILES: O=c1cc(C(F)(F)F)oc2cc(O)ccc12] | *—* | 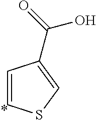 | b | 2 | 11.4 |
| IV-36 | 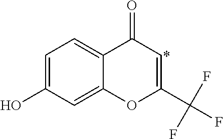 [SMILES: O=c1cc(C(F)(F)F)oc2cc(O)ccc12] | *—* | 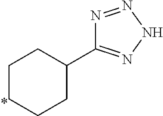 [SMILES: C1CCC(c2nn[nH]n2)CC1] | a | 2 | 12.3 |
| IV-37 | 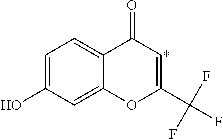 [SMILES: O=c1cc(C(F)(F)F)oc2cc(O)ccc12] | *—* | 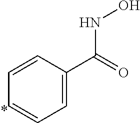 | b | 2 | 12.1 |
| IV-38 | 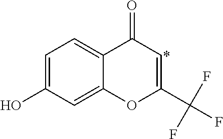 [SMILES: O=c1cc(C(F)(F)F)oc2cc(O)ccc12] | *—* | 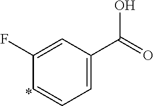 | a | 2 | 12.1 |
| IV-39 | 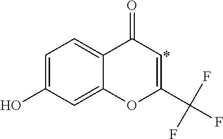 [SMILES: O=c1cc(C(F)(F)F)oc2cc(O)ccc12] | *—* | 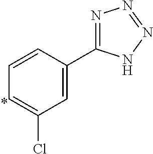 [SMILES: Clc1cccc(-c2nnn[nH]2)c1] | a | 2 | 12.2 |
| IV-40 | 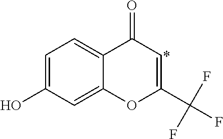 [SMILES: O=c1cc(C(F)(F)F)oc2cc(O)ccc12] | *—* | 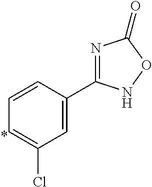 [SMILES: O=c1nc(-c2cccc(Cl)c2)[nH]o1] | a | 2 | 12.1 |
| IV-41 | 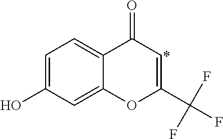 [SMILES: O=c1cc(C(F)(F)F)oc2cc(O)ccc12] | *—* | 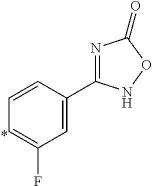 [SMILES: O=c1nc(-c2cccc(F)c2)[nH]o1] | a | 2 | 12.0 |
| IV-42 | 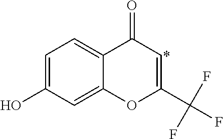 [SMILES: O=c1cc(C(F)(F)F)oc2cc(O)ccc12] | *—* | 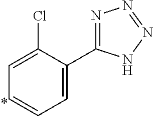 [SMILES: Clc1ccccc1-c1nnn[nH]1] | a | 2 | 12.2 |
| IV-43 | 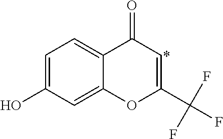 [SMILES: O=c1cc(C(F)(F)F)oc2cc(O)ccc12] | *—* | 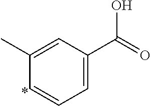 | a | 2 | 12.1 |
| IV-44 | 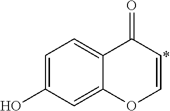 [SMILES: O=c1ccoc2cc(O)ccc12] | *—* | 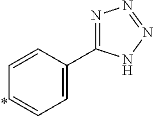 [SMILES: c1ccc(-c2nnn[nH]2)cc1] | a | 2 | 12.2 |
| IV-45 | 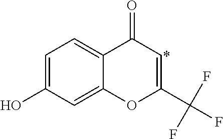 [SMILES: O=c1cc(C(F)(F)F)oc2cc(O)ccc12] | *—* | 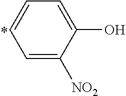 | a | 2 | 12.0^{†} |
| IV-46 | 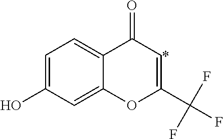 [SMILES: O=c1cc(C(F)(F)F)oc2cc(O)ccc12] | *—* | 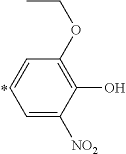 | a | 2 | 11.9^{†} |

^{†}signifies that for the cases where the acidic moiety is nitrophenol, the distance calculation is taken from the O of the hydroxyl on the bicyclic ring to the atom connected to the 4 position of phenyl.

Table 5 below lists compounds of Formula 1 that have been synthesized and an IC₅₀ value was obtained for each (see Example 3 for method) and is represented below by a, b, or c as described in Example 1 before the tables. Synthetic details and supporting data for these compounds can be found in Example 2.

TABLE 5

| | | | | | linear | Distance |
|---|---|---|---|---|---|---|
| | | | | | rotatable | after MM2 |
| # | HO—Cy₁ | linker | Cy₂-acidic moiety | IC₅₀ | bonds | min. |
| V-1 | 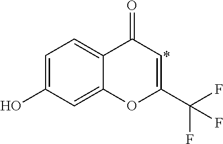 [SMILES: O=c1cc(C(F)(F)F)oc2cc(O)ccc12] | 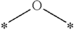 | 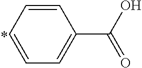 | b | 3 | 12.5 |
| V-2 | 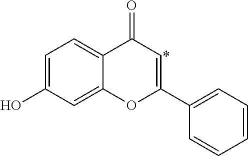 [SMILES: O=c1cc(-c2ccccc2)oc2cc(O)ccc12] | 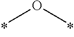 | 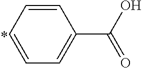 | c | 3 | 12.2 |
| V-3 | 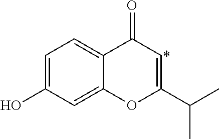 | 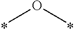 | 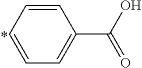 | c | 3 | 12.1 |
| V-4 | 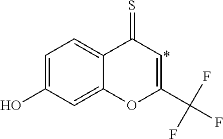 [SMILES: Oc1ccc2c(=S)cc(C(F)(F)F)oc2c1] | 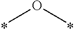 | 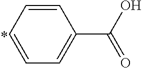 | b | 3 | 12.3 |
| V-5 | 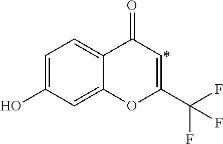 [SMILES: O=c1cc(C(F)(F)F)oc2cc(O)ccc12] | 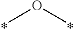 | 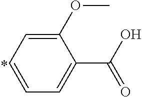 | c | 3 | 12.3 |
| V-6 | 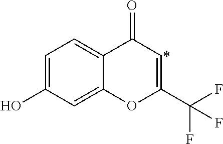 [SMILES: O=c1cc(C(F)(F)F)oc2cc(O)ccc12] | 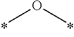 | 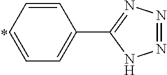 [SMILES: c1ccc(-c2nnn[nH]2)cc1] | c | 3 | 12.6 |
| V-7 | 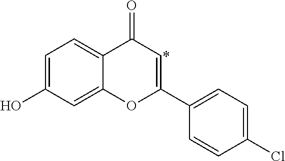 [SMILES: O=c1cc(-c2ccc(Cl)cc2)oc2cc(O)ccc12] | 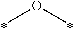 | 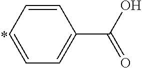 | c | 3 | 12.3 |
| V-8 | 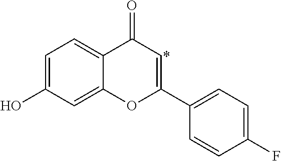 [SMILES: O=c1cc(-c2ccc(F)cc2)oc2cc(O)ccc12] | 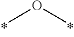 | 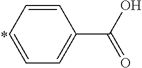 | c | 3 | 12.6 |
| V-9 | 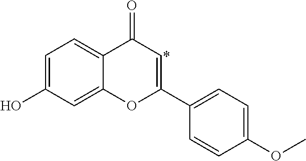 [SMILES: COc1ccc(-c2cc(=O)c3ccc(O)cc3o2)cc1] | 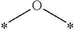 | 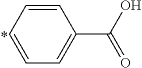 | c | 3 | 12.0 |
| V-10 | 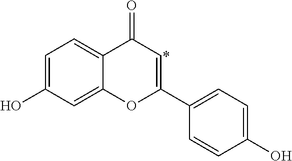 [SMILES: O=c1cc(-c2ccc(O)cc2)oc2cc(O)ccc12] | 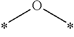 | 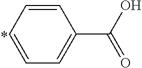 | c | 3 | 12.3 |
| V-11 | 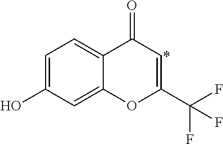 [SMILES: O=c1cc(C(F)(F)F)oc2cc(O)ccc12] | 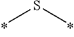 | 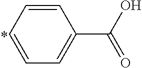 | c | 3 | 12.5 |
| V-12 | 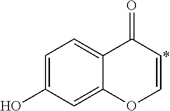 [SMILES: O=c1ccoc2cc(O)ccc12] | 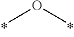 | 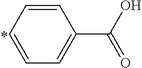 | c | 3 | 12.4 |
| V-13 | 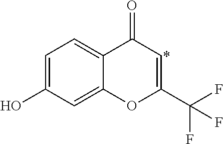 [SMILES: O=c1cc(C(F)(F)F)oc2cc(O)ccc12] | 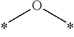 | 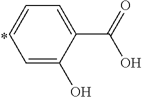 | b | 3 | 12.5 |
| V-14 | 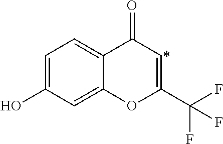 [SMILES: O=c1cc(C(F)(F)F)oc2cc(O)ccc12] | 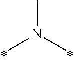 | 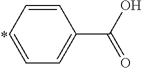 | c | 3 | 11.2 |
| V-15 | 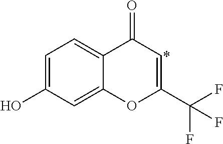 [SMILES: O=c1cc(C(F)(F)F)oc2cc(O)ccc12] | 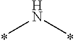 | 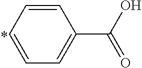 | c | 3 | 13.2 |
| V-16 | 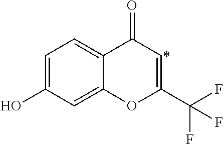 [SMILES: O=c1cc(C(F)(F)F)oc2cc(O)ccc12] | 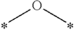 | 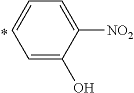 | a | 3 | 11.3^{†} |
| V-17 | 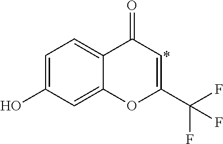 [SMILES: O=c1cc(C(F)(F)F)oc2cc(O)ccc12] | 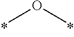 | 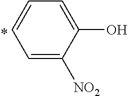 | b | 3 | 11.0^{†} |

^{†}signifies that for the cases where the acidic moiety is nitrophenol, the distance calculation is taken from the O of the hydroxyl on the bicyclic ring to the atom connected to the 4 position of phenyl.

Table 6 below lists compounds of Formula 1 that have been synthesized and an IC₅₀ value was obtained for each (see Example 3 for method) and is represented below by a, b, or c as described in Example 1 before the tables. Synthetic details and supporting data for these compounds can be found in Example 2.

TABLE 6

| | | | | | linear | Distance |
|---|---|---|---|---|---|---|
| | | | | | rotatable | after MM2 |
| # | HO—Cy₁ | linker | Cy₂-acidic moiety | IC₅₀ | bonds | min. |
| VI-1 | 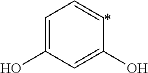 | 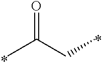 | 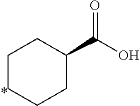 | b | 4 | 8.8 |
| VI-2 | 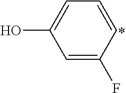 | 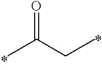 | 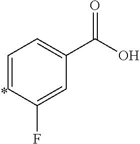 | a | 4 | 7 |
| VI-3 | 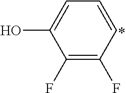 | 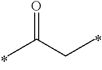 | 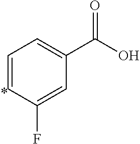 | a | 4 | 7.1 |
| VI-4 | 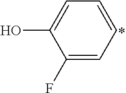 | 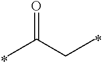 | 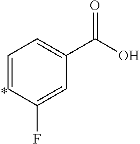 | a | 4 | 7.5 |
| VI-5 | 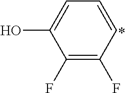 | 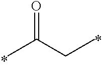 | 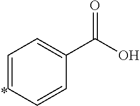 | a | 4 | 7.3 |
| VI-6 | 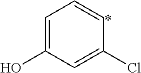 | 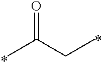 | 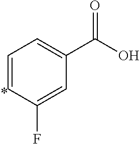 | a | 4 | 7.1 |
| VI-7 | 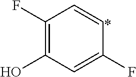 | 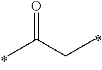 | 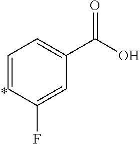 | b | 4 | 7.1 |
| VI-8 | 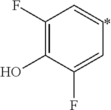 | 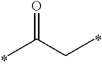 | 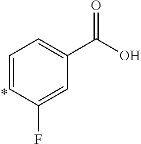 | b | 4 | 7.7 |
| VI-9 | 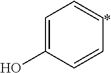 | 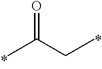 | 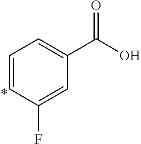 | a | 4 | 6.9 |
| VI-10 | 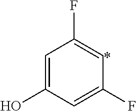 | 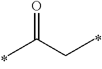 | 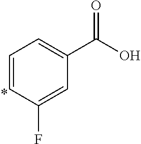 | b | 4 | 7.2 |
| VI-11 | 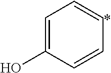 | 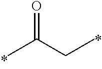 | 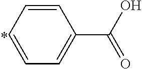 | b | 4 | 6.2 |
| VI-12 | 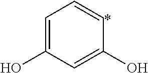 | 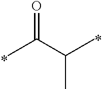 | 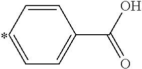 | c | 4 | 11.2 |
| VI-13 | 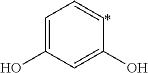 | 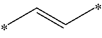 | 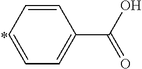 | c | 3 | 7.8 |
| VI-14 | 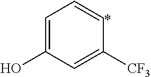 [SMILES: Oc1cccc(C(F)(F)F)c1] | 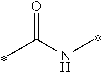 | 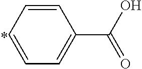 | b | 4 | 8.8 |
| VI-15 | 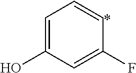 | 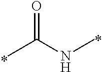 | 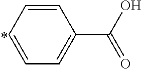 | b | 3 | 7.4 |
| VI-16 | 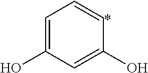 | 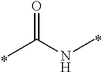 | 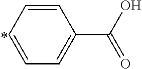 | c | 3 | 8.4 |
| VI-17 | 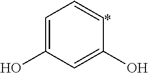 | 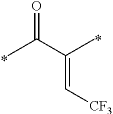 | 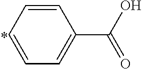 | a | 5 | 11.9 |
| VI-18 | 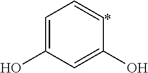 | 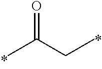 | 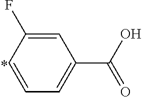 | b | 4 | 7.2 |
| VI-19 | 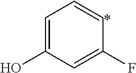 | 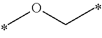 | 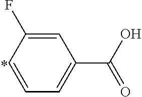 | b | 4 | 12.2 |
| VI-20 | 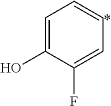 | 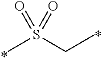 | 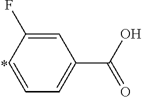 | a | 4 | 6.4 |
| VI-21 | 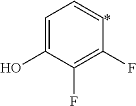 | 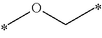 | 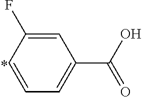 | a | 4 | 12.2 |
| VI-22 | 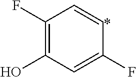 | 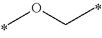 | 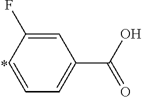 | b | 4 | 12.1 |
| VI-23 | 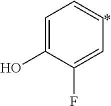 | 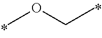 | 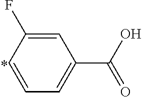 | b | 4 | 12.1 |
| VI-24 | 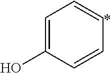 | 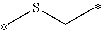 | 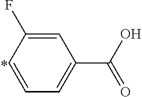 | b | 4 | 12.3 |
| VI-25 | 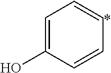 | 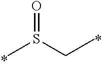 | 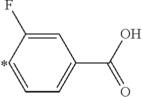 | b | 4 | 8.5 |
| VI-26 | 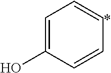 | 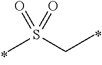 | 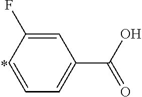 | a | 4 | 6.3 |
| VI-27 | 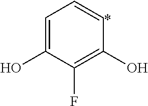 | 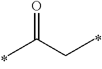 | 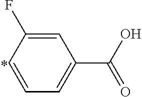 | b | 4 | 6.1 |
| VI-28 | 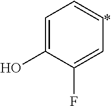 | 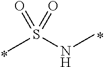 | 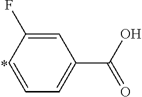 | c | 4 | 12.5 |
| VI-29 | 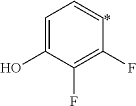 | 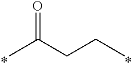 | 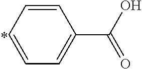 | c | 5 | 13.1 |
| VI-30 | 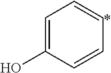 | 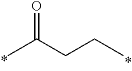 | 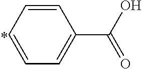 | c | 5 | 12.9 |
| VI-31 | 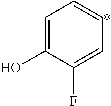 | 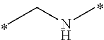 | 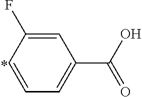 | c | 4 | 12.2 |
| VI-32 | 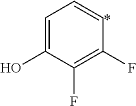 | 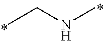 | 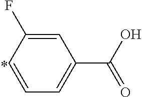 | b | 4 | 12.2 |
| VI-33 | 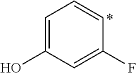 | 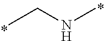 | 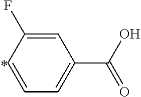 | c | 4 | 12.2 |

Table 7 below lists compounds of Formula 1 that have been synthesized and an IC₅₀ value was obtained for each (see Example 3 for method) and is represented below by a, b, or c as described in Example 1 before the tables. Synthetic details and supporting data for these compounds can be found in Example 2.

TABLE 7

| | | | | | linear | distance |
|---|---|---|---|---|---|---|
| | | | | | rotatable | after MM2 |
| # | HO—Cy₁ | linker | Cy₂-acidic moiety | IC₅₀ | bonds | min. |
| VII-1 | 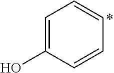 | 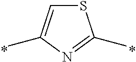 | 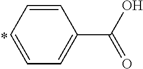 | c | 3 | 12.6 |
| VII-2 | 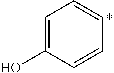 | 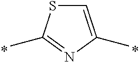 | 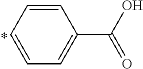 | b | 3 | 12.7 |
| VII-3 | 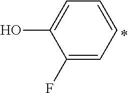 | 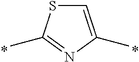 | 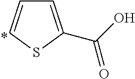 | a | 3 | 12.7 |
| VII-4 | 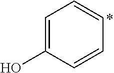 | 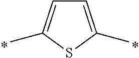 | 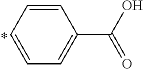 | b | 3 | 13.7 |
| VII-5 | 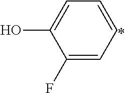 | 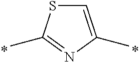 | 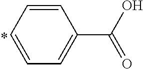 | b | 3 | 11.6 |
| VII-6 | 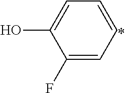 | 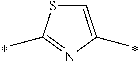 | 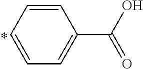 | b | 3 | 12.7 |
| VII-7 | 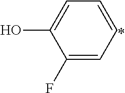 | 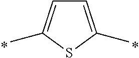 | 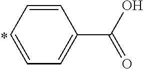 | b | 3 | 13.7 |
| VII-8 | 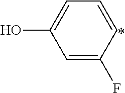 | 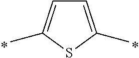 | 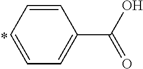 | b | 3 | 13.7 |
| VII-9 | 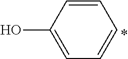 | 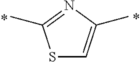 | 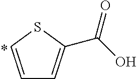 | a | 3 | 12.7 |
| VII-10 | 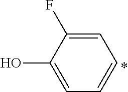 | 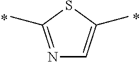 | 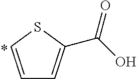 | a | 3 | 13.4 |
| VII-11 | 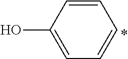 | 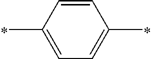 | 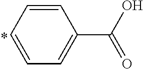 | b | 3 | 14.5 |
| VII-12 | 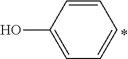 | 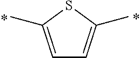 | 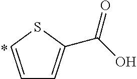 | b | 3 | 13.5 |
| VII-13 | 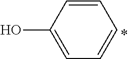 | 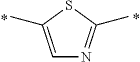 | 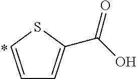 | b | 3 | 13.1 |
| VII-14 | 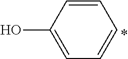 | 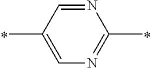 | 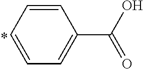 | c | 3 | 14.3 |
| VII-15 | 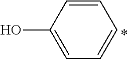 | 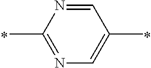 | 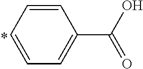 | c | 3 | 14.3 |
| VII-16 | 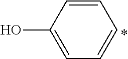 | 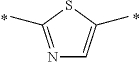 | 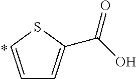 | a | 3 | 13.4 |
| VII-17 | 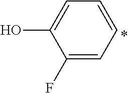 | 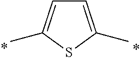 | 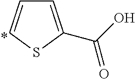 | a | 3 | 13.5 |
| VII-18 | 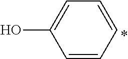 | 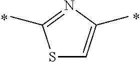 | 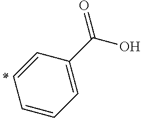 | c | 3 | 11.7 |
| VII-19 | 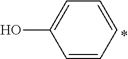 | 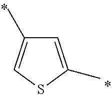 | 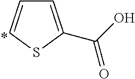 | b | 3 | 12.1 |
| VII-20 | 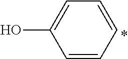 | 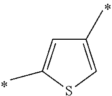 | 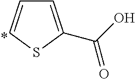 | b | 3 | 12.1 |
| VII-21 | 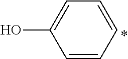 | 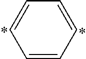 | 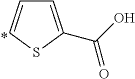 | b | 3 | 13.9 |
| VII-22 | 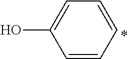 | 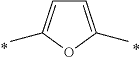 | 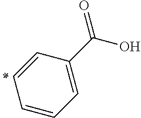 | c | 3 | 12.8 |
| VII-23 | 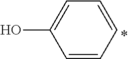 | 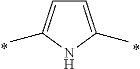 | 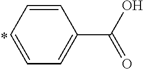 | c | 3 | 12.9 |
| VII-24 | 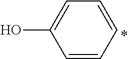 | 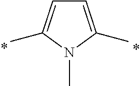 | 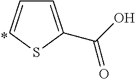 | b | 3 | 12.8 |
| VII-25 | 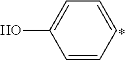 | 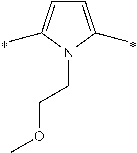 | 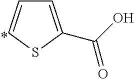 | a | 3 | 13.0 |
| VII-26 | 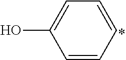 | 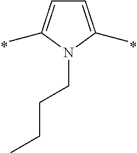 | 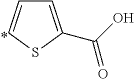 | b | 3 | 12.9 |
| VII-27 | 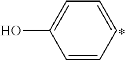 | 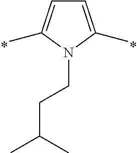 | 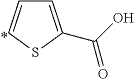 | a | 3 | 12.9 |
| VII-28 | 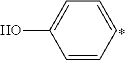 | 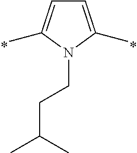 | 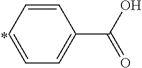 | a | 3 | 13.5 |
| VII-29 | 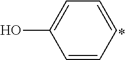 | 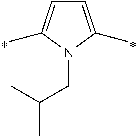 | 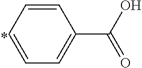 | a | 3 | 13.5 |
| VII-30 | 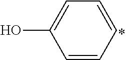 | 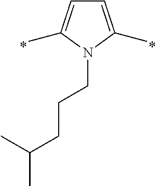 | 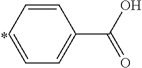 | a | 3 | 13.7 |
| VII-31 | 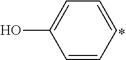 | 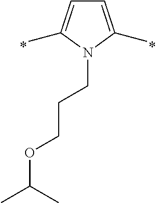 | 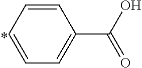 | a | 3 | 13.3 |
| VII-32 | 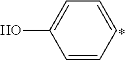 | 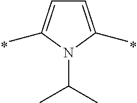 | 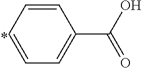 | a | 3 | 13.7 |
| VII-33 | 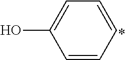 | 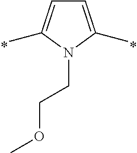 | 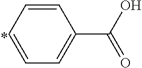 | b | 3 | 13.7 |
| VII-34 | 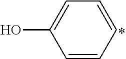 | 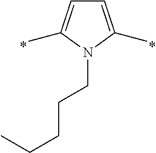 | 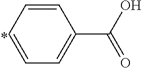 | a | 3 | 13.6 |
| VII-35 | 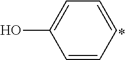 | 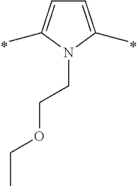 | 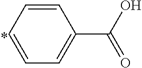 | a | 3 | 13.7 |
| VII-36 | 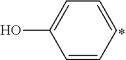 | 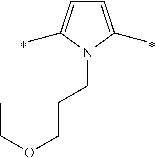 | 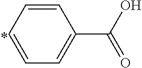 | a | 3 | 13.6 |
| VII-37 | 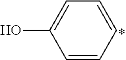 | 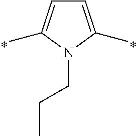 | 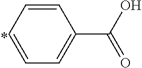 | a | 3 | 13.7 |

Table 8 below lists compounds of Formula 1 that have been synthesized and an IC₅₀ value was obtained for each (see Example 3 for method) and is represented below by a, b, or c as described in Example 1 before the tables. Synthetic details and supporting data for these compounds can be found in Example 2.

TABLE 8

| | | | | | linear | Distance |
|---|---|---|---|---|---|---|
| | | | | | rotatable | after MM2 |
| # | HO—Cy₁ | linker | Cy₂-acidic moiety | IC₅₀ | bonds | min. |
| VIII-1 | 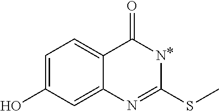 | *—* | 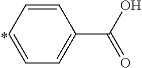 | c | 2 | 12.1 |
| VIII-2 | 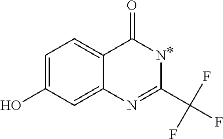 [SMILES: O=c1[nH]c(C(F)(F)F)nc2cc(O)ccc12] | *—* | 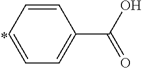 | b | 2 | 11.7 |
| VIII-3 | 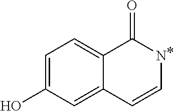 [SMILES: O=c1[nH]ccc2cc(O)ccc12] | *—* | 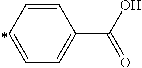 | a | 2 | 12 |
| VIII-4 | 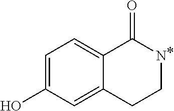 [SMILES: O=C1NCCc2cc(O)ccc21] | *—* | 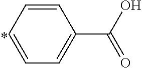 | a | 2 | 12.1 |
| VIII-5 | 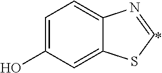 | *—* | 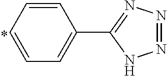 [SMILES: c1ccc(-c2nnn[nH]2)cc1] | b | 2 | 11.9 |
| VIII-6 | 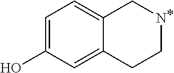 [SMILES: Oc1ccc2c(c1)CCNC2] | *—* | 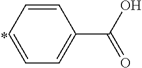 | c | 2 | 11.2 |
| VIII-7 | 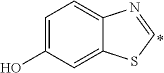 | *—* | 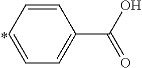 | b | 2 | 11.9 |
| VIII-8 | 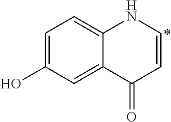 [SMILES: O=c1cc[nH]c2ccc(O)cc12] | *—* | 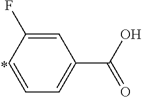 | c | 2 | 12.2 |
| VIII-9 | 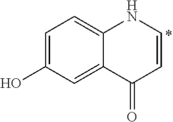 [SMILES: O=c1cc[nH]c2ccc(O)cc12] | *—* | 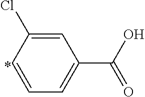 | c | 2 | 12.2 |
| VIII-10 | 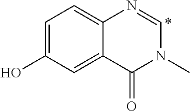 | *—* | 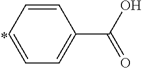 | a | 2 | 12 |
| VIII-11 | 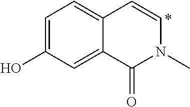 | *—* | 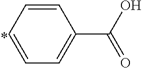 | b | 2 | 12.1 |
| VIII-12 | 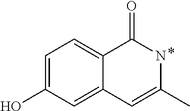 | 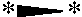 | 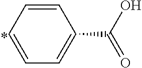 | b | 2 | 12.3 |
| VIII-13 | 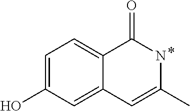 | *—* | 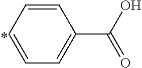 | b | 2 | 12.2 |
| VIII-14 | 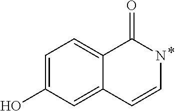 [SMILES: O=c1[nH]ccc2cc(O)ccc12] | 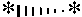 | 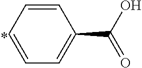 | c | 2 | 12.2 |
| VIII-15 | 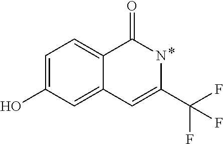 [SMILES: O=c1[nH]c(C(F)(F)F)cc2cc(O)ccc12] | 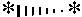 | 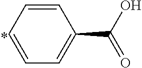 | a | 2 | 12.2 |
| VIII-16 | 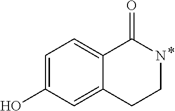 [SMILES: O=C1NCCc2cc(O)ccc21] | 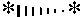 | 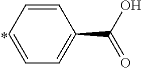 | c | 2 | 12.2 |
| VIII-17 | 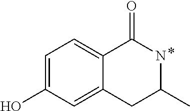 | 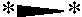 | 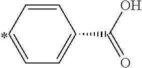 | c | 2 | 12 |

### Example 2

### Synthetic Description

### Synthetic Details for Compounds of Table 1

### Intermediate 1-1, 2-chloro-6-methoxyquinolin-4-amine

### Step 1

A mixture of p-anisidine (100 g, 0.813 mol), and malonic acid (85.0 g, 0.817 mol) in POCl₃ (500 mL) was refluxed for 6 hours. The excess POCl₃ was removed under reduced pressure and the residue was neutralized with 8 M NaOH to pH 7. The aqueous layer was extracted with CH₂Cl₂ (300 mL×3), the combined organic layer was washed with brine (500 mL), dried over Na₂SO₄ and concentrated in vacuo to give the crude product, which was purified by column chromatography on silica gel (PE/EtOAc=15/1) to give 2,4-dichloro-6-methoxyquinoline (35.0 g, yield: 19%) as a white solid.

### Step 2

A suspension of 2,4-dichloro-6-methoxyquinoline (5.00 g, 22.0 mmol) in NH₃ (g)/MeOH (saturated, 40 mL) was heated to 150° C. for 16 hours in a sealed tube. The solvent was removed under reduced pressure and the residue was diluted with MeOH (20 mL). The mixture was filtered off and the filtrate was concentrated to give the crude product, which was purified by column chromatography on silica gel (PE/EtOAc=2/1) to give 2-chloro-6-methoxyquinolin-4-amine (7.50 g, yield: 55%) as a yellow solid.

### Intermediate 1-2: 2-chloro-8-fluoroquinolin-6-yl acetate

### Step 1

4-Amino-3-fluorophenol (3.4 g) was mixed with 3-chloropropanoyl chloride (3.56 g) in acetone (60 mL) and heated at reflux over 3 hours. After aqueous work-up with EtOAc/water, the isolated organic layers were dried over anhydrous Na2SO4 and concentrated in vacuo. The crude product was purified with a flash silica gel chromatography to afford 3-chloro-N-(2-fluoro-4-hydroxyphenyl)propanamide (2.95 g) as light brown solids.

### Step 2

3-Chloro-N-(2-fluoro-4-hydroxyphenyl)propanamide (2.1 g) was mixed with anhydrous AlCl3 (7 g) and heated at 160° C. overnight. The resultant mixture was treated with 1N HCl and extracted with EtOAc. After isolation of the organic layer and removal of solvents under reduced pressure, the desired crude product-8-fluoro-6-hydroxy-3,4-dihydroquinolin-2(1H)-one (1.8 g) was collected as light brown solids.

### Step 3

Crude 8-fluoro-6-hydroxy-3,4-dihydroquinolin-2(1H)-one (0.574 g) was treated with acetyl chloride (330 mg) and TEA (0.68 mL) in DCM (8 mL) over 3 h. After aqueous work-up with EtOAc/water, the crude product was purified with a flash column chromatography to afford the desired product-8-fluoro-2-oxo-1,2,3,4-tetrahydroquinolin-6-yl acetate (382 mg) as colorless solids.

### Step 4

To a solution of 8-fluoro-2-oxo-1,2,3,4-tetrahydroquinolin-6-yl acetate (718 mg) in toluene (8 mL) was added DDQ (1.2 g). The resultant solution was heated at 70° C. over 48 h. After aqueous work-up with EtOAc, the crude product was purified by a flash silica column chromatography to afford the pure product-8-fluoro-2-hydroxyquinolin-6-yl acetate (550 mg) as colorless solids.

### Step 5

To a solution of 8-fluoro-2-hydroxyquinolin-6-yl acetate (550 mg) in DMF (6 mL) was added POC13 (0.6 mL). Then, the mixture was heated at 80° C. over a couple of hours. After aqueous work-up, the desired product, 2-chloro-8-fluoroquinolin-6-yl acetate (380 mg) was obtained by a flash silica column chromatography.

### Synthesis of Compound I-45, 4-(5-bromo-6-hydroxyquinolin-2-yl)benzoic acid

### Step 1

A mixture of 2-chloro-6-methoxyquinoline (see U.S. 61/391,225 for synthesis) (200 mg, 1.0 mmol), 4-(methoxycarbonyl) phenylboronic acid (205 mg, 1.1 mmol), Pd(dppf)Cl₂ (366 mg, 0.5 mmol) and sodium carbonate (212 mg, 2.0 mmol) in 1,4-dioxane/water (3 mL/0.6 mL) was heated to 120° C. by microwave for 1 h. The precipitates were filtered; washed with EtOAc (10 mL), acetone (10 mL) and water (10 mL) separately; dried to afford methyl 4-(6-methoxyquinolin-2-yl)benzoate as black solids. (120 mg, 40.9%).

### Step 2

To a solution of methyl 4-(6-methoxyquinolin-2-yl)benzoate (630 mg, 2.15 mmol) in DCM (9 mL) was added Br₂ (0.3 mL, 6.45 mmol). The reaction mixture was stirred at room temperature overnight. Then the mixture was partitioned with brine and DCM. The precipitate was filtered and dried to afford methyl 4-(5-bromo-6-methoxyquinolin-2-yl)benzoate as a solid (800 mg, 100%). MS (ESI): m/z=373.0 [M+1]⁺.

### Step 3

To a solution of the product from above (150 mg, 0.40 mmol) in DCM (5 mL) was added BBr₃ (0.38 mL, 4.0 mmol). The reaction mixture was stirred at room temperature overnight. Water (20 mL) was added carefully, the mixture was extracted with EtOAc (20 mL×3). The combined organic phase was concentrated and purified by prep-HPLC to afford Compound I-45 as a grey powder (40 mg, 29.2%). MS (ESI): m/z=346.0 [M+1]⁺. 1H-NMR (DMSO-d⁶ 500 MHz): 8.48 (d, J=8.5 Hz, 1H), 8.35 (d, J=8.0 Hz, 2H), 8.25 (d, J=9.0 Hz, 1H), 8.10 (d, J=7.5 Hz, 2H), 8.01 (d, J=8.5 Hz, 1H), 7.60 (d, J=9.0 Hz, 1H) ppm.

### Synthesis of Compound I-46, 3-bromo-4-(6-hydroxyquinolin-2-yl)benzoic acid

### Step 1

To a mixture of compound 2-chloro-6-methoxyquinoline (1.70 g, 8.78 mmol), 2-amino-4-(methoxycarbonyl)phenylboronic acid (2.05 g, 10.5 mmol), and K₂CO₃ (2.43 g, 17.6 mmol) in ethylene glycol monomethyl ether/H₂O (35 mL/5 mL) was added Pd(dppf)Cl₂ (158 mg, 0.193 mmol) under N₂ atmosphere. Then the mixture was heated to 80° C. for 3 hours. H₂O (80 mL) was added to the reaction mixture, and the resulting mixture was extracted with EtOAc (30 mL×3). The combined organic layers were washed with brine (30 mL), dried over anhydrous Na₂SO₄, filtered and concentrated in vacuo to give the crude product. The crude product was purified by silica gel column (PE/EtOAc=15/1 to 3/1) to give methyl 3-amino-4-(6-methoxyquinolin-2-yl)benzoate (500 mg, yield 19%) as a yellow solid.

### Step 2

To a mixture of the above product (200 mg, 0.649 mmol) in HBr (40%)/H₂O (5 mL/5 mL) was added NaNO₂ (44.8 mg, 0.649 mmol) in H₂O (3 mL) dropwise at 0° C., and the reaction mixture was stirred at 0° C. for 30 min, CuBr (186 mg, 1.30 mmol) was added to the reaction mixture. Then the mixture was stirred at 25° C. for 2 hours. The reaction mixture was basified with aqueous NaOH (2M) to pH=7-8, extracted with EtOAc (30 mL×3). The combined organic layers were washed with brine (30 mL), dried over anhydrous Na₂SO₄, filtered and concentrated under reduced pressure to give methyl 3-bromo-4-(6-methoxyquinolin-2-yl)benzoate (205 mg, yield 85%) as a yellow solid.

### Step 3

To a solution of the above product (205 mg, 0.550 mmol) in anhydrous DCM (6 mL) was added BBr₃ (0.26 mL, 2.8 mmol, d=2.64 g/mL) dropwise at 0° C. The resulting mixture was stirred at 25° C. for 48 hour. The reaction mixture was quenched with H₂O (30 mL) and filtered, the filter cake was washed with EtOAc (10 mL) to give compound I-46 (90 mg, yield 48%) as a yellow solid. ¹H NMR (DMSO-d₆ 400 MHz): δ 10.27 (brs, 1H), 8.36 (d, J=8.8 Hz, 1H), 8.24 (d, J=1.6 Hz, 1H), 8.06 (dd, J=8.0, 1.6 Hz, 1H), 7.95 (d, J=9.2 Hz, 1H), 7.76 (d, J=8.0 Hz, 1H), 7.71 (d, J=8.8 Hz, 1H), 7.41 (dd, J=9.2, 2.4 Hz, 1H), 7.26 (d, J=2.4 Hz, 1H). LC-MS purity: 94.8%. MS (ESI): m/z 343.9 [M+H]⁺.

### Synthesis of Compound I-47, 4-(4-(dimethylamino)-6-hydroxyquinolin-2-yl)benzoic acid

4-(4-fluoro-6-hydroxyquinolin-2-yl)benzoic acid (Compound I-26, 30 mg) was mixed with 1 mmol Me₂NH.HCl and 0.5 ml DIEA in 3 ml DMF and heated to 160° C. for 30 minutes. The solvent was evaporated to dryness and water was added. The solid was filtered and washed with water and dried. The crude was triturated with acetone to obtain 10.5 mg of Compound I-47. ¹H NMR (DMSO-d₆ 300 MHz TMS): δ 13.2 (b, 1H), 10.28 (s, 1H), 8.22 (m, 2H), 8.11 (d, 1H), 7.99 (d, 1H), 7.50 (s, 1H), 7.39 (d, 1H), 7.26 (s, 1H), 3.24 (s, 6H), MS (ESI): m/z=309.30 [M+1]+.

### Synthesis of Compound I-48, 4-(4-fluoro-6-hydroxyquinolin-2-yl)-3-methoxybenzoic acid

### Step 1

A mixture solution of 2-chloro-4-fluoro-6-methoxyquinoline (see U.S. 61/391,225 for synthesis) (280 mg, 1.32 mmol) and BBr₃ (0.3 mL, 3.2 mmol, 2.64 g/mL) in DCM (5 mL) was stirred at 20° C. for 12 hours. The mixture was quenched with water (20 mL) and extracted with DCM (10 mL×3). The organic layers were dried over anhydrous Na₂SO₄ and concentrated in vacuo to give the crude product, which was purified by silica gel column (PE/EtOAc=50/1) to give 2-chloro-4-fluoroquinolin-6-ol (177 mg, yield 69%) as a white solid.

### Step 2

A mixture of the above product (133 mg, 0.670 mmol), 4-(dihydroxyboryl)-3-methoxybenzoic acid (156 mg, 0.801 mmol), K₂CO₃ (278 mg, 2.01 mmol), Pd(dppf)Cl₂ (30 mg, 0.026 mmol) in DMF (3 mL) and H₂O (0.6 mL) was stirred under N₂ atmosphere at 130° C. for 2.5 hours. The mixture was cooled to room temperature, acidified with aqueous HCl (1M) until pH=6 and extracted with EtOAc (30 mL×3). The combined organic layer was washed with brine (50 mL), dried over anhydrous Na₂SO₄ and concentrated in vacuo. The residue was purified by silica gel column (DCM/MeOH=15/1) to give Compound I-48 (10.5 mg, yield 5%) as an off-white solid. ¹H NMR (CD₃OD 400 MHz TMS): δ 8.01 (dd, J=8.8, 1.2 Hz, 1H), 7.88-7.76 (m, 3H), 7.63 (d, J=11.2 Hz, 1H), 7.43 (dd, J=9.2, 2.8 Hz, 1H), 7.33 (d, J=2.8 Hz, 1H), 3.98 (s, 3H). MS (ESI): m/z 313.8 [M+H]⁺.

### Synthesis of Compound I-49, 3-cyano-4-(6-hydroxyquinolin-2-yl)benzoic acid

### Step 1

Followed the coupling procedure described in step 1 of Compound I-46, starting from 2-chloro-6-methoxyquinoline (1.70 g, 8.78 mmol) and 2-amino-4-(methoxycarbonyl)phenylboronic acid (2.05 g, 10.5 mmol). Note: Ester exchange occurred between desired compound and solvent. 2-Methoxyethyl 3-amino-4-(6-methoxyquinolin-2-yl)benzoate was obtained (1.10 g, yield 35%) as a yellow solid.

### Step 2

To a mixture of the above product (500 mg, 1.42 mmol) in HBr (40%)/H₂O (10 mL/10 mL) was added NaNO₂ (97.9 mg, 1.42 mmol) in H₂O (5 mL) dropwise at 0° C., and the reaction mixture was stirred at 0° C. for 30 min. CuBr (407 mg, 2.84 mmol) was added to the reaction mixture. The mixture was stirred at 25° C. for 2 hours, then basified with aqueous NaOH (2M) to pH=7-8, and extracted with EtOAc (20 mL×3). The combined organic layers were washed with brine (20 mL), dried over anhydrous Na₂SO₄, filtered and concentrated in vacuo to give 2-methoxyethyl 3-bromo-4-(6-methoxyquinolin-2-yl)benzoate (580 mg, yield 98%) as a yellow solid.

### Step 3

To a solution of the above product (580 mg, 1.40 mmol) in DMF (15 mL) was added Zn(CN)₂ (329 mg, 2.80 mmol) and Pd(PPh₃)₄ (162 mg, 0.140 mmol). The resulting mixture was stirred at 120° C. under N₂ atmosphere for 16 hours. After cooling to room temperature, the mixture was filtered and the filtrate was diluted with EtOAc (60 mL), washed with H₂O (20 mL×3) and brine (20 mL), dried over anhydrous Na₂SO₄, filtered and concentrated under reduced pressure to give the crude product. The crude product was purified by silica gel column (PE/EtOAc=50/1 to 10/1) to give 2-methoxyethyl 3-cyano-4-(6-methoxyquinolin-2-yl)benzoate (180 mg, yield 36%) as a yellow solid.

### Step 4

To a solution of the above product (180 mg, 0.497 mmol) in anhydrous DCM (10 mL) was added BBr₃ (0.24 mL, 2.5 mmol, d=2.64 g/mL) dropwise at 0° C. The resulting mixture was stirred at 25° C. for 2 hours. The reaction mixture was quenched with H₂O (20 mL) and basified with aqueous NaOH (2M) to pH=7˜8, then extracted with DCM (30 mL×3). The combined organic layers were washed with brine (30 mL), dried over anhydrous Na₂SO₄, filtered and concentrated in vacuo to give 2-hydroxyethyl 3-cyano-4-(6-hydroxyquinolin-2-yl)benzoate (100 mg, yield 60%) as an off-white solid.

### Step 5

To a solution of the above product (100 mg, 0.299 mmol) in MeOH (5 mL) and THF (5 mL) was added LiOH H₂O (25.1 mg, 0.598 mmol). The mixture was stirred at 25° C. for 16 hours. The reaction mixture was acidified with 1N HCl to pH=5˜6, and the resulting mixture was extracted with EtOAc (20 mL×3). The combined organic layers were washed with brine, dried over anhydrous Na₂SO₄, filtered and concentrated. The crude product was washed with EtOAc (10 mL) to give Compound I-49 (35 mg, yield 45%) as a yellow solid. ¹H NMR (DMSO-d₆ 400 MHz): δ 13.66 (brs, 1H), 10.32 (brs, 1H), 8.40 (d, J=1.6 Hz, 1H), 8.37 (d, J=8.4 Hz, 1H), 8.32 (dd, J=8.0, 1.6 Hz, 1H), 8.16 (d, J=8.0 Hz, 1H), 8.02-7.90 (m, 2H), 7.42 (dd, J=9.2, 2.8 Hz, 1H), 7.25 (d, J=2.8 Hz, 1H). MS (ESI): m/z 290.6 [M+H]⁺.

### Synthesis of Compound I-50, 2-(4-carboxy-2-chlorophenyl)-6-hydroxyquinoline 1-oxide

To a solution of 3-chloro-4-(6-hydroxyquinolin-2-yl)benzoic acid (Compound I-8) (620 mg) in HOAc (8 mL) was added 3-chloroperbenzoic acid (77% pure, 1.19 g). The resultant mixture was heated at 90° C. over 3 hour. After removal of HOAc under reduced pressure, the resultant mixture was trituated with DCM and recrystallized from EtOH/water twice to afford the desired product (245 mg) as colorless solids. ¹H NMR (DMSO-d₆ 300 MHz TMS): δ 10.49 (1H, s), 8.43 (1H, d, J=9 Hz), 8.06 (1H, d, J=3 Hz), 8.01 (1H, dd, J=9 and 3 Hz), 7.82 (1H, d, J=6 Hz), 7.69 (1H, d, J=6 Hz), 7.47 (1H, d, J=9 Hz), 7.37 (1H, dd, J=9 and 3 Hz), 7.31 (1H, d, J=3 Hz) ppm; MS (ESI): m/z 316, [M+H]⁺.

### Synthesis of Compound I-51, 4-(4-amino-6-hydroxyquinolin-2-yl)benzoic acid

### Step 1

Followed the coupling procedure described in step 2 of Compound I-48 starting with Intermediate 1-1 and 4-methoxycarbonylphenylboronic acid to give methyl 4-(4-amino-6-methoxyquinolin-2-yl)benzoate (3.20 g, yield 46%) as an off-white solid.

### Step 2

Followed BBr₃ deprotection method described in step 3 of Compound I-46, wherein after quenching with water the mixture was extracted with DCM, dried over anhydrous Na₂SO₄ and concentrated. Trituration with MeOH (20 mL) gave Compound I-51 as a yellow solid. ¹H NMR (DMSO-d₆ 400 MHz TMS): δ 13.52 (brs, 1H), 10.53 (brs, 1H), 8.74 (brs, 2H), 8.20 (d, J=8.4 Hz, 2H), 8.01-7.96 (m, 3H), 7.65 (d, J=1.6 Hz, 1H), 7.56 (dd, J=9.2, 2.4 Hz, 1H), 6.98 (s, 1H). MS (ESI): m/z 280.9 [M+H]⁺.

### Synthesis of Compound I-52, 4-(3-cyano-6-hydroxyquinolin-2-yl)benzoic acid

### Step 1

To a mixture of 2-chloroquinoline-3-carboxaldehyde (1.00 g, 4.50 mmol) in THF (30 mL) was added NH₃.H₂O (30 mL, 25%) and I₂ (1.26 g, 4.90 mmol), the mixture was stirred at 20° C. for 8 hours. H₂O (80 mL) was added, and the resulting mixture was extracted with EtOAc (30 mL×3), organic layers were washed with brine (30 mL), dried over anhydrous Na₂SO₄, filtered and concentrated. Purification by silica gel column (PE/EtOAc=10/1 to 2/1) gave 2-chloro-6-methoxyquinoline-3-carbonitrile (370 mg, yield 38%) as a yellow solid.

### Step 2

Followed the coupling procedure described in step 2 of Compound I-48 starting with the above product and 4-Carboxyphenylboronic acid (where reaction was heated to 100° C. for 3 hours). Obtained the desired 4-(3-cyano-6-methoxyquinolin-2-yl)benzoic acid (45.0 mg, yield 42%) as a yellow solid.

### Step 3

Followed the BBr₃ deprotection described for Compound I-45 (step 3) (where reaction here was stirred at 30° C. for 24 h). Obtained the desired Compound I-52 (6.0 mg, yield 8%) as a white solid. ¹H NMR (MeOD-d₆ 400 MHz): δ 8.78 (s, 1H), 8.22 (d, J=8.0 Hz, 2H), 8.05-8.01 (m, 3H), 7.56 (dd, J=9.2, 2.8 Hz, 1H), 7.28 (d, J=2.4 Hz, 1H). MS (ESI): m/z 290.8 [M+H]⁺.

### Synthesis of Compound I-53, 4-(5-fluoro-6-hydroxyquinolin-2-yl)benzoic acid

### Step 1

To a solution of methyl 4-(6-methoxyquinolin-2-yl)benzoate (see Compound I-45, step 1 for synthesis) (250 mg, 0.85 mmol) in MeCN (5 mL) was added selectfluoro (453 mg, 1.28 mmol). The reaction mixture was heated at 50° C. overnight. The solvent was removed under reduced pressure, and the residue was partitioned with water and DCM, and extracted with DCM (20 mL×2). The combined organic phase was washed with brine, dried over Na₂SO₄ and concentrated to afford methyl 4-(5-fluoro-6-methoxyquinolin-2-yl)benzoate as a brown solid (300 mg, 113%).

### Step 2

To a solution of the above product (300 mg, 0.96 mmol) in DCM (2 mL) was added BBr₃ (2.4 g, 9.6 mmol). The reaction was stirred at room temperature overnight. H₂O (40 mL) was added carefully. The precipitates were collected and purified by prep-HPLC to give Compound I-53 as a brown powder (76.8 mg, 25.9%). ¹H-NMR (DMSO-d₆ 500 MHz TMS): 8.45 (d, J=9.0 Hz, 1H), 8.35 (d, J=8.5 Hz, 2H), 8.22 (d, J=9.0 Hz, 1H), 8.10 (d, J=8.5 Hz, 2H), 7.84 (d, J=9.5 Hz, 1H), 7.56 (t, J=9.5 Hz, 1H) ppm. MS (ESI): m/z=284.1 [M+H]⁺.

### Synthesis of Compound I-54, 4-(8-fluoro-6-hydroxyquinolin-2-yl)benzoic acid

### Step 1

2-chloro-8-fluoroquinolin-6-yl acetate (Intermediate 1-2) (89.3 mg) was treated with (4-(methoxycarbonyl)phenyl)boronic acid (74 mg), Pd(dppf)Cl₂ (cat.) and sodium bicarbonate (69 mg) in Dioxane (2 mL) and water (0.4 mL) at 100° C. with microwave heating over 2 hours. After aqueous work-up, a flash silica gel column purification afforded a mixture of methyl 4-(8-fluoro-6-hydroxyquinolin-2-yl)benzoate and methyl 4-(6-acetoxy-8-fluoroquinolin-2-yl)benzoate (120 mg) as light brown solids.

The above mixture (120 mg) was hydrolyzed with 2N NaOH (4 mL) in MeOH (4 mL). The desired Compound I-54 (65 mg) was collected by filtration after acidification with 12N HCl. ¹H NMR (DMSO-d₆ 300 MHz TMS): δ 8.36-8.33 (3H, m), 8.18 (1H, d, J=9 Hz), 8.09 (2H, d, J=9 Hz), 7.24 (1H, dd, J=12 and 3 Hz), 7.09 (1H, d, J=3 Hz) ppm, MS (ESI): m/z 284, [M+H⁺].

### Synthesis of Compound I-55, 3-hydroxy-4-(6-hydroxyquinolin-2-yl)benzoic acid

70 mg of 4-(6-hydroxyquinolin-2-yl)-3-methoxybenzoic acid (Compound I-48) was suspended in 10 ml DCM and 0.25 ml BBr₃ was added. The mixture was stirred at room temperature for 2 days. Water (20 ml) was added to quench the reaction. DCM was removed by evaporation and the precipitate was filtered and washed with water and dried to obtain 29 mg of 3-hydroxy-4-(6-hydroxyquinolin-2-yl)benzoic acid. 1H NMR (DMSO-d6 300 MHz TMS): δ 10.34 (s, 1H), 8.45 (d, 1H), 8.30 (d, 1H), 8.24 (d, 1H), 7.97 (d, 1H), 7.46 (m, 3H), 7.26 (d, 1H), MS (ESI): m/z=282.30 [M+H]⁺.

### Synthesis of Compound I-59, 4-(6-hydroxyquinolin-2-yl)-2-methoxybenzoic acid

2-chloroquinolin-6-ol (1 mmol, 0.18 g), (3-methoxy-4(methoxycarbonyl)phenyl) boronic acid (2 mmol, 0.42 g), 5% (Ph₃P)₄Pd, NaHCO₃ (6 mmol), and 20 ml 50% dioxane/water were combined. The reaction was degassed 3 times by evacuation and argon filling and was allowed to stir overnight at 95° C. The reaction was diluted with 10 ml water, and then filtered. The filtrate was acidified to pH 3-4 with 1N HCl. The resulting filtrate was isolated and the desired product (hydrolyzed) was obtained. Isolated 200 mg. ¹H NMR (DMSO-d₆, 300 MHz): δ 12.68 (b, 1H), 10.14 (s, 1H), 8.13 (d, 1H), 7.92 (d, 1H), 7.84 (m, 2H), 7.78 (m, 2H), 7.38 (d, 1H), 7.20 (s, 1H), 3.97 (s, 3H). MS (ESI): m/z 294.2 [M−H]⁻.

### Synthesis of Compound I-60, 2-hydroxy-4-(6-hydroxyquinolin-2-yl)benzoic acid

Compound I-59 (100 mg, 0.34 mmol) was dissolved in 15 ml dichloromethane and was cooled to 0° C. BBr₃ (97 uL) was added dropwise. The reaction was allowed to warm to room temperature and stirred overnight. Water (10 ml) was added and the solvent was removed. A precipitate formed and was isolated via centrifugation. The isolated product (90 mg) was the desired product as a gold solid. ¹H NMR (DMSO-d6, 300 MHz): δ 8.43 (d, 1H), 8.15 (d, 1H), 8.02 (d, 1H), 7.95 (d, 1H), 7.69 (s, 1H), 7.68 (d, 1H), 7.46 (d, 1H), 7.28 (s, 1H). MS (ESI): m/z 280.2 [M−H]⁻.

### Synthesis of Compound I-61, 2-(4-hydroxy-3-nitrophenyl)quinolin-6-ol

Followed the procedure described for Compound I-59 where the boronic acid used was 2-nitro-4-(4,4,5,5-tetramethyl-1,3,2-dioxaborolan-2-yl)phenol (1.5 equiv). After the desired solid was isolated, the compound was purified by a dichloromethane trituration and isolated via centrifugation. MS (ESI): m/z 281.3 [M−H]⁻.

### Synthetic Details for Compounds of Table 2

### Intermediate 2-1, 3-chloro-7-methoxy-4-methylisoquinoline

### Step 1

To a solution of i-Pr₂NH (1.95 g, 19.3 mmol) in anhydrous THF (40 mL) was added dropwise n-BuLi (7.72 mL, 19.3 mmol, 2.50 M in hexane) at −78° C. under N₂ atmosphere. After 30 minutes, 1,3-dichloro-7-methoxyisoquinoline (synthesis described in U.S. 61/423,799) (4.00 g, 17.5 mmol) in anhydrous THF (20 mL) was added to the reaction. After 15 minutes, MeI (4.97 g, 35.0 mmol) was added dropwise. The resulting mixture was warmed to 25° C. and allowed to stir for 16 hours. The reaction was quenched with H₂O (150 mL) followed by an ethyl acetate extraction/workup to give the crude product, which was purified by prep-HPLC (0.1% TFA as additive). Most of CH₃CN was removed under reduced pressure and extracted with EtOAc (50 mL×3). The combined organic layers were washed with H₂O (50 mL) and brine (50 mL), dried over anhydrous Na₂SO₄, filtered and concentrated in vacuo to give 1,3-dichloro-7-methoxy-4-methylisoquinoline (2.0 g, yield 47%) as an off-white solid.

### Step 2

To a solution of the above product (1.00 g, 4.13 mmol) in CH₃COOH (18 mL) and HCl (6 mL, 36%) was added Sn (1.46 g, 12.4 mmol) at 25-30° C. The resulting mixture was stirred at 60° C. for 2.5 hours. The reaction mixture was basified with aqueous NaOH (2M) to pH=7˜8, and the resulting mixture was extracted with EtOAc, followed by purification via silica gel column (PE/EtOAc=50/1 to 10/1) to give Intermediate 2-1 (470 mg, yield 55%) as a solid. ¹H NMR (CDCl₃ 400 MHz): δ 8.82 (s, 1H), 7.89 (d, J=9.6 Hz, 1H), 7.39 (dd, J=9.6, 2.8 Hz, 1H), 7.19 (d, J=2.8 Hz, 1H), 3.95 (s, 3H), 2.69 (s, 3H).

### Synthesis of Compound II-24, 3-fluoro-4-(7-hydroxyisoquinolin-3-yl)benzoic acid

### Step 1

A mixture of 3-chloro-7-methoxyisoquinoline (see U.S. 61/423,799 for synthesis) (300 mg, 1.55 mmol), 4-carboxy-2-fluorophenylboronic acid (285 mg, 1.55 mmol), K₂CO₃ (642 mg, 4.65 mmol) and Pd(dppf)Cl₂ (30 mg, 0.037 mmol) in DEGME (7 mL) and water (2 mL) was stirred under N₂ atmosphere at 120° C. for 3 hours. The resulting mixture was cooled to room temperature and filtered. The filtrate was acidified with aqueous HCl (2M) until pH=6 and filtered. The solid was dried under reduced pressure to give 3-fluoro-4-(7-methoxyisoquinolin-3-yl)benzoic acid (140 mg, yield: 30%) as off-white solid.

### Step 2

A mixture of the above product (70 mg, 0.24 mmol) and BBr₃ (0.2 mL, 2.1 mmol) in anhydrous DCM (2 mL) was stirred at 15° C. overnight. The resulting mixture was quenched with water (10 mL) and basified with aqueous sat. NaHCO₃ until pH=8. The mixture was concentrated in vacuo and purified by prep-HPLC (0.1% TFA as additive) to give Compound II-24 (12 mg, yield 18%) as yellow solid. ¹H NMR (CD₃OD 400 MHz TMS): δ 9.41 (s, 1H), 8.43 (s, 1H), 8.10 (d, J=8.8 Hz, 1H), 8.08-7.96 (m, 2H), 7.93 (dd, J=11.6, 0.8 Hz, 1H), 7.65 (dd, J=8.8, 2.4 Hz, 1H), 7.56 (d, J=2.4 Hz, 1H). MS (ESI): m/z 283.7 [M+H]⁺.

### Synthesis of Compound II-25, 4-(7-hydroxy-4-methylisoquinolin-3-yl)benzoic acid

### Step 1

To a mixture of 3-chloro-7-methoxy-4-methylisoquinoline (Intermediate 2-1) (470 mg, 2.27 mmol), 4-carboxyphenylboronic acid (451 mg, 2.72 mmol) and K₂CO₃ (627 mg, 4.54 mmol) in DEGME/H₂O (7 mL/1 mL) was added Pd(dppf)Cl₂ (40.8 mg, 0.050 mmol) under N₂ atmosphere. Then the mixture was heated to 120° C. for 16 hours. H₂O (30 mL) was added, and the resulting mixture was extracted with EtOAc (15 mL×2). The aqueous layer was neutralized with 1N aqueous HCl to pH=7, extracted with EtOAc (20 mL×3). The combined organic layers were washed with H₂O (20 mL) and brine (20 mL), dried over anhydrous Na₂SO₄, filtered and concentrated. Purification by silica gel column (PE/EtOAc=20/1 to 1/1, then EtOAc) gave 4-(7-methoxy-4-methylisoquinolin-3-yl)benzoic acid (85.0 mg, yield 13%).

### Step 2

To a solution of the above product (85.0 mg, 0.290 mmol) in anhydrous DCM (2 mL) was added BBr₃ (0.20 mL, 2.1 mmol, d=2.64 g/mL) dropwise at 0° C., then stirred at 25° C. for 16 hours. The reaction mixture was quenched with H₂O (20 mL) and neutralized with aqueous NaOH (2M) to pH=6˜7, followed by DCM extraction/workup. The crude product was tritutrated with EtOAc (15 mL) to give Compound II-25 (20 mg, yield 25%) as an off-white solid. ¹H NMR (MeOD₄ 400 MHz): δ 8.94 (s, 1H), 8.15 (d, J=8.0 Hz, 2H), 8.08 (d, J=8.8 Hz, 1H), 7.63 (d, J=8.0 Hz, 2H), 7.46 (dd, J=9.2, 2.4 Hz, 1H), 7.32 (d, J=2.4 Hz, 1H), 2.59 (s, 3H). MS (ESI): m/z 279.6 [M+H]⁺.

### Synthesis of Compound II-26, 3-fluoro-4-(6-hydroxy-3-(trifluoromethyl)quinoxalin-2-yl)benzoic acid

### Step 1

Followed the coupling procedure described in Step 1 of Compound I-46, where the starting materials were 2-chloro-6-methoxy-3-(trifluoromethyl)quinoxaline (synthesis described in U.S. 61/423,799) and 4-carboxy-2-fluorobezeneboronic acid and where the reaction here was heated to 120° C. for 2 h). After H₂O quench, the aqueous layer was acidified with 0.1M HCl to pH=1, followed by EtOAc/aqueous workup and column purification to give 3-fluoro-4-(6-methoxy-3-(trifluoromethyl)quinoxalin-2-yl)benzoic acid (50 mg, yield 19%).

### Step 2

Followed the BBr₃ deprotection method described in Compound II-24 (step 2). Compound II-26 (16 mg, yield 33%) was isolated as an off-white solid. ¹H NMR (MeOD 400 MHz): δ 8.09 (d, J=9.2 Hz, 1H), 8.07-7.99 (m, 1H), 7.88 (d, J=8.4 Hz, 1H), 7.70-7.62 (m, 2H), 7.49 (d, J=2.8 Hz, 1H). LC-MS purity: 100%. MS (ESI): m/z 352.9 [M+H]⁺.

### Synthesis of Compound II-27, (trans)-4-(6-hydroxy-3-(trifluoromethyl)quinoxalin-2-yl)cyclohexanecarboxylic acid

### Step 1

Followed the coupling procedure described in Step 1 of Compound I-46, where the starting materials were ethyl 4-(4,4,5,5-tetramethyl-1,3,2-dioxaborolan-2-yl)cyclohex-3-enecarboxylate and 2-chloro-6-methoxy-3-(trifluoromethyl)quinoxaline (synthesis described in U.S. 61/423,799) and where the reaction here was stirred under N₂ atmosphere at 100° C. for 3 hours. Ethyl 4-(6-methoxy-3-(trifluoromethyl)quinoxalin-2-yl)cyclohex-3-enecarboxylate was isolated (800 mg, yield 55%) as yellow solid.

### Step 2

A mixture of the above product (200 mg, 0.526 mmol) and 10% Pd/C (100 mg, 50% wet) in EtOH (10 mL) was stirred under H₂ (50 psi) at 30° C. for 2 days. The mixture was filtered and the filtrate was concentrated to give crude ethyl 4-(6-methoxy-3-(trifluoromethyl)-1,2,3,4-tetrahydroquinoxalin-2-yl)cyclohexanecarboxylate (190 mg).

### Step 3

A mixture of the above product (crude 190 mg) and MnO₂ (200 mg, 2.30 mmol) in DCM (20 mL) was stirred at 10° C. overnight. The mixture was filtered and the filtrate was concentrated followed by purification by silica gel column (PE/EtOAc=40/1) to give ethyl 4-(6-methoxy-3-(trifluoromethyl)quinoxalin-2-yl)cyclohexanecarboxylate (180 mg, 2-step yield: 90%) as colorless oil.

### Step 4

Followed the BBr₃ deprotection method described in Compound II-25 (step 2) to give Compound II-27 (trans) (23 mg, yield 12%) as off-white solid and Compound II-28 (cis) (22 mg, yield 12%) as off-white solid after prep-HPLC. ¹H NMR of Compound 27 (DMSO-d₆ 400 MHz): δ 7.97 (d, J=9.2 Hz, 1H), 7.88-7.78 (m, 1H), 7.12 (s, 1H), 3.10-2.98 (m, 1H), 2.48-2.38 (m, 2H), 2.40-2.20 (m, 1H), 2.10-1.90 (m, 2H), 1.53-1.51 (m, 2H), 1.51-1.46 (m, 2H). MS (ESI): m/z 340.8 [M+H]⁺.

### Synthesis of Compound II-28, (cis)-4-(6-hydroxy-3-(trifluoromethyl)quinoxalin-2-yl)cyclohexanecarboxylic acid

See Compound II-27 for synthesis. ¹H NMR (DMSO-d₆ 400 MHz): δ 12.12 (brs, 1H), 10.84 (brs, 1H), 7.99 (d, J=9.2 Hz, 1H), 7.55 (dd, J=9.2, 2.4 Hz, 1H), 7.31 (d, J=2.4 Hz, 1H), 3.08-2.94 (m, 1H), 2.36-2.28 (m, 1H), 2.10-2.00 (m, 2H), 1.92-1.70 (m, 4H), 1.54-1.38 (m, 2H). MS (ESI): m/z 340.7 [M+H]⁺.

### Synthetic Details for Compounds of Table 3

### Intermediate 3-1: methyl 4-(4,4,5,5-tetramethyl-1,3,2-dioxaborolan-2-yl)-3-(trifluoromethyl)benzoate

To a mixture of methyl 4-bromo-3-(trifluoromethyl)benzoate (2.00 g, 7.07 mmol), bis(pinacolato)diboron (3.60 g, 14.1 mmol), and KOAc (2.08 g, 21.2 mmol) in DMSO (30 mL) was added Pd(PPh₃)₄ (1.63 g, 1.41 mmol) under N₂ atmosphere. Then the mixture was heated to 120° C. for 3 hours. The reaction mixture was diluted with EtOAc (150 mL). The organic phase was separated, washed with H₂O (50 mL×3) and brine (50 mL), dried over anhydrous Na₂SO₄, filtered and concentrated to give the crude product (5.2 g) as a yellow oil.

### Intermediate 3-2: 3-fluoro-2-iodo-6-methoxynaphthalene

### Step 1

A mixture of 7-methoxynaphthalen-2-amine (84.0 g, 485 mmol) and Boc₂O (116 g, 534 mmol) in THF (500 mL) was stirred at 65° C. overnight. The mixture was concentrated, then was purified by silica gel column (PE/EtOAc=50/1) to give tert-butyl (7-methoxynaphthalen-2-yl)carbamate (95.0 g, yield 71%) as off-white solid.

### Step 2

To a mixture of the above product (20.0 g, 73.2 mmol) in anhydrous THF (1000 mL) was added t-BuLi (350 mL, 455 mmol, 1.3 M in pentane) dropwise at −20° C. under N₂ atmosphere. The reaction mixture was stirred at −10° C. for 30 minutes. Then 1,2-diiodoethane (51.6 g, 183 mmol) was added and the mixture was stirred at 20° C. for 1 hour. The mixture was quenched with water (1000 mL) and extracted with ethyl acetate (1000 mL×3). The combined organic layers were dried over anhydrous Na₂SO₄, concentrated, and then purified by silica gel column (PE/EtOAc=200/1˜5/1) to give tert-butyl (6-iodo-7-methoxynaphthalen-2-yl)carbamate (8.15 g, yield 28%) and a mixture of isomers (8.55 g) as off-white solid.

### Step 3

A mixture of tert-butyl (6-iodo-7-methoxynaphthalen-2-yl)carbamate with other isomer (8.00 g from above) and TFA (30 mL) in DCM (90 mL) was stirred at 20° C. for 3 hours, then concentrated. Aqueous saturated NaHCO₃ (200 mL) was added, then extracted with ethyl acetate (200 mL×3). The combined organic layers were dried over anhydrous Na₂SO₄ and concentrated. The residue was purified by silica gel column (PE/EtOAc=5/1) to give 3-iodo-7-methoxynaphthalen-2-amine (3.50 g, 2-step yield 16%) as off-white solid.

### Step 4

To a solution of the above product (1.50 g, 5.01 mmol) in water (20 mL) and conc. HCl (20 mL) was added a solution of NaNO₂ (345 mg, 5.01 mmol) in water (10 mL) dropwise at 0° C. The reaction was stirred at at 0° C. for 1 hour. Then HBF₄ (10 mL) was added and the mixture was stirred for 10 minutes. The mixture was filtered and the solid was washed with water (50 mL), dried under reduced pressure. The solid was dissolved in xylene (20 mL) and refluxed for 1 hour. The mixture was cooled to room temperature, diluted with water (50 mL), extracted with ethyl acetate (50 mL×3). The combined organic layers were dried over anhydrous Na₂SO₄ and concentrated under reduced pressure. The residue was purified by silica gel column (PE/EtOAc=100/1) to give 3-fluoro-2-iodo-6-methoxynaphthalene (1.20 g, yield 79%). ¹H NMR (CDCl₃ 400 MHz TMS): δ 8.19 (d, J=6.0 Hz, 1H), 7.63 (d, J=9.2 Hz, 1H), 7.38 (d, J=9.2 Hz, 1H), 7.10 (dd, J=9.2, 2.4 Hz, 1H), 7.03 (d, J=2.4 Hz, 1H), 3.92 (s, 3H).

### Intermediate 3-3, 3-chloro-2-iodo-6-methoxynaphthalene

To a solution of 3-iodo-7-methoxynaphthalen-2-amine (see Intermediate 3-2, product of step 3) (1.0 g, 3.34 mmol) in water (20 mL) and conc. HCl (20 mL) was added a solution of NaNO₂ (230 mg, 3.34 mmol) in water (10 mL) dropwise at 0° C. and the reaction was stirred at 0° C. for 1 hour. Then CuCl (400 mg, 4.04 mmol) was added and the mixture was stirred at 20° C. for 2 hours. An aqueous/EtOAc workup was followed by purification by silica gel column (PE/EtOAc=100/1) to give 3-chloro-2-iodo-6-methoxynaphthalene (900 mg, yield 85%) as off-white solid. ¹H NMR (CDCl₃ 300 MHz TMS): δ 8.29 (s, 1H), 7.84 (s, 1H), 7.60 (d, J=9.0 Hz, 1H), 7.13 (dd, J=9.0, 2.7 Hz, 1H), 6.99 (d, J=2.4 Hz, 1H), 3.91 (s, 3H).

### Intermediate 3-4, MPHT

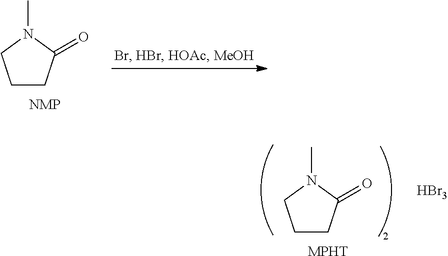

To MeOH (200 mL) was added HBr (97.2 g, 1.20 mol) in HOAc (120 mL) dropwise followed by the addition of Br₂ (190 g, 1.20 mol). The mixture was stirred at 10° C. for 10 minutes. Then NMP (257 g, 2.60 mol) was added dropwise. The reaction mixture was stirred at 10° C. for 1 hour. Then the mixture was filtered. The solid was washed with MTBE (200 mL) and dried under vacuum to give MPHT (361 g, yield 41%) as an orange solid. ¹H NMR (CDCl₃ 400 MHz): δ 3.72 (t, J=7.2 Hz, 4H), 3.07 (s, 6H), 2.92 (t, J=8.0 Hz, 4H), 2.32-2.18 (m, 4H).

### Intermediate 3-5, 6-(benzyloxy)-2-bromo-1-methoxynaphthalene

### Step 1

A mixture of 6-hydroxy-1-tetralone (50.0 g, 308 mmol), K₂CO₃ (64.0 g, 434 mmol) and BnBr (58.0 g, 340 mmol) in DMF (400 mL) was stirred at 25° C. for 16 hours. The mixture was diluted in water (1000 mL), extracted with EtOAc (1000 mL×3). The combined organic layers were washed with brine (500 mL×3), dried over anhydrous Na₂SO₄ and concentrated under reduced pressure to give 6-(benzyloxy)-3,4-dihydronaphthalen-1(2H)-one as a brown solid (54.0 g, yield 69%).

### Step 2

To a mixture of the above product (10.1 g, 40.0 mmol) in MeCN (20 mL) was added a solution of MPHT (Intermediate 3-4) (35.1 g, 80.0 mmol) in MeCN (130 mL) dropwise at 80° C. Then the reaction mixture was stirred at 80° C. for 1.5 hours. The mixture was quenched with aqueous saturated Na₂S₂O₃ (200 mL) and extracted with EtOAc (300 mL×3). The combined organic layers were washed with 5% aqueous HCl (100 mL×3), dried over anhydrous Na₂SO₄ and concentrated in vacuo. The residue was purified by silica gel column (PE/EtOAc=50/1) to give 6-(benzyloxy)-2,2-dibromo-3,4-dihydronaphthalen-1(2H)-one (7.18 g, yield 44%) as a white solid.

### Step 3

A mixture of the above product (7.18 g, 17.5 mmol) and TEA (50 mL) in anhydrous CHCl₃ (30 mL) was stirred at 25° C. for 4 hours. The mixture was quenched with aqueous saturated Na₂S₂O₃ (200 mL) and extracted with DCM (300 mL×3). The combined organic layers were washed with 5% aqueous HCl (100 mL×3), dried over anhydrous Na₂SO₄ and concentrated in vacuo. The residue was purified by silica gel column (PE) to give 6-(benzyloxy)-2-bromonaphthalen-1-ol (450 mg, yield 8%) as a white solid.

### Step 4

A mixture of the above product (500 mg, 1.51 mmol), K₂CO₃ (415 mg, 3.00 mmol) and CH₃I (0.75 mL, 14.8 mmol, 2.80 g/mL) in DMF (10 mL) under N₂ atmosphere was stirred at 25° C. for 18 hours. The mixture was diluted in water (50 mL) and extracted with EtOAc (50 mL×3), dried over anhydrous Na₂SO₄ and concentrated. The residue was purified by prep-TLC (PE/EtOAc=100/1) to give 6-(benzyloxy)-2-bromo-1-methoxynaphthalene (321 mg, yield 59%) as an off-white solid. ¹H NMR (CDCl₃ 300 MHz TMS): δ 8.04 (d, J=9.0 Hz, 1H), 7.56-7.46 (m, 3H), 7.46-7.34 (m, 4H), 7.30-7.24 (m, 1H), 7.19 (d, J=2.4 Hz, 1H), 5.17 (s, 2H), 3.99 (s, 3H).

### Intermediate 3-6, 3-bromonaphthalene-2,7-diol

To a solution of 2,7-dihydroxynaphthalene (8.00 g, 50.0 mmol) in AcOH (30 mL) was added Br₂ (16.0 g, 100 mmol) in AcOH (30 mL) dropwise over 20 minutes at 10-15° C. The mixture was stirred at the same temperature for 1 hour. Then Sn powder (12.4 g, 130 mmol) and H₂O (25 mL) was added and the mixture was stirred at 80° C. for 1 hour. After an aqueous/EtOAc workup, the crude was purified by column chromatography on silica gel (PE/EtOAc=5/1) and then prep-HPLC (0.1% TFA as additive) to give 3-bromonaphthalene-2,7-diol (8.2 g, yield 68%) as off-white solid. ¹H NMR (Acetone-d₆ 300 MHz TMS): δ 8.79 (brs, 1H), 7.98 (s, 1H), 7.63 (d, J=8.7 Hz, 1H), 7.16 (s, 1H), 7.04-6.92 (m, 2H).

### Intermediate 3-7, 6-(benzyloxy)-2-bromo-3-(methoxymethoxy)naphthalene

### Step 1

To a solution of 3-bromonaphthalene-2,7-diol (Intermediate 3-6) (4.00 g, 16.7 mmol) in MeCN (40 mL) was added K₂CO₃ (2.02 g, 14.5 mmol). The mixture was degassed for three times and MOMCl (1.87 g, 23.4 mmol) was added at −18° C. over 2 hours via syringe pump. The mixture was stirred at −18° C. for 2 hours and then quenched with water (50 mL). The mixture was acidified with aqueous HCl (2M) until pH=6 and extracted with EtOAc (50 mL×3), washed with brine (100 mL), dried over Na₂SO₄ and concentrated to give the crude product, which was purified by column chromatography on silica gel (PE/EtOAc=10/1) to give 6-bromo-7-(methoxymethoxy)naphthalen-2-ol (1.5 g, yield 32%) as off-white solid.

### Step 2

To a solution of the above product (1.50 g, 5.30 mmol) in DMF (15 mL) was added K₂CO₃ (1.47 g, 10.6 mmol) and BnBr (1.18 g, 6.89 mmol). The mixture was stirred at 80° C. overnight. Water (5 mL) was added and the mixture was acidified with aqueous HCl (0.1M) until pH=7 carefully and extracted with EtOAc (30 mL×3). The combined organic layers were washed with brine (50 mL), dried over Na₂SO₄ and concentrated in vacuo to give 6-(benzyloxy)-2-bromo-3-(methoxymethoxy)naphthalene (1.98 g, yield 100%) as yellow solid.

### Intermediate 3-8, 1-fluoro-6-methoxynaphthalen-2-yl trifluoromethanesulfonate

### Step 1

To a solution of 1-bromo-6-methoxynaphthalen-2-ol (described in U.S. 61/423,799) (2.90 g, 11.5 mmol) in MeOH (20 mL) and THF (20 mL) was added K₂CO₃ (3.18 g, 23.0 mmol) and MOMCl (1.10 g, 13.8 mmol). The resulting mixture was stirred at 20° C. for 48 hours and stirred at 30-40° C. for 5 days. An aqueous/EtOAc workup was followed by purification by silica gel column chromatography (PE to PE/EtOAc=200/1) to give 1-bromo-6-methoxy-2-(methoxymethoxy)naphthalene (1.90 g, yield 56%) as an off-white solid.

### Step 2

To a solution of the above product (1.00 g, 3.38 mmol) in anhydrous THF (20 mL) was added n-BuLi (1.62 mL, 4.06 mmol, 2.50 M in hexane) dropwise at 0° C. The mixture was stirred at 0° C. for 30 min then cooled to −78° C. A solution of N-fluorobenzenesulfonimide (1.28 g, 4.06 mmol) in anhydrous THF (5 mL) was added to the reaction mixture. The resulting mixture was stirred at −78° C. for 1 hour, then stirred at 25° C. for 12 hours. The reaction mixture was quenched with aq. NH₄Cl (60 mL), and extracted with EtOAc (20 mL×2). The combined organic layers were washed with H₂O (20 mL) and brine (20 mL), dried over anhydrous Na₂SO₄, filtered and concentrated under reduced pressure. The crude product was purified by silica gel column (PE to PE/EtOAc=200/1) to give 1-fluoro-6-methoxy-2-(methoxymethoxy)naphthalene (280 mg, yield 35%) as an off-white solid.

### Step 3

A mixture of the above product (770 mg, 3.26 mmol) in HCl/dioxane (15 mL) was stirred at 25° C. for 2 hours. The reaction mixture was neutralized with aqueous NaOH (2M) to pH=7, followed by an aqueous/EtOAc workup. The crude product was purified by silica gel column (PE/EtOAc=200/1 to 100/1) to give 1-fluoro-6-methoxynaphthalen-2-ol (380 mg, yield 61%) as an off-white solid.

### Step 4

To a solution of the above product (50.0 mg, 0.260 mmol) and TEA (34.2 mg, 0.338 mmol) in anhydrous DCM (10 mL) was added Tf₂O (80.7 mg, 0.286 mmol) at −50° C. The reaction mixture was stirred at −50° C. for 0.5 hour. The resulting mixture was quenched with brine (30 mL) and extracted with DCM (20 mL×3). The combined organic layers were washed with H₂O (20 mL) and brine (20 mL), dried over anhydrous Na₂SO₄, filtered and concentrated under reduced pressure to give 1-fluoro-6-methoxynaphthalen-2-yltrifluoromethanesulfonate (80 mg, yield 95%) as an off-white solid. ¹H NMR (CDCl₃ 400 MHz): δ 8.04 (d, J=9.2 Hz, 1H), 7.55 (d, J=8.8 Hz, 1H), 7.38-7.21 (m, 2H), 7.15 (s, 1H), 3.94 (s, 3H).

### Intermediate 3-9, 6-bromo-8-fluoronaphthalen-2-ol

### Step 1

To a solution of 1-fluoro-7-methoxynaphthalene (2.83 g) in HOAc (15 mL) was added bromine (5.68 g) in HOAc (20 mL) stepwise. After the addition, the mixture was stirred overnight and heated at 70° C. over 4 hours. After aqueous work-up, extraction with EtOAc, and washing with NaHCO₃ (sat), the resultant mixtures (two products: 1,5-dibromo-8-fluoro-2-methoxynaphthalene and 1,6-dibromo-8-fluoro-2-methoxynaphthalene) was purified by silica gel purification, eluting with Hexane/EtOAc (10:1), to isolate the desired product 1,6-dibromo-8-fluoro-2-methoxynaphthalene.

### Step 2

Following a literature procedure, intermediate 3-9 was prepared by the treatment of 1,6-dibromo-8-fluoro-2-methoxynaphthalene with SnCl₂ in acetic acid at 80° C., then followed by treatment with BBr₃ in dichloromethane to give intermediate 3-9.

### Intermediate 3-10: 6-bromo-1,8-difluoronaphthalen-2-ol

To a solution of 6-bromo-8-fluoronaphthalen-2-ol (Intermediate 3-9, 220 mg) in acetonitrile added was Selectfluoro™ fluorinating reagent (323 mg). The resultant solution was stirred overnight and then additional Selectfluoro™ fluorinating reagent (80 mg) was added. After stifling over 4 hours, the mixture was concentrated, diluted with EtOAc and washed with 1H HCl, water, brine. After removal of solvents, the mixture was purified by silica gel column to afford the desired product (132 mg) as colorless solids. ¹H NMR (CDCl₃, 300 MHz): δ 7.73 (t, J=3, Hz, 1H), 7.47 (t, J=9, 3 Hz, 1H), 7.33-7.26 (m, 2H), 6.69 (d, J=6 Hz, 1H), ¹⁹F NMR (CD3OD, 300 MHz): δ−115.98-−116.22 (ddd, J=54, 12, 3 Hz, 1F), −147.92-−147.96 (m); MS (ESI): m/z 257 [M−H]⁻.

### Synthesis of Compound III-20, 4-(6-hydroxynaphthalen-2-yl)-3-(trifluoromethyl)benzoic acid

### Step 1

To a mixture of methyl 4-bromo-3-(trifluoromethyl)benzoate (Intermediate 3-1) (5.20 g), 2-bromo-6-methoxy-naphthalene (1.50 g, 6.33 mmol), and K₂CO₃ (1.75 g, 12.7 mmol) in DEGME/H₂O (70 mL/10 mL) was added Pd(dppf)Cl₂ (114 mg, 0.139 mmol) under N₂ atmosphere. The mixture was heated to 120° C. for 3 hours. H₂O (150 mL) was added, extracted with EtOAc (50 mL×2). The aqueous layer was acidified with 1N aqueous HCl to pH=3-4, extracted with EtOAc (50 mL×3), washed with brine (50 mL), dried over anhydrous Na₂SO₄, filtered and concentrated. The crude product was purified by silica gel column (PE/EtOAc=10/1 to 1/1) to give 4-(6-methoxynaphthalen-2-yl)-3-(trifluoromethyl)benzoic acid (1.10 g) as a brown solid.

### Step 2

To a solution of the above product (1.10 g, from above crude) in anhydrous DCM (15 mL) was added BBr₃ (2.0 mL, 21.1 mmol, d=2.64 g/mL) dropwise at 0° C. The mixture was stirred at 25° C. for 16 hours, then quenched with H₂O (90 mL) and extracted with DCM (30 mL×3). The combined organic layers were washed with brine (30 mL), dried over anhydrous Na₂SO₄, filtered and concentrated to give the crude product which was purified by prep-HPLC (0.1% TFA as additive). Most of CH₃CN was removed under reduced pressure and the remaining solvent was removed by lyophilization to give Compound III-20 (21 mg) as an off-white solid. ¹H NMR (MeOD 400 MHz): δ 8.40 (s, 1H), 8.27 (d, J=8.0 Hz, 1H), 7.74 (d, J=8.8 Hz, 1H), 7.72-7.68 (m, 2H), 7.56 (d, J=8.0 Hz, 1H), 7.34 (d, J=8.8 Hz, 1H), 7.19-7.08 (m, 2H). MS (ESI): m/z 331.0 [M−H]⁻.

### Synthesis of Compound III-21, 3-chloro-4-(3-fluoro-6-hydroxynaphthalen-2-yl)benzoic acid

### Step 1

Followed the coupling procedure described in Step 1 of Compound III-20, where the starting materials were 3-fluoro-2-iodo-6-methoxynaphthalene (Intermediate 3-2) and 4-carboxy-2-chlorophenylboronic acid and wherein the reaction was heated to 90° C. for 3 hours. The desired 3-chloro-4-(3-fluoro-6-methoxynaphthalen-2-yl)benzoic acid (380 mg, yield 70%) was isolated as a solid after workup.

### Step 2

Followed the BBr₃ deprotection described for Compound III-20, Step 2. Compound III-21 was isolated (45 mg, yield 12%) as an off-white solid. ¹H NMR (DMSO-d₆ 400 MHz): δ 13.41 (brs, 1H), 10.05 (brs, 1H), 8.06 (d, J=1.6 Hz, 1H), 7.99 (dd, J=8.0, 1.6 Hz, 1H), 7.85 (d, J=8.0 Hz, 2H), 7.68-7.56 (m, 2H), 7.17 (d, J=2.4 Hz, 1H), 7.10 (dd, J=8.8, 2.4 Hz, 1H). MS (ESI): m/z 315.0 [M−H]⁻.

### Synthesis of Compound III-22, 4-(3-chloro-6-hydroxynaphthalen-2-yl)benzoic acid

### Step 1

Followed the coupling procedure described in Step 1 of Compound III-20, where the starting materials were 3-chloro-2-iodo-6-methoxynaphthalene (Intermediate 3-3) and 4-carboxyphenylboronic acid and where the reaction was stirred under N₂ atmosphere at 90° C. for 4 hours. After workup, the residue was triturated with DCM (50 mL) to give 4-(3-chloro-6-methoxynaphthalen-2-yl)benzoic acid (220 mg, yield: 75%) as an off-white solid.

### Step 2

Followed the BBr₃ deprotection described for Compound I-45, Step 3. Compound III-22 was isolated as a solid (24 mg, yield 25%). ¹H NMR (DMSO-d₆ 400 MHz): δ 13.02 (brs, 1H), 10.04 (brs, 1H), 8.02 (d, J=8.0 Hz, 2H), 7.96 (s, 1H), 7.87 (s, 1H), 7.84 (d, J=8.8 Hz, 1H), 7.61 (d, J=8.0 Hz, 2H), 7.16-7.08 (m, 2H). MS (ESI): m/z 297.0 [M−H]⁻.

### Synthesis of Compound III-23, 4-(3-fluoro-6-hydroxynaphthalen-2-yl)benzoic acid

### Step 1

Followed the coupling procedure described in Step 1 of Compound III-20, where the starting materials were 3-fluoro-2-iodo-6-methoxynaphthalene (Intermediate 3-2) and 4-carboxyphenylboronic acid and the reaction was heated to 90° C. for 3 hours. 4-(3-fluoro-6-methoxynaphthalen-2-yl)benzoic acid was isolated after workup (80 mg, crude).

### Step 2

Followed the BBr₃ deprotection described for Compound III-20, Step 2. Compound III-23 (11 mg, 2-step yield 2%) was isolated as an off-white solid. ¹H NMR (MeOD 400 MHz): δ 8.10 (d, J=8.4 Hz, 2H), 7.89 (d, J=8.0 Hz, 1H), 7.78 (d, J=8.8 Hz, 1H), 7.71 (d, J=6.8 Hz, 2H), 7.40 (d, J=12.4 Hz, 1H), 7.09 (d, J=2.0 Hz, 1H), 7.05 (dd, J=8.8, 2.0 Hz, 1H). MS (ESI): m/z 281.0 [M−H]⁻.

### Synthesis of Compound III-24, 4-(6-hydroxy-1-methoxynaphthalen-2-yl)benzoic acid

### Step 1

Followed the coupling procedure described in Step 1 of Compound III-20, where the starting materials were 6-(benzyloxy)-2-bromo-1-methoxynaphthalene (Intermediate 3-5) and 4-carboxyphenylboronic acid and where the reaction was stirred at 130° C. for 5 hours. 4-(6-(benzyloxy)-1-methoxynaphthalen-2-yl)benzoic acid (96 mg, yield 43%) was isolated as a yellow solid.

### Step 2

A mixture of the above product (96 mg, 0.25 mmol) and 10% Pd/C (100 mg, 50% wet) in EtOAc (10 mL) was stirred under H₂ (15 psi) at 30° C. for 18 hours. The mixture was filtered and the filtrate was concentrated in vacuo. The residue was purified by prep-TLC (PE/EtOAc=2/1) to give Compound III-24 (13.5 mg, yield 19%) as an off-white solid. ¹H NMR (CD₃OD 400 MHz TMS): δ 8.18-8.06 (m, 3H), 7.78 (d, J=8.4 Hz, 2H), 7.51 (d, J=8.8 Hz, 1H), 7.42 (d, J=8.4 Hz, 1H), 7.14 (s, 1H), 7.13 (d, J=7.6 Hz, 1H), 3.55 (s, 3H). MS (ESI): m/z 293.0 [M−H]⁻.

### Synthesis of Compound III-25, 4-(3,6-dihydroxynaphthalen-2-yl)benzoic acid

Followed the coupling procedure described in Step 1 of Compound III-20, where the starting materials were 3-bromonaphthalene-2,7-diol (Intermediate 3-6) and 4-carboxyphenylboronic acid in DMF and water, and the reaction was stirred at 110° C. for 3 hours. Purification by prep-HPLC (0.1% TFA as additive) gave Compound III-25 (115 mg, yield 20%) as an off-white solid. ¹H NMR (DMSO-d₆ 400 MHz TMS): δ 9.89 (brs, 1H), 9.64 (brs, 1H), 7.97 (d, J=8.0 Hz, 2H), 7.76-7.70 (m, 3H), 7.66 (d, J=8.8 Hz, 1H), 7.03 (s, 1H), 6.88 (d, J=1.6 Hz, 1H), 6.84 (dd, J=8.8, 2.0 Hz, 1H). MS (ESI): m/z 279.0 [M−H]⁻.

### Synthesis of Compound III-26, 4-(1-fluoro-6-hydroxynaphthalen-2-yl)benzoic acid

### Step 1

A mixture of 1-fluoro-6-methoxynaphthalen-2-yl trifluoromethanesulfonate (Intermediate 3-8) (80.0 mg, 0.247 mmol), 4-methoxycarbonylphenylboronic acid (44.5 mg, 0.247 mmol) and aqueous Na₂CO₃ (2M, 0.27 mL, 0.54 mmol) in toluene/EtOH (4 mL/1 mL) was degassed three times under N₂ atmosphere. Then Pd(PPh₃)₄ (28.6 mg, 0.0247 mmol) was added and the mixture was stirred at 80° C. for 5 hours under N₂ atmosphere. An aqueous/EtOAc workup was followed by silica gel column chromatography (PE/EtOAc=200/1 to 100/1) to give methyl 4-(1-fluoro-6-methoxynaphthalen-2-yl)benzoate (50 mg, yield 65%) as an off-white solid.

### Step 2

To a solution of the above product (50.0 mg, 0.161 mmol) in anhydrous DCM (5 mL) was added BBr₃ (0.20 mL, 2.1 mmol, d=2.64 g/mL) dropwise at 0° C. The resulting mixture was stirred at 25° C. for 16 hours. An aqueous/DCM workup gave methyl 4-(1-fluoro-6-hydroxynaphthalen-2-yl)benzoate (50 mg, crude) as an off-white solid, which was used for next step directly. MS (ESI): m/z 295.0 [M−H]⁻.

### Step 3

To a solution of the above product (50 mg, 43.6% purity in LCMS) in MeOH (3 mL) was added aqueous NaOH (3 mL, 2M). The mixture was stirred at 25° C. for 16 hours. H₂O (30 mL) was added to the reaction mixture, and the resulting mixture was extracted with EtOAc (15 mL×2), the aqueous layer was acidified with 1N HCl to pH=1˜2, extracted with EtOAc (15 mL×3). The combined organic layers were washed with brine (20 mL), dried over anhydrous Na₂SO₄, filtered and concentrated to give the crude product. The crude product was purified by prep-HPLC (0.1% TFA as additive). Most of CH₃CN was removed under reduced pressure and the remaining solvent was removed by lyophilization to give Compound III-26 (5 mg, 2-step yield: 11%) as a white solid. ¹H NMR (MeOD 400 MHz): δ 8.12 (d, J=8.4 Hz, 2H), 8.02 (d, J=8.8 Hz, 1H), 7.75 (d, J=7.6 Hz, 2H), 7.58-7.47 (m, 2H), 7.21-7.12 (m, 2H). MS (ESI): m/z 281.0 [M−H]⁻.

### Synthesis of Compound III-27, 4-(6-hydroxy-3-methylnaphthalen-2-yl)benzoic acid

### Step 1

Followed the coupling procedure described in Step 1 of Compound III-20, where the starting materials were 6-(benzyloxy)-2-bromo-3-(methoxymethoxy)naphthalene (Intermediate 3-7) and 4-methoxycarbonylphenylboronic acid and where the reaction was stirred at 90° C. for 3 hours. 4-(6-(benzyloxy)-3-(methoxymethoxy)naphthalen-2-yl)benzoic acid (1.30 g, yield 72%) was obtained as an off-white solid.

### Step 2

A mixture of the above product (1.20 g, 2.90 mmol), K₂CO₃ (800 mg, 5.80 mmol) and CH₃I (824 mg, 5.80 mmol) in DMF (15 mL) was stirred at 10° C. for 4 hours. The mixture was neutralized with aqueous HCl (0.1M) until pH=7 and extracted with EtOAc (50 mL×3). The combined organic layers were washed with brine (100 mL), dried over Na₂SO₄ and concentrated under reduced pressure to give methyl 4-(6-(benzyloxy)-3-(methoxymethoxy)naphthalen-2-yl)benzoate (1.10 g, yield 89%) as an off-white solid.

### Step 3

To a solution of the above product (1.10 g, 2.57 mmol) in THF/MeOH (8 mL/2 mL) was added conc. HCl (0.2 mL). The mixture was refluxed for 3 hours. The mixture was concentrated under reduced pressure to give the crude product, which was washed with PE (50 mL) and EtOAc (30 mL) to give methyl 4-(6-(benzyloxy)-3-hydroxynaphthalen-2-yl)benzoate (900 mg, yield 91%) as white solid.

### Step 4

To a solution of the above product (615 mg, 1.60 mmol) and Et₃N (1.30 g, 12.9 mmol) in DCM (20 mL) was added Tf₂O (903 mg, 3.20 mmol) dropwise and then the mixture was stirred at 10° C. for 30 minutes. After an aqueous/EtOAc workup, the residue was purified by silica gel column (PE/EtOAc=40/1) to give methyl 4-(6-(benzyloxy)-3-(((trifluoromethyl)sulfonyl)oxy)naphthalen-2-yl)benzoate (450 mg, yield: 54%) as an off-white solid.

### Step 5

To a solution of ZnCl₂ (1.32 g, 9.68 mmol) in anhydrous THF (20 mL) was added MeMgCl (1.6 mL, 4.80 mmol, 3M in THF) and the mixture was stirred at under N₂ atmosphere at 10° C. for 1 hour. Then methyl 4-(6-(benzyloxy)-3-(((trifluoromethyl)sulfonyl)oxy)naphthalen-2-yl)benzoate (500 mg, 0.968 mmol) and Pd(PPh₃)₄ (100 mg, 0.0865 mmol) was added and the mixture was stirred at 60° C. under N₂ atmosphere for 3 hours. After an aqueous/EtOAc workup, the residue was purified by silica gel column (PE/EtOAc=40/1) to give methyl 4-(6-(benzyloxy)-3-methylnaphthalen-2-yl)benzoate (250 mg, yield 68%) as an off-white solid.

### Step 6

A mixture of the above product (150 mg, 0.392 mmol) and aqueous NaOH (10 mL, 2M) in MeOH (10 mL) was refluxed overnight. The mixture was cooled to room temperature, acidified with aqueous HCl (2M) until pH=5 and extracted with EtOAc (50 mL×3), dried over Na₂SO₄ and concentrated under reduced pressure to give 4-(6-(benzyloxy)-3-methylnaphthalen-2-yl)benzoic acid (100 mg, yield 69%) as off-white solid.

### Step 7

A mixture of the above product (100 mg, 0.271 mmol) and 10% Pd/C (50 mg, 50% wet) in EtOAc (10 mL) was stirred at 20° C. for 2 days. The mixture was filtered and the filtrate was concentrated under reduced pressure. The residue was purified by prep-HPLC (0.1% TFA as additive) to give Compound III-27 (60 mg, yield 79%) as off-white solid. ¹H NMR (CD₃OD 400 MHz TMS): δ 8.09 (d, J=8.0 Hz, 2H), 7.69 (d, J=8.8 Hz, 1H), 7.58 (s, 1H), 7.54 (s, 1H), 7.49 (d, J=8.0 Hz, 2H), 7.06 (d, J=2.4 Hz, 1H), 7.02 (dd, J=8.4, 2.4 Hz, 1H), 2.35 (s, 3H). MS (ESI): m/z 277.0 [M−H]⁻.

### Synthesis of Compound III-34, 4-(6-hydroxynaphthalen-2-yl)-2-methoxybenzoic acid

6-chloronaphthalen-2-ol (1 mmol, 0.223 g), (3-methoxy-4-(methoxycarbonyl) phenyl) boronic acid (2 mmol, 0.42 g), 5% (Ph₃P)₄Pd, NaHCO₃ (6 mmol), and 20 ml 50% dioxane/water were combined. The reaction was degassed 3 times by evacuation and argon filling and was allowed to stir overnight at 95° C. The reaction was diluted with 10 ml water, and then filtered. The filtrate was acidified to pH 3-4 with 1N HCl. The resulting filtrate was isolated and the desired product (hydrolyzed) was obtained. The isolated solid was triturated with acetone and isolated via centrifugation to give 62 mg of desired product. ¹H NMR (DMSO-d6, 300 MHz): δ 12.56 (b, 1H), 9.88 (s, 1H), 8.20 (s, 1H), 7.75 (d, 1H), 7.75 (m, 3H), 7.38 (m, 2H), 7.14 (m, 2H), 3.96 (s, 3H). MS (ESI): m/z 293.3 [M−1]⁻.

### Synthesis of Compound III-35, 2-hydroxy-4-(6-hydroxynaphthalen-2-yl)benzoic acid

Compound III-34 (30 mg, 0.10 mmol) was dissolved in 5 ml dichloromethane and was cooled to 0° C. BBr₃ (28 uL, 0.3 mmol) was added dropwise. The reaction was allowed to warm to room temperature and stirred 5 hours. Water (5 ml) was added and the solvent was removed. A precipitate formed and was isolated via centrifugation to give the desired product (25 mg). ¹H NMR (DMSO-d6, 300 MHz): δ 11.30 (b, 1H), 9.82 (b, 1H), 8.20 (s, 1H), 7.85 (d, 2H), 7.78 (s, 2H), 7.37 (m, 2H), 7.16 (m, 2H), MS (ESI): m/z 279.3 [M−1]⁻.

### Synthesis of Compound III-36, 6-(4-hydroxy-3-nitrophenyl)naphthalen-2-ol

Followed the procedure described for Compound III-34 where the boronic acid used was 2-nitro-4-(4,4,5,5-tetramethyl-1,3,2-dioxaborolan-2-yl)phenol (1.5 equiv). After the desired solid was isolated, the compound was purified by a dichloromethane trituration and isolated via centrifugation to give 40 mg of desired product. MS (ESI): m/z 280.3 [M−H]⁻.

### Synthesis of Compound III-37, 4-(6-hydroxynaphthalen-2-yl)-3-methylbenzoic acid

Followed the procedure described for Compound III-34 where the boronic acid used was methyl 3-methyl-4-(4,4,5,5-tetramethyl-1,3,2-dioxaborolan-2-yl)benzoate (1.5 equiv). After the desired solid (hydrolyzed) was isolated, the compound was purified by column chromatography (gradient of 2-60% hexanes/[AcOH:MeOH:EtoAc 1:5:94]) to give 20 mg of desired product. MS (ESI): m/z 277.23 [M−H]⁻.

### Synthesis of Compound III-38, 3-fluoro-4-(4-fluoro-6-hydroxynaphthalen-2-yl)benzoic acid

Following a general Suzuki coupling reaction condition similar to Compound III-34, the title compound was prepared from the coupling of 6-bromo-8-fluoronaphthalen-2-ol (Intermediate 3-9) with 4-borono-3-fluorobenzoic acid. ¹H NMR (CD3OD, 300 MHz): δ 7.94 (dd, J=6, 3, Hz, 1H), 7.89-7.81 (m, 3H), 7.72 (t, J=9, 1H), 7.52 (dt, J=12, 3 Hz, 1H), 7.31 (d, J=3 Hz, 1H), 7.20 (dd, J=9, 3 Hz, 1H), ¹⁹F NMR (CD3OD, 300 MHz): δ−118.99-−119.07 (m, 1F), −126.74 (dd, J=12, 3 Hz); MS (ESI): m/z 301 [M+H]⁺.

### Synthesis of Compound III-39, 4-(4-fluoro-6-hydroxynaphthalen-2-yl)benzoic acid

Following a general Suzuki coupling reaction condition similar to Compound III-34, the title compound was prepared from the coupling of 6-bromo-8-fluoronaphthalen-2-ol (Intermediate 3-9) with (4-(methoxycarbonyl)phenyl)boronic acid. This was followed by basic hydrolysis. ¹H NMR (CD₃OD, 300 MHz): δ 8.12 (d, J=9 Hz, 2H), 7.95 (s, br, 1H), 7.89 (dd, J=9, 3 Hz, 1H), 7.58 (d, J=9, 2H), 7.52 (dd, J=12, 3 Hz, 1H), 7.30 (d, J=3 Hz, 1H), 7.19 (dd, J=9, 3 Hz, 1H), ¹⁹F NMR (CD₃OD, 300 MHz): δ−126.38 (dd, J=12, 3 Hz) ppm; MS (ESI): m/z 283 [M+H⁺]⁺.

### Synthesis of Compound III-40, 4-(4,5-difluoro-6-hydroxynaphthalen-2-yl)-3-fluorobenzoic acid

Following a general Suzuki coupling reaction condition similar to Compound III-34, the title compound was prepared from the coupling of 6-bromo-1,8-difluoronaphthalen-2-ol (Intermediate 3-10) with 4-borono-3-fluorobenzoic acid. ¹H NMR (D6-DMSO, 300 MHz): δ 13.38 (s, br, 1H), 8.01 (s, b, 1H), 7.84-7.78 (m, 3H), 7.58 (dt, J=12, 3 Hz, 1H), 7.39 (t, J=9, 1H), ¹⁹F NMR (CD3OD, 300 MHz): δ−117.01-−117.08 (m, 1F), −118.94 (dd, J=12, 3 Hz, 1F), −145.90 (dd, J=54, 9 Hz, 1F) ppm; MS (ESI): m/z 319 [M+H]⁺.

### Synthesis of Compound III-41, 4-(4,5-difluoro-6-hydroxynaphthalen-2-yl)-3-methylbenzoic acid

Following a general Suzuki coupling reaction condition similar to Compound III-34, the title compound was prepared from the coupling of 6-bromo-1,8-difluoronaphthalen-2-ol (Intermediate 3-10) with 4-borono-3-methylbenzoic acid. ¹H NMR (D6-DMSO, 300 MHz): δ 13.01 (s, br, 1H), 10.38 (s, b, 1H), 7.91 (m, 1H), 7.85 (dd, J=9, 3 Hz, 1H), 7.76-7.72 (m, 2H), 7.44 (d, J=6 Hz, 1H), 7.40-7.34 (m, 2H), 2.35 (s, 3H), ¹⁹F NMR (CD3OD, 300 MHz): δ−119.46 (ddd, J=54, 15, 3 Hz, 1F), −146.08 (dd, J=54, 9 Hz, 1F) ppm; MS (ESI): m/z 315 [M+H]⁺.

### Synthesis of Compound III-42, 4-(4,5-difluoro-6-hydroxynaphthalen-2-yl)benzoic acid

Following a general Suzuki coupling reaction condition similar to Compound III-34, the title compound was prepared from the coupling of 6-bromo-1,8-difluoronaphthalen-2-ol (Intermediate 3-10) with (4-(methoxycarbonyl)phenyl)boronic acid, followed by base hydrolysis. ¹H NMR (D6-DMSO, 300 MHz): δ 13.03 (s, br, 1H), 10.43 (s, br, 1H), 8.17 (t, J=3 Hz, 1H), 8.05 (d, J=9 Hz, 2H), 7.96 (d, J=9 Hz, 2H), 7.81-7.73 (m, 2H), 7.37 (t, J=9 Hz, 1H), 7.39 (t, J=9, 1H), ¹⁹F NMR (CD3OD, 300 MHz): δ−118.65 (ddd, J=54, 15, 3 Hz, 1F), −146.00 (dd, J=54, 9 Hz, 1F) ppm; MS (ESI): m/z 301 [M+H]⁺.

### Synthesis of Compound III-43, 4-(4,5-difluoro-6-hydroxynaphthalen-2-yl)-3-methoxybenzoic acid

Following a general Suzuki coupling reaction condition similar to Compound III-34, the title compound was prepared from the coupling of 6-bromo-1,8-difluoronaphthalen-2-ol (Intermediate 3-10) with 4-borono-3-methoxybenzoic acid. ¹H NMR (D6-DMSO, 300 MHz): δ 13.10 (s, br, 1H), 10.37 (s, 1H), 7.86 (t, J=3 Hz, 1H), 7.75 (d, J=9 Hz, 1H), 7.67-7.63 (m, 2H), 7.56 (d, J=9 Hz, 1H), 7.59 (dd, J=15, 3 Hz, 1H), 7.35 (dd, J=9, 6 Hz, 1H), 3.88 (s, 3H), ¹⁹F NMR (CD3OD, 300 MHz): δ−120.16-−120.40 (ddd, J=54, 15, 3 Hz, 1F), −146.06-−146.27 (dd, J=54, 15 Hz, 1F) ppm; MS (ESI): m/z 331 [M+H]⁺.

### Synthetic Details for Compounds of Table 4

### Intermediate 4-1, 1-(2,4-dihydroxyphenyl)-2-(4-hydroxy-3-nitrophenyl)ethanone

### Step 1

To a solution of 2-(4-hydroxy-3-nitrophenyl)acetic acid (2 g, 10.2 mmol) in DCM (30 mL) was added one drop of DMF. Then (COCl)₂ (17.5 mL, 203.2 mmol) was added with stifling. The mixture was stirred at room temperature for 30 min. The volatiles were removed to afford crude 2-(4-hydroxy-3-nitrophenyl)acetyl chloride (2.18 g, about 99%) as a yellow solid. MS(ESI): m/z 212.0 [M+1]⁺.

### Step 2

To a flask was added the above product (2.18 g, 10.14 mmol), 1,3-dimethoxybenzene (2.10 g, 15.22 mmol), AlCl₃ (2.02 g, 15.18 mmol) and DCM (30 mL). The reaction was stirred at room temperature overnight. The mixture was poured into 50 mL of ice-water, extracted with EtOAc (30 mL×2), and washed with water (30 mL×3), dried over Na₂SO₄, concentrated and purified by silica gel chromatography (PE:EA=10:1) to afford 1-(2,4-dimethoxyphenyl)-2-(4-hydroxy-3-nitrophenyl)ethanone as a yellow solid (1.0 g, 31%).

### Step 3

To a solution of the above product (500 mg, 1.58 mmol) in DCM (30 mL) was added BBr₃ (2.5 mL, 31.60 mmol). The reaction was stirred at room temperature overnight. A standard aqueous/EtOAc workup (see step 2) was followed by purification by silica gel chromatography (PE:EA=5:1) to afford Intermediate 4-1 as a yellow solid (110 mg, 24%). MS (ESI): m/z 290.0 [M+1]⁺.

### Intermediate 4-2, 1-(2,4-dihydroxyphenyl)-2-(3-ethoxy-4-hydroxyphenyl)ethanone

To a flask was added 2-(3-ethoxy-4-hydroxyphenyl)acetic acid (1 g, 5.102 mmol), resorcinol (0.842 g, 7.655 mmol) and BF₃-Et₂O (20 mL). The reaction mixture was heated to 95° C. and stirred at that temperature for 4 h. The mixture was cooled to room temperature, poured into aqueous Na₂CO₃ solution (30 mL), extracted with EA (30 mL×3), washed with water (30 mL) and brine (30 mL). The organic phase was dried over Na₂SO₄ and concentrated to give crude product, which was purified by silica gel chromatography (PE:EA=5:1) to afford 1-(2,4-dihydroxyphenyl)-2-(3-ethoxy-4-hydroxyphenyl)ethanone as a yellow solid (1.17 g, 80%). MS (ESI): m/z 289.1 [M+1]⁺.

### Synthesis of Compound IV-45, 7-hydroxy-3-(4-hydroxy-3-nitrophenyl)-2-(trifluoromethyl)-4H-chromen-4-one

To a solution of 1-(2,4-dihydroxyphenyl)-2-(4-hydroxy-3-nitrophenyl)ethanone (Intermediate 4-1) (110 mg, 0.38 mmol) in DCM (10 mL) was added TEA (308 mg, 3.05 mmol). After stifling for 5 min, TFAA (400 mg, 1.91 mmol) was added. The mixture was stirred at room temperature for 30 min. The volatiles were removed, extracted with EtOAc (30 mL), washed with 5% HCl (20 mL) and brine (20 mL). The organic phase was dried over Na₂SO₄ and concentrated to give crude product. The crude was washed with DCM (5 mL) to afford Compound IV-45 (40 mg, 29%). ¹H NMR (MeOH-d₄ 500 MHz TMS): 8.04 (d, J=1.5 Hz, 1H), 8.03 (s, 1H), 7.54 (dd, J=2.0, 8.5 Hz, 1H), 7.25 (d, J=8.5 Hz, 1H), 7.03 (dd, J=2.0, 8.5 Hz, 1H), 6.95 (d, J=2.5 Hz, 1H); MS (ESI): m/z 368.0 [M+1]⁺.

### Synthesis of Compound IV-46, 3-(3-ethoxy-4-hydroxy-5-nitrophenyl)-7-hydroxy-2-(trifluoromethyl)-4H-chromen-4-one

### Step 1

To a solution of 1-(2,4-dihydroxyphenyl)-2-(3-ethoxy-4-hydroxyphenyl)ethanone (1 g, 3.472 mmol) in DCM (10 mL) was added TEA (2.81 g, 25.55 mmol). After stirring for 5 min, TFAA (3.65 g, 17.38 mmol) was added. The mixture was stirred at room temperature for 30 min. The volatiles were removed. The residue was extracted with EtOAc (100 mL), washed with 5% HCl (50 mL) and brine (50 mL). The organic phase was dried over Na₂SO₄ and concentrated to give crude product, which was rinsed with DCM to afford 3-(3-ethoxy-4-hydroxyphenyl)-7-hydroxy-2-(trifluoromethyl)-4H-chromen-4-one (1.2 g, 95%).

### Step 2

Fuming HNO₃ (1 mL, 16.00 mmol) was added dropwise to a solution of the above product (1.2 g, 3.279 mmol) in CH₃COOH (3 mL). The reaction mixture was stirred for 20 min while the temperature was maintained below 15° C., The mixture was diluted with 20 mL of H₂O, extracted with EtOAc (30 mL) and washed with water (20 mL×3). The organic phase was dried over Na₂SO₄ and concentrated. Purified by prep-HPLC to afford 40 mg of Compound IV-46 as a brown solid. ¹H NMR (DMSO-d₆ 500 MHz TMS): 11.19 (s, 1H), 10.52 (s, 1H), 7.95 (d, J=9.0 Hz, 1H), 7.40 (d, J=1.0 Hz, 1H), 7.25 (d, J=1.5 Hz, 1H), 7.04-7.02 (dd, J=2.0, 9.0 Hz, 1H), 6.97 (d, J=2.0 Hz, 1H), 4.10 (q, J=6.5 Hz, 2H), 1.35 (t, J=7.0 Hz, 3H); MS (ESI): m/z 412.0 [M+1]⁺.

### Synthetic Details for Compounds of Table 5

Schemes 5-1 and 5-2 are generic methods for preparing Compounds of Table V. Scheme 5-1 is the generic synthesis of Intermediates. Scheme 5-2 shows the generic synthesis of the final products.

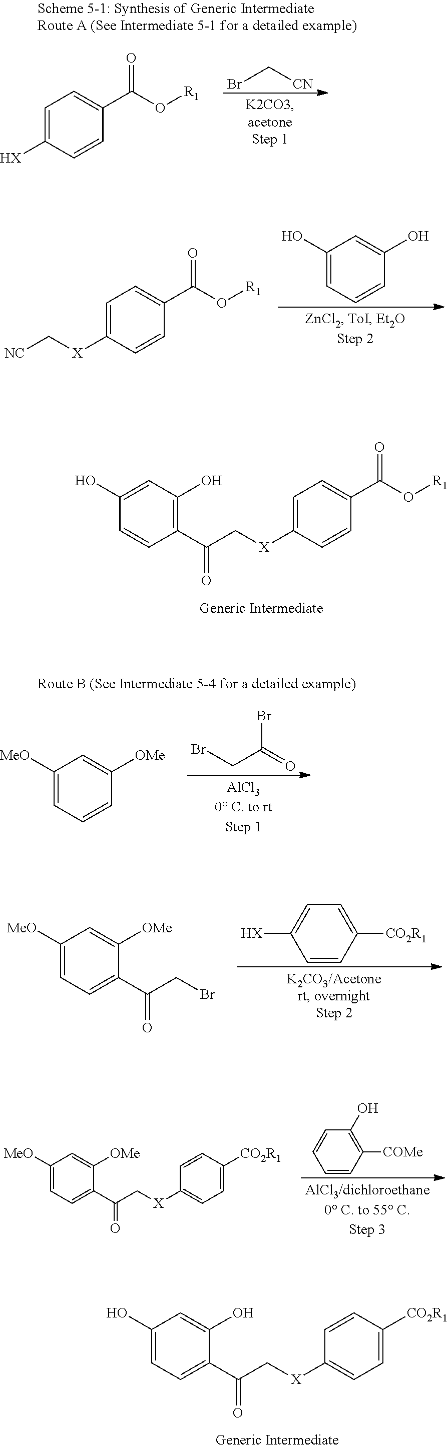

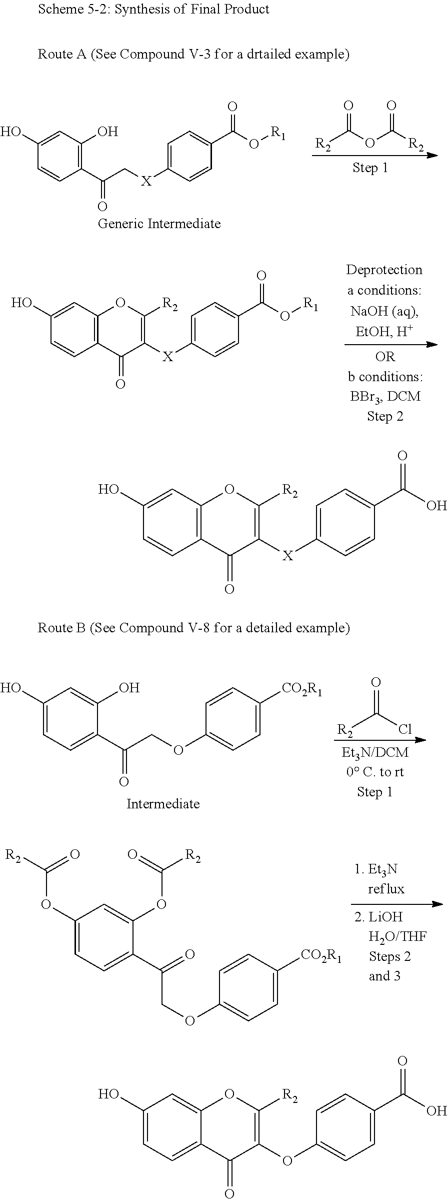

### Synthesis of Intermediate 5-1: ethyl 4-(2-(2,4-dihydroxyphenyl)-2-oxoethoxy)benzoate

Followed Scheme 5-1, Route A

### Step 1

To a mixture of ethyl 4-hydroxybenzoate (10.92 g, 65.7 mmol) and potassium carbonate (18.21 g, 131.4 mmol) in acetone (250 mL) was added 2-bromoacetonitrile (7.9 g, 65.7 mmol), and the mixture was stirred at 60° C. for 11 h. The acetone was removed under reduced pressure, and the residue was diluted with water. The resulting mixture was extracted with ethyl acetate (160 mL×2), dried over sodium sulfate (10 g), and concentrated under reduced pressure to give ethyl 4-(cyanomethoxy)benzoate (13.4 g, yield 99.3%).

### Step 2

To a solution of ethyl 4-(cyanomethoxy)benzoate (12 g, 58.48 mmol) in absolute toluene (150 mL) at 0° C. was bubbled with dry hydrogen chloride gas for 1 h, then a solution of resorcinol (7.73 g, 70.18 mmol) and fresh zinc chloride (3.99 g, 29.24 mmol) in dry ether (30 mL) was added, and the bubbling of the hydrogen chloride was continued for 2 h. Then the reaction mixture was stirred at room temperature overnight, filtered, and the filter cake was washed with hot water (300 mL). The aqueous mixture was heated at reflux for 2 h. The precipitate was collected by filtration, washed was water until the pH=7, and purified by column chromatography on silica gel (petroleum ether/ethyl acetate=6:1) to give ethyl 4-(2-(2,4-dihydroxyphenyl)-2-oxoethoxy)benzoate (2.6 g, yield 14%).

### Synthesis of Intermediate 5-2: benzyl 4-(2-(2,4-dihydroxyphenyl)-2-oxoethoxy)benzoate

Followed the procedure described in Scheme 5-1, Route A, starting from benzyl 4-hydroxybenzoate and wherein in the first step the reaction was heated to 70° C. for 2 h. ¹H NMR of Intermediate 5-2 (DMSO-d₆ 300 MHz): δ 11.59 (s, 1H), 10.63 (s, 1H), 7.92 (d, J=8.7 Hz, 2H), 7.75 (d, J=8.7 Hz, 1H), 7.46-7.36 (m, 5H), 7.04 (d, J=8.7 Hz, 2H), 6.38 (d, J=8.7 Hz, 1H), 6.33 (d, J=1.8 Hz, 1H), 5.52 (s, 2H), 5.31 (s, 2H).

### Synthesis of Intermediate 5-3: 4-(2-(2,4-dihydroxyphenyl)-2-oxoethoxy)benzonitrile

Followed a procedure similar to that described in the synthesis of Intermediate 5-1, starting from 4-hydroxybenzonitrile and wherein step 1 was refluxed for 4 hours and purified by column chromatography on silica gel (PE:EtOAc=10:1). Step 2 was isolated as a solid and taken on without purification.

### Synthesis of Intermediate 5-4: methyl 4-(2-(2,4-dihydroxyphenyl)-2-oxoethoxy)benzoate

Example of Scheme 5-1, Route B

### Step 1

To a solution of 1,3-dimethoxybenzene (30 g, 217 mmol) and bromoacetylbromide (43.2 g, 217 mmol) was added AlCl₃ (28.80 g, 217 mmol) in 6 portions below 0° C. After the addition, the mixture was allowed to warm to room temperature and stirred for 12 h. The mixture was poured carefully into 6 N HCl (1000 mL) and extracted with EA (200 mL×3). The combined organic layers were washed with brine (200 mL), dried with magnesium sulfate, filtered and concentrated to afford brown oil, which was recrystallized from ether (50 mL) to afford 2-bromo-1-(2,4-dimethoxyphenyl)ethanone as a light pink solid (29 g, 52%).

### Step 2

To a solution of the above product (20 g, 77.5 mmol) and methyl 4-hydroxybenzoate (14.1 g, 93.0 mmol) in acetone (200 mL) was added potassium carbonate (12.8 g, 93.0 mmol). The mixture was stirred at room temperature for 12 h. Acetone was evaporated and DCM (100 mL) was added. The organic layer was washed with 1 N NaOH (100 mL×2), dried (Na₂SO₄), filtered and concentrated to afford a light red solid, which was recrystallized from ethyl ether to give methyl 4-(2-(2,4-dimethoxyphenyl)-2-oxoethoxy)benzoate (18 g, 71%).

### Step 3

To a solution of the above product (8 g, 23.8 mmol) in 1,2-dichloroethane (80 mL) was added AlCl₃ (32 g, 238 mmol) at 0° C. At this temperature 2′-hydroxyacetophenone was added dropwise in 30 min. When the addition was complete, the mixture was warmed to room temperature for 30 min and then heated to 55° C. for 2 h. The mixture was poured carefully to 6 N HCl (200 mL) and extracted with EA (200 mL×3). The combined organic layers were washed with brine (150 mL×2), dried over anhydrous Na₂SO₄, filtered and concentrated to afford a yellow slurry, which was recrystallized from EA/PE=1/5 (50 mL) to obtain Intermediate 5-4 as light yellow solid (4.5 g, 62%).

### Synthesis of Intermediate 5-5, 4-((2-(2,4-dihydroxyphenyl)-2-oxoethyl)thio)benzoic acid

### Step 1

Followed scheme 5-1, Route B, Step 2 wherein the starting materials were 2-bromo-1-(2,4-dimethoxyphenyl)ethanone (see Intermediate 5-4, step 1 for synthesis) and 4-mercaptobenzoic acid to give 4-((2-(2,4-dimethoxyphenyl)-2-oxoethyl)thio)benzoic acid.

### Step 2

A solution of the above product (2.3 g, 6.92 mmol) in DCM (30 mL) was cooled to −65° C. with stirring. BBr₃ (4 mL, 41.52 mmol) was added dropwise. The mixture was stirred at −65° C. for 2 h and warmed to room temperature overnight. The reaction mixture was cooled to −65° C. and quenched with 1N HCl solution (30 mL). EtOH (150 mL) was added to the mixture followed by concentration and filtration. The filter cake was washed with water, dried in vacuo and purified by Combi-Flash to give Intermediate 5-5 as a yellow solid (1.7 g, 81%).

### Synthesis of Intermediate 5-6, 4-(2-(2,4-dihydroxyphenyl)-2-oxoethoxy)-2-hydroxybenzoic acid

### Step 1

Followed a procedure similar to Scheme 5-1, Route B, step 2 starting from methyl 2,4-dihydroxybenzoate and 2-bromo-1-(2,4-dimethoxyphenyl)ethanone (see Intermediate 5-4, step 1 for synthesis), and wherein the base used was 3 equivalents of cesium carbonate. The crude product was purified after workup by silica gel column chromatography (PE:EA=9:1 to 1:1) to give methyl 4-(2-(2,4-dimethoxyphenyl)-2-oxoethoxy)-2-hydroxybenzoate.

### Step 2

To a solution of the above product (500 mg, 1.44 mmol) in DCM (10 mL) was added dropwise BBr₃ (3.62 g, 14.4 mmol) in DCM (5 mL) over 25 min at −78° C. The reaction was then warmed up to 0° C. and stirred for another 4.5 h. After an aqueous/EtOAc workup, the crude was purified by silica gel column chromatography (PE:EA=1:3) to afford Intermediate 5-6 as a brown solid (280 mg, 64%).

### Synthesis of Intermediate 5-7, methyl 4-((2-(2,4-dihydroxyphenyl)-2-oxoethyl)(methyl)amino)benzoate

### Step 1

A mixture of methyl 4-methylaminobenzoate (6.6 g, 40 mmol) and 2-bromoacetic acid (5.6 g, 40 mmol) was heated to 100° C. for 30 min under the protection of nitrogen. The mixture was cooled to room temperature and water (50 mL) was added. The precipitant was filtered, dried in vacuo and recrystallized from DCM (20 mL) to afford 2-((4-(methoxycarbonyl)phenyl)(methyl)amino)acetic acid (3.1 g, 35%) as a grey solid.

### Step 2

To the solution of the above product (4.4 g, 19.7 mmol) and DMF (1 d) in dried DCM (40 mL) was added dropwise oxalyl chloride (12 mL, 98.6 mmol) at 0˜5° C. with stirring. After the addition was complete, the mixture was warmed to rt for 2 h. TLC (PE:EA=3:1, quenched with MeOH) indicated that the reaction is complete, the volatile was evaporated to get methyl 4-((2-chloro-2-oxoethyl)(methyl)amino)benzoate as a yellow oil (4.6 g).

### Step 3

To a suspension of N,O-dimethylhydroxylamine hydrochloride (2.9 g, 29.6 mmol) and TEA (10 mL, 68.9 mmol) in dried DCM (50 mL) was added the above product (4.6 g) at 0˜5° C. When the addition was complete, the mixture was allowed to warm to room temperature and stirred for 2 h. The mixture was washed with water (10 mL), 5% HCl (aqueous, 10 mL) and brine (10 mL) respectively. The organic phase was dried with anhydrous sodium sulfate, filtered and concentrated to afford yellow oil, which was purified by combiflash (PE:EA=3:1) to obtain methyl 4-((2-(methoxy(methyl)amino)-2-oxoethyl)(methyl)amino)benzoate (1.1 g, 22%) as a yellow solid.

### Step 4

To a solution of 2,4-dimethoxy-1-bromobenzene (567 mg, 3 mmol) in dried THF (5 mL) was added n-BuLi (1.5 mL, 3.6 mmol) at −78° C. for 30 min under the protection of nitrogen. When the addition was complete, the mixture was allowed to warm to room temperature and stirred for 2 hours. A solution of the product from Step 3 (814 mg, 3 mmol) in dried THF (5 mL) was added dropwise to the above solution over 30 min at −78° C. The resulting solution was allowed to warm to room temperature and quenched with saturated ammonium chloride (5 mL). The organic layer was separated and the aqueous phase was extracted with EA (5 mL×2). The combined organic phase was dried with anhydrous sodium sulfate, filtered and concentrated to afford yellow oil, which was purified by silica gel chromatography (PE:EA=3:1) to obtain methyl 4-((2-(2,4-dimethoxyphenyl)-2-oxoethyl)(methyl)amino)benzoate (230 mg, 22%) as a yellow solid.

### Step 5

A suspension of the above product (30 mg, 0.088 mmol) and AlCl₃ (175 mg, 1.31 mmol) in dried DCM (3 mL) was stirred at room temperature overnight. The mixture was quenched with 1N HCl (aq., 3 mL) and extracted with EA (10 mL×3). The combined organic phase was dried over anhydrous sodium sulfate, filtered and concentrated to afford a yellow solid, which was purified by prep-TLC (PE:EA=3:1) to obtain methyl 4-((2-(2,4-dihydroxyphenyl)-2-oxoethyl)(methyl)amino)benzoate (5 mg, 18%) as a yellow solid. ¹H NMR (CDCl₃ 500 MHz TMS): δ 11.85 (s, 1H), 10.64 (s, 1H), 7.83 (d, J=9 Hz, 1H), 7.72 (d, J=9 Hz, 2H), 6.68 (d, J=9 Hz, 2H), 6.40 (1H), 6.31 (d, d, J=2 Hz, 1H), 5.01 (s, 2H), 3.75 (s, 3H), 3.07 (s, 3H).

### Synthesis of Intermediate 5-8, 1-(2,4-dihydroxyphenyl)-2-(3-fluoro-4-nitrophenoxy)ethanone

### Step 1

Followed a procedure similar to that described in Intermediate 5-1, step 1, where the starting materials were 3-fluoro-4-nitrophenol and 2-bromoacetonitrile. Reaction was refluxed for 4 hours. After workup, crude was purified by column chromatography to give 2-(3-fluoro-4-nitrophenoxy)acetonitrile.

### Step 2

Followed a procedure similar to that described in Intermediate 5-1, step 2, using dry diethyl ether as the solvent. The crude product was purified by crystallization from isopropyl alcohol to give Intermediate 5-8.

### Synthesis of Intermediate 5-9, 1-(2,4-dihydroxyphenyl)-2-(4-fluoro-3-nitrophenoxy)ethanone

Followed the two step procedure described for the synthesis of Intermediate 5-8, starting from 4-fluoro-3-nitrophenol and 2-bromoacetonitrile.

### Synthesis of Compound V-3, 4-(7-hydroxy-2-isopropyl-4-oxo-4H-chromen-3-yloxy)benzoic acid, a detailed example of Scheme 5-2 (b conditions for Step 2)

Followed Scheme 5-2, Route A

### Step 1

A mixture of the Intermediate 5-1 (200 mg, 0.632 mmol) and isobutyric anhydride (200.1 mg, 1.265 mmol), and triethylamine (0.49 mL, 3.53 mmol) was heated at 120° C. for 7 h. After being cooled down to room temperature, the reaction mixture was poured into ice-water (10 mL), and the resulting mixture was extracted with ethyl acetate (10 mL×3). The combined organic layer was dried over Na₂SO₄, filtered, and concentrated to give ethyl 4-((7-(isobutyryloxy)-2-isopropyl-4-oxo-4H-chromen-3-yl)oxy)benzoate (300 mg, yield 100%).

### Step 2

The above product (300 mg, 0.684 mmol) was added to a solution of sodium hydroxide (109.5 mg, 2.737 mmol) in a mixture of water (10 mL) and ethanol (10 mL). The reaction mixture was stirred at 35° C. for 7 h, HCl aqueous solution (1N) was added until pH=5. The resulting mixture was extracted with ethyl acetate (10 mL×3), and the combined organic layer was washed with brine (10 mL), dried over sodium sulfate, and concentrated under reduced pressure. The residue was purified by preparative HPLC to give Compound V-3 (33 mg, yield 14.2%). H-NMR (DMSO-d₆ 400 MHZ): δ 12.73 (s, 1H), 10.89 (s, 1H), 7.89-7.86 (m, 3H), 7.05 (d, J=9.2 Hz, 2H), 6.96-6.93 (m, 2H), 3.20-3.13 (m, 1H), 1.23 (d, J=7.2 Hz, 6H), MS (ESI): m/z 340.9 [M+H]+.

### Synthesis of Compound V-2, 4-((7-hydroxy-4-oxo-2-phenyl-4H-chromen-3-yl)oxy)benzoic acid

Prepared following the 2 step procedure described in Scheme 5-2, starting from Intermediate 5-1 and benzoic anhydride. Step 2 used b conditions as described in V-3. ¹H-NMR (DMSO-d₆ 400 MHz): δ 10.98 (s, 1H), 7.89-7.82 (m, 6H), 7.51 (s, 3H), 7.07 (d, J=8.4 Hz, 2H), 7.02 (s, 1H), 6.96 (d, J=8.8 Hz, 1H); MS (ESI): m/z 374.9 [M+H]⁺.

### Synthesis of Compound V-4, 4-((7-hydroxy-4-thioxo-2-(trifluoromethyl)-4H-chromen-3-yl)oxy)benzoic acid

### Step 1

Followed step 1 of Scheme 5-2, wherein Intermediate 5-2 and 4 equiv. of 2,2,2-trifluoroacetic anhydride was used to prepare benzyl 4-((7-hydroxy-4-oxo-2-(trifluoromethyl)-4H-chromen-3-yl)oxy)benzoate.

### Step 2

To a solution of the above product (300 mg, 0.66 mmol) in toluene (25 mL) was added P₂S₅ (146 mg, 0.66 mmol), and the mixture was heated at 80° C. for 10 h. The reaction mixture was concentrated in vacuo, and purified by preparative TLC to afford benzyl 4-((7-hydroxy-4-thioxo-2-(trifluoromethyl)-4H-chromen-3-yl)oxy)benzoate (150 mg, yield 50%).

### Step 3

To a solution of the above product (100 mg, 0.21 mmol) in dichloromethane (15 mL) was added tribromoborane (210 mg, 0.85 mmol) at 0° C., and the mixture was stirred at 0° C. for 1 h. The reaction mixture was poured into water, and the resulting mixture was extracted with dichloromethane (20 mL×2). The combined organic layers were washed with brine (30 mL×2), dried over anhydrous sodium sulfate, filtered, and concentrated in vacuo. The residue was purified by preparative HPLC to afford Compound V-4 (20 mg, yield 25%). ¹H NMR (acetone-d₆ 400 MHz): δ 8.37 (d, J=9.2 Hz, 1H), 7.97 (d, J=8.8 Hz, 2H), 7.17-7.12 (m, 4H); MS (ESI): m/z 382.9 [M+H]⁺.

### Synthesis of Compound V-6, 3-(4-(1H-tetrazol-5-yl)phenoxy)-7-hydroxy-2-(trifluoromethyl)-4H-chromen-4-one

### Step 1

A mixture of compound of Intermediate 5-3 (500 mg, 1.86 mmol), TFAA (1.5 mL), and pyridine (2 mL) was stirred at 110° C. overnight. The mixture was purified by column chromatography on silica gel (PE:EtOAc=8:1˜2:1) to give 4-((7-hydroxy-4-oxo-2-(trifluoromethyl)-4H-chromen-3-yl)oxy)benzonitrile (220 mg, yield 31.7%).

### Step 2

Under N₂, a mixture of the above product (350 mg, 0.94 mmol), NaN₃ (184 mg, 2.82 mmol), and LiCl (80 mg, 1.87 mmol) in 2-(2-methoxy-ethoxy)-ethanol (2 mL) was stirred at 120° C. overnight. The mixture was cooled to room temperature, and basified with aqueous NaHCO₃ solution until Ph=9. The resulting solution was extracted with a mixture of CH₂Cl₂ and i-PrOH (3/1), and the combined organic layer was dried over Na₂SO₄, concentrated in vacuo, and purified by HPLC (32-62% acetonitrile+0.1% trifluoroacetic acid in water, over 15 min.) to afford Compound V-6 (17.8 mg, yield 4.6%). ¹H NMR (CD₃OD 400 MHz TMS): 7.89-7.82 (m, 3H), 6.96 (d, J=8.8 Hz, 2H), 6.82 (d, J=8.8 Hz, 1H), 6.72 (s, 1H); MS (ESI): m/z 391.1 [M+1]⁺.

### Synthesis of Compound V-8, 4-(2-(4-fluorophenyl)-7-hydroxy-4-oxo-4H-chromen-3-yloxy)benzoic acid

Followed Scheme 5-2, Route B

To a solution of 4-((2-(4-fluorophenyl)-7-hydroxy-4-oxo-4H-chromen-3-yl)oxy)benzoic acid (Intermediate V-4) (100 mg, 0.33 mmol) and TEA (83 mg, 0.83 mmol) in DCM (3 mL) was added 4-fluorobenzoyl chloride (105 mg, 0.66 mmol) at 0° C. for 30 min and the resultant solution was warmed to room temperature for 1 h. The volatiles were evaporated and the residue was purified by prep-TLC (PE/EA=3/1) to afford 44244-(methoxycarbonyl)phenoxy)acetyl)-1,3-phenylene bis(4-fluorobenzoate) as a light yellow oil (125 mg, 70%).

### Step 2

A solution of the above product (120 mg, 0.22 mmol) in TEA (3 mL) was heated to reflux for 12 h. The volatiles were evaporated and the residue was purified by prep-TLC (PE/EA=2/1) to afford methyl 4-((2-(4-fluorophenyl)-7-hydroxy-4-oxo-4H-chromen-3-yl)oxy)benzoate as a yellow oil (65 mg, 73%).

### Step 3

To a solution of the above product (60 mg, 0.15 mmol) in THF (0.5 mL) and water (0.5 mL) was added LiOH H₂O (19 mg, 0.45 mmol). The reaction mixture was stirred at room temperature for 14 h. THF was evaporated and the residue was acidified to pH=5 with 5% HCl aq. The solid was filtered and dried in vacuo to afford Compound V-8 as a white solid (18 mg, 31%). MS (ESI): m/z 393.0 [M+1]⁺.

### Synthesis of Compound V-7, 4-(2-(4-chlorophenyl)-7-hydroxy-4-oxo-4H-chromen-3-yloxy)benzoic acid

Followed Scheme 5-2, Route B, starting with Intermediate 5-4 and 4-chlorobenzoyl chloride. MS (ESI): m/z 408.9 [M+1]⁺.

### Synthesis of Compound V-9, 4-(7-hydroxy-2-(4-methoxyphenyl)-4-oxo-4H-chromen-3-yloxy)benzoic acid

Followed Scheme 5-2, Route B, starting with Intermediate 5-4 and 4-methoxybenzoyl chloride. MS (ESI): m/z 405.0 [M+1]⁺.

### Synthesis of Compound V-10, 4-(7-hydroxy-2-(4-hydroxyphenyl)-4-oxo-4H-chromen-3-yloxy)benzoic acid

To a suspension of Compound V-9 (107 mg, 0.27 mmol) in DCM (5 mL) was added dropwise BBr₃ (2 mL, 1.33 mmol) at room temperature and stirred for 3 h at this temperature. The reaction mixture was quenched with methanol (3 mL) and filtered. The filtrate was evaporated to dryness and the residue was purified by prep-TLC to afford Compound V-10 as a white solid (34 mg, 27%). MS (ESI): m/z 391.3 [M+1]⁺.

### Synthesis of Compound V-11, 4-(7-hydroxy-4-oxo-2-(trifluoromethyl)-4H-chromen-3-ylthio)benzoic acid

Followed Step 1 of Scheme 5-2, Route A wherein the TFAA was added at 0° C. and the reaction was stirred at room temperature for 2 hours. After workup, compound was purified by prep-TLC, washed with DCM and filtered to give Compound V-11. MS (ESI): m/z 383.0 [M+1]⁺.

### Synthesis of Compound V-12, 4-(7-hydroxy-4-oxo-4H-chromen-3-yloxy)benzoic acid

### Step 1

To a solution of Intermediate 5-3 (567.6 mg, 1.5 mmol) in N,N-dimethylformamide (3 mL) was added BF₃-Et₂O (887 mg, 3.0 mmol) and phosphorus pentachloride (375 mg, 1.8 mmol). The mixture was heated at 60° C. for 5 h, poured into water (50 mL), and boiled for 1 h. After being cooled down to room temperature, the mixture was extracted with ethyl acetate (60 mL). The organic layers were washed with brine (50 mL), dried over sodium sulfate, evaporated, and purified by column chromatography on silica gel (petroleum ether/ethyl acetate=15:1) to give benzyl 4-((7-hydroxy-4-oxo-4H-chromen-3-yl)oxy)benzoate (260 mg, yield 44.6%).

### Step 2

To a solution of the above compound (260 mg, 0.67 mmol) in dry dichloromethane (10 mL) was added BBr₃ (0.63 mL, 6.7 mmol) at 0° C., and the mixture was stirred at this temperature for 1 h. The mixture was poured into ice water (30 mL), and filtered. The filter cake was washed with water (10 mL), and dried under vacuum to afford Compound V-12 (122.5 mg, yield 61.3%). ¹H NMR (DMSO-d₆ 400 MHZ): δ 10.95 (s, 1H), 8.67 (s, 1H), 7.94-7.90 (m, 3H), 7.07 (d, J=8.8 Hz, 2H), 6.99-6.95 (m, 2H); MS (ESI): m/z 298.9 [M+H]⁺.

### Synthesis of Compound V-13, 2-hydroxy-4-(7-hydroxy-4-oxo-2-(trifluoromethyl)-4H-chromen-3-yloxy)benzoic acid

To a solution of Intermediate 5-6 (35 mg, 0.115 mmol) in Et₃N (1 mL) was added TFAA (96 mg, 0.460 mmol) at 0° C. After addition, the mixture was stirred at room temperature overnight, concentrated and purified by prep-HPLC to afford Compound V-13 as a white solid (5.5 mg, 11%). MS (ESI): m/z 383.0 [M+H]⁺.

### Synthesis of Compound V-14, 4-((7-hydroxy-4-oxo-2-(trifluoromethyl)-4H-chromen-3-yl)(methyl)amino)benzoic acid

### Step 1

To a solution of Intermediate 5-7 (120 mg, 0.387 mmol) and TEA (1.1 mL, 7.74 mmol) in anhydrous DCM (3 mL) was added dropwise TFAA (800 mg, 3.87 mmol). The mixture was stirred at room temperature for 2 h. The volatiles were removed in vacuo, the residue was purified by prep-TLC (PE:EA=1:1) to give methyl 4-((7-hydroxy-4-oxo-2-(trifluoromethyl)-4H-chromen-3-yl)(methyl)amino)benzoate as a yellow solid (140 mg, 92%).

### Step 2

A solution of the above product (46 mg, 0.117 mmol) in dioxane (0.5 mL) and conc. HCl (0.5 mL) was heated at 70° C. overnight. Dioxane was removed under reduced pressure and the residue diluted with water (5 mL). The precipitate was filtered, washed with EtOAc (0.5 mL) and dried in vacuo to afford Compound V-14 as a yellow solid (10 mg, 23%). ¹H NMR (MeOH-d₄ 500 MHz TMS): δ 8.02 (d, J=9 Hz, 1H), 7.88 (d, J=9 Hz, 2H), 7.03 (1H), 7.00 (s, 1H), 6.75 (d, J=9 Hz, 2H), 3.27 (s, 3H). MS (ESI): m/z 380.0 [M+1]⁺.

### Synthesis of Compound V-15, 4-(7-hydroxy-4-oxo-2-(trifluoromethyl)-4H-chromen-3-ylamino)benzoic acid

To a solution of methyl 4-((7-hydroxy-4-oxo-2-(trifluoromethyl)-4H-chromen-3-yl)(methyl)amino)benzoate (see Compound V-14 step 1 for synthesis) (90 mg, 0.23 mmol) in DCM (9 mL) was added BBr₃ (9 mL, 36.6 mmol) at 0° C. Then the mixture was allowed to warm to room temperature overnight, quenched with 1N HCl (aq., 10 mL). The resulting mixture was extracted with EA (20 mL×3). The combined organic phase was dried with anhydrous sodium sulfate, filtered and concentrated to afford yellow oil, which was purified by prep-HPLC to obtain Compound 15 (8 mg, 9%) as a yellow solid. ¹H NMR (MeOH-d₄ 500 MHz TMS): δ 8.02 (d, J=9 Hz, 1H), 7.83 (d, J=11 Hz, 2H), 7.02 (dd, J=11 Hz 3 Hz, 1H), 6.96 (1H), 6.70 (d, J=11 Hz, 2H). MS (ESI): m/z 366.0 [M+1]⁺.

### Synthesis of Compound V-16, 7-hydroxy-3-(3-hydroxy-4-nitrophenoxy)-2-(trifluoromethyl)-4H-chromen-4-one

### Step 1

Followed a procedure similar to that described in Compound V-3, step 1, where the starting materials were 1-(2,4-dihydroxyphenyl)-2-(3-fluoro-4-nitrophenoxy)ethanone (Intermediate 5-8) and TFAA and the reaction was heated at 130° C. for two hours. Purification by column chromatography gave 3-(3-fluoro-4-nitrophenoxy)-7-hydroxy-2-(trifluoromethyl)-4H-chromen-4-one.

### Step 2

A solution of benzyl alcohol (0.5 mL) in DMSO (5 mL) was cooled at 0° C., NaH (60%, 47 mg, 1.2 mmol) was added portion-wise, and the mixture was warmed at room temperature for 30 minutes. The product from Step 1 (300 mg, 0.78 mmol) was added, and the mixture was stirred at room temperature for two hours. The mixture was cooled to 0° C., and saturated aqueous NH₄Cl solution was added to quench the reaction. The aqueous layer was extracted with EtOAc (20 mL×3), and the combined organic layer was washed with brine, dried over Na₂SO₄, filtered, and concentrated to give 3-(3-(benzyloxy)-4-nitrophenoxy)-7-hydroxy-2-(trifluoromethyl)-4H-chromen-4-one.

### Step 3

Followed the BBr₃ deprotection described in Step 3 of Compound V-4 to give 7-hydroxy-3-(3-hydroxy-4-nitrophenoxy)-2-(trifluoromethyl)-4H-chromen-4-one as an off-white solid. ¹H NMR (DMSO-d₆ 400 MHz TMS): δ 11.27 (s, 1H), 11.03 (s, 1H), 7.93-7.97 (m, 2H), 7.03-7.07 (m, 2H), 6.78-6.83 (m, 2H); MS (ESI): m/z 384.0 [M+1]⁺.

### Synthesis of Compound V-17, 7-hydroxy-3-(4-hydroxy-3-nitrophenoxy)-2-(trifluoromethyl)-4H-chromen-4-one

Followed the three step procedure described for Compound V-16, starting from 3-(4-fluoro-3-nitrophenoxy)-7-hydroxy-2-(trifluoromethyl)-4H-chromen-4-one (Intermediate 5-9) and TFAA. ¹H NMR (DMSO-d₆ 400 MHz TMS): δ 11.21 (s, 1H), 10.73 (s, 1H), 7.92 (d, 1H, J=8.4 Hz), 7.64 (d, 1H, J=1.2 Hz), 7.42 (dd, 1H, J=3.2, 8.8 Hz), 7.09 (d, 1H, J=9.2 Hz), 2.05 (m, 2H). MS (ESI): m/z 384.0 [M+1]⁺.

### Synthetic Details for Compounds of Table 6

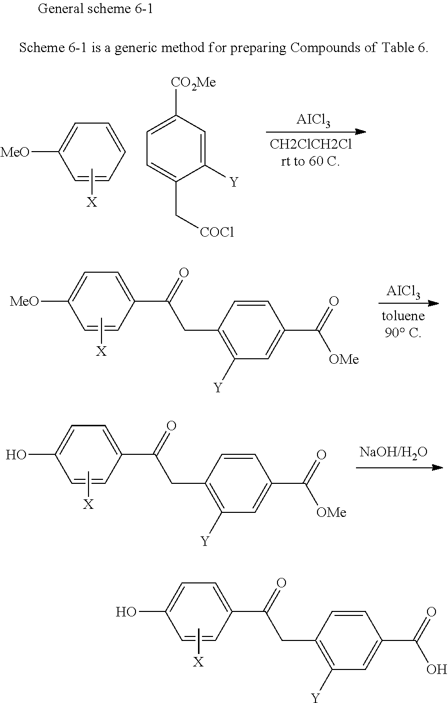

### Intermediate 6-1: Synthesis of methyl 4-(2-chloro-2-oxoethyl)-3-fluorobenzoate (an intermediate for compounds of Table 6)

### Step 1

Methyl 4-(cyanomethyl)-3-fluorobenzoate (9 g) was heated at 80° C. in 8NHCl (100 mL) over night until all starting materials were consumed. All solvents were removed under reduced pressure and dried under vacuum to afford colorless solids-4-(carboxymethyl)-3-fluorobenzoic acid (9 g). MS (ESI): m/z 199 [M+H⁺].

### Step 2

The solids from step 1 were suspended in anhydrous MeOH (60 mL) and treated with 4NHCl in dioxane (8 mL) at 60° C. over a couple of hours until all di-acids were converted to the di-methyl esters. Removal all solvents and drying under vacuum afforded methyl 3-fluoro-4-(2-methoxy-2-oxoethyl)benzoate (10.3 g) as colorless solids. MS (ESI): m/z 227 [M+H⁺]

### Step 3

Methyl 3-fluoro-4-(2-methoxy-2-oxoethyl)benzoate (8.19 g) was dissolved in THF (100 mL) and treated with LiOH (1.24 g) in water (15 mL) over night. After acidification and extraction with EtOAc (40 mL×2), the organic layers were dried over anhy. Na2SO4 and concentrated to dryness to afford the desired product (8.5 g) as colorless solids with a purity of 90%, which was used in the next step without further purification. MS (ESI): m/z 213 {M+H⁺]

### Step 4

2-(2-Fluoro-4-(methoxycarbonyl)phenyl)acetic acid (3.57 g) was suspended in DCM (40 mL) and treated with oxalyl chloride (2.56 g) and DMF (0.2 ml) over 5 h. Removal of solvents afforded the desired product-methyl 4-(2-chloro-2-oxoethyl)-3-fluorobenzoate (3.6 g) as light brown oil.

### Representative Example of General Scheme 6-1, Compound VI-2

### Synthesis of Compound VI-1, (trans)-4-(2-(2,4-dihydroxyphenyl)-2-oxoethyl)cyclohexanecarboxylic acid

Trans-ethyl 4-(2-(2,4-dihydroxyphenyl)-2-oxoethyl)cyclohexanecarboxylate (synthesized as described in PCT US2011/024353) was hydrolyzed by NaOH to afford the title compound trans-4-(2-(2,4-dihydroxyphenyl)-2-oxoethyl)cyclohexanecarboxylic acid. ¹H NMR (DMSO-d₆ 300 MHz TMS): 12.77 (1H, s), 12.01 (1H, s), 10.64 (1H, s), 7.80 (1H, d, J=9 Hz), 6.39 (1H, dd, J=9 and 3 Hz), 6.24 (1H, d, J=3 Hz), 2.24 (2H, d, J=6 Hz), 2.50 (3H, m), 2.12 (1H, dt, J=12 and 3 hz), 1.95-1.65 (5H, m), 1.35-1.23 (2H, m), 1.11-1.09 (2H, m) ppm; MS (ESI): m/z=279 [M+H⁺].

### Synthesis of Compound VI-2, 3-fluoro-4-(2-(2-fluoro-4-hydroxyphenyl)-2-oxoethyl)benzoic acid

### Step 1: Friedel-Crafts Acylation

Methyl 4-(2-chloro-2-oxoethyl)-3-fluorobenzoate (200 mg) was dissolved in 1,2-dichloroethane (6 mL) and mixed with AlCl₃ (200 mg), chilled with an ice-water bath. The resultant mixture was stirred over 30 min, and then 3-fluoroanisol (150 mg) in 1,2-dichloroethane (3 mL) was added. The reaction mixture was stirred at room temperature overnight, then quenched with 1N HCl and extracted with EtOAc (60 mL). The organic layer was washed with brine and dried over anhy. Na₂SO₄. Removal of solvents and column purification with flash silica gel chromatography afforded the desired product—methyl 3-fluoro-4-(2-(2-fluoro-4-methoxyphenyl)-2-oxoethyl)benzoate (90 mg) as colorless solids. MS (ESI): m/z 321 [M+H⁺].

### Step 2: De-Protection

Methyl 3-fluoro-4-(2-(2-fluoro-4-methoxyphenyl)-2-oxoethyl)benzoate (90 mg) was dissolved in toluene and treated with AlCl3 at 90° C. over 2 h. The reaction was quenched with 1NHCl. Extraction with EtOAc, followed by column purification with flash gel chromatography afforded the desired product methyl 3-fluoro-4-(2-(2-fluoro-4-hydroxyphenyl)-2-oxoethyl)benzoate (45 mg) as colorless solids. MS (ESI): m/z 307 [M+H⁺].

### Step 3: Basic Hydrolysis

Methyl 3-fluoro-4-(2-(2-fluoro-4-hydroxyphenyl)-2-oxoethyl)benzoate (45 mg) was treated with NaOH (4N, 1 mL) in MeOH (3 mL) over a couple of hours until the ester was completed hydrolyzed. The desired product was precipitated out by adding 2N HCl and collected by either filtration or centrifuge. Rinsing with H₂O and drying under vacuum afforded the final product-3-fluoro-4-(2-(2-fluoro-4-hydroxyphenyl)-2-oxoethyl)benzoic acid (N91261) (15 mg) as white solids. ¹H NMR (DMSO-d6 300 MHz TMS): δ 7.80 (t, 1H, J=9 Hz), 7.72 (1H, dd, J=9 and 3 Hz), 7.64 (1, dd, J=9, and 3 Hz), 7.45 (1H, t, J=9 Hz), 6.74 (1H, dd, J=12 and 3 Hz), 6.68 (1H, dd, J=12 and 3 Hz), 4.37 (s, 2H); MS (ESI): m/z 293, [M+H⁺].

### Synthesis of Compound VI-3, 4-(2-(2,3-difluoro-4-hydroxyphenyl)-2-oxoethyl)-3-fluorobenzoic acid

Followed the same synthetic procedure as described for the synthesis of Compound VI-2, starting with 1,2-difluoro-3-methoxybenzene and methyl 4-(2-chloro-2-oxoethyl)-3-fluorobenzoate (Intermediate 6-1). ¹H NMR (DMSO-d6 300 MHz TMS): δ 7.76 (d, 1H), 7.66 (d, 2H), 7.45 (t, 1H), 6.90 (t, 1H), 4.41 (s, 2H). MS (ESI): m/z=311.43 [M+1]⁺.

### Synthesis of Compound VI-4, 3-fluoro-4-(2-(3-fluoro-4-hydroxyphenyl)-2-oxoethyl)benzoic acid

Followed the same procedure described for the synthesis of Compound VI-2, starting with 1-fluoro-2-methoxybenzene and methyl 4-(2-chloro-2-oxoethyl)-3-fluorobenzoate (Intermediate 6-1). ¹H NMR (CD₃OD, 300 MHz TMS): δ 7.85 (m, 3H), 7.65 (dd, 1H), 7.45 (t, 1H), 7.13 (t, 1H), 4.48 (s, 2H). MS (ESI): m/z=293.34 [M+1]⁺.

### Synthesis of Compound VI-5 4-(2-(2,3-difluoro-4-hydroxyphenyl)-2-oxoethyl)benzoic acid

Followed the same synthetic approach as described for the synthesis of Compound VI-2, starting with methyl 4-(2-chloro-2-oxoethyl)benzoate and 2,3-difluoroanisol. ¹H NMR (DMSO-d₆ 300 MHz TMS): δ 7.88 (2H, d, J=9 Hz), 7.62 (1H, dt, J=9 and 3 Hz), 7.35 (2H, d, J=9, and 3 Hz), 6.93 (1H, dd, J=9 and 3 Hz), 4.37 (2H, d, J=3 Hz) ppm; MS (ESI): m/z 293, [M+H⁺].

### Synthesis of VI-6, 4-(2-(2-chloro-4-hydroxyphenyl)-2-oxoethyl)-3-fluorobenzoic acid

Followed the same procedure described for the synthesis of Compound VI-2, starting with 1-chloro-3-methoxybenzene and methyl 4-(2-chloro-2-oxoethyl)-3-fluorobenzoate (Intermediate 6-1). ¹H NMR (DMSO-d6 300 MHz TMS): δ 10.71 (s, 1H), 7.87 (d, 1H), 7.76 (dd, 1H), 7.66 (dd, 1H), 7.49 (t, 1H), 6.95 (m, 2H), 4.44 (s, 2H). MS (ESI): m/z=309.41 [M+1]⁺.

### Synthesis of VI-7, 4-(2-(2,5-difluoro-4-hydroxyphenyl)-2-oxoethyl)-3-fluorobenzoic acid

Followed the same procedure described for the synthesis of Compound VI-2, starting with 1,4-difluoro-2-methoxybenzene and methyl 4-(2-chloro-2-oxoethyl)-3-fluorobenzoate (Intermediate 6-1). ¹H NMR (DMSO-d₆ 300 MHz TMS): 7.73 (m, 3H), 7.48 (t, 1H) 6.95 (q, 1H), 4.40 (s, 2H). MS (ESI): m/z=311.43 [M+1]⁺.

### Synthesis of Compound VI-8, 4-(2-(3,5-difluoro-4-hydroxyphenyl)-2-oxoethyl)-3-fluorobenzoic acid

Followed the same procedure described for the synthesis of Compound VI-2, starting with 1,3-difluoro-2-methoxybenzene and methyl 4-(2-chloro-2-oxoethyl)-3-fluorobenzoate (Intermediate 6-1). ¹H NMR (DMSO-d₆ 300 MHz TMS): 7.79 (dt, 3H), 7.66 (dd, 1H), 7.47 (t, 1H), 4.52 (s, 2H). MS (ESI): m/z=311.23 [M+1]⁺.

### Synthesis of Compound VI-9, 3-fluoro-4-(2-(4-hydroxyphenyl)-2-oxoethyl)benzoic acid

Followed the same procedure described for the synthesis of Compound VI-2, starting with anisole and methyl 4-(2-chloro-2-oxoethyl)-3-fluorobenzoate (Intermediate 6-1). ¹H NMR (DMSO-d₆ 300 MHz TMS): 10.46 (bs, 1H), 7.96 (dd, 2H), 7.72 (dd, 1H), 7.65 (dd, 1H), 7.48 (t, 1H), 6.90 (dd, 2H), 4.46 (s, 2H). MS (ESI): m/z=275.45 [M+1]⁺.

### Synthesis of Compound VI-10, 4-(2-(2,6-difluoro-4-hydroxyphenyl)-2-oxoethyl)-3-fluorobenzoic acid

Followed same procedure described for the synthesis of Compound VI-2, starting with 1,3-difluoro-5-methoxybenzene and methyl 4-(2-chloro-2-oxoethyl)-3-fluorobenzoate (Intermediate 6-1). ¹H NMR (DMSO-d₆ 300 MHz TMS): 7.76 (dd, 1H), 7.65 (dd, 1H), 7.49 (t, 1H), 6.59 (d, 2H), 4.30 (s, 2H); MS (ESI): m/z=311.22 [M+1]⁺.

### Synthesis of Compound VI-11, 4-(2-(4-hydroxyphenyl)-2-oxoethyl)benzoic acid

### Step 1: Friedel-Crafts Acylation

Followed Scheme 6-1 wherein the acyl chloride, methyl 4-(2-chloro-2-oxoethyl)benzoate, was formed in situ [To a solution of 2-(4-(methoxycarbonyl)phenyl)acetic acid (2 g, 10.3 mmol) in DCM (10 mL) was added oxalyl chloride (6 mL, 10.0 mmol) and one drop of DMF. The mixture was stirred at rt for 1 h and concentrated.] The acid chloride was taken up in DCM (5 mL). AlCl₃ (2.04 g, 15.45 mmol) and anisole (1.33 g, 12.36 mmol) were added and the reaction was stirred at room temperature overnight. Water was added, followed by extraction with EtOAc. The combined organic phase was washed with brine, dried over Na₂SO₄ and concentrated to give 1.5 g of crude methyl 4-(2-(4-methoxyphenyl)-2-oxoethyl)benzoate (24% yield). MS (ESI): m/z 285.1 [M+1]⁺.

### Step 2: Deprotection

To a solution of the above product (700 mg, 2.5 mmol) in CH₂Cl₂ (30 mL) was added AlCl₃ (10 g, 75 mmol). The solution was stirred at ambient temperature for 3 days. Aqueous workup with EtOAc extraction was followed by purification by prep-TLC (PE:EA=1: 1) to gave 180 mg of methyl 4-(2-(4-hydroxyphenyl)-2-oxoethyl)benzoate (27%).

### Step 3: Acidic Hydrolysis

To a solution of methyl 4-(2-(4-hydroxyphenyl)-2-oxoethyl)benzoate (180 mg, 0.67 mmol) in dioxane (3 mL) was added conc. HCl (3 mL). The mixture was stirred at 80° C. overnight. The reaction was concentrated in vacuo to give a pink solid, which was purified by prep-HPLC to afford Compound VI-11 as a white solid (33 mg, 19%). ¹H NMR (MeOH-d4 500 MHz TMS): δ 7.98 (d, J=8.5 Hz, 4H), 7.39 (d, J=8.0 Hz, 2H), 6.87 (d, J=10.0 Hz, 2H), 4.38 (s, 2H); MS (ESI): m/z 257.1 [M+1]⁺.

### Synthesis of Compound VI-12, 4-(1-(2,4-dihydroxyphenyl)-1-oxopropan-2-yl)benzoic acid

### Step 1

Following the reference—J. Med. Chem. 2002, 45 (24), 5358-5364, methyl 4-(2-(2,4-dimethoxyphenyl)-2-oxoethyl)benzoate (synthesis described in PCT US2011/024353) was treated with iodomethane and potassium tert-butyloxide in Et₂O to afford the desired product-methyl 4-(1-(2,4-dimethoxyphenyl)-1-oxopropan-2-yl)benzoate.

### Step 2

BBr₃ deprotection of the above product was accomplished following step 2 of Compound VI-17 to give Compound VI-12. ¹H NMR (DMSOd₆, 500 MHz TMS): 12.92 (1H, s), 12.59 (1H, s), 10.71 (1H, s), 7.89 (2H, d, J=8 Hz), 7.87 (1H, d, J=9 Hz), 7.47 (2H, d, J=8 Hz), 6.31 (1H, dd, J=9 and 2 Hz), 6.23 (1H, d, J=2 Hz), 5.01 (1H, q, J=6.5 Hz), 1.41 (3H, d, J=6.5 Hz) ppm; MS (ESI): m/z 287 [M+H⁺].

### Synthesis of Compound VI-13, (E)-4-(2,4-dihydroxystyryl)benzoic acid

### Step 1

Methyl 4-(2-(2,4-dimethoxyphenyl)-2-oxoethyl)benzoate (synthesis described in PCT US2011/024353) was treated with NaBH₄ in ethanol/THF. After the ketone was reduced, the reaction mixture was acidified with 12N HCl and gently heated at 60° C. for a couple of hours. The crude product was extracted with EtOAc and purified by a silica gel column purification to afford the desired product-(E)-methyl 4-(2,4-dimethoxystyryl)benzoate.

### Step 2

BBr₃ deprotection of the above product was accomplished following step 2 of Compound VI-17 to give Compound VI-13. ¹H NMR (DMSO-d₆, 500 MHz TMS): 9.80-9.76 (2H, broad), 7.90 (2H, d, J=8 Hz), 7.58 (2H, d, J=8 Hz), 7.46-7.41 (2H, m), 7.09-7.06 (1H, d, J=16 Hz), 6.61 (1H, d, J=1.5 Hz), 6.28 (1H, d, J=8 Hz) ppm; MS (ESI): m/z 257 [M+H⁺].

### Synthesis of Compound VI-14, 4-(4-hydroxy-2-(trifluoromethyl)benzamido)benzoic acid

### Step 1

Acid chloride prep: 4-methoxy-2-(trifluoromethyl)benzoic acid (2.27 mmol, 500 mg) was taken up in DCM. Oxalyl chloride (0.9 mL) and 2 drops of DMF were added. The mixture was stirred for 2.5 hours, followed by concentration in vacuo. The resulting acid chloride was then re-dissolved in 3 mL of DCM and cooled to 0° C. under Ar(g). Methyl 4-aminobenzoate (2.06 mmol, 311 mg) was dissolved in 5 mL of DCM and 0.17 mL of pyridine was added. The solution containing the aniline was then slowly added to the acid chloride solution and stirred for 2 hours while slowly warming to room temperature. Aqueous workup with EtOAc extraction yielded 565 mg of methyl 4-(4-methoxy-2-(trifluoromethyl)benzamido)benzoate.

### Step 2

Methyl 4-(4-methoxy-2-(trifluoromethyl)benzamido)benzoate (0.71 mmol, 250 mg) was dissolved in 2 mL of NMP and Na₂S (0.71 mmol, 55 mg) was added. The mixture was heated at 140° C. in a microwave reactor for 2 hours. The crude reaction mixture was then quenched with 10 mL of 0.5N HCl, basified with 1N NaOH to pH=10, back extracted with EtOAc, and finally acidified to pH=4 with concentrated HCl. The resulting solid was filtered and dried to give 150 mg of crude product. The crude material was pulled through a silica plug with 10% MeOH in DCM and concentrated in vacuo affording 101 mg of 4-(4-hydroxy-2-(trifluoromethyl)benzamido)benzoic acid as a tan powder. ¹H-NMR (DMSO-d₆ 300 MHz TMS): δ 12.73 (bs, 1H), 10.72 (s, 1H), 10.54 (s, 1H), 7.94 (d, 2H), 7.81 (d, 2H), 7.59 (d, 1H), 7.16 (d, 1H), 7.14 (dd, 1H). MS (ESI): m/z=326.04 [M+1]+.

### Synthesis of Compound VI-15, 4-(2-fluoro-4-hydroxybenzamido)benzoic acid

### Step 1

Followed Step 1 of Compound VI-14, using starting materials: 2-fluoro-4-methoxybenzoyl chloride and methyl 4-aminobenzoate to give methyl 4-(2-fluoro-4-methoxybenzamido)benzoate.

### Step 2

Methyl 4-(2-fluoro-4-methoxybenzamido)benzoate (0.712 mmol, 216 mg) was cooled to 0° C. in an ice bath neat. BBr₃ (4.5 mL) was added and the reaction was stirred for 3 days at ambient temperature. The solution was then basified with 1N NaOH to a pH=10 and back extracted with DCM (10 mL). The aqueous was collected and acidified to pH=3 with conc. HCl and the resulting solid was filtered and purified via silica gel chromatography with gradient of 0-5% MeOH in EtOAc to give 80 mg of Compound VI-15 as a white powder. ¹H-NMR (DMSO-d₆ 300 MHz TMS): δ 10.38 (s, 1H), 7.93 (d, 2H), 7.83 (d, 2H), 7.59 (t, 1H), 6.74 (dd, 1H), 6.70 (dd, 1H). MS (ESI): m/z=276.07 [M+1]+.

### Synthesis of Compound VI-16, 4-(2,4-dihydroxybenzamido)benzoic acid

### Step 1

Followed Step 1 of Compound VI-14, using starting materials: 2,4-dimethoxybenzoyl chloride and methyl 4-aminobenzoate to give methyl 4-(2-fluoro-4-methoxybenzamido)benzoate.

### Step 2

Methyl 4-(2,4-dimethoxybenzamido)benzoate (671 mg, 2.13 mmol) was dissolved in 7 mL of DCM and cooled to 0° C. in an ice bath. 17.2 mL of BBr3 was slowly added and the reaction was stirred for 6 hours while allowing the ice bath to expire. The solids were filtered and dried in vacuo. 50 mg of the resulting ester (0.174 mmol) was dissolved in 5 mL of a 4:1 mixture of H₂O:THF and 16 mg of LiOH was added. The mixture was stirred overnight followed by acidification to pH=4 with conc. HCl. The resulting solid was filtered, washed with H₂O, and dried in vacuo to afford 30 mg of 4-(2,4-dihydroxybenzamido)benzoic acid as a white powder. ¹H-NMR (DMSO-d₆ 300 MHz TMS): δ 10.41 (s, 1H), 10.24 (bs, 1H), 7.95 (d, 2H), 7.90 (d, 1H), 7.83 (d, 2H), 6.41 (dd, 1H), 6.36 (d, 1H). MS (ESI): m/z=274.06 [M+1]+.

### Synthesis of Compound VI-17, (E)-4-(1-(2,4-dihydroxyphenyl)-4,4,4-trifluoro-1-oxobut-2-en-2-yl)benzoic acid

### Step 1

To a mixture of trifluoroacetaldehyde monohydrate (1.5 g, 12.9 mmol) in DCM (20 mL) was added sodium bicarbonate (1.6 g, 12.9 mmol) and anhydrous magnesium sulfate (1.6 g, 12.9 mmol). The resulting mixture was stirred at room temperature for 30 min, filtered and the filtrate was cooled to 0° C. To the filtrate was added methyl 4-(2-(2,4-dimethoxyphenyl)-2-oxoethyl)benzoate (synthesis described in PCT US2011/024353) (500 mg, 1.59 mmol), followed by DBU (3 mL). The solution was stirred at room temperature for 2 days and then heated to reflux for 5 h. The volatiles were removed under reduced pressure and the residue was purified by Combiflash (PE/EA=3/1) to obtain (E)-methyl 4-(1-(2,4-dimethoxyphenyl)-4,4,4-trifluoro-1-oxobut-2-en-2-yl)benzoate as a yellow oil (mixture of isomers, 250 mg, 39.9%).

### Step 2

To a solution of the above product (250 mg, 0.63 mmol) in DCM (3 mL) was added dropwise BBr₃ (0.8 mL, 9.45 mmol) at room temperature for 24 h. The mixture was poured into saturated sodium carbonate (25 mL), extracted with DCM (15 mL). The aqueous phase was separated, acidified to pH=3 with 1 N HCl (aq.) and extracted with EA (10 mL×3). The combined organic phase was dried with anhydrous sodium sulfate, filtered and concentrated followed by purification by prep-HPLC to afford 4-(1-(2,4-dihydroxyphenyl)-4,4,4-trifluoro-1-oxobut-2-en-2-yl)benzoic acid (3.4 mg, 1.5%) as light yellow oil (This sample contains two isomers, E/Z=1: 0.6, identified from HNMR).

### Synthesis of Compound VI-18, 4-(2-(2,4-dihydroxyphenyl)-2-oxoethyl)-3-fluorobenzoic acid

### Step 1

Hydrolysis of methyl 4-(2-(2,4-dihydroxyphenyl)-2-oxoethyl)-3-fluorobenzoate following basic conditions (see step 3 of Compound VI-2 for similar procedure) gave the desired VI-18. ¹H NMR (DMSO-d₆ 300 MHz TMS): 12.21 (1H, s), 7.92 (1H, d, J=6 Hz), 7.75 (1H, dd, J=6 and 3 Hz), 7.64 (1H, dd, J=9 and 3), 7.47 (1H, t, J=9 Hz), 6.43 (1H, dd, J=9 and 3 Hz), 6.30 (1H, d, J=3 Hz), 4.30 (s, 2H) ppm; MS (ESI): m/z=291 [M+H⁺].

### Synthesis of Compound VI-19, 3-fluoro-4-((2-fluoro-4-hydroxyphenoxy)methyl)benzoic acid

### Step 1

Methyl 4-(bromomethyl)-3-fluorobenzoate (2.36 g) was treated with 2-fluorophenol (0.86 g) and K₂CO₃ (1.32 g) in DMSO (8 ml) over 4 h. The reaction mixture was diluted with water and extracted with EtOAc. Purification by column chromatography gave Methyl 3-fluoro-4-((2-fluorophenoxy)methyl)benzoate (1.04 g).

### Step 2

Methyl 3-fluoro-4-((2-fluorophenoxy)methyl)benzoate (1.04 g) in 1,2-dichloroethane (8 ml) was added into a pre-mixed solution of acetyl chloride (0.422 mL, 2 eq.) and AlCl₃ (997 mg). The resultant solution was stirred overnight at room temperature. After quenched with 1N HCl, the reaction mixture was extracted with EtOAc. Removal of solvents and purification by column chromatography afforded the desired product-methyl 4-((4-acetyl-2-fluorophenoxy)methyl)-3-fluorobenzoate (800 mg) as colorless solids. [M+H⁺]: 321.

### Step 3

Methyl 4-((4-acetyl-2-fluorophenoxy)methyl)-3-fluorobenzoate (435 mg) was treated with mCPBA (366 mg) in DCM at 38° C. over 2 days. After the reaction mixture was diluted with EtOAc (80 mL), the organic solution was washed with sat. NaHCO₃ and brine and dried over anhydrous Na₂SO₄. Removal of solvents and purification by column chromatography gave the desired product-methyl 4-((4-acetoxy-2-fluorophenoxy)methyl)-3-fluorobenzoate (110 mg) as colorless solids. MS (ESI): m/z=337 [M+H⁺].

### Step 4

Methyl 4-((4-acetoxy-2-fluorophenoxy)methyl)-3-fluorobenzoate (110 mg) was treated with 2N NaOH (4 mL) and MeOH (2 mL) over 2 hours. The resultant solution was acidified with 12 N HCl to precipitate the desired product, which was collected by centrifuge, rinsed with water and dried under vacuum to give Compound VI-19 as a solid (55 mg). ¹H NMR (DMSO-d₆ 300 MHz TMS): δ 13.33 (1H, s), 9.56 (1H, s), 7.80 (1H, dd, J=9, 3 Hz), 7.68-7.62 (2H, m), 7.05 (1H, t, J=9 Hz), 6.64 (1H, dd, J=12 and 3 Hz), 6.53 (1H, dd, J=9 and 3 Hz), 5.15 (s, 2H) ppm; MS (ESI): m/z=281 [M+H⁺].

### Synthesis of Compound VI-20, 3-fluoro-4-(((3-fluoro-4-hydroxyphenyl)sulfonyl)methyl)benzoic acid

### Step 1

Followed a similar procedure to that described in VI-19 step 1, starting from methyl 4-(bromomethyl)-3-fluorobenzoate and 3-fluoro-4-methoxybenzenethiol to give methyl 3-fluoro-4-(((3-fluoro-4-methoxyphenyl)thio)methyl)benzoate.

### Step 2

Oxidation of the above product (145 mg) with mCPBA (200 mg) in DCM (8 mL) was achieved with stifling at room temperature for 2 days. Followed workup/purification described for Compound VI-19, step 3 to give methyl 3-fluoro-4-(((3-fluoro-4-methoxyphenyl)sulfonyl)methyl)benzoate.

### Step 3

BBr₃ deprotection of the above product was accomplished following the procedure described in step 2 of Compound VI-17 to give Compound VI-20. ¹H NMR (DMSO-d₆ 300 MHz TMS): 7.75 (1H, d, J=3 Hz), 7.58 (1H, d, J=12 Hz), 7.54 (1H, dd, J=9 and 3 Hz), 7.29-7.32 (2H, m), 7.08 (1H, t, J=9 Hz), 4.76 (s, 2H) ppm; MS (ESI): m/z=329 [M+H⁺].

### Synthesis of Compound VI-21, 4-((2,3-difluoro-4-hydroxyphenoxy)methyl)-3-fluorobenzoic acid

Following the synthesis described for Compound VI-19, 4-((2,3-difluoro-4-hydroxyphenoxy)methyl)-3-fluorobenzoic acid (54 mg) was synthesized from 2,3-difluorophenol (0.474 g) and methyl 4-(bromomethyl)-3-fluorobenzoate (1.12 g) over 4 steps. ¹H NMR (DMSO-d₆ 300 MHz TMS): δ 13.46 (1H, s), 9.95 (1H, s), 7.81 (1H, dd, J=9, 3 Hz), 7.70 (1H, dd, 12 and 3 Hz), 7.64 (1H, d, J=9 Hz), 6.92 (1H, dt, J=9 and 3 Hz), 6.70 (1H, dt, J=9 and 3 Hz), 5.21 (s, 2H) ppm; MS (ESI): m/z=299 [M+H⁺].

### Synthesis of Compound VI-22, 4-((2,5-difluoro-4-hydroxyphenoxy)methyl)-3-fluorobenzoic acid

Following the synthesis described for Compound VI-19, 4-((2,5-difluoro-4-hydroxyphenoxy)methyl)-3-fluorobenzoic acid was synthesized from 2,5-difluorophenol and methyl 4-(bromomethyl)-3-fluorobenzoate over 4 steps. ¹H NMR (DMSO-d₆ 300 MHz TMS): δ 13.35 (1H, s), 9.98 (1H, s), 7.81 (1H, dd, J=6, 3 Hz), 7.63-7.61 (2H, m), 7.25 (1H, dd, J=12 and 9 Hz), 6.90 (1H, dd, J=12 and 9 Hz), 5.19 (s, 2H) ppm; MS (ESI): m/z=299 [M+H⁺].

### Synthesis of Compound VI-23, 3-fluoro-4-((3-fluoro-4-hydroxyphenoxy)methyl)benzoic acid

### Step 1

Following the synthesis described for Compound VI-19, 3-fluoro-4-((3-fluoro-4-hydroxyphenoxy)methyl)benzoic acid was synthesized from 3-fluorophenol and methyl 4-(bromomethyl)-3-fluorobenzoate over 4 steps. ¹H NMR (DMSO-d₆ 300 MHz TMS): δ 7.86 (1H, dd, J=9, 3 Hz), 7.71 (1H, d, J=9 and 3 Hz), 7.63 (1H, t, J=9 Hz), 6.84 (1H, dd, J=12 and 3 Hz), 6.80 (1H, d, J=12 Hz), 6.67-6.61 (1H, m), 5.15 (s, 2H) ppm; MS (ESI): m/z=281 [M+H⁺].

### Synthesis of Compound VI-24, 3-fluoro-4-(((4-hydroxyphenyl)thio)methyl)benzoic acid

### Step 1

To a solution of methyl 4-(bromomethyl)-3-fluorobenzoate (1 g) and 4-mercaptophenol (0.79 g) in THF (15 mL) was added TEA (3 mL). After the mixture was stirred overnight, it was diluted with EtOAc and water. The isolated organic layer was dried with anhydrous Na₂SO₄ and removed under reduced pressure. A flash silica gel column purification gave the desired product-methyl 3-fluoro-4-(((4-hydroxyphenyl)thio)methyl)benzoate (0.9 g) as colorless solids.

### Step 2

Following the general basic hydrolysis, the final product—3-fluoro-4-(((4-hydroxyphenyl)thio)methyl)benzoic acid (90 mg) was obtained from methyl 3-fluoro-4-(((4-hydroxyphenyl)thio)methyl)benzoate (120 mg) with NaOH treatment. ¹H NMR (CD₃OD, 300 MHz): δ 7.64 (1H, dd, J=9 and 3 Hz), 7.59 (1H, dd, J=9 and 3 Hz), 7.15 (2H, d, J=9 Hz), 7.11 (1H, t, J=9 Hz), 6.69 (2H, d, J=9 Hz), 3.99 (2H, s) ppm; MS (ESI): m/z 279, [M+H⁺].

### Synthesis of Compound VI-25, 3-fluoro-4-(((4-hydroxyphenyl)sulfinyl)methyl)benzoic acid

### Step 1

To a solution of methyl 3-fluoro-4-(((4-hydroxyphenyl)thio)methyl)benzoate (for synthesis see Step 1 of VI-24) (340 mg) in DCM (5 ml) was added 3-chloroperbenzoic acid (77% pure, 270 mg). After the mixture was stirred over 6 hours, the organic solvents were removed. The residue was suspended in NaCHO₃ aqueous solution. After filtration and drying under reduced pressure, the crude product was purified by a flash silica gel column to afford the desired product, methyl 3-fluoro-4-(((4-hydroxyphenyl)sulfinyl)methyl)benzoate (225 mg) as colorless solids.

### Step 2

Following the general basic hydrolysis method, 190 mg of the final product-3-fluoro-4-(((4-hydroxyphenyl)sulfinyl)methyl)benzoic acid was obtained from a basic treatment of methyl 3-fluoro-4-(((4-hydroxyphenyl)sulfinyl)methyl)benzoate (225 mg). ¹H NMR (CD₃OD, 300 MHz TMS): δ 7.71 (1H, dd, J=9 and 3 Hz), 7.59 (1H, dd, J=9 and 3 Hz), 7.32 (2H, d, J=9 Hz), 7.15 (2H, t, J=9 Hz), 6.89 (2H, d, J=9 Hz), 4.6 (2H, br, s) ppm; MS (ESI): m/z 295, [M+H⁺].

### Synthesis of Compound VI-26, 3-fluoro-4-(((4-hydroxyphenyl)sulfonyl)methyl)benzoic acid

### Step 1

To a solution of methyl 3-fluoro-4-(((4-hydroxyphenyl)thio)methyl)benzoate (for synthesis see Step 1 of VI-24) (246 mg) in DCM (6 ml) was added 3-chloroperbenzoic acid (77% pure, 470 mg). After the mixture was stirred over 6 hours, the organic solvents were removed. The residue was suspended in NaCHO₃ aqueous solution. After filtration, rinsing with water and drying under reduced pressure, methyl 3-fluoro-4-(((4-hydroxyphenyl)sulfonyl)methyl)benzoate (270 mg) was obtained as colorless solids.

### Step 2

Following the general basic hydrolysis method, 205 mg of the final product-3-fluoro-4-(((4-hydroxyphenyl)sulfonyl)methyl)benzoic acid was obtained from a basic treatment of methyl 3-fluoro-4-(((4-hydroxyphenyl)sulfonyl)methyl)benzoate (270 mg). ¹H NMR (CD₃OD, 300 MHz TMS): δ 7.77 (1H, dd, J=9 and 3 Hz), 7.60 (1H, dd, J=9 and 3 Hz), 7.502 (2H, d, J=9 Hz), 7.34 (2H, t, J=9 Hz), 6.88 (2H, d, J=9 Hz), 4.56 (2H, s) ppm; MS (ESI): m/z 311 [M+H⁺].

### Synthesis of Compound VI-27, 3-fluoro-4-(2-(3-fluoro-2,4-dihydroxyphenyl)-2-oxoethyl)benzoic acid

### Step 1

Isopropyl 4-(2-chloro-2-oxoethyl)-2-fluorobenzoate (freshly made from 2-(3-fluoro-4-(isopropoxycarbonyl)phenyl)acetic acid (2.49 g) and excess oxyl chloride in DCM with a catalytic amount of DMF) was suspended with AlCl₃ (1.5 g) and ZnCl₂ (154 mg) in DCE and stirred over 1 h at room temperature. Then, 2-fluoro-1,3-dimethoxybenzene (1.98 g) was introduced and the reaction mixture was heated at 80° C. over 40 h. The reaction was quenched with 0.5 N HCl and the mixture were extracted with EtOAc (25 mL×3). The crude product was purified by silica gel column to afford the desired product-isopropyl 3-fluoro-4-(2-(3-fluoro-2-hydroxy-4-methoxyphenyl)-2-oxoethyl)benzoate (450 mg) as yellow solids.

### Step 2

Isopropyl 3-fluoro-4-(2-(3-fluoro-2-hydroxy-4-methoxyphenyl)-2-oxoethyl)benzoate (164 mg) was treated with AlCl3 in toluene at 90° C. over 3 h. The mixture was treated with a diluted HCl and extracted with EtOAc. The crude product was trituated with isopropyl ether to afford the desired product (70 mg) as yellow solids. ¹H NMR (CD₃OD, 300 MHz): δ 7.82 (d, J=6 Hz, 1H), 7.74-7.69 (m, 2H), 7.42 (dd, J=9, 6 Hz, 1H), 6.55 (dd, J=9, 6 Hz, 1H), 4.45 (s, 2H); MS (ESI): m/z 309 [M+H⁺]⁺.

### Synthesis of Compound VI-28, 4-(3-fluoro-4-hydroxyphenylsulfonamido)benzoic acid

### Step 1

3-Fluoro-4-methoxybenzene-1-sulfonyl chloride (150 mg) and methyl 4-aminobenzoate (106 mg) were dissolved in DCM (8 mL) and mixed with pyridine (0.2 mL). The resultant solution was stirred overnight. After removal of solvents, the mixture was diluted with EtOAc (50 mL) and the organic solution was washed with 1NHCl (2×20 mL), brine and dried over Na₂SO₄ (anhy). Removal of solvents afforded the desired product (190 mg)—methyl 4-(3-fluoro-4-methoxyphenylsulfonamido)benzoate as a pink solid, and was taken on without purification.

### Step 2

Methyl 4-(3-fluoro-4-methoxyphenylsulfonamido)benzoate (90 mg) was dissolved in DCM (5 mL) and treated with BBr3 (0.19 ml) at room temperature over 48 h. After removal of solvents, the residue was treated with water and stirred for a couple of hours. The resultant solids were collected by filtration and rinsed with water to afford the pure product (60 mg). ¹H NMR (D6-DMSO, 300 MHz): δ 7.9 (d, J=9, 2H), 7.56-7.48 (m, 3H), 7.20 (d, J=9 Hz, 2H), 6.99 (t, J=9 Hz, 1H), ¹⁹F NMR (D₆-DMSO, 300 MHz): δ−136.62-136.68 (m); MS (ESI): m/z 312 [M+H⁺]⁺.

### Synthesis of Compound VI-29, 4-(3-(2,3-difluoro-4-hydroxyphenyl)-3-oxopropyl)benzoic acid

Following a similar procedure to the two-step procedure described for Compound VI-27, 4-(3-(2,3-difluoro-4-hydroxyphenyl)-3-oxopropyl)benzoic acid was prepared starting from methyl 4-(3-chloro-3-oxopropyl)benzoate and 1,2-difluoro-3-methoxybenzene. ¹H NMR (D6-DMSO, 300 MHz): δ 8.2-7.83 (m, 4H), 7.39 (d, J=9 Hz, 2H), 3.45 (m 2H), 3.04 (t, J=6 Hz, 2H); MS (ESI): m/z 307 [M+H⁺]⁺.

### Synthesis of Compound VI-30, 4-(3-(4-hydroxyphenyl)-3-oxopropyl)benzoic acid

Following a similar procedure to the two-step procedure described for Compound VI-27, 4-(3-(4-hydroxyphenyl)-3-oxopropyl)benzoic acid was prepared starting from methyl 4-(3-chloro-3-oxopropyl)benzoate and anisole. ¹H NMR (D6-DMSO, 300 MHz): δ 7.88-7.83 (m, 4H), 7.39 (d, J=9 Hz, 2H), 6.82 (dd, J=9, 3 Hz, 2H), 3.34-3.27 (m 2H), 2.98 (t, J=6 Hz, 2H); MS (ESI): m/z 271 [M+H⁺]⁺.

### Synthesis of Compound VI-31, 3-fluoro-4-((3-fluoro-4-hydroxybenzyl)amino)benzoic acid

Following a similar procedure to that described for the preparation of Compound VI-32, the title compound was prepared from 3-fluoro-4-hydroxybenzaldehyde and 4-amino-3-fluorobenzoic acid. ¹H NMR (D6-DMSO, 300 MHz): δ 7.52 (dd, J=9, 3 Hz, 1H), 7.50 (dd, J=12, 3 Hz, 1H), 7.10 (dd, J=12, 3 Hz, 1H), 6.99-6.85 (m, 3H), 6.62 (t, J=9 Hz, 1H), 4.30 (d, J=6 Hz; 2H), MS (ESI): m/z 280 [M+H⁺]⁺.

### Synthesis of Compound VI-32, 4-((2,3-difluoro-4-hydroxybenzyl)amino)-3-fluorobenzoic acid

2,3-Difluoro-4-hydroxybenzaldehyde (211 mg) and 4-amino-3-fluorobenzoic acid (251 mg) were dissolved in MeOH (8 mL) and treated with NaBH₃CN (168 mg) over 24 h. Aqueous work up and purification with silica gel column afforded the desired product (50 mg). ¹H NMR (D6-DMSO, 300 MHz): δ 7.52 (dd, J=9, 3 Hz, 1H), 7.48 (dd, J=12, 3 Hz, 1H), 6.96-6.83 (m, 2H), 6.77-6.70 (m, 1H), 6.46 (t, J=9 Hz, 1H), 4.36 (d, J=6 Hz; 2H), MS (ESI): m/z 298 [M+H⁺]⁺.

### Synthesis of Compound VI-33, 3-fluoro-4-((2-fluoro-4-hydroxybenzyl)amino)benzoic acid

Following a similar procedure to that described for the preparation of Compound VI-32, the title compound was prepared from 2-fluoro-4-hydroxybenzaldehyde and 4-amino-3-fluorobenzoic acid. ¹H NMR (D6-DMSO, 300 MHz): δ 7.54 (dd, J=9, 3 Hz, 1H), 7.50 (dd, J=12, 3 Hz, 1H), 7.13 (t, J=9 Hz, 1H), 6.80 (m, 1H), 6.62 (t, J=9 Hz, 1H), 6.557-6.53 (m, 2H), 4.32 (d, J=6 Hz; 2H), MS (ESI): m/z 280 [M+H⁺]⁺.

### Synthetic Details for Compounds of Table 7

### Intermediate 7-1: Synthesis of 4-(4-(4-hydroxyphenyl)-4-oxobutanoyl)benzoic acid

### Step 1: Synthesis of methyl 4-(3-(4-acetoxybenzoyl)-4-ethoxy-4-oxobutanoyl)benzoate

Ethyl 3-(4-acetoxyphenyl)-3-oxopropanoate (650 mg, 2.59 mmol) was dissolved in 10 mL of anhydrous THF under an argon atmosphere. The solution was cooled to 0° C. in an ice bath and 100 mg of NaH (4.17 mmol) was added and stirred for 1 h. Methyl 4-(2-bromoacetyl)benzoate (614 mg, 2.39 mmol) was then added and the solution was stirred overnight was warming to room temperature. The solution was quenched with 10 mL of H₂O and the organics were extracted with EtOAc (25 mL). The organics were washed with brine, dried over sodium sulfate, and concentrated in vacuo. The crude was flashed in a 0%-50% EtOAc in Hexanes solution, resulting in 600 mg (59% yield) of the desired methyl 44344-acetoxybenzoyl)-4-ethoxy-4-oxobutanoyl)benzoate.

### Step 2: Synthesis of Intermediate 7-1

Methyl 4-(3-(4-acetoxybenzoyl)-4-ethoxy-4-oxobutanoyl)benzoate (3 g, 7.03 mmol) was suspended in 25 mL of H₂O. 5.62 mL of a 4N solution of NaOH in H₂O was added and the solution was heated to reflux for 1 h. The solution was then cooled to room temperature and the pH was adjusted to 4.0 and the resulting solids were filtered and dried to yield 4-(4-(4-hydroxyphenyl)-4-oxobutanoyl)benzoic acid (1 g, 48% yield).

### Intermediate 7-2: Synthesis of 5-(4-(4-hydroxyphenyl)-4-oxobutanoyl)thiophene-2-carboxylic acid

### Step 1: Synthesis of methyl 5-(3-(4-acetoxybenzoyl)-4-ethoxy-4-oxobutanoyl)thiophene-2-carboxylate

Ethyl 3-(4-acetoxyphenyl)-3-oxopropanoate (1 g, 3.99 mmol) was dissolved in 20 mL of anhydrous THF under an argon atmosphere and cooled to 0° C. 192 mg (4.79 mmol) of NaH was then added and stirred for 1 h. Methyl 5-(2-bromoacetyl)thiophene-2-carboxylate (1.05 g, 3.99 mmol) was then added and the solution was stirred over night while allowing to warm to room temperature. The solution was then quenched with H₂O (20 mL) and the organics were extracted with EtOAc (50 mL). The organics were washed with brine, dried over sodium sulfate, and concentrated in vacuo. The crude was then flashed in a 0%-50% EtOAc in Hexanes solution, resulting in 1 g (57% yield) of methyl 5-(3-(4-acetoxybenzoyl)-4-ethoxy-4-oxobutanoyl)thiophene-2-carboxylate.

### Step 2: Synthesis of Intermediate 7-2

Methyl 5-(3-(4-acetoxybenzoyl)-4-ethoxy-4-oxobutanoyl)thiophene-2-carboxylate (1 g, 2.31 mmol) was suspended in 8 mL of H₂O. 1.85 mL of a 4N solution of NaOH in H₂O was added and the mixture was heated to reflux for 1 h. The solution was cooled to room temperature and the pH was adjusted to 4.0. The resulting solids were filtered and dried to yield 200 mg (28% yield) of 5-(4-(4-hydroxyphenyl)-4-oxobutanoyl)thiophene-2-carboxylic acid.

General Suzuki Coupling Method:

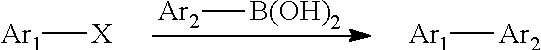

1 mmol of boronic acid and 1 mmol of aromatic bromide or chloride, 4.0 mmol Na₂CO₃, 5% (mol) of (Ph₃P)₄Pd were mixed in 100 ml round flask. 20 ml of 50% dioxane/water was added. The mixture was degassed by evacuation and filling with argon. Repeat degassing three times. The mixture was heated at 100° C. over night. Usually the reaction was complete after overnight stirring. The mixture was diluted with 60 ml water and cooled to room temperature and filtered through fiberglass filter. The solid was washed with water (3×5 ml). The filtrate was acidified to pH=3 and the precipitate was filtered and washed with water (3×5 ml). (If the product does not precipitate out, then an EtOAc extraction is performed to isolate crude). The crude can be purified either by trituration with organic solvents or purified by column chromatography using AcOH:MeOH:Ethyl acetate as solvent B and hexane as solvent A.

General Pyrrole Procedure 1:

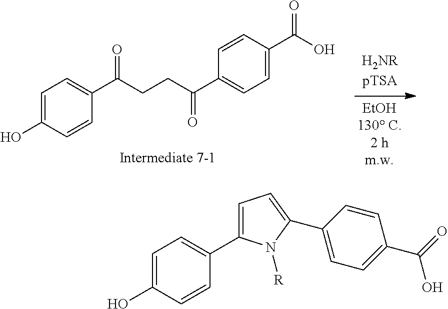

4-(4-(4-hydroxyphenyl)-4-oxobutanoyl)benzoic acid (Intermediate 7-1) 50 mg, 0.167 mmol) was mixed with 10-20 mg para-toluenesulfonic acid and suspended in 2 mL of anhydrous ethanol in a 2-5 mL microwave vessel. 1.67 mmol of amine was added and the mixture was heated to 130° C. for 2 hours while stirring in a microwave reactor. The reaction was diluted with 3-5 mL H₂O and the pH was adjusted to 3.5-4.5. The resulting solid was filtered, dried, and triterated with EtOH, resulting in the desired pyrrole products.

General Pyrrole Procedure 2:

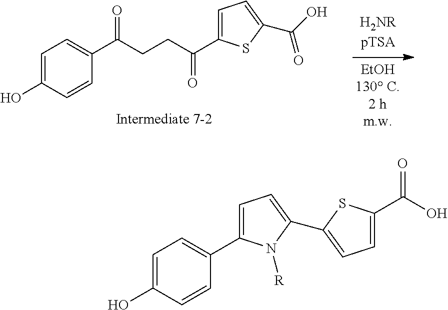

5-(4-(4-hydroxyphenyl)-4-oxobutanoyl)thiophene-2-carboxylic acid (30 mg, 0.0985 mmol) was mixed with 5-10 mg para-toluenesulfonic acid and suspended in 2 mL of anhydrous ethanol in a 2-5 mL microwave vessel. 0.985 mmol of amine was added and the mixture was heated to 130° C. for 2 hours while stirring in a microwave reactor. The reaction mixture was diluted with 3-5 mL H₂O and the pH was adjusted to 3.5-4.5. The resulting solid was filtered, dried, and triterated with EtOH, resulting in the desired pyrrole products.

### Synthesis of Compound VII-1, 4-(4-(4-hydroxyphenyl)thiazol-2-yl)benzoic acid

### Step 1

10 mmol of 2-bromo-1-(4-methoxyphenyl)ethanone and 10 mmol of 4-bromobenzothioamide were mixed in 10 ml ethanol and heated at 100° C. for 1 hour using microwave. The reaction was diluted with 50 ml ethanol and filtered, washed with ethanol to obtain 2.7 g of product (78% yield).

### Step 2

1 mmol of 2-(4-bromophenyl)-4-(4-methoxyphenyl)thiazole was mixed with 4 mmol of CuCN in 10 ml NMP and heated at 200° C. for 2 hour using microwave. The product was precipitated from water and filtered, washed with water and dried.

### Step 3

The material obtained from last step was suspended in 20 ml concentrate HCL and heated at 80° C. overnight. The product was precipitated after cooling to room temperature, filtered, washed with water and dried.

### Step 4

The solids were suspended in DCM and treated with BBr3 (excess) overnight. The reaction was quenched with water and DCM was removed by evaporation. The precipitate was filtered, washed with water and dried. The crude was purified by silica gel column chromatography using 0-100% gradient of A: hexanes and B: 1% AcOH, 5% MeOH in ethyl acetate. 130 mg of final product was obtained. 1H NMR (DMSO-d6 300 MHz TMS): δ 13.13 (s, 1H), 9.69 (s, 1H), 8.15 (m, 4H), 8.02 (s, 1H), 7.89 (d, 2H), 6.87 (d, 2H), MS (ESI): m/z=298.33 [M+1]+.

### Synthesis of Compound VII-2, 4-(2-(4-hydroxyphenyl)thiazol-4-yl)benzoic acid

### Step 1

10 mmol of 4-(2-bromoacetyl)benzonitrile and 10 mmol of 4-methoxybenzothioamide were mixed in 10 ml ethanol and heated at 100° C. for 1 hour using microwave. The reaction was diluted with 50 ml ethanol and filtered, washed with ethanol to obtain 1.9 g of product (66% yield).

### Step 2

500 mg of the material obtained from last step was suspended in 40 ml concentrate HCL and heated at 80° C. for overnight. The product was precipitated after cooling to room temperature, filtered, washed with water and dried.

### Step 3

The solids were suspended in DCM and treated with BBr3 (excess) overnight. The reaction was quenched with water and DCM was removed by evaporation. The precipitate was filtered, washed with water and dried. The crude was purified by silica gel column chromatography using 0-100% gradient of A: hexanes and B: 1% AcOH, 5% MeOH in ethyl acetate. 180 mg of final product was obtained. ¹H NMR (DMSO-d6 300 MHz TMS): δ 12.87 (s, 1H), 10.07 (s, 1H), 8.23 (s, 1H), 8.15 (d, 2H), 8.02 (d, 2H), 7.87 (d, 2H), 6.90 (d, 2H), MS (ESI): m/z=298.27 [M+1]+.

### Synthesis of Compound VII-3, 5-(2-(3-fluoro-4-hydroxyphenyl)thiazol-4-yl)thiophene-2-carboxylic acid

### Step 1

4 mmol of 2,4-dibromothiazole and 4 mmol of (3-fluoro-4-methoxyphenyl)boronic acid were mixed in 15 ml THF, 3 equivalents of K₃PO₄, 5% (mol) of Pd(OAc)₂ and 5% Xanphos were added and the mixture was degassed by evacuation and argon filling three times. The mixture was heated at 80° C. overnight. The reaction was cooled to room temperature and filtered, washed with acetone (3×20 ml). The filtrate was diluted with 200 ml water and the precipitate was filtered, washed with water and dried to obtain quantitative yield.

### Step 2

Followed general Suzuki coupling method using (5-(methoxycarbonyl)thiophen-2-yl)boronic acid wherein the mixture was heated at 90° C. for 3 hours. For workup: 5 ml of 1N NaOH was added followed by addition of 10 ml water prior to filtering. The filtrate was acidified to pH=3 and the resulting precipitate was filtered, washed with water (3×5 ml) and dried to obtain 74 mg of methyl 5-(2-(3-fluoro-4-methoxyphenyl)thiazol-4-yl)thiophene-2-carboxylate (45% yield).

### Step 3

The material obtained from last step was suspended in 10 ml dry DCM and 3 equivalents of BBr₃ were added while the reaction was cooled in ice water bath. The mixture was stirred at room temperature overnight and 0.2 ml more of BBr₃ was added and stirred for another day. The mixture was quenched with water. DCM was removed by evaporation. The precipitate was filtered, washed with water and dried. The crude was purified by silica gel column chromatography using 0-60% gradient of A: hexanes and B: 1% AcOH, 5% MeOH in ethyl acetate. 14 mg of final product was obtained. ¹H NMR (DMSO-d6 300 MHz TMS): δ 13.2 (s, 1H), 10.06 (s, 1H), 8.18 (s, 1H), 7.64 (m, 4H), 7.10 (t, 1H), MS (ESI): m/z=322.37 [M+1]+.

### Synthesis of Compound VII-4, 4-(5-(4-hydroxyphenyl)thiophen-2-yl)benzoic acid

Followed general Suzuki coupling method with 4-(5-bromothiophen-2-yl)benzoic acid and 4-hydroxyphenylboronic acid wherein the reaction was heated to 100° C. for 1 hour in the microwave reactor. The solution was diluted with H₂O (10 mL) and acidified with conc. HCl to a pH=4. The resulting solid was filtered and triturated with EtOAc to yield 16 mg 4-(5-(4-hydroxyphenyl)thiophen-2-yl)benzoic acid. 1H NMR (DMSO-d6 300 MHz TMS): δ 9.76 (bs, 1H), 7.95 (d, 2H), 7.77 (d, 2H), 7.65 (d, 1H), 7.55 (d, 2H), 7.40 (d, 1H), 6.85 (d, 2H). MS (ESI): m/z=295.37 [M−1]−.

### Synthesis of Compound VII-5, 3-(2-(3-fluoro-4-hydroxyphenyl)thiazol-4-yl)benzoic acid

Followed a procedure similar to steps 2 and 3 of Compound VII-3: 4-bromo-2-(3-fluoro-4-methoxyphenyl)thiazole (see step 1 of Compound VII-3) (100 mg) was treated with (3-(methoxycarbonyl)phenyl)boronic acid (63 mg), 5 mol % Pd(PPh₃)4 (20 mg) and NaHCO₃ (65 mg) in Dioxane/Water at 115° C. for 90 min with microwave to afford a yellow solid (55 mg) after column purification. The solids were suspended in DCM and treated with BBr3 (excess) overnight. The resultant mixture was purified by column chromatography to afford the desired product (10 mg) as light brown solids. ¹H NMR (DMSO-d6 300 MHz TMS): 8.97 (s, 1H), 8.24 (m, 2H), 7.95 (d, 2H, J=6 Hz), 7.80 (dd, 1H, J=3, 12 Hz), 7.70 (d, 1H, J=9 Hz), 7.60 (3H, dd, J=6, 9 Hz), 7.10 (1H, t, J=9 Hz); 19F NMR (DMSO-d6, 300 MHz): 135.42 (t, J=12 Hz); MS (ESI): m/z=316 [M+1]⁺.

### Synthesis of Compound VII-6, 4-(2-(3-fluoro-4-hydroxyphenyl)thiazol-4-yl)benzoic acid

Followed a procedure similar to steps 2 and 3 of Compound VII-3: 4-bromo-2-(3-fluoro-4-methoxyphenyl)thiazole (100 mg) was treated with (4-(methoxycarbonyl)phenyl)boronic acid (63 mg), 5% (mol) Pd(PPh3)4 (20 mg) and NaHCO₃ (65 mg) in Dioxane/Water at 115° C. C for 90 min with microwave to afford a yellow solid (55 mg) after column purification. The solids were suspended in DCM and treated with BBr3 (excess) overnight. The resultant mixture was purified by column chromatography to afford the desired product-4-(2-(3-fluoro-4-hydroxyphenyl)thiazol-4-yl)benzoic acid (10 mg) as light brown solids. ¹H NMR (DMSO-d6 300 MHz TMS): 8.97 (s, 1H), 8.18 (2H, d, J=6 Hz), 8.04 (d, 2H, J=6 Hz), 7.82 (dd, 1H, J=3, 12 Hz), 7.70 (m, 1H), 7.10 (1H, t, J=9 Hz); 19F NMR (DMSO-d6, 300 MHz): 135.42 (t, J=12 Hz); MS (ESI): m/z=316 [M+1]⁺.

### Synthesis of Compound VII-7, 4-(5-(3-fluoro-4-hydroxyphenyl)thiophen-2-yl)benzoic acid

### Step 1

4-(thiophen-2-yl)benzoic acid (2.45 mmol, 500 mg) was dissolved in 10 mL of DCM and NBS (2.45 mmol, 436 mg) was added. The mixture was stirred over night. The crude material was then concentrated in vacuo, suspended in H₂O (25 mL), and the organics were extracted with EtOAc (30 mL) to afford 690 mg of 4-(5-bromothiophen-2-yl)benzoic acid.

### Step 2

Followed general Suzuki coupling method with 4-(5-bromothiophen-2-yl)benzoic acid wherein the reaction was heated to 100° C. for 1 hour in a microwave reactor. Workup was followed by DCM trituration to yield 100 mg of 4-(5-(3-fluoro-4-methoxyphenyl)thiophen-2-yl)benzoic acid.

### Step 3

The above product was taken up in DCM and excess BBr₃ was added and stirred for 6 h. The solution was then concentrated in vacuo and triturated with EtOAc to give 10 mg of 4-(5-(3-fluoro-4-hydroxyphenyl)thiophen-2-yl)benzoic acid. ¹H NMR (DMSO-d6 300 MHz TMS): δ 12.99 (bs, 1H), 10.19 (s, 1H), 7.99 (d, 2H), 7.78 (d, 2H), 7.66 (d, 1H), 7.58 (dd, 1H), 7.48 (d, 1H), 7.33 (dd, 1H), 7.01 (t, 1H). MS (ESI): m/z=313.39 [M−1]⁻.

### Synthesis of Compound VII-8, 4-(5-(2-fluoro-4-hydroxyphenyl)thiophen-2-yl)benzoic acid

### Step 1

Followed general Suzuki coupling method with 4-(5-bromothiophen-2-yl)benzoic acid (see Step 1 of Compound VII-7 for synthesis) and 2-fluoro-4-methoxyphenylboronic acid wherein the reaction was heated for 1 hour in a microwave reactor. Workup was followed by DCM trituration to yield 50 mg of 4-(5-(2-fluoro-4-methoxyphenyl)thiophen-2-yl)benzoic acid.

### Step 2

4-(5-(2-fluoro-4-methoxyphenyl)thiophen-2-yl)benzoic acid (0.15 mmol, 50 mg) was taken up in 5 mL of DCM and excess BBr3 was added and stirred for 6 h. The solution was then concentrated in vacuo and triturated with EtOAc to give 10 mg of 4-(5-(2-fluoro-4-hydroxyphenyl)thiophen-2-yl)benzoic acid. ¹H NMR (DMSO-d6 300 MHz TMS): δ 13.01 (bs, 1H), 10.26 (s, 1H), 7.99 (d, 2H), 7.82 (d, 2H), 7.68 (d, 1H), 7.62 (d, 1H), 7.47 (d, 1H) 6.74 (m, 2H). MS (ESI): m/z=313.19 [M−1]⁻.

### Synthesis of Compound VII-9, 5-(2-(4-hydroxyphenyl)thiazol-4-yl)thiophene-2-carboxylic acid

### Step 1

2,4-dibromothiazole (2.06 mmol, 500 mg), 4-hydroxyphenylboronic acid (2.06 mmol, 284 mg), potassium phosphate (1.31 g), Xanphos (5 mol %), and Pd(OAc)2 (5 mol %) were mixed in a 100 mL round bottom flask. 50 mL of THF was added and the mixture was degassed ×3 in an ice bath and placed under an argon atmosphere. The mixture was then heated to 80° C. over night. The mixture was diluted with H₂O (50 mL) and the organics were extracted with EtOAc (50 mL). The organics were concentrated in vacuo and the crude material was triturated with DCM to yield 350 mg of 4-(4-bromothiazol-2-yl)phenol.

### Step 2

Followed general Suzuki coupling method with 5-boronothiophene-2-carboxylic acid wherein the reaction was heated to 100° C. for 2 hours in a microwave. The workup was followed by trituration with DCM to afford 15 mg of 5-(2-(4-hydroxyphenyl)thiazol-4-yl)thiophene-2-carboxylic acid. ¹H NMR (DMSO-d6 300 MHz TMS): δ 10.11 (s, 1H), 8.13 (s, 1H), 7.85 (d, 2H), 7.73 (dd, 2H), 6.92 (d, 2H). MS (ESI): m/z=302.32 [M−1]−.

### Synthesis of Compound VII-10, 5-(2-(3-fluoro-4-hydroxyphenyl)thiazol-5-yl)thiophene-2-carboxylic acid

### Step 1

Followed the general Suzuki coupling method with 2,5-dibromothiazole and (3-fluoro-4-methoxyphenyl)boronic acid, wherein the mixture was heated at 100° C. for 2 days.

### Step 2

Followed the general Suzuki coupling method with 5-boronothiophene-2-carboxylic acid. Following workup, the filtrate was acidified to pH=2 and the precipitate was filtered, washed with water (3×5 ml) and dried. The solid was dissolved in 2 ml DMSO and precipitated again from water and filtered, washed with water and dried. 30 mg of crude product was obtained.

### Step 3

The solids were suspended in DCM and treated with BBr3 (excess) for 2 days and quenched with water. DCM was removed by evaporation. The precipitate was filtered, washed with water and dried to afford 20 mg of final product. ¹H NMR (DMSO-d6 300 MHz TMS): δ 13.32 (s, 1H), 10.63 (s, 1H), 8.24 (s, 1H), 7.72 (m, 2H), 7.63 (d, 1H), 7.46 (d, 1H), 7.08 (t, 1H) MS (ESI): m/z=322.37 [M+1]⁺.

### Compound VII-11, 4″-hydroxy-[1,1′:4′,1″-terphenyl]-4-carboxylic acid

Followed general Suzuki coupling method with 4-hydroxyboronic acid and 4′-bromo-[1,1′-biphenyl]-4-carboxylic acid. ¹H NMR (DMSO-d6 300 MHz TMS): δ 13.0 (s, 1H), 9.62 (s, 1H), 8.02 (d, 2H), 7.87 (d, 2H), 7.80 (d, 2H), 7.70 (d, 2H), 7.57 (d, 2H), 6.88 (d, 2H), MS (ESI): m/z=289.36 [M−1]⁻.

### Synthesis of Compound VII-12, 5′-(4-hydroxyphenyl)-2,2′-bithiophene-5-carboxylic acid

### Step 1

Followed general Suzuki coupling method with 2,5-dibromothiophene and 5-boronothiophene-2-carboxylic acid with 10 mol % catalyst. Reaction was heated to 80° C. for 2 hours in a microwave reactor. After workup the filtrate was rinsed with MeCN and the mother liquor was collected and concentrated in vacuo to a volume of −10 mL. EtOAc (15 mL) was added and the solution was stirred for 10 minutes before being filtered, yielding 110 mg of desired 5′-bromo-2,2′-bithiophene-5-carboxylic acid.

### Step 2

Followed general Suzuki coupling method with 4-hydroxyphenylboronic acid and 10 mol % catalyst wherein reaction was heated to 100° C. for 1 hour in a microwave reactor. After workup and concentration, the crude desired was triturated with a aqueous bicarbonate solution to yield 10 mg of 5′-(4-hydroxyphenyl)-2,2′-bithiophene-5-carboxylic acid. ¹H NMR (DMSO-d6 300 MHz TMS): δ 7.48 (d, 2H), 7.25 (dd, 2H), 7.07 (s, 2H), 6.81 (d, 2H). MS (ESI): m/z=301.44 [M−1]⁻.

### Synthesis of VII-13, 5-(5-(4-hydroxyphenyl)thiazol-2-yl)thiophene-2-carboxylic acid

### Step 1

Followed general Suzuki coupling method with 2,5-dibromothiazole and 5-boronothiophene-2-carboxylic acid. After workup and concentration, the crude material was re-dissolved in 25 mL of a bicarbonate solution and acidified to pH=4 with conc. HCl. The resulting solid was filtered to yield 180 mg 5-(5-bromothiazol-2-yl)thiophene-2-carboxylic acid.

### Step 2

Followed general Suzuki coupling method with 4-hydroxyphenylboronic acid wherein the mixture was then heated to 100° C. for 1 hour in a microwave reactor. Workup was followed by trituration with a bicarbonate solution to yield 60 mg of 5-(5-(4-hydroxyphenyl)thiazol-2-yl)thiophene-2-carboxylic acid. ¹H NMR (DMSO-d₆ 300 MHz TMS): δ 7.97 (s, 1H), 7.5 (d, 2H), 7.41 (d, 1H), 7.16 (d, 1H), 6.84 (d, 2H). MS (ESI): m/z=304.41 [M+1]⁺.

### Compound VII-14, 4-(5-(4-hydroxyphenyl)pyrimidin-2-yl)benzoic acid

Followed general Suzuki coupling method with 4-hydroxyboronic acid and 4-(5-chloropyrimidin-2-yl)benzoic acid. ¹H NMR (DMSO-d6 300 MHz TMS): δ 9.18 (s, 2H), 8.83 (d, 2H), 8.04 (d, 2H), 7.72 (d, 2H), 6.93 (d, 2H), MS (ESI): m/z=293.57 [M+1]⁺.

### Compound VII-15, 4-(2-(4-hydroxyphenyl)pyrimidin-5-yl)benzoic acid

Followed general Suzuki coupling method with 4-(5-chloropyrimidin-2-yl)phenol and (4-(methoxycarbonyl)phenyl)boronic acid. ¹H NMR (DMSO-d₆ 300 MHz TMS): δ 13.1 (s, 1H), 10.1 (s, 1H), 9.22 (s, 2H), 8.31 (d, 2H), 8.07 (d, 2H), 7.98 (d, 2H), 6.91 (d, 2H), MS (ESI): m/z=293.5 [M+1]⁺.

### Synthesis of Compound VII-16, 5-(2-(4-hydroxyphenyl)thiazol-5-yl)thiophene-2-carboxylic acid

### Step 1

2,5-dibromothiazole (1.03 mmol, 250 mg), 4-hydroxyphenylboronic acid (1.03 mmol, 142 mg), potassium phosphate (656 mg), Xanphos (5 mol %, 30 mg), and Pd(OAc)2 (5 mol %, 11.6 mg) were combined in a 20 mL microwave reaction vessel and 15 mL of THF was added. The vessel was degassed 3 times in an ice bath and placed under an argon atmosphere. The mixture was stirred at 80° C. overnight, followed by filtration and concentration of the mother liquor in vacuo. The crude material suspended in 20 mL of 0.5N NaOH and filtered after sonication. The mother liquor was collected and acidified to pH=5 with conc. HCl and the resulting solid was collected and dried in vacuo to afford 50 mg of 4-(5-bromothiazol-2-yl)phenol which was used without further purification.

### Step 2

Followed general Suzuki coupling method wherein the mixture was heated to 100° C. in a microwave reactor for 1 hour. The crude reaction mixture was diluted with H₂O (10 mL) and the organics were extracted with EtOAc (20 mL). The organics were concentrated in vacuo and the crude was dissolved in 0.5N NaOH. The basic solution was acidified and the solids were collected and triturated with acetone to yield 10 mg 5-(2-(4-hydroxyphenyl)thiazol-5-yl)thiophene-2-carboxylic acid. ¹H NMR (DMSO-d6 300 MHz TMS): δ 13.3 (bs, 1H), 10.15 (s, 1H), 8.21 (s, 1H), 7.82 (d, 2H), 7.72 (d, 1H), 7.46 (d, 1H), 6.91 (d, 2H). MS (ESI): m/z=302.45 [M−1]⁻.

### Synthesis of Compound VII-17, 5′-(3-fluoro-4-hydroxyphenyl)-[2,2′-bithiophene]-5-carboxylic acid

### Step 1

Followed general Suzuki coupling method starting with 2,5-dibromothiophene and 5-boronothiophene-2-carboxylic acid and 10 mol % catalyst. Reaction was run at 80° C. for 2 hours in a microwave reactor. The crude mixture was then diluted with H₂O and filtered and the filtrate was washed with MeCN. The mother liquor was concentrated in vacuo to a volume of 10 mL and 15 mL of EtOAc was added and stirred for 30 minutes. This solution was filtered to give 110 mg of 5′-bromo-2,2′-bithiophene-5-carboxylic acid.

### Step 2

Followed general Suzuki coupling method starting with 5′-bromo-2,2′-bithiophene-5-carboxylic acid and 3-fluoro-4-hydroxyphenylboronic acid and 10 mol % catalyst. The reaction was heated to 80° C. for 1 hour in a microwave reactor. The crude mixture was then diluted with H₂O (10 mL) and filtered. The mother liquor was acidified to pH=4 and the solids were filtered and triturated with DCM to give Compound VII-17. ¹H NMR (DMSO-d₆ 300 MHz TMS): δ 13.20 (bs, 1H), 10.23 (s, 1H), 7.70 (d, 1H, J=6 Hz), 7.57 (dd, 1H, J=3, 12 Hz), 7.48 (dd, 2H, J=3.9 Hz), 7.37 (d, 1H, J=3 Hz), 7.33 (m, 1H), 7.03 (t, 1H, J=9 Hz). MS (ESI): m/z=319.46 [M−1]⁻.

### Synthesis of Compound VII-18, 3-(2-(4-hydroxyphenyl)thiazol-4-yl)benzoic acid

### Step 1

Followed general Suzuki coupling method starting with 2,4-dibromothiazole, 4-hydroxyphenylboronic acid, and 10 mol % catalyst. The reaction was heated to 90° C. for 1 hour in a microwave reactor. The crude mixture was then diluted with H₂O and filtered. The mother liquor was acidified to pH=7 with 1N HCl and the solids were filtered and dried in vacuo to give 4-(4-bromothiazol-2-yl)phenol.

### Step 2

Followed general Suzuki coupling method starting with 4-(4-bromothiazol-2-yl)phenol, 3-(methoxycarbonyl)phenylboronic acid, and 10 mol % catalyst. The reaction was heated to 90° C. for 1 hour in a microwave reactor. The crude mixture was then diluted with H₂O and extracted with EtOAc, washed with brine, and concentrated in vacuo. The crude was then dissolved in 6 mL of a 5:1 solution of 1N NaOH:THF and stirred over night. The solution was filtered, and the mother liquor was acidified to a pH=4 and the resulting solids were collected to yield 100 mg of 3-(2-(4-hydroxyphenyl)thiazol-4-yl)benzoic acid. ¹H NMR (DMSO-d₆ 300 MHz TMS): δ 13.13 (bs, 1H), 10.06 (bs, 1H), 8.60 (t, 1H, J=1.5 Hz), 8.28 (dt, 1H, J=1.5, 8.4 Hz), 8.19 (s, 1H), 7.95 (dt, 1H, J=1.5, 8.4 Hz), 7.89 (d, 2H, J=8.7 Hz), 7.65 (m, 6H), 6.93 (d, 2H, J=8.7 Hz). MS (ESI): m/z=298.47 [M+1]⁺.

### Synthesis of Compound VII-19, 4′-(4-hydroxyphenyl)-[2,2′-bithiophene]-5-carboxylic acid

### Step 1

Followed general Suzuki coupling method starting with 4-dibromothiophene, 5-boronothiophene-2-carboxylic acid, and 10 mol % catalyst. The reaction was heated to 90° C. for 1 hour in a microwave reactor. The crude mixture was then diluted with H₂O (10 mL) and filtered. The mother liquor was acidified to pH=4 and the solids were filtered and triturated with DCM to give 4′-bromo-2,2′-bithiophene-5-carboxylic acid.

### Step 2

Followed general Suzuki coupling method starting with 4′-bromo-2,2′-bithiophene-5-carboxylic acid, 4-hydroxyphenylboronic acid, and 10 mol % catalyst. The reaction was heated to 80° C. for 1 hour in a microwave reactor. The crude mixture was then diluted with H₂O (10 mL) and filtered. The mother liquor was acidified to pH=4 and the solids were filtered and triturated with DCM to give 4′-(4-hydroxyphenyl)-2,2′-bithiophene-5-carboxylic acid. ¹H NMR (CD₃OD 300 MHz TMS): δ 7.67 (d, 1H, J=3 Hz), 7.63 (d, 1H, J=1.5 Hz) 7.54 (d, 2H, J=9 Hz), 7.46 (d, 1H, J=1.5 Hz) 7.29 (d, 1H, J=6 Hz), 6.86 (d, 2H, J=9 Hz). MS (ESI): m/z=301.44 [M−1]⁻.

### Synthesis of Compound VII-20, 5′-(4-hydroxyphenyl)-[2,3′-bithiophene]-5-carboxylic acid

### Step 1

Followed general Suzuki coupling method starting with 2,4-dibromothiophene, 4-hydroxyphenylboronic acid, and 10 mol % catalyst. The reaction was heated to 80° C. for 1 hour in a microwave reactor. The crude mixture was then diluted with H₂O (10 mL) and filtered. The mother liquor was concentrated in vacuo and the crude solid was resuspended in water and filtered. The filtered solid was rinsed with acetone, and the acetone mother liquor was diluted with H₂O to a 3:1 H₂O:acetone mixture. The organics were extracted with EtOAc and purified using silica gel chromatography with a mobile phase gradient of 0 to 55% EtOAc in Hexanes to yield 200 mg of 4-(4-bromothiophen-2-yl)phenol.

### Step 2

Followed general Suzuki coupling method starting with 4-(4-bromothiophen-2-yl)phenol, 5-boronothiophene-2-carboxylic acid, and 10 mol % catalyst. The reaction was heated to 80° C. for 1 hour in a microwave reactor. The crude mixture was then diluted with H₂O and rinsed with MeCN. The mother liquor was concentrated in vacuo and the crude solid was resuspended in water and then filtered. The crude material was triturated with DCM to give 5′-(4-hydroxyphenyl)-2,3′-bithiophene-5-carboxylic acid. ¹H NMR (CD₃OD 300 MHz TMS): δ 7.52 (s, 1H), 7.49 (d, 3H, J=3 Hz), 7.43 (d, 1H, J=3 Hz), 7.24 (d, 1H, J=3 Hz), 6.85 (d, 2H, J=9 Hz). MS (ESI): m/z=301.44 [M−1]⁻.

### Synthesis of Compound VII-21, 5-(4′-hydroxy-[1,1′-biphenyl]-4-yl)thiophene-2-carboxylic acid

Followed general Suzuki coupling method described for compounds of Table 7 starting from 4′-bromo-[1,1′-biphenyl]-4-ol and 5-boronothiophene-2-carboxylic acid. MS (ESI): m/z 295.44 [M−1]⁻.

### Synthesis of Compound VII-22, 4-(5-(4-hydroxyphenyl)furan-2-yl)benzoic acid

4-(4-(4-hydroxyphenyl)-4-oxobutanoyl)benzoic acid (Intermediate 7-1, 50 mg, 0.168 mmol) was dissolved in 2 mL of anhydrous EtOH and 100 uL of 4N HCl in Dioxane was added. The solution was stirred for 30 minutes at 130° C. in a microwave reactor. The pH was neutralized and the resulting solid was filtered and triterated with EtOH to yield 20 mg (42% yield) of 4-(5-(4-hydroxyphenyl)furan-2-yl)benzoic acid. ¹H-NMR (300 MHz, CD₃OD, ppm): δ 8.08 (d, j=9 Hz, 2H), 8.05 (d, j=9 Hz, 2H), 7.67 (d, j=9 Hz, 2H), 7.02 (d, j=3 Hz, 1H), 6.89 (d, j=9 Hz, 2H), 6.73 (d, j=3 Hz, 1H); MS (EI), m/z: [M+1] 281.39, [M−1] 379.37.

### Synthesis of Compound VII-23, 4-(5-(4-hydroxyphenyl)-1H-pyrrol-2-yl)benzoic acid

Intermediate 7-1 (4-(4-(4-hydroxyphenyl)-4-oxobutanoyl)benzoic acid) (50 mg, 0.168 mmol) was dissolved in 2 mL of anhydrous EtOH and was mixed with 10-20 mg para-toluenesulfonic acid. 1.68 mmol of ammonia (7N solution in MeOH) was added. The mixture was stirred at 130° C. in a microwave reactor for 1 h. The solution was cooled to room temperature and the pH was adjusted to 4.2 and diluted with water. The resulting solid was filtered and triterated with EtOH to yield 20 mg of 4-(5-(4-hydroxyphenyl)-1H-pyrrol-2-yl)benzoic acid (43% yield). ¹H-NMR (300 MHz, DMSO-d₆, ppm): δ 11.22 (bs, 1H), 7.91 (q, j=9 Hz, 4H), 7.62 (d, j=9 Hz, 2H), 6.81 (d, j=9 Hz, 2H), 6.73 (dd, j=3 Hz, 1H), 6.46 (dd, j=3 Hz, 1H); MS (EI), m/z: [M+1] 280.36, [M−1] 278.36.

### Synthesis of Compound VII-24, 5-(5-(4-hydroxyphenyl)-1-methyl-1H-pyrrol-2-yl)thiophene-2-carboxylic acid

Followed general pyrrole procedure 2 for compounds of Table 7, starting with 0.164 mmol of Intermediate 7-2 and 1.64 mmol methylamine. Obtained 20 mg (41% yield) of the desired product. ¹H-NMR (300 MHz, DMSO-d₆, ppm): δ 9.64 (bs, 1H), 7.63 (dt, j=9, 3 Hz, 2H), 7.44 (d, j=6 Hz, 1H), 7.30 (dt, j=9, 3 Hz, 2H), 7.24 (d, j=6 Hz, 1H), 6.49 (d, j=3 Hz, 1H), 6.16 (d, j=3 Hz, 1H), 3.66 (s, 3H); MS (EI), m/z: [M+1] 300.35, [M−1] 298.33.

### Synthesis of Compound VII-25, 5-(5-(4-hydroxyphenyl)-1-(2-methoxyethyl)-1H-pyrrol-2-yl)thiophene-2-carboxylic acid

Followed general pyrrole procedure 2, starting with 0.164 mmol of Intermediate 7-2 and 1.64 mmol 2-methoxyethanamine. 47 mg (83% yield) of the desired product was obtained. ¹H-NMR (300 MHz, DMSO-d₆, ppm): δ 9.65 (bs, 1H), 7.71 (d, j=6 Hz, 1H), 7.28 (m, 3H), 6.86 (d, j=9 Hz, 2H), 6.45 (d, j=3 Hz, 1H), 6.11 (d, j=3 Hz, 1H), 4.30 (t, j=6 Hz, 2H), 3.22 (t, j=6 Hz, 2H), 2.96 (s, 3H). MS (EI), m/z: [M+1] 344.39, [M−1] 342.50.

### Synthesis of Compound VII-26, 5-(1-butyl-5-(4-hydroxyphenyl)-1H-pyrrol-2-yl)thiophene-2-carboxylic acid

Followed general pyrrole procedure 2, starting with butan-1-amine and Intermediate 7-2. 15 mg (57% yield) of the desired product was obtained. ¹H-NMR (300 MHz, CD₃OD, ppm): δ 7.71 (d, j=3 Hz, 1H), 7.39 (d, j=9 Hz, 2H), 7.14 (d, j=3 Hz, 1H), 7.12 (d, j=6 Hz, 1H), 6.78 (d, j=9 Hz, 2H), 6.62 (d, j=3 Hz, 1H), 4.16 (t, j=6 Hz, 2H), 1.74 (m, 2H), 1.36 (m, 2H), 0.95 (t, j=6 Hz, 3H); MS (EI), m/z: [M+1] 342.31, [M−1] 340.48.

### Synthesis of Compound VII-27, 5-(5-(4-hydroxyphenyl)-1-isopentyl-1H-pyrrol-2-yl)thiophene-2-carboxylic acid

Followed general pyrrole procedure 2, starting with 3-methylbutan-1-amine and Intermediate 7-2. 20 mg (57% yield) of the desired product was obtained. ¹H-NMR (300 MHz, DMSO-d⁶, ppm): δ 9.65 (s, 1H), 7.70 (d, j=6 Hz, 1H), 7.26 (m, 3H), 6.87 (d, j=9 Hz, 2H), 6.44 (d, j=3 Hz, 1H), 6.10 (d, j=6 Hz, 1H), 4.15 (t, j=3 Hz, 2H), 0.87 (m, 3H), 0.60 (d, j=6 Hz, 6H); MS (EI), m/z: [M+1] 356.42, [M−1] 354.47.

### Synthesis of Compound VII-28, 4-(5-(4-hydroxyphenyl)-1-isopentyl-1H-pyrrol-2-yl)benzoic acid

Followed procedure similar to general pyrrole procedure 1 for compounds of Table 7, starting with Intermediate 7-1 (100 mg, 0.334 mmol) and 3.34 mmol of 3-methylbutan-1-amine. Obtained 60 mg (51% yield) of the desired product. ¹H-NMR (300 MHz, DMSO-d₆, ppm): δ 9.59 (bs, 1H), 8.00 (d, j=9 Hz, 2H), 7.59 (d, j=9 Hz, 2H), 7.28 (d, j=9 Hz, 2H), 6.86 (d, j=9 Hz, 2H), 6.31 (d, j=3 Hz, 1H), 6.12 (d, j=3 Hz, 1H) 4.10 (t, j=3 Hz, 2H), 0.99 (m, 3H), 0.47 (d, j=6 Hz, 6H); MS (EI), m/z: [M+1] 350.31, [M−1] 348.42.

### Synthesis of Compound VII-29, 4-(5-(4-hydroxyphenyl)-1-isobutyl-1H-pyrrol-2-yl)benzoic acid

Followed general pyrrole procedure 1, starting with 2-methylpropan-1-amine and Intermediate 7-1. Obtained 40 mg (71% yield) of the desired product. ¹H-NMR (300 MHz, CD₃OD, ppm): δ 8.08 (d, j=6 Hz, 2H), 7.58 (d, j=9 Hz, 2H), 7.30 (d, j=9 Hz, 2H), 6.88 (d, j=6 Hz, 2H), 6.33 (d, j=3 Hz, 1H), 6.15 (d, j=3 Hz, 1H), 3.99 (d, j=9 Hz, 2H), 1.44 (m, 1H), 0.40 (d, j=6 Hz, 6H); MS (EI), m/z: [M+1] 336.45, [M−1] 334.50.

### Synthesis of Compound VII-30, 4-(5-(4-hydroxyphenyl)-1-(4-methylpentyl)-1H-pyrrol-2-yl)benzoic acid

Followed general pyrrole procedure 1, starting with 4-methylpentan-1-amine and Intermediate 7-1. Obtained 40 mg (66% yield) of the desired product. ¹H-NMR (300 MHz, CD₃OD, ppm): δ 8.08 (d, j=9 Hz, 2H), 7.56 (d, j=9 Hz, 2H), 7.29 (d, j=6 Hz, 2H), 6.88 (d, j=6 Hz, 2H), 6.30 (d, j=3 Hz, 1H), 6.13 (d, j=3 Hz, 1H), 4.13 (t, j=9 Hz, 2H), 1.17 (m, 3H), 0.69 (m, 2H), 0.61 (d, j=6 Hz, 6H); MS (EI), m/z: [M+1] 364.24, [M−1] 362.47.

### Synthesis of Compound VII-31, 4-(5-(4-hydroxyphenyl)-1-(3-isopropoxypropyl)-1H-pyrrol-2-yl)benzoic acid

Followed general pyrrole procedure 1, starting with 3-isopropoxypropan-1-amine and Intermediate 7-1. Obtained 31 mg (49% yield) of the desired product. ¹H-NMR (300 MHz, CD₃OD, ppm): δ 8.09 (d, j=9 Hz, 2H), 7.60 (d, j=9 Hz, 2H), 7.32 (d, j=9 Hz, 2H), 6.89 (d, j=9 Hz, 2H), 6.34 (d, j=3 Hz, 1H), 6.16 (d, j=3 Hz, 1H), 4.30 (t, j=6 Hz, 2H), 3.22 (m, 1H), 2.95 (t, j=6 Hz, 2H), 1.40 (m, 2H), 0.88 (d, j=6 Hz, 6H); MS (EI), m/z: [M+1] 380.43, [M−1] 378.47.

### Synthesis of Compound VII-32, 4-(5-(4-hydroxyphenyl)-1-isopropyl-1H-pyrrol-2-yl)benzoic acid

Followed general pyrrole procedure 1, starting with propan-2-amine and Intermediate 7-1. Obtained 14 mg (26% yield) of the desired product. ¹H-NMR (300 MHz, CD₃OD, ppm): δ 8.08 (d, j=9 Hz, 2H), 7.54 (d, j=9 Hz, 2H), 7.27 (d, j=9 Hz, 2H), 6.85 (d, j=9 Hz, 2H), 6.16 (d, j=3 Hz, 1H), 6.02 (d, j=3 Hz, 1H), 4.52 (m, 1H), 1.26 (d, j=9 Hz, 6H); MS (EI), m/z: [M+1] 322.28, [M−1] 320.39.

### Synthesis of Compound VII-33, 4-(5-(4-hydroxyphenyl)-1-(2-methoxyethyl)-1H-pyrrol-2-yl)benzoic acid

Followed general pyrrole procedure 1, starting with 2-methoxyethanamine and Intermediate 7-1. Obtained 32 mg (57% yield) of the desired product. ¹H-NMR (300 MHz, CD₃OD, ppm): δ 8.09 (d, j=9 Hz, 2H), 7.60 (d, j=9 Hz, 2H), 7.32 (d, j=9 Hz, 2H), 6.89 (d, j=9 Hz, 2H), 6.32 (d, j=3 Hz, 1H), 6.16 (d, j=3 Hz, 1H), 4.30 (t, j=6 Hz, 2H), 3.09 (t, j=6 Hz, 2H), 2.92 (s, 3H); MS (EI), m/z: [M+1] 338.28 [M−1] 336.45.

### Synthesis of Compound VII-34, 4-(1-hexyl-5-(4-hydroxyphenyl)-1H-pyrrol-2-yl)benzoic acid

Followed general pyrrole procedure 1, starting with hexan-1-amine and Intermediate 7-1. Obtained 12 mg (20% yield) of the desired product. ¹H-NMR (300 MHz, CD₃OD, ppm): δ 8.09 (d, j=9 Hz, 2H), 7.58 (d, j=9 Hz, 2H), 7.30 (d, j=9 Hz, 2H), 6.89 (d, j=9 Hz, 2H), 6.31 (d, j=3 Hz, 1H), 6.14 (d, j=3 Hz, 1H), 4.16 (t, j=6 Hz, 2H), 1.18 (q, j=6 Hz, 2H), 1.04 (q, j=6 Hz, 2H), 0.90 (m, 4H), 0.73 (t, j=6 Hz, 3H); MS (EI), m/z: [M+1] 364.42, [M−1] 362.53.

### Synthesis of Compound VII-35, 4-(1-(2-ethoxyethyl)-5-(4-hydroxyphenyl)-1H-pyrrol-2-yl)benzoic acid

Followed general pyrrole procedure 1, starting with 2-ethoxyethanamine and Intermediate 7-1. Obtained 22 mg (37% yield) of the desired product. ¹H-NMR (300 MHz, DMSO-d₆, ppm): δ 9.60 (s, 1H), 8.00 (d, j=9 Hz, 2H), 7.61 (d, j=9 Hz, 2H), 7.30 (d, j=9 Hz, 2H), 6.86 (d, j=9 Hz, 2H), 6.31 (d, j=3 Hz, 1H), 6.13 (d, j=3 Hz, 1H), 4.22 (t, j=6 Hz, 2H), 4.05 (s, 1H), 3.04 (t, j=6 Hz, 2H), 2.98 (q, j=6 Hz, 2H), 0.84 (t, j=6 Hz, 3H). MS (EI), m/z: [M+1] 352.20, [M−1] 350.44.

### Synthesis of Compound VII-36, 4-(1-(3-ethoxypropyl)-5-(4-hydroxyphenyl)-1H-pyrrol-2-yl)benzoic acid

Followed general pyrrole procedure 1, starting with 3-ethoxypropan-1-amine and Intermediate 7-1. Obtained 18 mg (29% yield) of the desired product. ¹H-NMR (300 MHz, DMSO-d₆, ppm): δ 9.57 (s, 1H), 7.99 (d, j=9 Hz, 2H), 7.60 (d, j=9 Hz, 2H), 7.30 (d, j=9 Hz, 2H), 6.85 (d, j=6 Hz, 2H), 6.33 (d, j=3 Hz, 1H), 6.14 (d, j=3 Hz, 1H), 4.21 (t, j=6 Hz, 2H), 3.03 (q, j=6 Hz, 2H), 2.84 (t, j=6 Hz, 2H), 1.33 (q, j=6 Hz, 2H), 0.82 (t, j=6 Hz, 3H); MS (EI), m/z: [M+1] 366.44, [M−1] 364.55.

### Synthesis of Compound VII-37, 4-(5-(4-hydroxyphenyl)-1-propyl-1H-pyrrol-2-yl)benzoic acid

Followed general pyrrole procedure 1, starting with propan-1-amine and Intermediate 7-1. Obtained 17 mg (32% yield) of the desired product. ¹H-NMR (300 MHz, CD₃OD, ppm): δ 9.64 (s, 1H), 8.00 (d, j=9 Hz, 2H), 7.59 (d, j=9 Hz, 2H), 7.27 (d, j=9 Hz, 2H), 6.87 (d, j=9 Hz, 2H), 6.32 (d, j=3 Hz, 1H), 6.12 (d, j=3 Hz, 1H), 4.04 (t, j=6 Hz, 2H), 1.11 (m, 2H), 0.38 (t, j=6 Hz, 3H). MS (EI), m/z: [M+1] 322.46. [M−1] 320.39.

### Synthetic Details for Compounds of Table 8

### Intermediate 8-1: 2-amino-5-hydroxy-N-methylbenzamide

### Step 1

To a solution of 2-amino-5-hydroxybenzoic acid (2.0 g, 1.31 mmol) and TEA (2.6 g, 26.2 mmol) in DCM (20 mL) was added (Boc)₂O (4.4 g, 15.7 mmol) at 0° C. The mixture was warmed up to room temperature and stirred overnight. The solvent was washed with water (50 mL×3), dried over Na₂SO₄ and removed by vacuo to give 2-((tert-butoxycarbonyl)amino)-5-hydroxybenzoic acid (2.5 g, 75.6%).

### Step 2

A mixture of the above product (2.0 g, 7.9 mmol), methane amine (2.6 g, 39.5 mmol), HATU (3.3 g, 8.7 mmol) and DIPEA (21.24 g, 17.4 mmol) in THF (20 mL) was stirred at room temperature for 24 h. An aqueous workup with EtOAc extraction was followed by column chromatography (PE:EA=50%:50%) to give tert-butyl (4-hydroxy-2-(methylcarbamoyl)phenyl)carbamate as a white solid (2.0 g, 95.1%).

### Step 3

A mixture of the above product (1.0 g, 3.7 mmol), TFA (3 mL) and DCM (20 mL) was stirred overnight. Then water (20 mL) was added, adjusted the PH to 8 with saturated NaHCO₃ solution, washed with water (30 mL), dried over Na₂SO₄, concentrated to give Intermediate 8-1 (560 mg, 89.7%). MS (ESI): m/z=167.0 [M+1]⁺.

### Intermediate 8-2: 3-chloro-7-methoxy-2-methylisoquinolin-1(2H)-one

### Step 1

To a solution of 1,3-dichloro-7-methoxyisoquinoline (synthesis described in US provisional 61/423,805) (238 mg, 1.04 mmol) in toluene (10 mL) was added tBuOK (140 mg, 1.25 mmol), the resulting mixture was stirred at 80° C. for 24 hours. The reaction mixture was concentrated under reduced pressure to afford a residue, which was dissolved in HCOOH (10 mL) and stirred at 25° C. for 16 hours. Aqueous workup with EtOAc extraction gave the crude product which was purified by silica gel column (PE/EtOAc=3/1) to give 3-chloro-7-methoxyisoquinolin-1-ol (200 mg, yield 91%) as a yellow solid.

### Step 2

To a solution of the above product (200 mg, 0.957 mmol) in DMF (5 mL) was added NaH (46 mg, 1.15 mmol, 60% in mineral oil) in portions at 0° C. After addition, the mixture was stirred for 15 minutes. Then CH₃I (270 mg, 1.91 mmol) was added and the mixture was stirred at 0° C. for 30 minutes. The reaction mixture was quenched with ice-water. Extraction with EtOAc, followed by purification by silica gel column (PE/EtOAc=10/1) gave 3-chloro-7-methoxy-2-methylisoquinolin-1(2H)-one (180 mg, yield 85%) as an off-white solid. ¹H NMR (CDCl₃ 300 MHz): δ 7.71 (d, J=2.7 Hz, 1H), 7.30 (d, J=8.7 Hz, 1H), 7.20 (dd, J=8.7 Hz, 2.7 Hz, 1H), 6.58 (s, 1H), 3.88 (s, 3H), 3.72 (s, 3H).

### Intermediate 8-3: 4-acetyl-6-methoxyisochroman-1,3-dione

### Step 1

A mixture of 4-methoxy-2-methylbenzoic acid (20 g, 120 mmol) and dimethyl carbonate (20 ml, 241 mmol) in THF (dry, 200 ml) was added dropwise to LDA (240 ml of 2M in heptane/THF/ethylbenzene) at −78° C. After addition, the mixture was stirred at room temperature for 5 h. The reaction was quenched with 160 mL of water and stirred overnight. The organic layer was separated, and the aqueous solution was acidified with concentrated HCl to pH 2 and extracted with ethyl acetate. Recrystallization from EtOAc (hot)/hexane afforded 2-(carboxymethyl)-4-methoxybenzoic acid as a white solid (16.0 g, 63.2%).

### Step 2

Pyridine (1.0 mL) was added slowly to a suspension of the above product (2 g, 9.5 mmol) in acetic anhydride (15 mL) at 0° C. After stirring for 16 h, ether (50 mL) was added. The resulting solid is collected and dried to give 4-acetyl-6-methoxyisochroman-1,3-dione as a yellow powder (2 g, 90.9%). MS (ESI): m/z=235.1 [M+1]⁺.

### Intermediate 8-4: 6-methoxy-1-oxo-1H-isochromene-4-carboxylic acid

A mixture of acetic anhydride (4.3 mL) and formic acid (12 mL) was heated at 60° C. for 2 h under N₂. The mixture was cooled to 0° C., then 2-(carboxymethyl)-4-methoxybenzoic acid (see step 1 of Intermediate 8-3) (1.0 g, 4.76 mmol) was added. After that, pyridine (0.6 mL) was added carefully. The resultant mixture was stirred at room temperature overnight. Et₂O (30 mL) was added, and the mixture was stirred for 1 h. The mixture was filtered and the precipitate was washed by Et₂O (5 mL) and dried to give 6-methoxy-1-oxo-1H-isochromene-4-carboxylic acid (1.0 g, 95.5%) as a solid. MS (ESI): m/z=221.0, 220.0 [M+1, M]⁺. ¹H-NMR (DMSO-d₆ 500 MHz TMS): 8.82 (s, 1H), 8.10 (s, 1H), 7.94 (d, J=8.5 Hz, 1H), 6.87 (d, J=6.5 Hz, 1H), 3.84 (s, 3H) ppm. MS (ESI): m/z=221.0, 220.0 [M+1, M]⁺.

### Intermediate 8-5: 6-methoxy-4-(2,2,2-trifluoroacetyl)isochroman-1,3-dione

To a stifling solution of 2-(carboxymethyl)-4-methoxybenzoic acid (see step 1 of Intermediate 8-3) (5.0 g, 23.8 mmol) in TFAA (20 mL) was added slowly pyridine (3.0 mL, 38.1 mmol) at 0° C.; the mixture was stirred at room temperature overnight. Et₂O (50 mL) was added, stirred for 1 h, filtered and the precipitate was purified by column chromatography (PE:EA=75%:25%) to give a white solid (3.0 g, 43.8%). MS (ESI): m/z=289 [M+1]⁺.

### Intermediate 8-6: 6-methoxy-3-methyl-1H-isochromen-1-one

To a solution of 2-(carboxymethyl)-4-methoxybenzoic acid (see step 1 of Intermediate 8-3) (260 mg, 1.25 mmol) and Ac₂O (255 mg, 2.5 mmol) in EtOAc (5 mL) was added five drops of conc. HCl. The reaction mixture was heated at 80° C. for two days. The mixture was partitioned with EtOAc (10 mL) and sat. NaHCO₃ (10 mL). The aqueous phase was extracted with EA (20 mL×2). The combined organic phase was washed with brine (30 mL), dried over Na₂SO₄, concentrated to afford Intermediate 8-6 as a yellow powder (180 mg, 75.9%). MS (ESI): m/z=191.1 [M+1]⁺.

### Synthesis of Compound VIII-1, 4-(7-hydroxy-2-(methylthio)-4-oxoquinazolin-3(4H)-yl)benzoic acid

### Step 1

A solution of methyl 2-amino-4-methoxybenzoate (250 mg, 1.38 mmol) and ethyl 4-isothiocyanatobenzoate (286 mg, 1.38 mmol) in toluene (3 mL) was heated to reflux for 20 h under nitrogen. The mixture was diluted with PE (15 mL) and cooled to 0° C. The precipitate was filtered and dried in vacuo to afford ethyl 4-(2-mercapto-7-methoxy-4-oxoquinazolin-3(4H)-yl)benzoate (310 mg, 63%) as a yellow solid. MS (ESI): m/z 357.1 [M+1]⁺.

### Step 2

To a suspension of the above product (310 mg, 0.87 mmol) and potassium carbonate (360 mg, 2.63 mmol) in acetone (5 mL) was added dropwise iodomethane (618 mg, 4.35 mmol) at room temperature. The resulting mixture was stirred for 3 h, filtered and washed with acetone (10 mL). The filtrate was evaporated and the residue was recrystallized from PE:EA=10:1 (5 mL) to afford ethyl 4-(7-methoxy-2-(methylthio)-4-oxoquinazolin-3(4H)-yl)benzoate (290 mg, 90%) as a white solid.

### Step 3

To a solution of the above product (140 mg, 0.38 mmol) in DCM (0.5 mL) was added BBr₃ (0.4 mL, 0.77 mmol) and stirred at room temperature overnight. To the mixture was added dropwise water (5 drops). The volatiles were removed in vacuo and the residue was purified by prep-HPLC to afford Compound VIII-1 (33 mg, 27%) as a white solid. ¹H NMR (DMSO-d₆ 500 MHz): δ 13.6 (s, 1H), 10.66 (s, 1H), 8.08 (d, J=8 Hz, 2H), 7.92 (d, J=8 Hz, 1H), 7.57 (d, J=8 Hz, 1H), 6.91 (m, 2H), 2.47 (s, 3H). MS (ESI): m/z 329.0 [M+1]⁺.

### Synthesis of Compound VIII-2, 4-(7-hydroxy-4-oxo-2-(trifluoromethyl)quinazolin-3(4H)-yl)benzoic acid

### Step 1

2-Amino-4-methoxybenzoic acid (0.82 g) was suspended in DCM and treated with 1.9 g of trifluoroacetic anhydride over 2 hours. Concentration under reduced pressure gave the desired product, 7-methoxy-2-(trifluoromethyl)-4H-benzo[d][1,3]oxazin-4-one (1.18 g) as a tan solid.

### Step 2

To a solution of 7-methoxy-2-(trifluoromethyl)-4H-benzo[d][1,3]oxazin-4-one (600 mg) in HOAc (10 mL) was added 1.1 g of methyl 4-aminobenzoate. The resultant solution was heated at reflux over 15 h. After dilution with EtOAc (80 mL), the mixture was washed with water, sat. NaHCO₃ and brine, and then dried over anhydrous Na₂SO₄. Removal of the solvents and recrystallization from EtOAc/Hexane afforded the desired product, methyl 4-(7-methoxy-4-oxo-2-(trifluoromethyl)quinazolin-3(4H)-yl)benzoate (320 mg) as tan solids.

### Step 3

The above product (156 mg) was treated with 5 mL of 1N BBr₃ in DCM overnight. After removal of solvents, the crude product was washed with water and dried under vacuum. The desired product (108 mg), 4-(7-hydroxy-4-oxo-2-(trifluoromethyl)quinazolin-3(4H)-yl)benzoic acid was isolated after rinsing with Et₂O. ¹H NMR (DMSO-d₆ 300 MHz TMS): δ 10.96 (1H, br, s), 8.09-8.04 (3H, m), 7.66 (2H, d, J=9 Hz), 7.18 (1H, dd, J=9 and 3 Hz), 7.13 (1H, d, J=3 Hz) ppm; MS (ESI): m/z 351, [M+H⁺].

### Synthesis of Compound VIII-3, 4-(6-hydroxy-1-oxoisoquinolin-2(1H)-yl)benzoic acid

### Step 1

Methyl 4-iodobenzoate (1.17 g) was mixed with 6-methoxyisoquinolin-1(2H)-one (0.5 g), CuI (108 mg), 8-hydroxyquinoline (81 mg) and K₂CO₃ (0.58 g) in DMSO (8 mL). The resultant mixture was heated at 150° C. overnight. After the reaction was quenched with water, the mixture was extracted with EtOAc (80 mL). Removal of solvents and purification with column chromatography afforded the desired product—methyl 4-(6-methoxy-1-oxoisoquinolin-2(1H)-yl)benzoate (40 mg) as brown solids. [M+H⁺]: 310.

### Step 2

Product from above (40 mg) was dissolved and treated with BBr3 (0.1 mL). The reaction was stirred overnight and quenched with water. After extraction with EtOAc, the solvents were removed in vacuo to give brown solids, which was trituated with DCM to give the final product-4-(6-hydroxy-1-oxoisoquinolin-2(1H)-yl)benzoic acid (6 mg) as brown solids. ¹H NMR (DMSO-d₆ 300 MHz TMS): δ 13.1 (1H, br s), 10.44 (1H, s), 8.12-8.05 (3H, m), 7.60 (2H, d, J=9 Hz), 7.41 (1H, d, J=6 Hz), 7.01-6.96 (2H, m), 6.60 (1H, d, J=6 Hz) ppm. MS (ESI): m/z=282 [M+1]⁺.

### Synthesis of Compound VIII-4, 4-(6-hydroxy-1-oxo-3,4-dihydroisoquinolin-2(1H)-yl)benzoic acid

### Step 1

6-methoxy-3,4-dihydroisoquinolin-1(2H)-one (1.13 mmol, 200 mg), methyl 4-iodobenzoate (2.25 mmol, 592 mg), CuI (20 mol %, 43 mg), and K₂CO3 (1.13 mmol, 156 mg) were combined and 5 mL of DMF was added. The mixture was heated to 150° C. for 18 hours, after which 20 mol % of 8-hydroxyquinoline and an additional 20 mol % of CuI was added and stirred for an additional 1.5 hours. The crude mixture was then filtered through celite and the filtrate was rinsed with MeOH. The mother liquor was concentrated in vacuo and then suspended in H₂O (25 mL). The organics were extracted with EtOAc, concentrated in vacuo, and purified via silica gel chromatography with a gradient of 0 to 50% EtOAc in Hexanes to afford 230 mg of methyl 4-(6-methoxy-1-oxo-3,4-dihydroisoquinolin-2(1H)-yl)benzoate.

### Step 2

The above product (0.739 mmol, 230 mg) was dissolved in 4 mL of DCM and BBr₃ (0.42 mL) was added and stirred for 2 days. The crude material was concentrated in vacuo, suspended in H₂O (10 mL), and filtered to give 199 mg of crude material. This was then dissolved in MeOH and stirred over night at 40° C. after addition of 4N HCl in Dioxane (0.2 mL). The crude ester mixture was cooled and filtered. The filtrate was triturated with EtOAc to afford 40 mg of ester, which was taken up in 3 mL of 2:1 H₂O:THF. 6.4 mg of LiOH was added and stirred at room temperature for 2 hours. The solution was acidified to pH=4 and the resulting solid was filtered to afford 17 mg of 4-(6-hydroxy-1-oxo-3,4-dihydroisoquinolin-2(1H)-yl)benzoic acid. ¹H NMR (DMSO-d₆ 300 MHz TMS): δ 7.97 (d, 2H), 7.79 (d, 1H), 7.53 (d, 2H), 6.78 (dd, 1H), 6.70 (d, 1H), 3.99 (t, 2H), 3.06 (t, 2H). MS (ESI): m/z=284.10 [M+1]+.

### Synthesis of Compound VIII-5, 2-(4-(1H-tetrazol-5-yl)phenyl)benzo[d]thiazol-6-ol

### Step 1

2-chloro-6-methoxybenzo[d]thiazole (96 mg, 0.48 mmol), 4-(1H-tetrazol-5-yl)phenylboronic acid (91.2 mg, 0.48 mmol), K₂CO₃ (200 mg), and PdCl₂(dppf) (18 mg) were taken up in 2 mL of a 3:1 solution of DEGME:H₂O under an argon atmosphere and degassed ×3. The solution was stirred for 1.5 hours in a microwave reactor at 150° C. The crude solution was filtered through celite and the filtrate washed with 1N NaOH (15 mL). The basic aqueous was back extracted with EtOAc (25 mL) and then acidified to pH=4. The desired was then extracted from the aqueous solution with EtOAc (25 mL), and the organics were washed with brine, dried over sodium sulfate, and concentrated in vacuo to afford 190 mg of crude 2-(4-(1H-tetrazol-5-yl)phenyl)-6-methoxybenzo[d]thiazole.

### Step 2

2-(4-(1H-tetrazol-5-yl)phenyl)-6-methoxybenzo[d]thiazole (190 mg, 0.61 mmol) was dissolved in 2 mL of NMP and Na2S (71 mg, 0.915 mmol) was added. The solution was heated to 140° C. in a microwave reactor for 4.5 hours. The crude was concentrated in vacuo and then dissolved in 0.5N NaOH (10 mL). This aqueous solution was acidified to pH=5 with 1N HCl and the resulting solid was filtered and washed with H₂O to yield 22.5 mg of 2-(4-(1H-tetrazol-5-yl)phenyl)benzo[d]thiazol-6-ol. ¹H NMR (DMSO-d₆ 300 MHz TMS): δ 10.00 (s, 1H), 8.26 (q, 4H, J=9 Hz, 3 Hz), 7.92 (d, 1H, J=6 Hz), 7.47 (d, 1H, J=3 Hz), 7.06 (dd, 1H, J=9 Hz, 3 Hz). MS (ESI): m/z=295.98 [M+1]+.

### Synthesis of Compound VIII-6, 4-(6-hydroxy-3,4-dihydroisoquinolin-2(1H)-yl)benzoic acid

### Step 1

A mixture of 6-methoxy-1,2,3,4-tetrahydroisoquinoline hydrochloride (500 mg, 2.50 mmol), 4-methoxycarbonylphenylboronic acid (1.35 g, 7.50 mmol), Cu(OAc)₂ (450 mg, 2.50 mmol), pyridine (990 mg, 12.5 mmol) and 4A MS in anhydrous CH₂Cl₂ (10 mL) was stirred at 30° C. for 2 days. The mixture was diluted with CH₂Cl₂/MeOH (v/v=10/1, 100 mL) and filtered off, the filtrate was washed with H₂O (40 mL), dried over Na₂SO₄, filtered and concentrated. Purification by silica gel column (PE/EtOAc=20/1) gave methyl 4-(6-methoxy-3,4-dihydroisoquinolin-2(1H)-yl)benzoate (140 mg, yield 19%) as a yellow solid.

### Step 2

To a solution of the above product (110 mg, 0.370 mmol) in THF (8 mL) and MeOH (4 mL) was added aqueous LiOH (1 M, 8 mL). The mixture was stirred at 30° C. for 15 hours. The mixture was acidified by 2N HCl to pH 6-7 and the aqueous layer was extracted with EtOAc/MeOH (v/v=10/1, 30 mL×3), the combined organic layer was washed with brine (30 mL), dried over Na₂SO₄, filtered and concentrated. Purification by silica gel column (PE/EtOAc=1/1) gave 4-(6-methoxy-3,4-dihydroisoquinolin-2(1H)-yl)benzoic acid (100 mg, yield 96%) as a yellow solid.

### Step 3

A mixture of the above product (100 mg, 0.350 mmol) and AlCl₃ (400 mg, 3.00 mmol) in anhydrous dichloroethane (5 mL) was refluxed for 2 days. The reaction was quenched with ice water, the aqueous layer was extracted with CH₂Cl₂/MeOH (v/v=10/1, 30 mL×3), the combined organic layer was washed with brine (30 mL), dried over Na₂SO₄, filtered and concentrated. Purification by prep-HPLC (0.1% TFA as additive) gave Compound VIII-6 (17 mg, yield 18%) as a yellow solid. ¹H NMR (MeOD 400 MHz): δ 7.92 (d, J=8.8 Hz, 2H), 7.05 (d, J=8.4 Hz, 1H), 7.00 (d, J=9.2 Hz, 2H), 6.68-6.65 (m, 2H), 4.46 (s, 2H), 3.65 (t, J=5.6 Hz, 2H), 2.95 (t, J=6.0 Hz, 2H). MS (ESI): m/z 270.1 [M+H]⁺.

### Synthesis of Compound VIII-7, 4-(6-hydroxybenzo[d]thiazol-2-yl)benzoic acid

### Step 1

2-chloro-6-methoxybenzo[d]thiazole (100 mg, 0.5 mmol), 4-(methoxycarbonyl)phenylboronic acid (90 mg, 0.5 mmol), K₂CO₃ (90 mg), and Pd(PPh₃)₄ (28 mg, 5 mol %) were suspended in a 1:1 mixture of Dioxane: H₂O (5 mL). The solution was degassed ×3 under an Argon atmosphere and heated to 100° C. for 4 hours in a microwave reactor. The solution was diluted with H₂O (10 mL) and filtered. The filtered solids were washed with EtOAc (15 mL) and dried in vacuo to yield 65 mg of methyl 4-(6-methoxybenzo[d]thiazol-2-yl)benzoate.

### Step 2

Methyl 4-(6-methoxybenzo[d]thiazol-2-yl)benzoate (65 mg) was dissolved in 10 mL of DCM and excess BBr₃ was added. The solution was stirred at room temperature over night. It was then concentrated in vacuo and the crude solid was suspended in H₂O (10 mL) and stirred for 4 hours. The solids were then filtered and dried in vacuo to yield 56 mg of 4-(6-hydroxybenzo[d]thiazol-2-yl)benzoic acid. ¹H NMR (CD₃OD 300 MHz TMS): δ 8.14 (m, 4H), 7.89 (dd, 1H, J=0.6, 8.4 Hz), 7.37 (dd, 1H, J=0.3, 2.1 Hz), 7.07 (dd, 1H, J=2.4, 6.3 Hz). MS (ESI): m/z=272.48 [M+1]⁺.

### Synthesis of Compound VIII-8, 3-fluoro-4-(6-hydroxy-4-oxo-1,4-dihydroquinolin-2-yl)benzoic acid

To a solution of 3-fluoro-4-(4-fluoro-6-hydroxyquinolin-2-yl)benzoic acid (Compound I-40, see US provisional 61/423,805) (100 mg, 0.332 mmol) in THF/H₂O (10 mL/10 mL) was added TFA (0.1 mL). The mixture was stirred at 30° C. for 2 days. The aqueous layer was neutralized with sat. NaHCO₃ to pH 6. The aqueous layer was extracted with EtOAc/MeOH (v/v=10/1, 30 mL×3), the combined organic layer washed with brine (30 mL), dried over Na₂SO₄, filtered and concentrated. Purification by prep-HPLC (0.1% TFA as additive) gave Compound VIII-8 (30 mg, 30%) as a yellow solid. ¹H NMR (DMSO 400 MHz TMS): δ 13.50 (brs, 1H), 11.90 (brs, 1H), 9.80 (brs, 1H), 7.95-7.75 (m, 3H), 7.55 (d, J=6.0 Hz, 1H), 7.40 (s, 1H), 7.20 (d, J=8.4 Hz, 1H), 6.10 (s, 1H). MS (ESI): m/z 299.9 [M+1]⁺.

### Synthesis of Compound VIII-9, 3-chloro-4-(6-hydroxy-4-oxo-1,4-dihydroquinolin-2-yl)benzoic acid

Followed procedure described for Compound VIII-9, starting from 3-chloro-4-(4-fluoro-6-hydroxyquinolin-2-yl)benzoic acid (Compound I-37, see US provisional 61/423,805) to give Compound VIII-9. ¹H NMR (DMSO-d₆ 400 MHz TMS): δ 11.00 (brs, 1H), 8.10 (d, J=1.6 Hz, 1H), 8.02 (dd, J=8.0, 1.6 Hz, 1H), 7.80 (d, J=8.0 Hz, 1H), 7.61 (d, J=8.8 Hz, 1H), 7.45 (d, J=2.8 Hz, 1H), 7.30 (dd, J=8.8, 2.4 Hz, 1H), 6.30 (s, 1H). MS (ESI): m/z 315.9 [M+H]⁺.

### Synthesis of Compound VIII-10, 4-(6-hydroxy-3-methyl-4-oxo-3,4-dihydroquinazolin-2-yl)benzoic acid

### Step 1

To a solution of 2-amino-5-hydroxy-N-methylbenzamide (Intermediate 8-1) (350 mg, 2.11 mmol) in DMSO-D₆ (3 mL) was added methyl 4-formylbenzoate (346 mg, 2.11 mmol) and NaHSO₃ (329 mg, 3.16 mmol). The mixture was heated at 150° C. for 2 h and cooled to room temperature then poured into water, filtered and washed with water, dried to give methyl 4-(6-hydroxy-3-methyl-4-oxo-3,4-dihydroquinazolin-2-yl)benzoate as a white solid (486 mg, 74.4%). MS (ESI): m/z=311.0 [M+1]⁺.

### Step 2

A mixture of the above product (100 mg, 0.32 mmol), NaOH (39 mg, 0.97 mmol) and THF/H₂O (4/1 mL) was stirred at room temperature for 5 h. The solvent was removed by vacuo, the residue was purified by HPLC to afford a white solid (41 mg, 42.9%). ¹H-NMR (DMSO-d⁶ 500 MHz TMS): 10.18 (s, 1H), 8.08˜8.10 (d, J=8.0 Hz, 2H), 7.80˜7.82 (d, J=8.5 Hz, 2H), 7.56˜7.58 (d, J=8.5 Hz, 1H), 7.47˜7.48 (d, J=2.5 Hz, 1H), 7.29˜7.31 (dd, J=3.0, 8.5 Hz, 1H), 3.91 (s, 3H), 3.34˜3.35 (d, J=6.0 Hz, 3H). MS (ESI): m/z=297.0 [M+1]⁺.

### Synthesis of Compound VIII-11, 4-(7-hydroxy-2-methyl-1-oxo-1,2-dihydroisoquinolin-3-yl)benzoic acid

### Step 1

Followed general Suzuiki Coupling method, starting from 3-chloro-7-methoxy-2-methylisoquinolin-1(2H)-one (Intermediate 8-2) and 4-carboxyphenylboronic acid, using Pd(dppf)Cl₂ (2%) as catalyst and DEGME/H₂O as solvent. The reaction was stirred at 120° C. for 2 hours. The desired 4-(7-methoxy-2-methyl-1-oxo-1,2-dihydroisoquinolin-3-yl)benzoic acid was isolated after aqueous/EtOAc workup as a brown solid in 96% yield.

### Step 2

Followed the AlCl₃ deprotection procedure described for Compound VIII-6, step 3. Compound was isolated after prep-HPLC, then lyophilization to give Compound VIII-11 as an off-white solid (10% yield). ¹H NMR (MeOD 400 MHz): δ 8.14 (d, J=8.0 Hz, 2H), 7.67 (d, J=2.4 Hz, 1H), 7.60 (d, J=8.4 Hz, 2H), 7.53 (d, J=8.4 Hz, 1H), 7.23 (dd, J=8.8 Hz, 2.8 Hz, 1H), 6.62 (s, 1H), 3.43 (s, 3H). MS (ESI): m/z 295.7 [M+H]⁺.

### Synthesis of Compound VIII-12, (trans)-4-(6-hydroxy-3-methyl-1-oxoisoquinolin-2(1H)-yl)cyclohexanecarboxylic acid

### Step 1

A mixture of 4-acetyl-6-methoxyisochroman-1,3-dione (Intermediate 8-3) (1 g, 1.0 mmol) and (trans)-4-aminocyclohexanecarboxylic acid (733 mg, 5.13 mmol) in DMF (8 mL) was heated at 100° C. for 24 h. The mixture was partitioned with EtOAc (20 mL) and water (20 mL). The aqueous phase was extracted with EtOAc (20 mL×2), washed with brine (50 mL), dried over Na₂SO₄, concentrated and the residue was purified by combi-flash (PE:EA=50%:50%) to afford (trans)-4-(6-methoxy-3-methyl-1-oxoisoquinolin-2(1H)-yl)cyclohexanecarboxylic acid as brown solid (150 mg, 11.2%).

### Step 2

To a solution of the above product (150 mg, 0.48 mmol) in DCM (4 mL) was added BBr₃ (595 mg, 2.38 mmol), and was heated at 30° C. overnight. Water (20 mL) was added carefully, and the mixture was extracted with EA (20 mL×3). The combined organic phase was concentrated and purification by prep-HPLC gave VIII-12 as a white powder (55 mg, 38.5%). ¹H-NMR (MeOD-d⁴ 500 MHz TMS): 8.05 (d, J=9.0 Hz, 1H), 6.89 (dd, J=2.5 Hz, 8.5 Hz, 1H), 6.74 (d, J=2.5 Hz, 1H), 6.38 (s, 1H), 4.14 (t, J=11.5 Hz, 1H), 2.96˜2.88 (m, 2H), 2.47 (s, 4H), 2.14 (d, J=12.5 Hz, 2H), 1.81 (d, J=12.0 Hz, 2H), 1.56˜1.64 (m, 2H) ppm. MS (ESI): m/z=302.1 [M+1]⁺.

### Synthesis of Compound VIII-13, 4-(6-hydroxy-3-methyl-1-oxoisoquinolin-2(1H)-yl)benzoic acid

### Step 1

A mixture of 4-acetyl-6-methoxyisochroman-1,3-dione (Intermediate 8-3) (500 mg, 2.14 mmol) and methyl 4-aminobenzoate (387 mg, 2.56 mmol) in DMF (4 mL) was heated at 130° C. overnight. The mixture was acidified with 1N HCl to pH 5, extracted with EA (20 mL×3), washed with brine (30 mL), dried over Na₂SO₄, and concentrated. Purification by column chromatography (PE:EA=85%:15%) gave methyl 4-(6-methoxy-3-methyl-1-oxoisoquinolin-2(1H)-yl)benzoate as yellow oil (50 mg, 7.2%). MS (ESI): m/z=324.2 [M+1]⁺.

### Step 2

BBr₃ deprotection of the above product following the procedure described for step 2 of Compound VIII-12 gave Compound VIII-13 as a yellow powder (10.8 mg, 6.6%). ¹H-NMR (MeOD-d₄ 500 MHz TMS): 8.22 (d, J=7.0 Hz, 2H), 8.11 (d, J=9.0 Hz, 1H), 7.39 (d, J=8.0 Hz, 2H), 6.96 (dd, J=2.0 Hz, 9.0 Hz, 1H), 6.87 (d, J=2.0 Hz, 1H), 6.55 (s, 1H), 2.01 (s, 3H) ppm. MS (ESI): m/z=296.1 [M+1]⁺.

### Synthesis of Compound VIII-14, (trans)-4-(6-hydroxy-1-oxoisoquinolin-2(1H)-yl)cyclohexanecarboxylic acid

### Step 1

A mixture of 6-methoxy-1-oxo-1H-isochromene-4-carboxylic acid (Intermediate 4) (1.0 g, 4.55 mmol), (trans)-4-aminocyclohexanecarboxylic acid (780 mg, 5.45 mmol) and DMF (8 mL) was heated at 110° C. overnight. Then EtOAc (80 mL) and water (50 mL) were added, the organic layer was washed with water, dried (Na₂SO₄), concentrated and the residue was purified by column chromatography (PE:EA=55%:45%) to give a white solid (325 mg, 23.8%). MS (ESI): m/z=302 [M+1]⁺.

### Step 2

BBr3 deprotection of the above product following the procedure described for step 2 of Compound VIII-12 gave Compound VIII-14 as a white solid (120 mg, 38.7%). MS (ESI): m/z=288 [M+1]⁺. 1H-NMR (DMSO-d₆ 500 MHz TMS): 8.05 (d, J=8.5 Hz, 1H), 7.40 (d, J=7.5 Hz, 1H), 6.92 (dd, J=2.0 Hz, 8.5 Hz, 1H), 6.85 (d, J=2.0 Hz, 1H), 6.47 (d, J=7.5 Hz, 1H), 4.72˜4.79 (m, 1s), 2.28 (t, J=12.0 Hz, 1H), 2.04 (d, J=12.5 Hz, 2H), 1.74 (d, J=9.0 Hz, 4H), 1.50 (brs, 2H) ppm.

### Synthesis of Compound VIII-15, (trans)-4-(6-hydroxy-1-oxo-3-(trifluoromethyl)isoquinolin-2(1H)-yl)cyclohexanecarboxylic acid

### Step 1

Followed procedure described in Step 1 of Compound VIII-14 to give (trans)-4-(6-methoxy-1-oxo-3-(trifluoromethyl)isoquinolin-2(1H)-yl)cyclohexanecarboxylic acid, where the starting materials were 6-methoxy-4-(2,2,2-trifluoroacetyl)isochroman-1,3-dione (Intermediate 5) and (trans)-4-aminocyclohexanecarboxylic acid, and the reaction was heated at 140° C. overnight.

### Step 2

BBr₃ deprotection of the above product was accomplished following the procedure described for step 2 of Compound VIII-12 to give Compound VIII-15 as a white solid. ¹H-NMR (MEOD-d₄ 500 MHz TMS): 8.17 (d, J=9.0 Hz, 1H), 7.16 (s, 1H), 7.15 (dd, J=2.5 Hz, 9.0 Hz, 1H), 7.02 (d, J=2.5 Hz, 1H), 3.98˜4.04 (m, 1H), 2.90˜2.97 (m, 2H), 2.37 (t, J=13.0 Hz, 2H), 2.17 (d, J=11.0 Hz, 2H), 1.83 (d, J=11.0 Hz, 2H), 1.50˜1.58 (m, 2H) ppm. MS (ESI): m/z=356 [M+1]⁺.

### Synthesis of Compound VIII-16, (trans)-4-(6-hydroxy-1-oxo-3,4-dihydroisoquinolin-2(1H)-yl)cyclohexanecarboxylic acid

Compound VIII-14 (80 mg, 0.279 mmol), Pd(OH)₂ (30 mg) and EtOH (20 mL) were added to a 100 mL pressure container, and the system was filled with hydrogen until the pressure was 5 atm. The mixture was heated at 80° C. for 4 h, then cooled to room temperature. The resultant mixture was filtered and the filtrate was concentrated to give a white solid (45 mg, 55.9%). ¹H-NMR (MeOD-d₄ 500 MHz TMS): 7.79 (d, J=8.5 Hz, 1H), 6.73 (dd, J=1.5 Hz, 8.5 Hz, 1H), 6.63 (d, J=3.0 Hz, 1H), 4.53˜4.58 (m, 1s), 3.49 (t, J=6.5 Hz, 2H), 2.88 (t, J=6.0 Hz, 2H), 2.27 (brs, 1H), 2.12 (d, J=13.5 Hz, 2H), 1.78 (d, J=12.5 Hz, 2H), 1.58˜1.72 (m, 4H) ppm. MS (ESI): m/z=290.0 [M+1]⁺.

### Synthesis of Compound VIII-17, (trans)-4-(6-hydroxy-3-methyl-1-oxo-3,4-dihydroisoquinolin-2(1H)-yl)cyclohexanecarboxylic acid

### Step 1

A mixture of 6-methoxy-3-methyl-1H-isochromen-1-one (Intermediate 8-6) (180 mg, 0.95 mmol) and (trans)-4-aminocyclohexanecarboxylic acid (203 mg, 1.42 mmol) in DMF (3 mL) was heated at 140° C.-160° C. for two days. The mixture was partitioned with EtOAc (20 mL) and water (15 mL). The precipitate was filtered and dried to afford (trans)-4-(6-methoxy-3-methyl-1-oxoisoquinolin-2(1H)-yl)cyclohexanecarboxylic acid as a white powder (100 mg, 33.6%). MS (ESI): m/z=316.1 [M+1]⁺

### Step 2

A mixture of the above product (100 mg, 0.32 mmol) and Pd(OH)₂ (30 mg) in EtOH (30 mL) was heated at 80° C. under hydrogen (H₂, 5 atm) for 4 h. Filtered and the filtrate was concentrated to afford (trans)-4-(6-methoxy-3-methyl-1-oxo-3,4-dihydroisoquinolin-2(1H)-yl)cyclohexanecarboxylic acid as an oil (90 mg, 90%). MS (ESI): m/z=318 [M+1]⁺.

### Step 3

BBr₃ deprotection of the above product was accomplished following the procedure described for step 2 of Compound VIII-12 to give Compound VIII-17 as a white solid. 1H-NMR (MeOD-d₄ 500 MHz TMS): 7.75 (d, J=8.5 Hz, 1H), 6.73 (dd, J=2.0 Hz, 8.5 Hz, 1H), 6.64 (d, J=1.5 Hz, 1H), 4.48˜4.42 (m, 1H), 4.02˜3.97 (m, 1H), 3.15˜3.19 (m, 1H), 2.65 (d, J=16.0 Hz, 1H), 2.34˜2.29 (m, 1H), 2.16˜2.11 (m, 2H), 1.93˜1.90 (m, 1H), 1.83˜1.74 (m, 3H), 1.65˜1.56 (m, 2H), 1.14 (d, J=6.0 Hz, 3H) ppm. MS (ESI): m/z=304.1 [M+1]⁺.

### Example 3

### GSNOR Assays

Various compounds were tested in vitro for their ability to inhibit GSNOR activity. IC₅₀ ranges were presented in Tables 1-8 in Example 1 for GSNOR inhibitor compounds of the invention. GSNOR activity is described as a range in the tables in the following manner: an IC₅₀ value <100 nM is designated the letter a, an IC₅₀ range of 100 nM-1 μM is designated the letter b, and an IC₅₀ range of 1 μM-10 μM is designated the letter c.

GSNOR expression and purification is described in Biochemistry 2000, 39, 10720-10729.

GSNOR Fermentation:

Pre-cultures were grown from stabs of a GSNOR glycerol stock in 2XYT media containing 100 ug/ml ampicillin after an overnight incubation at 37° C. Cells were then added to fresh 2XYT (4 L) containing ampicillin and grown to an OD (A₆₀₀) of 0.6-0.9 at 37° C. before induction. GSNOR expression was induced with 0.1% arabinose in an overnight incubation at 20° C.

GSNOR Purification:

E. coli cell paste was lysed by nitrogen cavitation and the clarified lysate purified by Ni affinity chromatography on an AKTA FPLC (Amersham Pharmacia). The column was eluted in 20 mM Tris pH 8.0/250 mM NaCl with a 0-500 mM imidazole gradient. Eluted GSNOR fractions containing the Smt-GSNOR fusion were digested overnight with Ulp-1 at 4° C. to remove the affinity tag then re-run on the Ni column under the same conditions. GSNOR was recovered in the flowthrough fraction and for crystallography is further purified by Q-Sepharose and Heparin flowthrough chromatography in 20 mM Tris pH 8.0, 1 mM DTT, 10 uM ZnSO₄.

GSNOR assay: GSNO and enzyme/NADH Solutions are made up fresh each day. The solutions are filtered and allowed to warm to room temperature. GSNO solution: 100 mM NaPO4 (pH 7.4), 0.480 mM GSNO. 396 μL of GSNO Solution is added to a cuvette followed by 8 μL of test compound in DMSO (or DMSO only for full reaction control) and mixed with the pipette tip. Compounds to be tested are made up at a stock concentration of 10 mM in 100% DMSO. 2 fold serial dilutions are done in 100% DMSO. 8 μL of each dilution are added to an assay so that the final concentration of DMSO in the assay is 1%. The concentrations of compounds tested range from 100 to 0.003 μM. Enzyme/NADH solution: 100 mM NaPO₄ (pH 7.4), 0.600 mM NADH, 1.0 μg/mL GSNO Reductase. 396 μL of the Enzyme/NADH solution is added to the cuvette to start the reaction. The cuvette is placed in the Cary 3E UV/Visible Spectrophotometer and the change in 340 nm absorbance/min at 25° C. is recorded for 3 minutes. The assays are done in triplicate for each compound concentration. IC₅₀'s for each compound are calculated using the standard curve analysis in the Enzyme Kinetics Module of SigmaPlot.

Final assay conditions: 100 mM NaPO₄, pH 7.4, 0.240 mM GSNO, 0.300 mM NADH, 0.5 μg/mL GSNO Reductase, and 1% DMSO. Final volume: 800 μL/cuvette.

### Example 4

### Crystallography

Crystals of S-nitrosoglutathione reductase (GSNOR) were grown in the presence of NAD+ by vapor diffusion against a crystallization condition of 19% (w/v) PEG-8000/100 mM potassium phosphate pH 7.0/100 μM zinc sulfate/1 m M DTT. These native GSNOR crystals were soaked in 100 μM GSNOR inhibitors to prepare crystals of GSNOR complexes with GSNOR inhibitors. The crystals were cryo-protected with a solution that contains crystallant solution with 25% ethylene glycol. X-ray diffraction data were collected on the GSNOR candidate complex crystals using synchrotron radiation at the BCDB beamline 5.0.1 at the Advanced Light Source (ALS; Berkeley, Calif.). The data were reduced with the HKL2000 package (Otwinowski, Z et al., W., Methods Enzymol., 276, 307-326, (1997)). The native structure of GSNOR (PDB ID 1M6H (Sanghani, P. C. et al., Biochemistry, 41, 10778-10786, (2002)) containing NAD+, zinc, potassium, and Tris was refined directly with REFMAC5 (Murshudov, G. N. et al., Acta Crystallogr., Sect. D: Biol. Crystallogr., 53, 240-255, (1997)) against the X-ray diffraction data. Structures that showed ligand density were refined in iterative cycles of real space density fitting with COOT (Emsley, P. et al., Acta Crystallogr., Sect. D: Biol. Crystallogr., 60, 2126-2132, (2004)) and reciprocal space refinement with REFMAC5.

### Example 5

### Efficacy of GSNORi in Experimental Asthma

Experimental Asthma Model:

A mouse model of ovalbumin (OVA)-induced asthma was used to screen GSNOR inhibitors for efficacy against methacholine (MCh)-induced bronchoconstriction/airway hyper-responsiveness. This is a widely used and well characterized model that presents with an acute, allergic asthma phenotype with similarities to human asthma. Efficacy of GSNOR inhibitors was assessed using a protocol in which GSNOR inhibitors were administered after OVA sensitization and airway challenge, and prior to challenge with MCh. Bronchoconstriction in response to challenge with increasing doses of MCh was assessed using whole body plethysmography (Pₑₙₕ; Buxco). The amount of eosinophil infiltrate into the bronchoaveolar lavage fluid (BALF) was also determined as a measure of lung inflammation. The effects of GSNOR inhibitors were compared to vehicles and to Combivent (inhaled; IH) as the positive control.

Materials and Method

Allergen Sensitization and Challenge Protocol

OVA (500 μg/ml) in PBS was mixed with equal volumes of 10% (w/v) aluminum potassium sulfate in distilled water and incubated for 60 min. at room temperature after adjustment to pH 6.5 using 10 N NaOH. After centrifugation at 750×g for 5 min, the OVA/alum pellet was resuspended to the original volume in distilled water. Mice received an intraperitoneal (IP) injection of 100 μg OVA (0.2 mL of 500 μg/mL in normal saline) complexed with alum on day 0. Mice were anesthetized by IP injection of a 0.2-mL mixture of ketamine and xylazine (0.44 and 6.3 mg/mL, respectively) in normal saline and were placed on a board in the supine position. Two hundred fifty micrograms (100 μl of a 2.5 mg/ml) of OVA (on day 8) and 125 μg (50 μl of 2.5 mg/ml) OVA (on days 15, 18, and 21) were placed on the back of the tongue of each animal.

Pulmonary Function Testing (Penh)

In vivo airway responsiveness to methacholine was measured 24 h after the last OVA challenge in conscious, freely moving, spontaneously breathing mice with whole body plethysmography using a Buxco chamber (Wilmington, N.C.). Mice were challenged with aerosolized saline or increasing doses of methacholine (5, 20, and 50 mg/mL) generated by an ultrasonic nebulizer for 2 min. The degree of bronchoconstriction was expressed as enhanced pause (Pₑₙₕ), a calculated dimensionless value, which correlates with the measurement of airway resistance, impedance, and intrapleural pressure in the same mouse. Pₑₙₕ readings were taken and averaged for 4 min. after each nebulization challenge. Pₑₙₕ is calculated as follows: Pₑₙₕ=[(Tₑ/Tᵣ−1)×(PEF/PIF)], where Tₑ is expiration time, Tᵣ is relaxation time, PEF is peak expiratory flow, and PIF is peak inspiratory flow×0.67 coefficient. The time for the box pressure to change from a maximum to a user-defined percentage of the maximum represents the relaxation time. The Tᵣ measurement begins at the maximum box pressure and ends at 40%.

Eosinophil Infiltrate in BALF

After measurement of airway hyper-reactivity, the mice were exsanguinated by cardiac puncture, and then BALF was collected from either both lungs or from the right lung after tying off the left lung at the mainstem bronchus. Total BALF cells were counted from a 0.05 mL aliquot, and the remaining fluid was centrifuged at 200×g for 10 min at 4° C. Cell pellets were resuspended in saline containing 10% BSA with smears made on glass slides. Eosinophils were stained for 5 min. with 0.05% aqueous eosin and 5% acetone in distilled water, rinsed with distilled water, and counterstained with 0.07% methylene blue. Alternatively, eosinophils and other leukocytes were stained with DiffQuik.

GSNOR Inhibitors and Controls

GSNOR inhibitors were reconstituted in phosphate buffered saline (PBS), pH 7.4, or 0.5% w/v carboxy methylcellulose at concentrations ranging from 0.00005 to 3 mg/mL. GSNOR inhibitors were administered to mice (10 mL/kg) as a single dose or multiple dose either intravenously (IV) or orally via gavage. Dosing was performed from 30 min. to 72 h prior to MCh challenge. Effects of GSNOR inhibitors were compared to vehicle dosed in the same manner.

Combivent was used as the positive control in all studies. Combivent (Boehringer Ingelheim) was administered to the lung using the inhaler device supplied with the product, but adapted for administration to mice, using a pipet tip. Combivent was administered 48 h, 24 h, and 1 h prior to MCh challenge. Each puff (or dose) of Combivent provides a dose of 18 μg ipatropium bromide (IpBr) and 103 μg albuterol sulfate or approximately 0.9 mg/kg IpBr and 5 mg/kg albuterol.

Statistical Analyses

Area under the curve values for Pₑₙₕ across baseline, saline, and increasing doses of MCh challenge were calculated using GraphPad Prism 5.0 (San Diego, Calif.) and expressed as a percent of the respective (IV or orally administered) vehicle control. Statistical differences among treatment groups and the respective vehicle control group within each study were calculated using one-way ANOVA, Dunnetts or Bonferroni post-hoc tests or t-test (JMP 8.0, SAS Institute, Cary, N.C. or Microsoft Excel). A p value of <0.05 among the treatment groups and the respective vehicle control group was considered significantly different.

Results

In the OVA model of asthma, Compound I-8 of Table 1 significantly (p<0.05) decreased eosinophil infiltration in BAL by 37% of vehicle control when given via three oral doses of 10 mg/kg at 48 h, 24 h, and 1 h prior to assessment.

In the OVA model of asthma, Compound III-2 of Table 3 significantly (p<0.05) decreased eosinophil infiltration in BAL by 44% of vehicle control when given via three oral doses of 10 mg/kg at 48 h, 24 h, and 1 h prior to assessment.

In the OVA model of asthma, Compound I-3 of Table 1 significantly (p<0.05) decreased eosinophil infiltration in BAL by 42% of vehicle control when given via three oral doses of 10 mg/kg at 48 h, 24 h, and 1 h prior to assessment.

In the OVA model of asthma, Compound I-27 significantly (p<0.05) decreased eosinophil infiltration in BAL by 23% of vehicle control when given via three oral doses of 10 mg/kg at 48 h, 24 h, and 1 h prior to assessment.

In the OVA model of asthma, the compound of Compound IV-25 decreased the AUC for Penh (p<0.05) and eosinophil infiltration into BALF by 43% and 42%, respectively, of vehicle control when given via a single oral dose of 10 mg/kg at 24 h prior to assessment. In another study, Compound IV-25 decreased eosinophil infiltration in BALF by 12% of vehicle control when given via three oral doses of 10 mg/kg at 48 hours, 24 hours, and 1 h prior to assessment.

In the OVA model of asthma, the compound of Compound IV-23 decreased the AUC for Penh (p<0.05) and eosinophil infiltration into BALF by 20% to 39% and 0% to 31%, respectively, of vehicle control when given via a single oral dose of 10 mg/kg at 24 h prior to assessment. Compound IV-23 significantly decreased the AUC for Penh by 39% of vehicle control when given via a single IV dose of 10 mg/kg at 24 h prior to assessment.

In the OVA model of asthma, the compound of Compound IV-31 significantly decreased the AUC for Penh and eosinophil infiltration into BALF by 18% and 82%, respectively, of vehicle control when given via a single oral dose of 10 mg/kg at 24 h prior to assessment.

In the OVA model of asthma, the compound of Compound IV-38 significantly (p<0.05) decreased eosinophil infiltration in BAL by 36% of vehicle control when given via three oral doses of 10 mg/kg at 48 hours, 24 hours, and 1 h prior to assessment.

### Example 6

### Mouse Pharmacokinetic (PK) Study

Experimental Model

The mouse was used to determine the pharmacokinetics of compounds of the invention. This species is widely used to assess the bioavailability of compounds by administering both oral (PO) and intravenous (IV) test articles. Efficacy of the compounds of the invention was compared by assessing plasma exposure in male BALB/c mice either via IV or PO administration at the times of peak activity.

Materials and Methods

IV Administration of Compounds of the Invention

Compounds of the invention were reconstituted in a phosphate buffered saline (PBS)/10% Solutol (HS 15) clear solution resulting in a concentration of 0.2 mg/mL and administered to mice (2 mg/kg) as a single IV dose. Animals were dosed via the lateral tail vein. Blood samples were collected at designated time points (0.083, 0.25, 0.5, 1, 2, 4, 8, 16, 24 hours) by cardiac puncture under isoflurane anesthesia (up to 1 mL blood per animal). The blood was collected into tubes containing Li-Heparin. The blood samples were kept on ice until centrifugation within approximately 30 minutes of collection. The plasma was transferred into labeled polypropylene tubes and frozen at −70° C. until analyzed by LC/MS/MS.

PO Administration of Compounds of the Invention

The compounds of the invention were reconstituted in 40% Propylene Glycol/40% Propylene Carbonate/20% of a 5% Sucrose clear solution resulting in a concentration of 2 mg/mL and administered to mice (10 mg/kg) as a single oral dose via gavage. Blood samples were collected at 0.25, 0.5, 1, 2, 4, 8, 12, 16, 20 and 24 hours post dose by cardiac puncture under isoflurane anesthesia. The blood was collected in tubes containing Li-Heparin. The blood samples were kept on ice until centrifugation within approximately 30 minutes of collection. The plasma was transferred into labeled polypropylene tubes and frozen at −70° C. until analyzed by LC/MS/MS.

LC/MS/MS Analysis

Plasma samples at each timepoint were analyzed using a LC-MS/MS with a lower limit of quantification (LLOQ) of 1 ng/mL. Plasma was analyzed to determine the amount of the compound of the invention in each sample and regression curves generated for each compounds of the invention in the relevant matrixes.

WinNonlin analysis was used for calculating PK parameters for both the IV and PO administrations:

PK parameters for IV portion—AUCₗₐₛₜ; AUC_{INF}; T1/2; Cl; Vss; Cₘₐₓ; MRT

PK parameters for PO portion—AUCₗₐₛₜ; AUC_{INF}; T1/2; Cₘₐₓ; Cl, MRT.

In addition to the above PK parameters, bioavailability (% F) was calculated.

Compounds I-3, I-4, I-8, I-13, I-19, I-27, and I-28 of Table 1, Compounds III-1 and III-2 of Table 3, and Compounds IV-10, IV-23, IV-25, IV-31, IV-38 of Table 4, were tested and all had an oral bioavailability of greater than 9%. Compounds I-3, I-8, I-13, I-27, and III-1 had an oral bioavailability of greater than 45%.

### Example 7

### Efficacy of GSNOR Inhibitors in Experimental Inflammatory Bowel Disease (IBD)

Overview of the Models:

Acute and chronic models of dextran sodium sulfate (DSS)-induced IBD in mice were used to explore efficacy of GSNORi against this disease. Acute and chronic DSS-induced IBD are widely used and well characterized models that induce pathological changes in the colon similar to those observed in the human disease. In these models and in human disease, epithelial cells within the crypts of the colon are disrupted, leading to dysfunction of the epithelial barrier and the ensuing tissue inflammation, edema, and ulceration. GSNORi therapy may benefit IBD by restoring s-nitrosoglutathione (GSNO) levels, and thus prevent or reverse the epithelial barrier dysfunction.

Acute Prophylactic Model:

Experimental IBD was induced by administration of DSS in the drinking water of male C57Bl/6 mice (N=8 to 10 mice per group) for 6 consecutive days. GSNORi was dosed orally at doses of 0.1 to 10 mg/kg/day for 10 days starting two days prior to and continuing two days post DSS exposure. Two days post DSS exposure, the effect of GSNORi was assessed in a blinded fashion via endoscopy and histopathology using a five point scale ranging from a score=0 (normal tissue) through a score=4 (ulcerative tissue damage and marked pathological changes). Levels of circulating cytokines involved in inflammatory pathways were also assessed. The effect of GSNORi was compared to vehicle treated controls. The corticosteroid, prednisolone, was used as the positive control in this study and was administered daily at 3 mg/kg/day via oral dosing. Naïve mice (N=5) were also assessed as a normal tissue control.

Chronic Treatment Model:

Experimental IBD was induced by administration of DSS in the drinking water of male C57Bl/6 mice (N=10 to 12 mice per group) for 6 consecutive days. GSNORi was dosed orally at doses of 10 mg/kg/day for 14 days starting one day after cessation of DSS exposure. Efficacy of GSNORi was assessed in a blinded fashion via endoscopy after 7 days and 14 days of GSNORi dosing and via histopathology after 14 days of GSNORi dosing using a five point scale ranging from a score=0 (normal tissue) through a score=4 (ulcerative tissue damage and marked pathological changes). Levels of circulating cytokines involved in inflammatory pathways were also assessed. The effect of GSNORi was compared to vehicle treated controls. The corticosteroid, prednisolone, was used as the positive control in this study and was administered daily at 3 mg/kg/day via oral dosing. Naïve mice (N=5) were also assessed as a normal tissue control.

Results:

Compound I-3 attenuated colon injury and lowered levels of cytokines involved in inflammatory responses in a mouse model of acute DSS-induced IBD. The percent of mice presenting with severe colon injury scores via endoscopy and histopathology assessments was significantly (p<0.05) decreased by 38% to 88% of vehicle control after oral treatment with Compound I-3 at 0.1, 1, or 10 mg/kg/day for 10 consecutive days using a prophylactic dosing regimen. Compound I-3 also restored circulating inflammatory cytokines towards levels observed in untreated naïve mice. These effects of Compound I-3 were comparable to or greater than those observed for prednisolone.

Compound I-8 attenuated colon injury in a mouse model of acute DSS-induced IBD. The percent of mice presenting with severe colon injury scores via endoscopy or histopathology assessments was decreased by 44% or 26%, respectively, of vehicle control after oral treatment with Compound I-8 at 10 mg/kg/day for 10 consecutive days using a prophylactic dosing regimen.

Compound I-19 attenuated colon injury in a mouse model of acute DSS-induced IBD. The percent of mice presenting with severe colon injury scores via endoscopy assessment was decreased by 31% of vehicle control after oral treatment with Compound I-19 at 10 mg/kg/day for 10 consecutive days using a prophylactic dosing regimen.

Compound I-13 attenuated colon injury in a mouse model of chronic DSS-induced IBD. The percent of mice presenting with severe colon injury scores via endoscopy or histopathology assessment was significantly (p<0.05) decreased by 52% or 53%, respectively, of vehicle control after oral treatment with Compound I-13 at 10 mg/kg/day for up to 14 consecutive days using a treatment dosing regimen.

Compound III-1 attenuated colon injury in mouse models of acute and chronic DSS-induced IBD. In the acute model, the percent of mice presenting with severe colon injury scores via endoscopy or histopathology assessment was decreased by 17% or 21%, respectively, of vehicle control after oral treatment with Compound III-1 at 1 mg/kg/day for 10 consecutive days using a prophylactic dosing regimen. Compound III-1 dosed orally at 10 mg/kg/day for 10 days, significantly (p<0.05) decreased the percent of mice presenting with severe endoscopy scores by 72% of vehicle control. In the chronic model, the percent of mice presenting with severe colon injury scores via endoscopy or histopathology assessment was decreased by 50% or 17%, respectively, of vehicle control after oral treatment with Compound III-1 at 10 mg/kg/day for up to 14 consecutive days using a treatment dosing regimen.

Compound III-2 attenuated colon injury in a mouse model of acute DSS-induced IBD. The percent of mice presenting with severe colon injury scores via endoscopy or histopathology assessments was decreased by 58% (p<0.05) or 26%, respectively, of vehicle control after oral treatment with Compound III-2 at 10 mg/kg/day for 10 consecutive days using a prophylactic dosing regimen.

Compound IV-25 attenuated colon injury in a mouse model of acute DSS-induced IBD. The percent of mice presenting with severe colon injury scores via endoscopy assessment was decreased by 38% or 25% of vehicle control after oral treatment with 0.1 or 1 mg/kg/day, respectively, of Compound IV-25 for 10 consecutive days using a prophylactic dosing regimen. The percent of mice presenting with severe colon injury scores via pathology assessment was decreased by 12% or 33% of vehicle control after oral treatment with 0.1 or 1 mg/kg/day, respectively, of Compound IV-25 for 10 days.

Compound IV-23 attenuated colon injury in a mouse model of acute DSS-induced IBD. The percent of mice presenting with severe colon injury scores via endoscopy or histopathology assessment was decreased by 75% or 17%, respectively, of vehicle control after oral treatment with 10 mg/kg/day of Compound IV-23 for 10 consecutive days using a prophylactic dosing regimen.

Compound IV-38 attenuated colon injury in a mouse model of acute DSS-induced IBD. The percent of mice presenting with severe colon injury scores via endoscopy or histopathology assessments was decreased by 58% or 15%, respectively, of vehicle control after oral treatment with the compound of Compound IV-38 at 10 mg/kg/day for 10 consecutive days using a prophylactic dosing regimen.

### Example 8

### Efficacy of GSNOR Inhibitors in Experimental Chronic Obstructive Pulmonary Disease (COPD)

Short Duration Cigarette Smoke COPD Models

The efficacy of GSNOR inhibitors was assessed in a mouse model of chronic obstructive pulmonary disease (COPD) induced by short duration (4 days or 11 days) of exposure to cigarette smoke. Infiltration of inflammatory cells into the bronchoalveolar lavage fluid (BALF) and BALF levels of chemokines involved in inflammation and tissue turnover/repair were measured to assess the influences of GSNOR inhibitors on some of the early events associated with the initiation and progression of COPD.

Overview of the Models:

Efficacy of GSNOR inhibitors against COPD was explored using acute (4 day) and subchronic (11 day) models of cigarette smoke-induced COPD in mice. Exposure of animals to cigarette smoke provides a model of COPD in which injury is induced by the same causative agent as in human disease and in which injury exhibits similarities to the human disease, including airway obstruction, airspace enlargement, and involvement of inflammatory responses in these pathologies. In animal models, changes in lung pathology are only evident after extended (several months) duration of exposure to cigarette smoke, thus making chronic models prohibitive as effective screening tools. More recently, models exploring inflammatory responses after short duration (2 weeks or less) of smoke exposure in mice have been utilized as tools for screening efficacy and mechanisms of action of novel therapeutics against COPD. The key roles of inflammation in the initiation and progression of COPD, make these short duration models relevant for initial tests of efficacy of novel therapeutics.

Acute (4 Day) Smoke Exposure Model:

Female C57Bl/6 mice (N=8 per group) were exposed to cigarette smoke using a whole body exposure chamber. Mice were exposed daily for 4 consecutive days to 4 cycles of smoke from 6 sequential cigarettes (Kentucky 3R4F without filter) with a 30 minute smoke free interval between cycles. GSNOR inhibitors were administered daily via oral dosing at 10 mg/kg/day for 7 days starting 2 days prior to smoke exposure and continuing 1 day post-exposure. The effects of GSNOR inhibitors were assessed by quantitating the numbers of total cells, leukocytes, and leukocytes differentials in the BALF via light microscopy and the levels of BALF chemokines via ELISA at approximately 24 h after the last smoke exposure. The effect of GSNOR inhibitors were compared to vehicle treated controls. The PDE4 inhibitor, roflumilast, was used as the positive control for the study. A group of naïve mice (N=8) was exposed to air and used as a negative control for the study.

Subchronic (11 Day) Smoke Exposure Model:

Female C57Bl/6 mice (N=10 per group) were exposed to cigarette smoke generated from Marlboro 100 cigarettes without filters. Exposure times were 25 min. on study day 1, 35 min. on study day 2, and 45 min. on study days 3 to 11. GSNOR inhibitors were administered one hour prior to smoke exposure on each day. GSNOR inhibitors were dosed orally at 1 to 10 mg/kg/day for 11 days. The effects of GSNOR inhibitors were assessed by quantitating the number of total cells, and leukocytes differentials in the BALF via light microscopy at 24 h after the last exposure. The effect of GSNOR inhibitors were compared to vehicle treated controls and expressed as percent inhibition of the cigarette smoke induced increases in BALF cell numbers. Roflumilast was used as the positive control for the study and was dosed at 5 mg/kg/day. A group of naïve mice (N=10) was exposed to air and dosed with vehicle as a negative control for the study.

Results:

Compound I-3 attenuated the smoke-induced changes in BALF cellular infiltrate and BALF inflammatory chemokines. Compound I-3 significantly (p<0.05) decreased total cells, leukocytes, macrophages, neutrophils, and eosinophils in BALF by 66%, 80%, 75%, 84%, and 95%, respectively, compared to vehicle treated controls when dosed orally at 10 mg/kg/day for 7 days in the acute 4 day smoke model. These effects of Compound I-3 were comparable to or greater than those observed for roflumilast. Compound I-3 also restored BALF chemokines towards levels observed in naïve mice. In the subchronic 11 day model, Compound I-3 inhibited the smoke-induced increase in total cells (p<0.05), macrophages, neutrophils (p<0.05), eosinophils, and lymphocytes (p<0.05) in BALF by 25%, 24%, 41%, 70%, and 49%, respectively, when dosed orally at 10 mg/kg/day for 11 days. When dosed orally at 5 mg/kg/day, Compound I-3 inhibited total cells, macrophages, neutrophils (p<0.05), and lymphocytes (p<0.05) in BALF by 22%, 23%, 29%, and 46%, respectively.

Compound I-8 significantly (p<0.05) inhibited the smoke-induced increase in total cells, macrophages, neutrophils, and lymphocytes in BAL by 35% to 48%, 24% to 43%, 41% to 70%, and 49 to 65%, respectively, when dosed orally at 1 to 10 mg/kg/day for 11 days in the subchronic 11 day model. There was no dose response of effect with 1, 5, or 10 mg/kg/day. The effects of Compound I-8 were comparable to those of roflumilast.

Compound III-1 inhibited the smoke-induced increase in total cells (p<0.05), macrophages (p<0.05), neutrophils (p<0.05), and lymphocytes in BAL by 40%, 40%, 49%, and 41%, respectively, when dosed orally at 10 mg/kg/day for 11 days in the subchronic 11 day model. These effects of Compound III-1 were comparable to those of roflumilast.

Compound I-13 significantly (p<0.05) inhibited the smoke-induced increase in total cells, macrophages, neutrophils, and lymphocytes in BAL by 56%, 53%, 67%, and 60%, respectively, when dosed orally at 1 mg/kg/day for 11 days in the subchronic 11 day model. These effects of Compound I-13 were comparable to those of roflumilast.

Compound I-27 significantly (p<0.05) inhibited the smoke-induced increase in total cells, macrophages, neutrophils, and lymphocytes in BAL by 44%, 41%, 64%, and 46%, respectively, when dosed orally at 1 mg/kg/day for 11 days in the subchronic 11 day model. These effects of Compound I-27 were comparable to those of roflumilast.

Compound IV-25 attenuated the smoke-induced changes in BALF cellular infiltrate and BALF inflammatory chemokines. Example 3 completely (100%) and significantly (p<0.05) inhibited the smoke-induced increase in total cells, leukocytes, macrophages, neutrophils, and eosinophils in BALF compared to vehicle treated controls when dosed orally at 10 mg/kg/day for 7 days in the acute 4 day smoke model. These effects of Compound IV-25 were comparable to or greater than those observed for roflumilast. Compound IV-25 also restored BALF chemokines towards levels observed in naïve mice. In the subchronic 11 day model, the compound of Compound IV-25 inhibited the smoke-induced increase in total cells (p<0.05), macrophages (p<0.05), neutrophils, eosinophils, and lymphocytes in BALF by 26%, 28%, 25%, 57%, and 24%, respectively, when dosed orally at 10 mg/kg/day for 11 days.

Compound IV-38 significantly (p<0.05) inhibited the smoke-induced increase in total cells, macrophages, neutrophils, and lymphocytes in BAL by 53%, 44%, 68%, and 62%, respectively, when dosed orally at 1 mg/kg/day for 11 days in the subchronic 11 day model. The effects of Compound IV-38 were comparable to those of roflumilast.

### Example 9

### An Exploratory Mouse Study of Acetaminophen Toxicity

S-nitrosoglutathione reductase (GSNOR) inhibition has been previously shown in our hands to ameliorate the negative manifestations of gastrointestinal injury in animal models. As an extension of these observations, the effects of S-nitrosoglutathione (GSNO) or GSNOR inhibitors (GSNORi) on acetaminophen (ACAP) induced liver toxicity can be evaluated in a mouse model of liver injury. Blood samples are collected for liver function assays and tissue samples are collected at the end of the study for histopathologic examination.

Materials and Methods

GSNORi, GSNO, acetaminophen (ACAP, Sigma) Vehicles (½ cc syringes for dosing), Isoflurane, 18 1 cc syringes w/26 g needles for blood collection, 90 serum separator tubes for clinical chemistry.

General Study Design:

Animals (5/group) are acclimated for at least 3 days prior to dosing. On Study Day 1, acetaminophen treatment (300 mg/kg PO) was given a single time=0 to fasted animals. Two hours later, GSNORi (10 mg/kg/dose) or GSNO (5 mg/kg/dose) are intravenously administered to the treatment groups. GSNORi or GSNO are given at 24 and 48 hours-post their initial administration to the treatment groups. Mice are observed for signs of clinical toxicity and blood was collected at 6, 24, and 72 hours post-ACAP administration for liver function tests: Alkaline phosphatase (ALK); Alanine aminotransferase (ALT); Aspartate aminotransferase (AST); Gamma glutamyltransferase (GGT) and Total bilirubin (TBILI). Livers are collected at 72 hours for histopathologic examination.

Study Outline

| | | | | Drug | | |
|---|---|---|---|---|---|---|
| Group | Treatment | Dose | | Concentration | | N |
| 1 | ACAP PO | 300 | mg/kg | 10 | ml/kg | 5 |
| 2 | Saline PO | 0 | mg/kg | 10 | ml/kg | 5 |
| 3 | GSNORi IV | 10 | mg/kg | 1 | mg/mL | 5 |
| 4 | GSNO IV | 5 | mg/kg | 1 | mg/mL | 5 |

| | | | | | |
|---|---|---|---|---|---|
| 5 | GSNORiIV + ACAP | 10 m/k/300 m/k | 1 | mg/mL | 5 |
| 6 | GSNO IV + ACAP | 5 m/k/300 m/k | 1 | mg/mL | 5 |

Study Calendar:

| | |
|---|---|
| Day −6 | Receive mice and place in regular cages |
| Day −1 | Fast animals overnight |
| Day 0 | Weigh, PO ACAP time = 0, time = 2 IV GSNO or GSNORi |
| | bleed all groups at at 6 hr post-ACAP |
| Day 1 | Weigh, bleed all groups for 24 hr LFTs, IV GSNO or GSNORi |
| Day 2 | Weigh, IV GSNO or GSNORi |
| Day 3 | Bleed for 72 hr LFTs, collect livers for weight and histology |

Vehicle, GSNO and GSNORi Preparation

The vehicle control article is Phosphate Buffered Saline (PBS) (not containing calcium, potassium, or magnesium) adjusted to pH 7.4. The vehicle components are weighed into a container on a tared balance and brought to volume with purified water (w/v). The 10× stock solution is mixed using a magnetic stirrer, as necessary. Thereafter, the 10× stock solution is diluted with deionized water at a ratio of 1:9 (v/v). GSNO is warmed to room temperature before preparation of solutions. Prior to use, the PBS solution is nitrogen sparged. 1 mg/mL GSNO solutions are kept cold (i.e., kept on an ice bath) and protected from light and used within 4 hours of preparation. GSNORi Preparation, the 1 mg/mL concentration is reconstituted in phosphate buffered saline (PBS), pH 7.4. GSNORi is administered to mice (10 mL/kg) as a single (IV) daily dose. Dosing is performed 2 hours post-ACAP administration and then 26 and 50 hours later. Effects of GSNO or GSNORi are compared to ACAP and saline vehicle dosed in the same manner.

Calculations:

Mean body weights, mean liver organ weights and clinical pathology endpoints (+/−SD) with T-test and ANOVA (alpha=0.05) comparison to vehicle control group. The clinical pathology data are prepared as mean values unless the data are not normally distributed, in which case, median values can be presented with the minimum and maximum value range.

### Example 10

### An Exploratory Study to Assess the Anti NASH Fibrotic Activity of GSNORi in STAM Mice

S-nitrosoglutathione reductase (GSNOR) inhibition has been previously shown in our hands to ameliorate the negative manifestations of gastrointestinal injury and ACAP injury in mouse models. As an extension of these observations, the effects of GSNOR inhibitors (GSNORi) ability to reverse fibrotic activity in nonalcoholic steatohepatitis (NASH)-induced liver disease is evaluated in STAM (signal transducing adaptor molecule) mice. In these mice sequential changes are seen from liver steatosis to fibrosis within two weeks and there are close similarities to human NASH histopathology.

Materials and Methods

GSNORi, Telmisartan, Vehicles (½ cc syringes for dosing), Isoflurane, 18 1 cc syringes w/26 g needles for blood collection, 90 serum separator tubes for clinical chemistry.

General Study Design:

Animals (6/group) are acclimated prior to beginning the Study. At 4 weeks of age the animals are put on a diet, group 1 (normal mice) receives a normal diet while groups 2-4 (STAM mice) are put on a high fat diet for the duration of the Study. At Study Week 7 the mice begin oral daily dosing with GSNORi and are sacrificed at Study Week 9. Mice are observed for signs of clinical toxicity and blood/tissue is collected for liver analyses: Plasma triglycerides (TG); Alanine aminotransferase (ALT); Aspartate aminotransferase (AST); Gene Expression: Timp-1, α-SMA, collagen 3, TNF-α and MCP-1 as well as histopathologic examination using HE staining for (NAFLD) activity score and Sirius-red staining (fibrosis area).

Study Outline

| | | | | Drug Con- | | |
|---|---|---|---|---|---|---|
| Group | Treatment | Diet | Dose | centration | | N |
| 1 | normal | ND | 0 mg/kg | 0 | ml/kg | 6 |
| 2 | STAM + vehicle | HFD | 10 mg/kg | 1 | mg/mL | 6 |
| 3 | STAM + GSNORi IV | HFD | 10 mg/kg | 1 | mg/mL | 6 |
| 4 | STAM + Telmisarten | HFD | 10 mg/kg | 1 | mg/mL | 6 |

ND: normal diet, HFD: high fat diet

Calculations:

Mean body weights, mean liver organ weights and clinical pathology endpoints (+/−SD) with T-test and ANOVA (alpha=0.05) comparison to vehicle control group. The clinical pathology data are prepared as mean values unless the data are not normally distributed, in which case, median values were presented with the minimum and maximum value range.

It will be apparent to those skilled in the art that various modifications and variations can be made in the methods and compositions of the present invention without departing from the spirit or scope of the invention.

What is claimed is:

1. What is claimed is:
A method of inhibiting GSNOR in a patient in need thereof by administering an effective amount of a compound of Formula 1 or a pharmaceutically acceptable salt thereof:
HO-Cy₁-linker-Cy₂-acidic moiety Formula 1
wherein
Cy₁ is selected from the group consisting of substituted and unsubstituted monocyclic aryl, substituted and unsubstituted bicyclic aryl, substituted and unsubstituted monocyclic heterocycle, substituted and unsubstituted bicyclic heterocycle, substituted and unsubstituted monocyclic heteroaryl, substituted and unsubstituted bicyclic heteroaryl, substituted and unsubstituted monocyclic cycloalkyl, and substituted and unsubstituted bicyclic cycloalkyl;
linker is selected from the group consisting of a direct bond, O, S, SO, SO₂, C═O, CR₅R₆, NR₇, substituted and unsubstituted (C₂-C₃) alkyl, substituted and unsubstituted (C₂-C₃) heteroalkyl, substituted and unsubstituted (C₂-C₃) alkene, substituted and unsubstituted 5 or 6 membered aryl, substituted and unsubstituted 5 or 6 membered heteroaryl, substituted and unsubstituted 3-6 membered cycloalkyl, and substituted and unsubstituted 3-6 membered saturated heterocyclyl; wherein R₅ and R₆ are independently selected from the group consisting of hydrogen, (C₁-C₆) alkyl, (C₁-C₆) heteroalkyl, halogen, (C₁-C₆) haloalkyl, cyano, and hydroxyl; R₇ is selected from the group consisting of hydrogen, (C₁-C₆)alkyl, (C₁-C₆) haloalkyl, and (C₁-C₆) heteroalkyl; substitutions for the (C₂-C₃) alkyl, (C₂-C₃) heteroalkyl, and (C₂-C₃) alkene are selected from the group consisting of ═O, halogen, (C₁-C₃) alkyl, (C₁-C₃) haloalkyl, (C₁-C₃) heteroalkyl, cyano, and hydroxyl, and wherein when the heteroalkyl group contains nitrogen or sulfur, the N and S atoms may be optionally oxidized; substitutions for aryl, heteroaryl, cycloalkyl and saturated heterocyclyl are selected from the group consisting of hydrogen, (C₁-C₈)alkyl, (C₁-C₈) haloalkyl, and (C₁-C₈)heteroalkyl; and
Cy₂ is selected from the group consisting of substituted and unsubstituted monocyclic aryl, substituted and unsubstituted monocyclic saturated heterocycle, substituted and unsubstituted monocyclic heteroaryl, and substituted and unsubstituted monocyclic cycloalkyl.

2. The method of claim 1 wherein,
linker is selected from the group consisting of a direct bond, O, S, SO, SO₂, C═O, CH₂, NH, NMe, substituted and unsubstituted (C₂-C₃) alkyl, substituted and unsubstituted (C₂-C₃) heteroalkyl, a 5 or 6 membered aryl, and a 5 or 6 membered heteroaryl group; wherein substitutions for the (C₂-C₃) alkyl and (C₂-C₃) heteroalkyl are selected from the group consisting of ═O, halogen, (C₁-C₃) alkyl, (C₁-C₃) haloalkyl, and hydroxyl, and wherein if the heteroalkyl group contains nitrogen or sulfur, they may be optionally oxidized.

3. The method of claim 1 wherein Cy₂-Acidic moiety is selected from the group consisting of 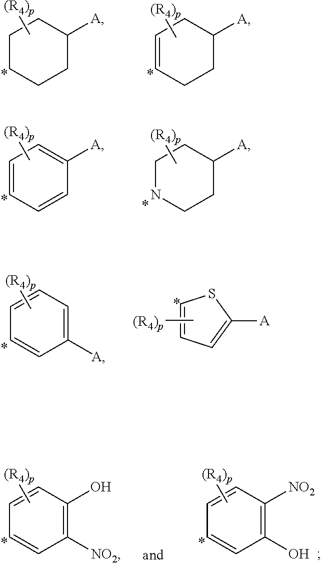
wherein
* represents the position on Cy₂ that is connected to the linker;
A is an acidic moiety, and is selected from the group consisting of 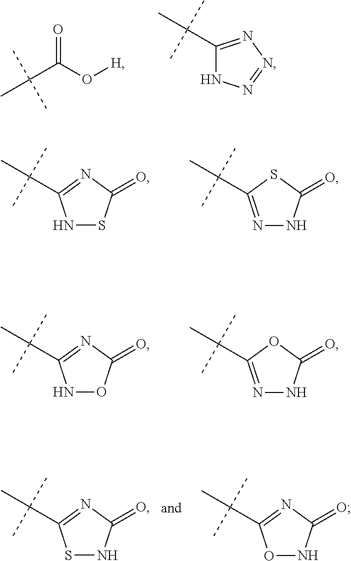
R₄ is selected from the group consisting of halogen, (C₁-C₆) alkyl, (C₁-C₆) haloalkyl, (C₁-C₆) alkoxy, cyano, and NR₈R_{8′} where R₈ and R_{8′} are independently selected from the group consisting of (C₁-C₃) alkyl, or R₈ when taken together with R_{8′} form a ring with 3 to 6 members;
and p is selected from the group consisting of 0, 1, 2, 3, and 4.

4. The method of claim 1 wherein,
linker is selected from a group consisting of a direct bond, O, S, SO, SO₂, C═O, CR₅R₆, NR₇, wherein R₅ and R₆ are independently selected from the group consisting of hydrogen, (C₁-C₆) alkyl, (C₁-C₆) heteroalkyl, halogen, (C₁-C₆) haloalkyl, cyano, and hydroxyl; R₇ is selected from the group consisting of hydrogen, (C₁-C₆)alkyl, (C₁-C₆) haloalkyl, and (C₁-C₆) heteroalkyl; and
Cy₁ is selected from the group consisting of substituted and unsubstituted bicyclic aryl, substituted and unsubstituted bicyclic heterocycle, substituted and unsubstituted bicyclic heteroaryl, and substituted and unsubstituted bicyclic cycloalkyl.

5. The method of claim 4 wherein HO-Cy₁ is selected from the group consisting of 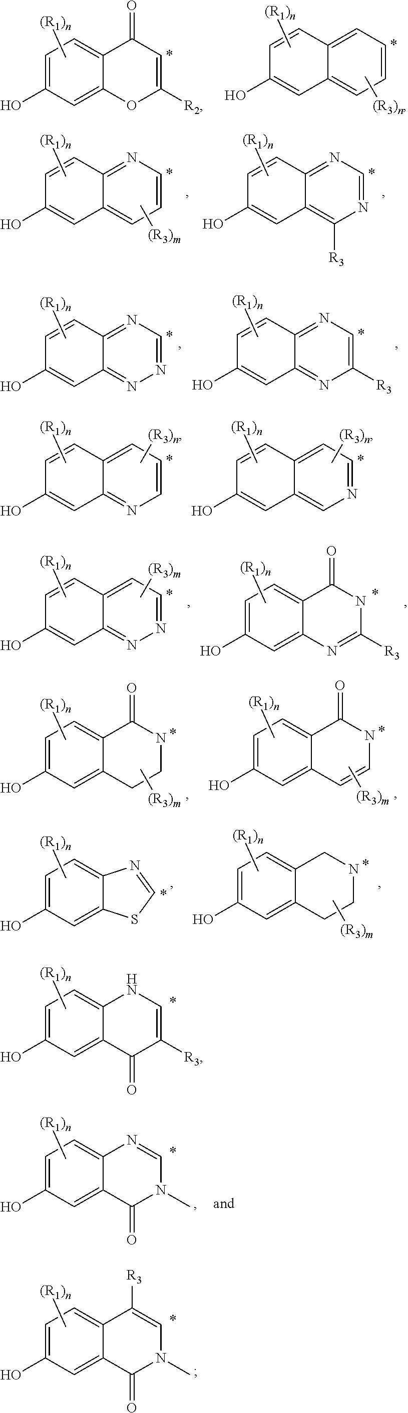
Wherein
* represents the position on Cy₁ that is connected to the linker;
R₁ is selected from the group consisting of halogen, methoxy, and cyano;
R₂ is selected from the group consisting of hydrogen, (C₁-C₆)alkyl, (C₃-C₇)cycloalkyl, (C₁-C₆)haloalkyl, unsubstituted aryl(C₁-C₄)alkyl, substituted aryl(C₁-C₆)alkyl, (C₁-C₆)heteroalkyl, substituted or unsubstituted aryl, and substituted or unsubstituted heteroaryl;
R₃ is selected from the group consisting of hydrogen, halogen, (C₁-C₃) alkyl, fluorinated (C₁-C₃) alkyl, cyano, C₁-C₃ alkoxy, SMe, and N(CH₃)₂;
n is selected from the group consisting of 0, 1, 2, and 3; and
m is selected from the group consisting of 0, 1, and 2.

6. The method of claim 5 wherein HO-Cy₁ is selected from the group consisting of

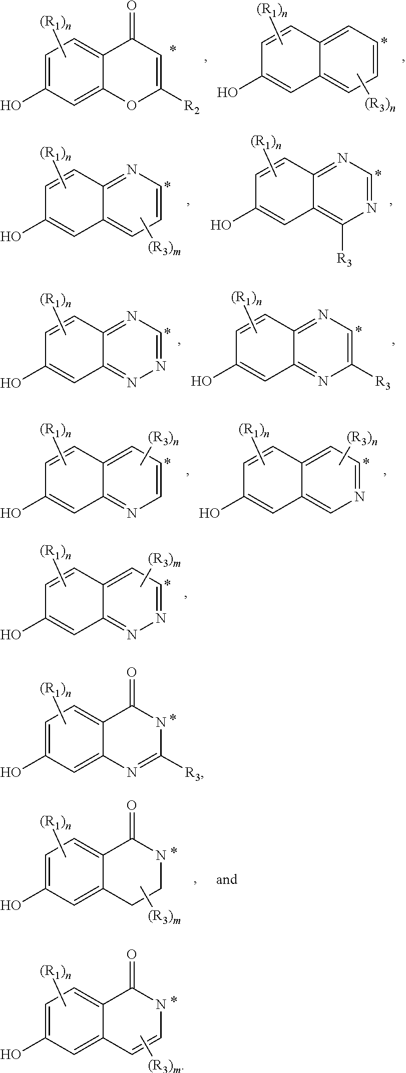

7. The method of claim 6 wherein Cy₂-Acidic moiety is selected from the group consisting of 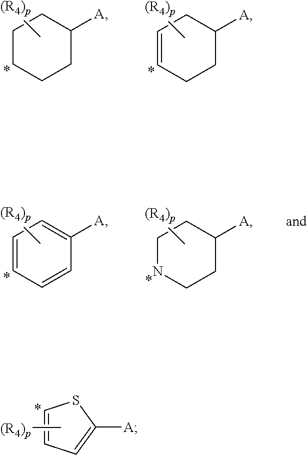
wherein
* represents the position on Cy₂ that is connected to the linker;
A is the acidic moiety, and is selected from the group consisting of 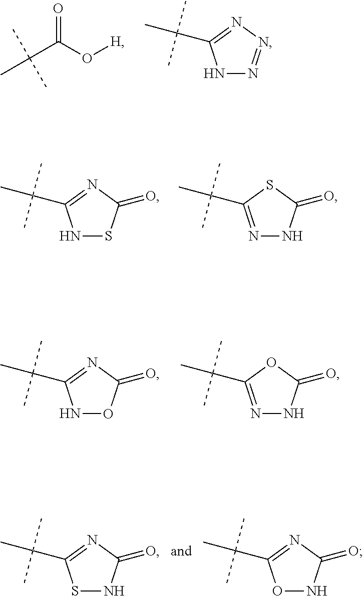
R₄ is selected from the group consisting of halogen, (C₁-C₆) alkyl, (C₁-C₆) haloalkyl, (C₁-C₆) alkoxy, cyano, and NR₈R_{8′} where R₈ and R_{8′} are independently selected from the group consisting of (C₁-C₃) alkyl, or R₈ when taken together with R_{8′} form a ring with 3 to 6 members;
and p is selected from the group consisting of 0, 1, 2, 3, and 4.

8. The method of claim 1 wherein
linker is selected from the group consisting of substituted and unsubstituted (C₂-C₃) alkyl, substituted and unsubstituted (C₂-C₃) heteroalkyl, and substituted and unsubstituted (C₂-C₃) alkene, wherein substitutions for (C₂-C₃) alkyl, (C₂-C₃) heteroalkyl, and (C₂-C₃) alkene are selected from the group consisting of ═O, halogen, (C₁-C₃) alkyl, (C₁-C₃) haloalkyl, (C₁-C₃) heteroalkyl, cyano, and hydroxyl, and wherein when the heteroalkyl group contains nitrogen or sulfur, the N and S atoms may be optionally oxidized; and
Cy₁ is selected from the group consisting of substituted and unsubstituted monocyclic aryl, substituted and unsubstituted monocyclic heterocycle, substituted and unsubstituted monocyclic heteroaryl, and substituted and unsubstituted monocyclic cycloalkyl.

9. The method of claim 8 wherein HO-Cy₁ is 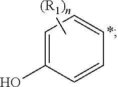
wherein R₁ is selected from the group consisting of halogen, methoxy, and cyano;
n is selected from the group consisting of 0, 1, 2, and 3;
Cy₂-Acidic moiety is selected from the group consisting of 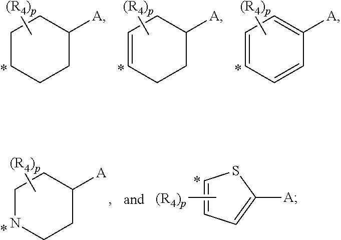
* represents the positions on Cy₁ and Cy₂ that are connected to the linker;
A is the acidic moiety, and is selected from the group consisting of 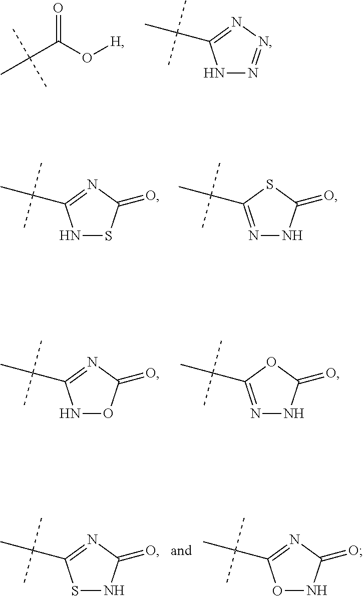
R₄ is selected from the group consisting of halogen, (C₁-C₆) alkyl, (C₁-C₆) haloalkyl, (C₁-C₆) alkoxy, cyano, and NR₈R_{8′} where R₈ and R_{8′} are independently selected from the group consisting of (C₁-C₃) alkyl, or R₈ when taken together with R_{8′} form a ring with 3 to 6 members;
and p is selected from the group consisting of 0, 1, 2, 3, and 4.

10. The method of claim 9 wherein linker is selected from the group consisting of substituted and unsubstituted C₂ alkyl, and substituted and unsubstituted C₂ heteroalkyl; wherein heteroalkyl consists of one heteroatom selected from the group consisting of O, S, SO, SO₂, NH, NMe, and wherein substitutions for C₂ alkyl and C₂ heteroalkyl are selected from the group consisting of ═O, R₅ and R₆.

11. The method of claim 10 wherein the GSNOR inhibitor has the structure shown in Formula 2 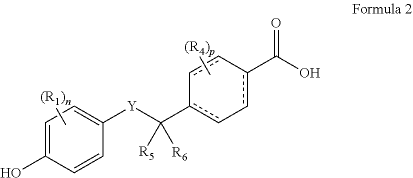
wherein Y is selected from the group consisting of CH₂, O, S, SO, SO₂, and NR₇; and
--- indicates the bond can be saturated or unsaturated.

12. The method of claim 10 wherein the GSNOR inhibitor has the structure shown in Formula 3 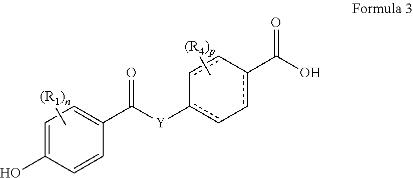
wherein Y is selected from the group consisting of CH₂ and NH
--- indicates the bond can be saturated or unsaturated.

13. A method of claim 1, wherein
linker is selected from the group consisting of substituted and unsubstituted 5 or 6 membered aryl, substituted and unsubstituted 5 or 6 membered heteroaryl; substitutions for aryl and heteroaryl are selected from the group consisting of hydrogen, (C₁-C₈)alkyl, (C₁-C₈) haloalkyl, and (C₁-C₈)heteroalkyl; and
Cy₁ is selected from the group consisting of a substituted or unsubstituted monocyclic aryl group, and a substituted or unsubstituted monocyclic heteroaryl group.

14. A method of claim 13 wherein the GSNOR inhibitor has the structure shown in Formula 4 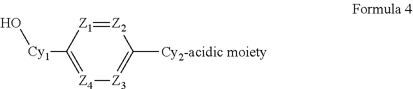
wherein
Z₁, Z₂, Z₃, and Z₄ are independently selected from the group consisting of CH and N;
Cy₂-Acidic moiety is selected from the group consisting of 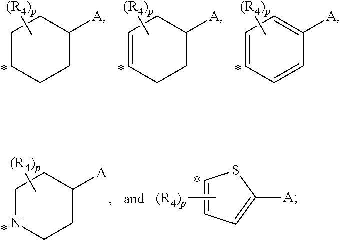
wherein
* represents the positions on Cy₁ and Cy₂ that are connected to the linker;
A is the acidic moiety, and is selected from the group consisting of 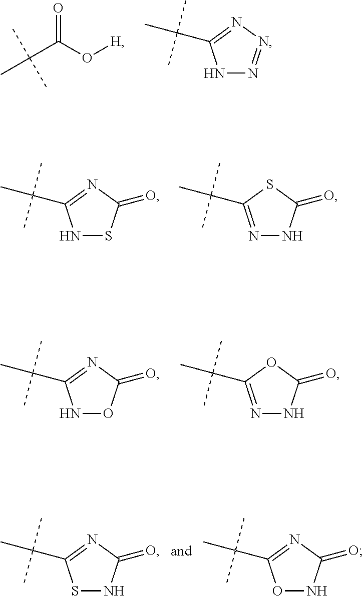
R₄ is selected from the group consisting of halogen, (C₁-C₆) alkyl, (C₁-C₆) haloalkyl, (C₁-C₆) alkoxy, cyano, and NR₈R_{8′} where R₈ and R_{8′} are independently selected from the group consisting of (C₁-C₃) alkyl, or R₈ when taken together with R_{8′} form a ring with 3 to 6 members;
and p is selected from the group consisting of 0, 1, 2, 3, and 4.

15. A method of claim 14 wherein
HO-Cy₁ is 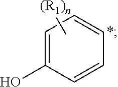
wherein R₁ is selected from the group consisting of halogen, methoxy, and cyano;
n is selected from the group consisting of 0, 1, 2, and 3.

16. The method of claim 15 wherein acidic moiety is a carboxylic acid.

17. A method of claim 13, wherein the GSNOR inhibitor has the structure shown in Formula 5 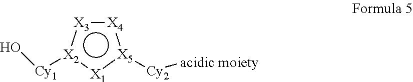
wherein
X₁, X₃, and X₄ are independently selected from the group consisting of N, NR₉, CR₁₀, S, and O;
X₂ and X₅ are independently selected from the group consisting of C, CH, and N;
R₉ and R₁₀ are independently selected from the group consisting of hydrogen, (C₁-C₈)alkyl, (C₁-C₈) haloalkyl, and (C₁-C₈)heteroalkyl;
HO-Cy₁ is 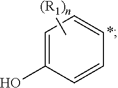
wherein R₁ is selected from the group consisting of halogen, methoxy, and cyano;
n is selected from the group consisting of 0, 1, 2, and 3;
Cy₂-Acidic moiety is selected from the group consisting of 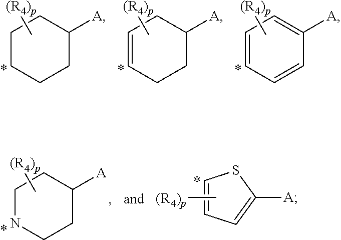
wherein
* represents the positions on Cy₁ and Cy₂ that are connected to the linker;
A is the acidic moiety, and is selected from the group consisting of 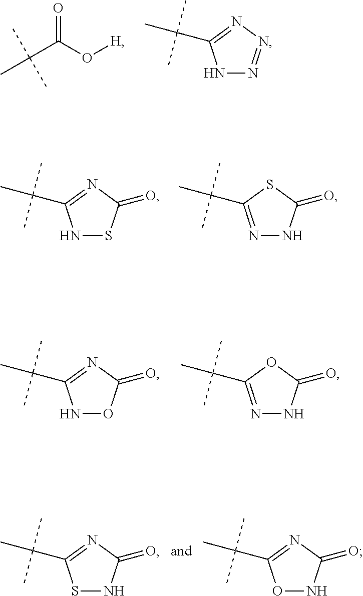
R₄ is selected from the group consisting of halogen, (C₁-C₆) alkyl, (C₁-C₆) haloalkyl, (C₁-C₆) alkoxy, cyano, and NR₈R_{8′} where R₈ and R_{8′} are independently selected from the group consisting of (C₁-C₃) alkyl, or R₈ when taken together with R_{8′} form a ring with 3 to 6 members;
and p is selected from the group consisting of 0, 1, 2, 3, and 4.

18. The method of claim 17 wherein the GSNOR inhibitor has the structure shown in Formula 6 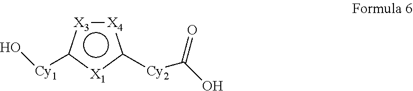
wherein
X₁, X₃, and X₄ are independently selected from the group consisting of N, NR₉, CR₁₀, S, and O.

19. The method of claim 18 wherein the GSNOR inhibitor has the structure shown in Formula 7 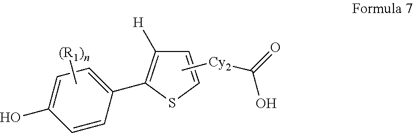
wherein
R₁ is selected from the group consisting of F, Cl, Br, OMe, and CN;
n is selected from the group consisting of 0 and 1;
Cy₂-COOH is selected from the group consisting of 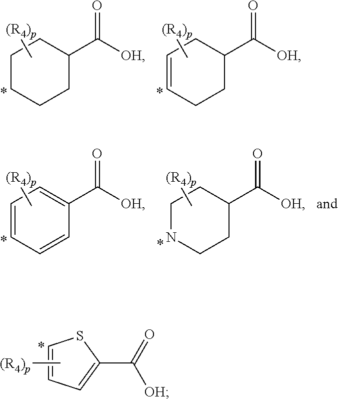
R₄ is selected from the group consisting of F, Cl, Br, CN, Me, OMe, N(Me)₂;
p is selected from the group consisting of 0, 1, and 2, and
* represents the position on Cy₂ that is connected to the compound of Formula 7.

20. The method of claim 18 wherein the GSNOR inhibitor has the structure shown in Formula 8 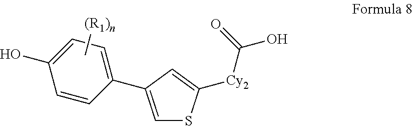
wherein
R₁ is selected from the group consisting of F, Cl, Br, OMe, and CN;
n is selected from the group consisting of 0 and 1;
Cy₂-COOH is selected from the group consisting of 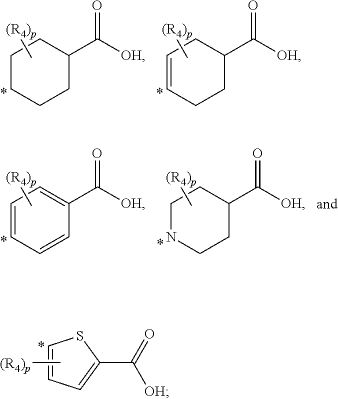
R₄ is selected from the group consisting of F, Cl, Br, CN, Me, OMe, N(Me)₂;
p is selected from the group consisting of 0, 1, and 2, and
* represents the position on Cy₂ that is connected to the compound of Formula 8.

21. The method of claim 18 wherein the GSNOR inhibitor has the structure shown in Formula 9 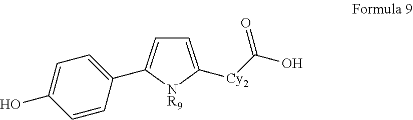
wherein
R₁ is selected from the group consisting of F, Cl, Br, OMe, and CN;
n is selected from the group consisting of 0 and 1;
Cy₂-COOH is selected from the group consisting of 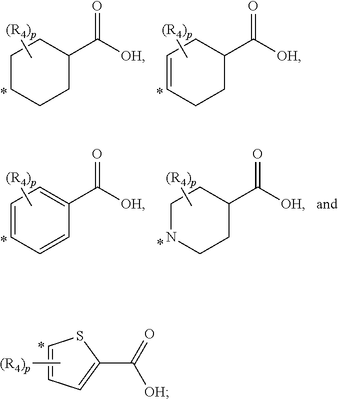
R₄ is selected from the group consisting of F, Cl, Br, CN, Me, OMe, N(Me)₂;
p is selected from the group consisting of 0, 1, and 2;
* represents the position on Cy₂ that is connected to the compound of formula 9; and
R₉ is selected from the group consisting of hydrogen, (C₁-C₈)alkyl, (C₁-C₈) haloalkyl, and (C₁-C₈) heteroalkyl.

22. The method of claim 18 wherein the GSNOR inhibitor has the structure shown in Formula 10 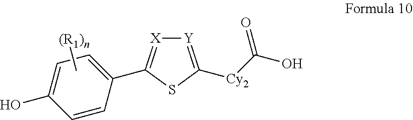
wherein
X is selected from CH and N,
Y is selected from CH and N, and wherein when X is CH, then Y is N, and when X is N, then Y is CH;
R₁ is selected from the group consisting of F, Cl, Br, OMe, and CN;
n is selected from the group consisting of 0 and 1;
Cy₂-COOH is selected from the group consisting of 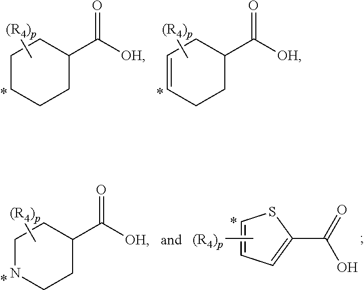
R₄ is selected from the group consisting of F, Cl, Br, CN, Me, OMe, N(Me)₂;
p is selected from the group consisting of 0, 1, and 2;
* represents the position on Cy₂ that is connected to the compound of formula 10.

23. The method of claim 18 wherein the GSNOR inhibitor has the structure shown in Formula 11 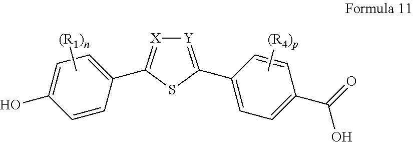
wherein
X is selected from CH and N,
Y is selected from CH and N, and wherein when X is CH, Y is N, and when X is N, then Y is CH;
R₁ is selected from the group consisting of F, Cl, Br, OMe, and CN;
n is selected from the group consisting of 0 and 1;
R₄ is selected from the group consisting of F, Cl, Br, CN, Me, OMe, N(Me)₂;
p is selected from the group consisting of 0, 1, and 2; and
n+p is greater than 0.

24. The method of claim 18 wherein the GSNOR inhibitor has the structure shown in Formula 12 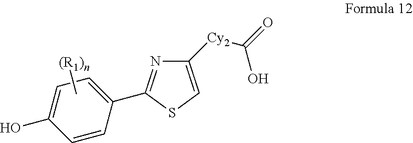
wherein
R₁ is selected from the group consisting of F, Cl, Br, OMe, and CN;
n is selected from the group consisting of 0 and 1;
Cy₂-COOH is selected from the group consisting of 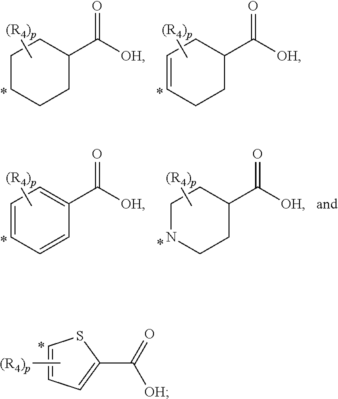
R₄ is selected from the group consisting of F, Cl, Br, CN, Me, OMe, N(Me)₂;
p is selected from the group consisting of 0, 1, and 2;
* represents the position on Cy₂ that is connected to the compound of formula 12.

25. The method of claim 18 wherein the GSNOR inhibitor has the structure shown in Formula 13 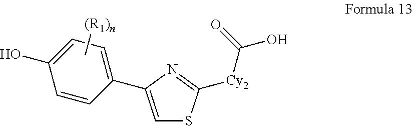
wherein
R₁ is selected from the group consisting of F, Cl, Br, OMe, and CN;
n is selected from the group consisting of 0 and 1;
Cy₂-COOH is selected from the group consisting of 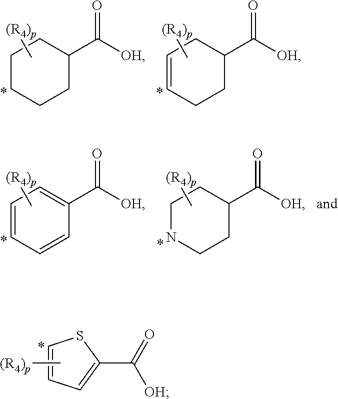
R₄ is selected from the group consisting of F, Cl, Br, CN, Me, OMe, N(Me)₂;
p is selected from the group consisting of 0, 1, and 2;
* represents the position on Cy₂ that is connected to the compound of formula 13.

26. The method of claim 18 wherein the GSNOR inhibitor has the structure shown in Formula 14 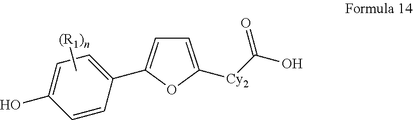
wherein
R₁ is selected from the group consisting of F, Cl, Br, OMe, and CN;
n is selected from the group consisting of 0 and 1;
Cy₂-COOH is selected from the group consisting of 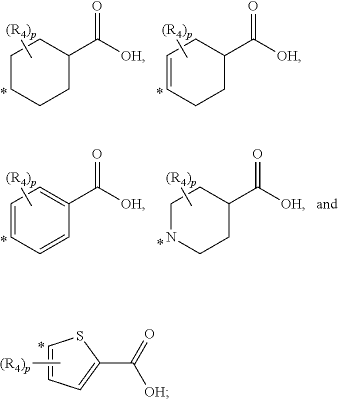
R₄ is selected from the group consisting of F, Cl, Br, CN, Me, OMe, N(Me)₂;
p is selected from the group consisting of 0, 1, and 2;
* represents the position on Cy₂ that is connected to the compound of formula 14.

27. The method of claim 1 wherein the patient has one or more of the following diseases, disorders, or conditions: pulmonary disorders associated with hypoxemia and/or smooth muscle constriction in the lungs and airways and/or lung infection and/or lung inflammation and/or lung injury; cardiovascular disease and heart disease; diseases characterized by angiogenesis; disorders where there is risk of thrombosis occurring; disorders where there is risk of restenosis occurring; inflammatory diseases; functional bowel disorders; diseases where there is risk of apoptosis occurring; impotence; sleep apnea; diabetic wound healing; cutaneous infections; treatment of psoriasis; obesity caused by eating in response to craving for food; stroke; reperfusion injury; and disorders where preconditioning of heart or brain for NO protection against subsequent ischemic events is beneficial, central nervous system (CNS) disorders; and infections caused by bacteria.

28. The method of claim 27 wherein the disease or condition is selected from one or more of the following, pulmonary hypertension, ARDS, asthma, pneumonia, pulmonary fibrosis/interstitial lung diseases, cystic fibrosis, COPD, hypertension, ischemic coronary syndromes, atherosclerosis, heart failure, glaucoma, coronary artery disease, AIDS related dementia, inflammatory bowel disease (IBD), Crohn's disease, colitis, and psoriasis, irritable bowel syndrome (IBS), heart failure, atherosclerosis, degenerative neurologic disorders, arthritis, and liver injury (drug induced, ischemic, alcoholic), traumatic muscle injury in heart or lung or crush injury, anxiety, depression, psychosis, and schizophrenia, tuberculosis, and C. difficile infections.
